(19) United States
(12) Patent Application Publication
(10) Pub. No.: US 2017/0008888 A1
(43) Pub. Date: Jan. 12, 2017

(54) HETEROCYCLIC COMPOUNDS

(71) Applicant: KYORIN PHARMACEUTICAL CO., LTD., Tokyo (JP)

(72) Inventors: Yi Hu, San Diego, CA (US); Emme C. K. Lin, San Diego, CA (US); Bei Li, San Diego, CA (US); Shyama Sidique, San Diego, CA (US); Jonathan S. Rosenblum, San Diego, CA (US); Oana M. Cociorva, San Diego, CA (US); Kevin R. Shreder, Del Mar, CA (US); Shigeki Seto, Tochigi (JP)

(21) Appl. No.: 15/121,282

(22) PCT Filed: Feb. 26, 2015
(86) PCT No.: PCT/JP2015/056757

§ 371 Date: Aug. 24, 2016

Related U.S. Application Data
(60) Provisional application No. 62/052,964, filed on Sep. 19, 2014.
(60) Provisional application No. 61/945,043, filed on Feb. 26, 2014.

(51) Int. Cl. C07D 471/04; A61K 45/06; A61K 31/7068; C07D 491/147; C07D 498/04; C07D 471/14; C07D 513/14; C07D 513/04; A61K 31/554; C07D 519/00; A61K 31/5513; A61K 31/551

(52) U.S. Cl. CPC C07D 471/04; A61K 31/5513; A61K 45/06; A61K 31/7068; C07D 491/147; A61K 31/551; C07D 471/14; C07D 513/14; C07D 513/04; A61K 31/554; C07D 519/00; C07D 498/04

(57) ABSTRACT

Provided herein are compounds of formula I and compositions containing the compounds. The compounds and compositions are useful in the methods of inhibiting the action of ERK5, a BET family protein or both. In certain embodiments, the compounds and compositions are useful in the prevention, amelioration or treatment of a ERK5-mediated disease, a BET protein-mediated disease or both.

### RELATED APPLICATIONS

This application claims benefit of U.S. Provisional Patent Application No. 62/052,964 filed on Sep. 19, 2014, and 61/945,043 filed Feb. 26, 2014, contents of both of which are hereby incorporated by reference herein in their entireties.

### FIELD

Compounds, compositions and methods for treating, preventing or ameliorating extracellular-signal-regulated kinase 5 (ERK5) mediated diseases are provided. Also provided are methods to treat diseases that are sensitive to a compound that binds to one or more bromodomains of BET family proteins, including BRD2, BRD3, BRD4 and BRDT.

### BACKGROUND

Extracellular signal-regulated kinase 5 (ERK5), also known as big mitogen-activated protein kinase (MAPK) 1, is a member of the MAPK family. ERK5 is activated in response to cell stress and growth factors through its selective phosphorylation by mitogen-activated protein kinase kinase 5 (MEK5).

ERK5 participates in several processes including proliferation, angiogenesis, neuronal differentiation and survival, and vasculature maintenance. ERK5 is known to mediate the effects of different oncogenes, and its signaling has been found altered in several human tumors. In particular, the role of ERK5 in angiogenesis and endothelial function has been clearly demonstrated in several experimental systems. It has been reported that stimulation of ERK5 can be employed to prevent and treat endothelial dysfunction related to oxidative stress and inflammation. It has also been suggested that ERK5 plays a role in diabetes mellitus, skeletal muscle disease, allergic asthma, psoriasis, rheumatoid arthritis, Alzheimer's disease and inflammatory pain peripheral neuropathies. See, Katsura et al., Journal of Neurochemistry, 2007, 102, 1614-1624; Xiao et al., Brain research, 2008, Vol. 1215, pages 76-86; Woo et al., J. Biol. Chem. 2006, 281:32164-32174; Drew et al., Biochimica et Biophysica Acta 1825 (2012) 37-48; and WO 94/21781.

The diseases in which ERK5 may participate include, but are not limited to inflammatory diseases, including inflammatory diseases in the airways, such as nonspecific bronchial hyper-reactivity, chronic bronchitis, cystic fibrosis, acute respiratory distress syndrome (ARDS), asthma and idiopathic lung fibrosis or idiopathic pulmonary fibrosis (IPF), pulmonary fibrosis, interstitial lung disease, psoriasis, chronic plaque psoriasis, psoriatic arthritis, acanthosis, atopic dermatitis, various forms of eczema, contact dermatitis (includes allergic dermatitis), systemic sclerosis (scleroderma), wound healing, atopic dermatitis (allergen-specific) and drug eruption, arthritis, osteoarthritis, pain and oncological disorders.

Further diseases in which ERK5, may participate are, for example, Alzheimer's disease (AD), mild cognitive impairment (MCI), age-associated memory impairment (AAMI), multiple sclerosis, Parkinson's disease, vascular dementia, senile dementia, AIDS dementia, Pick's disease, dementia caused by cerebrovascular disorders, corticobasal degeneration, amyotrophic lateral sclerosis (ALS), Huntington's disease and diminished CNS function associated with traumatic brain injury and others.

Therefore, there is a need for effective ERK5 inhibitors as therapeutics for treatment of various diseases.

The human BET family (bromodomain and extra C-terminal domain family) has four members (BRD2, BRD3, BRD4 and BRDT), which contain two related bromodomains and one extraterminal domain (Wu and Chiang, J. Biol. Chem., 2007, 282:13141-13145). The bromodomains are protein regions that recognize acetylated lysine residues. These acetylated lysines are often found at the N-terminal end of histones (e.g. histone 3 or histone 4) and are characteristic features of an open chromatin structure and active gene transcription (Kuo and Allis, Bioessays, 1998, 20:615-626). In addition, bromodomains can recognize other acetylated proteins. For example, BRD4 binds to RelA, which leads to stimulation of NF-κB and transcriptional activity of inflammatory genes (Huang et al., Mol. Cell. Biol., 2009, 29:1375-1387). The extraterminal domain of BRD2, BRD3 and BRD4 interacts with several proteins having a role in chromatin modulation and regulation of gene expression (Rahman et al., Mol. Cell. Biol., 2011, 31:2641-2652). BRD4 is essential for transcription elongation and recruits the elongation complex P-TEFb, which consists of CDK9 and cyclin T1, which leads to activation of RNA polymerase II (Yang et al., Mol. Cell, 2005, 19:535-545). Consequently there is stimulation of the expression of genes that are involved in cell proliferation, such as c-Myc and Aurora B for example (You et al., Mol. Cell. Biol., 2009, 29:5094-5103; Zuber et al., Nature, 2011, doi: 10.1038).

BET proteins play an important role in various types of tumours. See for example, French, Cancer Genet. Cytogenet., 2010, 203:16-20, Yan et al., J. Biol. Chem., 2011, 286:27663-27675, Filippakopoulos et al., Nature, 2010, 468:1067-1073, Zuber et al., Nature, 2011, doi:10.1038, Greenwall et al., Blood, 2005, 103:1475-1484. BET proteins are also involved in viral infections. See for example, Wu et al., Genes Dev., 2006, 20: 2383-2396, Viejo-Borbolla et al., J. Virol., 2005, 79:13618-13629; You et al., J. Virol., 2006, 80:8909-8919, and Bisgrove et al., Proc. Natl. Acad. Sci. USA, 2007, 104:13690-13695.

BET proteins are in addition involved in inflammatory processes. See for example, Wang et al., Biochem. J., 2009, 425:71-83 and Nicodeme et al., Nature, 2010, 468:1119-1123).

BRDT and possibly the other BET genes are required for proper spermatogenesis. See Berkovits et al. Curr Top Dev Biol. 2013; 102: 293-326. Therefore, inhibitors of the BET family of proteins could be useful as reversible male contraceptives.

Since BET proteins play an essential role in various pathologies, it is therefore important to find compounds that are inhibitors of one or more BET proteins, including BRD2, BRD3, BRD4 and BRDT.

### SUMMARY

In certain embodiments, provided herein are compounds that are ERK5 inhibitors, pharmaceutical compositions containing the compounds and methods of use thereof. In certain embodiments, provided herein are compounds that are inhibitors of one or more BET proteins, including BRD2, BRD3, BRD4 and BRDT, pharmaceutical compositions containing the compounds and methods of use thereof. In certain embodiments, the compounds provided herein have activity as inhibitors of ERK5 and one or more BET proteins, including BRD2, BRD3, BRD4 and BRDT.

In certain embodiments, the compounds for use in the compositions and methods provided herein are of Formula I:

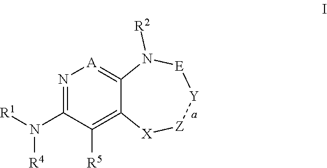

or pharmaceutically acceptable salts thereof, wherein the variables are chosen such that the resulting compounds show activity as ERK5 inhibitors and/or inhibitors of one or more BET proteins, including BRD2, BRD3, BRD4 and BRDT. In certain embodiments, the compounds for use in the compositions and methods provided herein are of Formula I or pharmaceutically acceptable salts thereof, wherein the variables are chosen such that the resulting compounds show activity as inhibitors of ERK5. In certain embodiments, the compounds of formula I show activity as inhibitors of one or more BET proteins, including BRD2, BRD3, BRD4 and BRDT.

Pharmaceutical compositions containing a compound of Formula I and a pharmaceutically acceptable carrier are provided herein. In certain embodiments, provided are methods for treating, preventing, or ameliorating one or more symptoms of ERK5-mediated diseases and/or diseases mediated by one or more BET proteins, including BRD2, BRD3, BRD4 and BRDT, by administering the compounds and compositions provided herein.

In certain embodiments, provided herein are methods for inhibiting an action of ERK5 by administering compounds and compositions provided herein. In other embodiments, provided herein are methods for treatment, prevention, or amelioration of one or more symptoms of diseases or conditions associated with ERK5 by administering compounds and compositions provided herein.

In certain embodiments, provided herein are methods for inhibiting an action of one or more BET protein, including BRD2, BRD3, BRD4 and BRDT, by administering compounds and compositions provided herein. In other embodiments, provided herein are methods for treatment, prevention, or amelioration of one or more symptoms of diseases or conditions associated with interaction of one or more BET family of proteins, including BRD2, BRD3, BRD4 and BRDT, and acetylated proteins by administering compounds and compositions provided herein.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

As used herein “subject” is an animal, such as a mammal, including human, as a patient.

The term “ERK5-mediated disease”, as used herein, means any disease or other deleterious condition or state in which ERK5 is known to play a role. Exemplary diseases or conditions include, without limitation, inflammatory diseases in the airways, such as nonspecific bronchial hyper-reactivity, chronic bronchitis, cystic fibrosis, acute respiratory distress syndrome (ARDS), asthma and idiopathic lung fibrosis or idiopathic pulmonary fibrosis (IPF), pulmonary fibrosis, interstitial lung disease, psoriasis, chronic plaque psoriasis, psoriatic arthritis, acanthosis, atopic dermatitis, various forms of eczema, contact dermatitis (includes allergic dermatitis), systemic sclerosis (scleroderma), wound healing, atopic dermatitis (allergen-specific) and drug eruption, rheumatoid arthritis, ankylosing spondylitis, Crohn's disease, erythematosus, lupus, osteoarthritis, and oncological disorders.

As used herein, the term “bromodomain inhibitor” denotes a compound which inhibits the binding of a bromodomain with its cognate acetylated proteins. In one embodiment, the bromodomain inhibitor is a compound which inhibits the binding of a bromodomain to acetylated lysine residues. In a further embodiment, the bromodomain inhibitor is a compound which inhibits the binding of a bromodomain to acetylated lysine residues on histones, particularly histones H3 and H4. In one embodiment, the bromodomain inhibitor is a compound that inhibits the binding of BET family bromodomains to acetylated lysine residues (hereafter referred to as a “BET family bromodomain inhibitor”). In one embodiment, the BET family bromodomain is BRD2, BRD3, BRD4 or BRDT.

The term “BET family mediated disease”, as used herein, means any disease or other deleterious condition or state in which one or more BET family proteins, including BRD2, BRD3, BRD4 and/or BRDT, are known to play a role. Exemplary diseases or conditions include, without limitation, hyper-proliferative diseases, for example, psoriasis, keloid and other hyperplasias that affect the skin, benign prostatic hyperplasias (BPH), solid tumours and haematological tumours, inflammatory or autoimmune diseases, viral diseases, neurodegenerative diseases, atherosclerosis, dyslipidaemia, hypercholesterolaemia, hypertriglyceridaemia, peripheral vascular diseases, cardiovascular diseases, angina pectoris, ischaemia, stroke, myocardial infarction, angioplastic restenosis, high blood pressure, thrombosis, adiposity, and endotoxaemia.

As used herein, biological activity refers to the in vivo activities of a compound or physiological responses that result upon in vivo administration of a compound, composition or other mixture. Biological activity, thus, encompasses therapeutic effects and pharmacokinetic behaviour of such compounds, compositions and mixtures. Biological activities can be observed in in vitro systems designed to test for such activities.

As used herein, pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamineand other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates.

As used herein, treatment means any manner in which one or more of the symptoms of a disease or disorder are ameliorated or otherwise beneficially altered. Treatment also encompasses any pharmaceutical use of the compositions herein.

As used herein, amelioration of the symptoms of a particular disorder by administration of a particular compound or pharmaceutical composition refers to any lessening, whether permanent or temporary, lasting or transient that can be attributed to or associated with administration of the compound or composition.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, the IC₅₀ refers to an amount, concentration or dosage of a particular test compound that achieves a 50% inhibition of a maximal response in an assay that measures such response.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. As such, one of skill in the art will recognize that administration of a compound in its (R) form is equivalent, for compounds that undergo epimerization in vivo, to administration of the compound in its (S) form.

As used herein, the nomenclature alkyl, alkoxy, carbonyl, etc. is used as is generally understood by those of skill in this art.

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, ethene, propene, butene, pentene, acetylene and hexyne. As used herein, lower alkyl, lower alkenyl, and lower alkynyl refer to carbon chains having from about 1 or about 2 carbons up to about 6 carbons.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion.

As used herein, “substituted alkyl,” “substituted alkenyl,” “substituted alkynyl,” “substituted cycloalkyl,” “substituted cycloalkenyl,” and “substituted cycloalkynyl” refer to alkyl, alkenyl, alkynyl, cycloalkyl, cycloalkenyl and cycloalkynyl groups, respectively, that are substituted with one or more substituents, in certain embodiments one to three or four substituents, where the substituents are as defined herein.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl, wherein the substituents, when present, are one or more substituents as defined herein.

As used herein, “heteroaryl” refers to a monocyclic or multicyclic aromatic ring system, in certain embodiments, of about 5 to about 15 members where one or more, in one embodiment, 1 to 3 of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3 of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three or four substituents, where the substituents are as defined herein.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, “haloalkyl” refers to an alkyl group in which one or more of the hydrogen atoms are replaced by halogen. Such groups include, but are not limited to, chloromethyl, trifluoromethyl and 1 chloro 2 fluoroethyl.

As used herein, “carboxy” refers to a divalent radical, —C(O)O—.

As used herein, “alkoxy” refers to RO, in which R is alkyl, including lower alkyl.

As used herein, “aryloxy” refers to RO—, in which R is aryl, including lower aryl, such as phenyl.

As used herein, “amine” or “amino” refers to a group having the formula —NR′R″ wherein R′ and R″ are each independently hydrogen, alkyl, haloalkyl, hydroxyalkyl or alkoxyalkyl or wherein R′ and R″, together with the nitrogen atom to which they are attached form a heterocyclyl optionally substituted with halo, oxo, hydroxy or alkoxy.

As used herein, “aminoalkyl” refers to an alkyl group in which one or more of the hydrogen atoms are replaced by amino. Such groups include, but are not limited to, —CH₂NH₂, —CH(NH₂)₂, —CH₂NH(CH₃) and —CH₂N(CH₃)₂.

As used herein, “deutero” or “deuterium” refers to the hydrogen isotope deuterium having the chemical symbol D.

Where the number of any given substituent is not specified (e.g., “haloalkyl”), there may be one or more substituents present. For example, “haloalkyl” may include one or more of the same or different halogens.

As another example, “C₁₋₃alkoxyphenyl” may include one or more of the same or different alkoxy groups containing one, two or three carbons.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

Compounds

In certain embodiments, the compounds for use in the compositions and methods provided herein are of formula I:

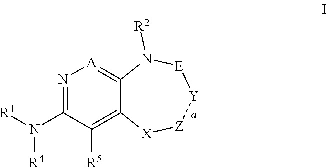

or a pharmaceutically acceptable salt thereof, wherein

bond a is a single bond or double bond;

R¹ and R⁴ are each independently selected from hydrogen, alkyl, alkenyl, alkynyl, aryl, heteroaryl, heterocyclyl and cycloalkyl;

R² is alkyl, deuteroalkyl, alkenyl, alkynyl, haloalkyl or cycloalkyl;

X is NR³, O, S(O)ₘ, or CR^{a}R^{b};

R^{a} and R^{b} are selected as follows:

(i) R^{a} and R^{b} are each independently selected from hydrogen, alkyl, alkenyl, alkynyl, haloalkyl, cycloalkyl, aryl, heterocyclyl and heteroaryl; or

(ii) R^{a} and R^{b} together form ═O;

R³ is alkyl, deuteroalkyl, alkenyl, alkynyl, haloalkyl, cycloalkyl, SO₂R¹⁹, COR², or —SO₂N(R¹⁴)(R¹⁵);

R⁵ is selected from hydrogen, alkyl, alkenyl, alkynyl and cycloalkyl;

A is CH, CR², or N;

E is CO, SO₂, CN(OR¹⁸), CN(CN), CS, CNR¹¹, or CR¹²CF₃;

Y is CR⁷ or CR⁷R⁸;

Z is CR⁹ or CR⁹R¹⁰;

R⁷ and R⁹ together with the atoms on which they are substituted form an optionally substituted 3 to 6-membered cycloalkyl, aryl, heterocyclyl or heteroaryl ring, where substituents, when present are selected from one or more Q¹ and Q³ groups;

R⁸ and R¹⁰, when present, are each independently selected from hydrogen, alkyl and cycloalkyl;

Q¹ is selected from alkyl, cycloalkyl, aryl and heteroaryl;

R¹¹ and R¹² are each independently selected from hydrogen, alkyl and cycloalkyl;

R¹⁸ is hydrogen, alkyl or cycloalkyl;

R¹⁹ is alkyl, alkenyl, alkynyl, haloalkyl, cycloalkyl, aryl or heteroaryl;

Q¹, R^{a}, R^{b}, R², R³, R⁴, R⁵, R⁸, R¹⁰ and R¹⁹ are optionally substituted with 1, 2, 3 or 4 substituents, each independently selected from Q², where Q² is selected from deutero, alkyl, alkenyl, alkynyl, haloalkyl, cycloalkyl, hydroxyl and halo;

R¹ is optionally substituted with 1, 2, 3 or 4 substituents Q³, each Q³ is independently selected from halo, cyano, oxo, thioxo, alkyl, haloalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}OR^{u}N(R^{y})(R^{z}), —R^{u}N(R^{y})(R^{z}), —R^{u}C(J)R^{x}, —R^{u}C(J)OR^{x}, —R^{u}C(J)N(R^{y})(R^{z}), —R^{u}S(O)ₜN(R^{y})(R^{z}), —R^{u}S(O)ₜR^{w}, —R^{u}N(R^{x})C(J)R^{x}, —R^{u}N(R^{x})C(J)OR^{x}, —R^{u}N(R^{x})S(O)ₜR^{w}, and —C(═NR^{y})N(R^{y})OR^{x}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;

each R^{u} is independently alkylene, alkenylene or a direct bond;

R^{w} is alkyl, haloalkyl, hydroxyalkyl, alkoxyalkyl, amino, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

each R^{x} is independently hydrogen, alkyl, haloalkyl, hydroxyalkyl, alkoxyalkyl, cyanoalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

R^{y} and R^{z} are each independently selected from (i) or (ii) below:

(i) R^{y} and R^{z} are each independently hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a heterocyclyl or heteroaryl, optionally substituted with one or more, in one embodiment, one, two or three Q⁵ groups; each Q⁵ is independently selected from halo, oxo, thioxo, hydroxy, cyano, amino, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}SR^{x}, —R^{u}C(J)R^{x}, —R^{u}N(R¹⁴)(R¹⁵), —R^{u}C(J)OR^{x}, —OC(J)R^{u}N(R¹⁴)(R¹⁵), —R^{u}C(J)R^{u}N(R¹⁴)(R¹⁵), —R^{u}S(O)ₜR^{w}, —R^{u}N(R^{x})C(J)R^{x}, —R^{u}N(R^{x})C(J)OR^{x}, —OP(O)(OH)₂, and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O, NR^{x} or S;

each t is independently an integer from 0-2; and

m is 0-2.

In certain embodiments, the compounds for use in the compositions and methods provided herein are of formula I or a pharmaceutically acceptable salt thereof, wherein

bond a is a single bond or double bond;

R¹ and R⁴ are each independently selected from hydrogen, alkyl, alkenyl, alkynyl, aryl, heteroaryl, heterocyclyl and cycloalkyl;

R² is alkyl, alkenyl, alkynyl, haloalkyl or cycloalkyl;

X is NR³, O, S(O)ₘ, or CR^{a}R^{b};

R^{a} and R^{b} are selected as follows:

(i) R^{a} and R^{b} are each independently selected from hydrogen, alkyl, alkenyl, alkynyl, haloalkyl, cycloalkyl, aryl, heterocyclyl and heteroaryl; or

(ii) R^{a} and R^{b} together form ═O;

R³ is alkyl, alkenyl, alkynyl, haloalkyl, cycloalkyl, SO₂R¹⁹, COR², or —SO₂N(R¹⁴)(R¹⁵);

R⁵ is selected from hydrogen, alkyl, alkenyl, alkynyl and cycloalkyl;

A is CH, CR², or N;

E is CO, SO₂, CN(OR¹⁸), CN(CN), CS, CNR¹¹, or CR¹²CF₃;

Y is CR⁷ or CR⁷R⁸;

Z is CR⁹ or CR⁹R¹⁰;

R⁷ and R⁹ together with the atoms on which they are substituted form an optionally substituted 3 to 6-membered cycloalkyl, aryl, heterocyclyl or heteroaryl ring, where substituents, when present are selected from one or more Q¹ and Q³ groups;

R⁸ and R¹⁰, when present, are each independently selected from hydrogen, alkyl and cycloalkyl;

Q¹ is selected from alkyl, cycloalkyl, aryl and heteroaryl;

R¹¹ and R¹² are each independently selected from hydrogen, alkyl and cycloalkyl;

R¹⁸ is hydrogen, alkyl or cycloalkyl;

R¹⁹ is alkyl, alkenyl, alkynyl, haloalkyl, cycloalkyl, aryl or heteroaryl;

Q¹, R^{a}, R^{b}, R², R³, R⁴, R⁵, R⁸, R¹⁰ and R¹⁹ are optionally substituted with 1, 2, 3 or 4 substituents, each independently selected from Q², where Q² is selected from deutero, alkyl, alkenyl, alkynyl, haloalkyl, cycloalkyl, hydroxyl and halo;

R¹ is optionally substituted with 1, 2, 3 or 4 substituents Q³, each Q³ is independently selected from halo, cyano, oxo, thioxo, alkyl, haloalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}OR^{u}N(R^{y})(R^{z}), —R^{u}N(R^{y})(R^{z}), —R^{u}SR^{x}, —R^{u}C(J)R^{x}, —R^{u}C(J)OR^{x}, —R^{u}C(J)N(R^{y})(R^{z}), —R^{u}S(O)ₜN(R^{y})(R^{z}), —R^{u}S(O)ₜR^{w}, —R^{u}N(R^{x})C(J)R^{x}, —R^{u}N(R^{x})C(J)OR^{x}, —R^{u}N(R^{x})S(O)ₜR^{w}, and —C(═NR^{y})N(R^{y})OR^{x}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;

each R^{u} is independently alkylene, alkenylene or a direct bond;

R^{w} is alkyl, haloalkyl, hydroxyalkyl, alkoxyalkyl, amino, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

each R^{x} is independently hydrogen, alkyl, haloalkyl, hydroxyalkyl, alkoxyalkyl, cyanoalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

R^{y} and R^{z} are each independently selected from (i) or (ii) below:

(i) R^{y} and R^{z} are each independently hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a heterocyclyl or heteroaryl, optionally substituted with one or more, in one embodiment, one, two or three Q⁵ groups; each Q⁵ is independently selected from halo, oxo, thioxo, hydroxy, cyano, amino, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}SR^{x}, —R^{u}C(J)R^{x}, —R^{u}N(R¹⁴)(R¹⁵), —R^{u}C(J)OR^{x}, —OC(J)R^{u}N(R¹⁴)(R¹⁵), —R^{u}C(J)R^{u}N(R¹⁴)(R¹⁵), —R^{u}S(O)ₜR^{w}, —R^{u}N(R^{x})C(J)R^{x}, —R^{u}N(R^{x})C(J)OR^{x}, —OP(O)(OH)₂, and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O, NR^{x} or S;

each t is independently an integer from 0-2; and

m is 0-2.

In certain embodiments, X is NR³, and R³ is alkyl, deuteroalkyl, alkenyl, alkynyl, haloalkyl, cycloalkyl, SO₂R¹⁹, COR², or —SO₂N(R¹⁴)(R¹⁵).

In certain embodiments, X is O.

In certain embodiments, X is S(O)ₘ. In certain embodiments, X is S(O)₂. In certain embodiments, X is SO. In certain embodiments, X is S.

In certain embodiments, X is CR^{a}R^{b};

R^{a} and R^{b} are selected as follows:

(i) R^{a} and R^{b} are each independently selected from hydrogen, alkyl, alkenyl, alkynyl, haloalkyl and cycloalkyl; or

(ii) R^{a} and R^{b} together form ═O.

In certain embodiments, the compounds for use in the compositions and methods provided herein are of formula IA:

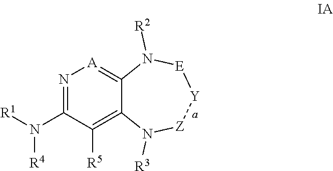

or a pharmaceutically acceptable salt thereof, wherein

bond a is a single bond or double bond;

R¹ and R⁴ are each independently selected from hydrogen, alkyl, alkenyl, alkynyl, aryl, heteroaryl, heterocyclyl and cycloalkyl;

R² is alkyl, deuteroalkyl, alkenyl, alkynyl, haloalkyl or cycloalkyl;

R³ is alkyl, deuteroalkyl, alkenyl, alkynyl, haloalkyl, cycloalkyl, SO₂R¹⁹, or COR²;

R⁵ is selected from hydrogen, alkyl, alkenyl, alkynyl and cycloalkyl;

A is CH, CR², or N;

E is CO, SO₂, CN(OR¹⁸), CN(CN), CS, CNR¹¹, or CR¹²CF₃;

Y is CR⁷ or CR⁷R⁸;

Z is CR⁹ or CR⁹R¹⁰;

R⁷ and R⁹ together with the atoms on which they are substituted form an optionally substituted 3 to 6-membered cycloalkyl, aryl, heterocyclyl or heteroaryl ring, where substituents, when present are selected from one or more Q¹ and Q³ groups;

R⁸ and R¹⁰, when present, are each independently selected from hydrogen, alkyl and cycloalkyl;

Q¹ is selected from alkyl, cycloalkyl, aryl and heteroaryl;

R¹¹ and R¹² are each independently selected from hydrogen, alkyl and cycloalkyl;

R¹⁸ is hydrogen, alkyl or cycloalkyl;

R¹⁹ is alkyl, alkenyl, alkynyl, haloalkyl, cycloalkyl, aryl or heteroaryl;

Q¹, R², R³, R⁴, R⁵, R⁸, R¹⁰ and R¹⁹ are optionally substituted with 1, 2, 3 or 4 substituents, each independently selected from Q², where Q² is selected from deutero, alkyl, alkenyl, alkynyl, haloalkyl, cycloalkyl, hydroxyl and halo;

R¹ is optionally substituted with 1, 2, 3 or 4 substituents Q³, each Q³ is independently selected from halo, cyano, oxo, thioxo, alkyl, haloalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}OR^{u}N(R^{y})(R^{z}), —R^{u}N(R^{y})(R^{z}), —R^{u}SR^{x}, —R^{u}C(J)R^{x}, —R^{u}C(J)OR^{x}, —R^{u}C(J)N(R^{y})(R^{z}), —R^{u}S(O)ₜN(R^{y})(R^{z}), —R^{u}S(O)ₜR^{w}, —R^{u}N(R^{x})C(J)R^{x}, —R^{u}N(R^{x})C(J)OR^{x}, —R^{u}N(R^{x})S(O)ₜR^{w}, and —C(═NR^{y})N(R^{y})OR^{x}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;

each R^{u} is independently alkylene, alkenylene or a direct bond;

R^{w} is alkyl, haloalkyl, hydroxyalkyl, alkoxyalkyl, amino, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

each R^{x} is independently hydrogen, alkyl, haloalkyl, hydroxyalkyl, alkoxyalkyl, cyanoalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

R^{y} and R^{z} are each independently selected from (i) or (ii) below:

(i) R^{y} and R^{z} are each independently hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a heterocyclyl or heteroaryl, optionally substituted with one or more, in one embodiment, one, two or three Q⁵ groups; each Q⁵ is independently selected from halo, oxo, thioxo, hydroxy, amino, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}SR^{x}, —R^{u}C(J)R^{x}, —R^{u}C(J)OR^{x}, R^{u}C(J)N(R¹⁴)(R¹⁵), —R^{u}S(O)ₜR^{w}, —R^{u}N(R^{x})C(J)R^{x}, —R^{u}N(R^{x})C(J)OR^{x}, and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O, NR^{x} or S; and

each t is independently an integer from 0-2.

In certain embodiments, the compounds for use in the compositions and methods provided herein are of formula IA or a pharmaceutically acceptable salt thereof, wherein

bond a is a single bond or double bond;

R¹ and R⁴ are each independently selected from hydrogen, alkyl, alkenyl, alkynyl, aryl, heteroaryl, heterocyclyl and cycloalkyl;

R² is alkyl, deuteroalkyl, alkenyl, alkynyl, haloalkyl or cycloalkyl;

R³ is alkyl, deuteroalkyl, alkenyl, alkynyl, haloalkyl, cycloalkyl, SO₂R¹⁹, COR², or —SO₂N(R¹⁴)(R¹⁵);

R⁵ is selected from hydrogen, alkyl, alkenyl, alkynyl and cycloalkyl;

A is CH, CR², or N;

E is CO, SO₂, CN(OH), CN(CN), CS, CNR¹¹, or CR¹²CF₃;

Y is CR⁷ or CR⁷R⁸;

Z is CR⁹ or CR⁹R¹⁰;

R⁷ and R⁹ together with the atoms on which they are substituted form an optionally substituted 3 to 6-membered cycloalkyl, aryl, heterocyclyl or heteroaryl ring, where substituents, when present are selected from one or more Q¹ and Q³ groups;

R⁸ and R¹⁰, when present, are each independently selected from hydrogen, alkyl and cycloalkyl;

Q¹ is selected from alkyl, cycloalkyl, aryl and heteroaryl;

R¹¹ and R¹² are each independently selected from hydrogen, alkyl and cycloalkyl;

Q¹, R², R³, R⁴, R⁵, R⁸, R¹⁰ and R¹⁹ are optionally substituted with 1, 2, 3 or 4 substituents, each independently selected from Q², where Q² is selected from deutero, alkyl, alkenyl, alkynyl, haloalkyl, cycloalkyl, hydroxyl and halo;

R¹ is optionally substituted with 1, 2, 3 or 4 substituents Q³, each Q³ is independently selected from halo, cyano, oxo, thioxo, alkyl, haloalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}OR^{u}N(R^{y})(R^{z}), —R^{u}N(R^{y})(R^{z}), —R^{u}SR^{x}, —R^{u}C(J)R^{x}, —R^{u}C(J)OR^{x}, —R^{u}C(J)N(R^{y})(R^{z}), —R^{u}S(O)ₜN(R^{y})(R^{z}), —R^{u}S(O)ₜR^{w}, —R^{u}N(R^{x})C(J)R^{x}, —R^{u}N(R^{x})C(J)OR^{x}, —R^{u}N(R^{x})S(O)ₜR^{w}, and —C(═NR^{y})N(R^{y})OR^{x}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;

each R^{u} is independently alkylene, alkenylene or a direct bond;

R^{w} is alkyl, haloalkyl, hydroxyalkyl, alkoxyalkyl, amino, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

each R^{x} is independently hydrogen, alkyl, haloalkyl, hydroxyalkyl, alkoxyalkyl, cyanoalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

R^{y} and R^{z} are each independently selected from (i) or (ii) below:

(i) R^{y} and R^{z} are each independently hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a heterocyclyl or heteroaryl, optionally substituted with one or more, in one embodiment, one, two or three Q⁵ groups; each Q⁵ is independently selected from halo, oxo, thioxo, hydroxy, cyano, amino, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}SR^{x}, —R^{u}C(J)R^{x}, —R^{u}N(R¹⁴)(R¹⁵), —R^{u}C(J)OR^{x}, —OC(J)R^{u}N(R¹⁴)(R¹⁵), —R^{u}C(J)R^{u}N(R¹⁴)(R¹⁵), —R^{u}S(O)ₜR^{w}, —R^{u}N(R^{x})C(J)R^{x}, —R^{u}N(R^{x})C(J)OR^{x}, —OP(O)(OH)₂, and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

R¹⁹ is alkyl, alkenyl, alkynyl, haloalkyl, cycloalkyl, aryl or heteroaryl;

J is O, NR^{x} or S; and

each t is independently an integer from 0-2.

In one embodiment, the compounds provided herein are of formula I, formula IA or a pharmaceutically acceptable salt thereof, wherein bond a is a single bond or double bond;

R¹ is hydrogen, alkyl, alkenyl, alkynyl, aryl, heteroaryl, heterocyclyl or cycloalkyl;

R² is alkyl, deuteroalkyl, alkenyl, alkynyl, haloalkyl or cycloalkyl;

R³ is alkyl, deuteroalkyl, alkenyl, alkynyl, haloalkyl, cycloalkyl, SO₂R¹⁹, COR², or —SO₂N(R¹⁴)(R¹⁵);

R⁴ is selected from hydrogen, alkyl, alkenyl, alkynyl and cycloalkyl;

R⁵ is selected from hydrogen, alkyl, alkenyl, alkynyl and cycloalkyl;

A is CH, CR², or N;

E is CO, SO₂, CN(OR¹⁸), CN(CN), CS, CNR¹¹, or CR¹²CF₃;

Y is CR⁷ or CR⁷R⁸;

Z is CR⁹ or CR⁹R¹⁰;

R⁷ and R⁹ together with the atoms on which they are substituted form an optionally substituted 3 to 6-membered cycloalkyl, aryl, heterocyclyl or heteroaryl ring, where substituents, when present are selected from one or more Q¹ and Q³ groups;

R⁸ and R¹⁰, when present, are each independently selected from hydrogen, alkyl and cycloalkyl;

Q¹ is selected from alkyl, cycloalkyl, aryl and heteroaryl;

R¹¹ and R¹² are each independently selected from hydrogen, alkyl and cycloalkyl;

R¹⁸ is hydrogen, alkyl or cycloalkyl;

R¹⁹ is alkyl, alkenyl, alkynyl, haloalkyl, cycloalkyl, aryl or heteroaryl;

Q¹, R², R³, R⁴, R⁵, R⁸, R¹⁰ and R¹⁹ are optionally substituted with 1, 2, 3 or 4 substituents, each independently selected from Q², where Q² is selected from deutero, alkyl, alkenyl, alkynyl, haloalkyl, cycloalkyl, hydroxyl and halo;

R¹ is optionally substituted with 1, 2, 3 or 4 substituents Q³, each Q³ is independently selected from halo, cyano, oxo, thioxo, alkyl, haloalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}OR^{u}N(R^{y})(R^{z}), —R^{u}N(R^{y})(R^{z}), —R^{u}SR^{x}, —R^{u}C(J)R^{x}, —R^{u}C(J)OR^{x}, —R^{u}C(J)N(R^{y})(R^{z}), —R^{u}S(O)ₜN(R^{y})(R^{z}), —R^{u}S(O)ₜR^{w}, —R^{u}N(R^{x})C(J)R^{x}, —R^{u}N(R^{x})C(J)OR^{x}, —R^{u}N(R^{x})S(O)ₜR^{w}, and —C(═NR^{y})N(R^{y})OR^{x}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;

each R^{u} is independently alkylene, alkenylene or a direct bond;

R^{w} is alkyl, haloalkyl, hydroxyalkyl, alkoxyalkyl, alkenyl, alkynyl, amino, cycloalkyl, cycloalkenyl, cycloalkylalkyl, cycloalkenylalkyl, heterocyclyl, heterocyclylalkyl, aryl, aralkyl, heteroaryl, or heteroaralkyl;

each R^{x} is independently hydrogen, alkyl, haloalkyl, hydroxyalkyl, alkoxyalkyl, cyanoalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

R^{y} and R^{z} are each independently selected from (i) or (ii) below:

(i) R^{y} and R^{z} are each independently hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a heterocyclyl or heteroaryl, optionally substituted with one or more, in one embodiment, one, two or three Q⁵ groups; each Q⁵ is independently selected from halo, oxo, thioxo, hydroxy, cyano, amino, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}SR^{x}, —R^{u}C(J)R^{x}, —R^{u}N(R¹⁴)(R¹⁵), —R^{u}C(J)OR^{x}, —OC(J)R^{u}N(R¹⁴)(R¹⁵), —R^{u}C(J)R^{u}N(R¹⁴)(R¹⁵), —R^{u}S(O)ₜR^{w}, —R^{u}N(R^{x})C(J)R^{x}, —R^{u}N(R^{x})C(J)OR^{x}, —OP(O)(OH)₂, and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O, NR^{x} or S; and

each t is independently an integer from 0-2.

In one embodiment, the compounds of Formula I or Formula IA are selected such that

bond a is a single bond or double bond;

R¹ is hydrogen, alkyl, aryl, heteroaryl, heterocyclyl or cycloalkyl;

R² is alkyl, deuteroalkyl, or cycloalkyl;

R³ is alkyl, deuteroalkyl, cycloalkyl, SO₂R¹⁹, COR², or —SO₂N(R¹⁴)(R¹⁵);

R⁴ and R⁵ are each independently selected from hydrogen and alkyl;

A is CH, CR², or N;

E is CO, SO₂, CN(OR¹⁸), CN(CN), CS, CNR¹¹, or CR¹²CF₃;

Y is CR⁷ or CR⁷R⁸;

Z is CR⁹ or CR⁹R¹⁰;

R⁷ and R⁹ together with the atoms on which they are substituted form an optionally substituted 3 to 6-membered cycloalkyl, aryl, heterocyclyl or heteroaryl ring, where substituents, when present are selected from one or more Q and Q³ groups;

R⁸ and R¹⁰, when present, are each independently selected from hydrogen, alkyl and cycloalkyl;

Q¹ is selected from alkyl and cycloalkyl;

R¹¹ and R¹² are each independently selected from hydrogen and alkyl;

R¹⁸ is hydrogen, alkyl or cycloalkyl;

R¹⁹ is alkyl, alkenyl, alkynyl, haloalkyl, cycloalkyl, aryl or heteroaryl;

Q¹, R², R³, R⁴, R⁵, R⁸, R¹⁰ and R¹⁹ are optionally substituted with 1 or 2 substituents, each independently selected from Q², where Q² is selected from deutero, alkyl and cycloalkyl;

R¹ is optionally substituted with 1, 2, 3 or 4 substituents Q³, each Q³ is independently selected from halo, cyano, alkyl, haloalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}N(R^{y})(R^{z}), —R^{u}C(J)OR^{x}, —R^{u}C(J)N(R^{y})(R^{z}), —R^{u}S(O)ₜN(R^{y})(R^{z}), —R^{u}N(R^{x})C(J)R^{x}, —R^{u}N(R^{x})C(J)OR^{x}, and —R^{u}N(R^{x})S(O)ₜR^{w}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;

each R^{u} is independently alkylene or a direct bond;

R^{w} is alkyl, haloalkyl, hydroxyalkyl, alkoxyalkyl, alkenyl, alkynyl, amino, cycloalkyl, cycloalkenyl, cycloalkylalkyl, cycloalkenylalkyl, heterocyclyl, heterocyclylalkyl, aryl, aralkyl, heteroaryl, or heteroaralkyl;

each R^{x} is independently hydrogen, alkyl, haloalkyl, hydroxyalkyl, alkoxyalkyl, cyanoalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;

R^{y} and R^{z} are each independently selected from (i) or (ii) below:

(i) R^{y} and R^{z} are each independently hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a 5 to 12, 5 to 10, 5 to 8 or 5 to 7 membered heterocyclyl or heteroaryl, optionally substituted with one or more, in one embodiment, one, two or three Q⁵ groups; each Q⁵ is independently selected from halo, hydroxy, cyano, oxo, amino, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}C(J)R^{x}, —R^{u}N(R¹⁴)(R¹⁵), —R^{u}C(J)OR^{x}, —OC(J)R^{u}N(R¹⁴)(R¹⁵), —R^{u}C(J)R^{u}N(R¹⁴)(R¹⁵), —R^{u}S(O)ₜR^{w}, —R^{u}N(R^{x})C(J)R^{x}, —R^{u}N(R^{x})C(J)OR^{x}, —OP(O)(OH)₂, and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;

R¹⁴ and R¹⁵ are each independently (i) or (ii) below:

(i) R¹⁴ and R⁵ is are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O; and

each t is independently an integer from 0-2.

In one embodiment, the compounds of Formula I or Formula IA are selected such that

bond a is a single bond or double bond;

R¹ is aryl, heteroaryl, heterocyclyl or cycloalkyl;

R² is alkyl, deuteroalkyl, or cycloalkyl;

R³ is alkyl, deuteroalkyl, cycloalkyl, SO₂R¹⁹, COR², or —SO₂N(R¹⁴)(R¹⁵);

R⁴ and R⁵ are each independently selected from hydrogen and alkyl;

A is CH, CR², or N;

E is CO or SO₂;

Y is CR⁷ or CR⁷R⁸;

Z is CR⁹ or CR⁹R¹⁰;

R⁷ and R⁹ together with the atoms on which they are substituted form a 3 to 6-membered cycloalkyl, aryl, heterocyclyl or heteroaryl ring; and R⁸ and R¹⁰, when present, are each independently selected from hydrogen, alkyl and cycloalkyl; R¹⁹ is alkyl, alkenyl, alkynyl, haloalkyl, cycloalkyl, aryl or heteroaryl; R², R³, R⁴, R⁵, R⁸, R¹⁰ and R¹⁹ are optionally substituted with 1 or 2 substituents, each independently selected from Q², where Q² is selected from deutero, alkyl and cycloalkyl;

R¹ is optionally substituted with 1, 2, 3 or 4 substituents Q³, each Q³ is independently selected from halo, cyano, alkyl, haloalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}N(R^{y})(R^{z}), —R^{u}C(J)OR^{x}, —R^{u}C(J)N(R^{y})(R^{z}), —R^{u}S(O)ₜN(R^{y})(R^{z}), —R^{u}N(R^{x})C(J)R^{x}, —R^{u}N(R^{x})C(J)OR^{x}, and —R^{u}N(R^{x})S(O)ₜR^{w}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;

each R^{u} is independently alkylene or a direct bond;

R^{w} is alkyl, haloalkyl, hydroxyalkyl, alkoxyalkyl, alkenyl, alkynyl, amino, cycloalkyl, cycloalkenyl, cycloalkylalkyl, cycloalkenylalkyl, heterocyclyl, heterocyclylalkyl, aryl, aralkyl, heteroaryl, or heteroaralkyl;

each R^{x} is independently hydrogen, alkyl, haloalkyl, hydroxyalkyl, alkoxyalkyl, cyanoalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; R^{y} and R^{z} are each independently selected from (i) or (ii) below:

(i) R^{y} and R^{z} are each independently hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, cyano, oxo, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}C(J)R^{x}, —R^{u}N(R¹⁴)(R¹⁵), —R^{u}C(J)OR^{x}, —OC(J)R^{u}N(R¹⁴)(R¹⁵), —R^{u}C(J)R^{u}N(R¹⁴)(R¹⁵), R^{u}S(O)ₜR^{w}, —R^{u}N(R^{x})C(J)R^{x}, —R^{u}N(R^{x})C(J)OR^{x}, —OP(O)(OH)₂, and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O; and

each t is independently an integer from 0-2.

In one embodiment, the compounds provided herein are of Formula I or Formula IA, wherein bond a is a single bond. In one embodiment, the compounds provided herein are of Formula I or Formula IA, wherein bond a is a double bond.

In one embodiment, the compounds provided herein are of Formula I or Formula IA, wherein R¹ is hydrogen, alkyl, alkenyl, alkynyl, aryl, heteroaryl, heterocyclyl or cycloalkyl. In one embodiment, the compounds provided herein are of Formula I or Formula IA, wherein R¹ is hydrogen, alkyl, aryl, heteroaryl, heterocyclyl or cycloalkyl. In one embodiment, the compounds provided herein are of Formula I or Formula IA, wherein R¹ is hydrogen or alkyl. In one embodiment, the compounds provided herein are of Formula I or Formula IA, wherein R¹ is aryl, heteroaryl, heterocyclyl or cycloalkyl.

In one embodiment, the compounds provided herein are of Formula I or Formula IA, wherein R² is alkyl, deuteroalkyl, or cycloalkyl. In one embodiment, R² is alkyl. In one embodiment, R² is methyl.

In one embodiment, the compounds provided herein are of Formula I or Formula IA, wherein R³ is alkyl, deuteroalkyl, or cycloalkyl. In one embodiment, R³ is alkyl. In one embodiment, R³ is methyl. In one embodiment, R³ is C₃-C₆ cycloalkyl. In one embodiment, R³ is cyclopropyl. In one embodiment, R³ is SO₂R¹⁹. In one embodiment, R³ is SO₂CH₃.

In one embodiment, R⁴ is hydrogen. In one embodiment, R⁵ is hydrogen.

In one embodiment, A is CH, CR², or N, where R² is lower alkyl. In one embodiment, A is CH or N. In one embodiment, A is CH.

In one embodiment, E is CO. In one embodiment, E is SO₂.

In one embodiment, E is CNOR¹⁸, and R¹⁸ is hydrogen, alkyl or cycloalkyl. In one embodiment, R¹⁸ is hydrogen, C₁₋₄alkyl or C₃₋₆cycloalkyl.

In one embodiment, Y is CR⁷ or CR⁷R⁸; Z is CR⁹ or CR⁹R¹⁰; wherein R⁷ and R⁹ together with the atoms on which they are substituted form a 3 to 6-membered cycloalkyl, aryl, heterocyclyl or heteroaryl ring; and R⁸ and R¹⁰, when present, are each hydrogen.

In one embodiment, Y is CR⁷; Z is CR⁹; and R⁷ and R⁹ together with the atoms on which they are substituted form a phenyl or furanyl ring.

In one embodiment, Q¹ is lower alkyl.

In one embodiment, the compounds provided herein are of Formula I or Formula IA, wherein R¹ is 5 to 7 membered aryl, heteroaryl, heterocyclyl or cycloalkyl. In one embodiment, R¹ is aryl. In one embodiment, R¹ is heteroaryl. In one embodiment, R¹ is cycloalkyl. In one embodiment, R¹ is heterocyclyl. In one embodiment, R¹ is phenyl, pyridinyl, cyclohexyl, tetrahydropyranyl or pyrazolyl. In one embodiment, R¹ is phenyl, cyclohexyl or pyridinyl. In one embodiment, R¹ is cyclohexyl, cyclopentyl or cyclobutyl.

In one embodiment, R¹ is optionally substituted with 1 or 2 substituents Q³, each Q³ is independently selected from halo, cyano, alkyl, haloalkyl, alkenyl, alkynyl, cycloalkyl, cycloalkenylalkyl, heteroaryl, heterocyclyl, heterocyclylalkyl, —COOH, —R^{u}OR^{x}, —R^{u}N(R^{y})(R^{z}), —R^{u}C(J)N(R^{y})(R^{z}), —R^{u}S(O)ₜN(R^{y})(R^{z}), —R^{u}N(R^{x})C(J)R^{x}, and —R^{u}N(R^{x})S(O)ₜR^{w}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;

each R^{u} is independently alkylene or a direct bond;

R^{w} is alkyl or amino;

each R^{x} is independently hydrogen, alkyl or hydroxyalkyl;

R^{y} and R^{z} are each independently selected from (i) or (ii) below:

(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, cyano, oxo, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —OC(J)R^{u}N(R¹⁴)(R¹⁵), —R^{u}N(R¹⁴)(R¹⁵), —R^{u}C(J)R^{u}N(R¹⁴)(R¹⁵), —R^{u}N(R^{x})C(J)OR^{x}, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O; and

t is an integer from 0-2.

In certain embodiments, the compounds provided herein are of formula IB:

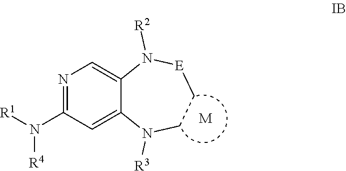

or a pharmaceutically acceptable salt thereof, wherein ring M is aryl, cycloalkyl, heterocyclyl or heteroaryl ring, and the other variables are as described elsewhere herein. In certain embodiments, the compounds provided herein are of formula IB, wherein ring M is optionally substituted with one, two or three groups selected from halo, haloalkyl, alkyl, hydroxyl or alkoxy, and the other variables are as described elsewhere herein. In certain embodiments, M is optionally substituted with one, two or three groups selected from fluoro, chloro, methyl, methoxy and ethoxy.

In certain embodiments, the compounds provided herein are of formula IB, wherein

E is CO, or SO₂;

M is

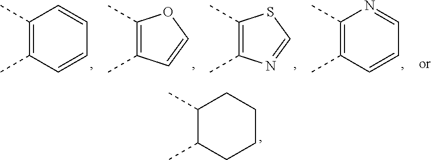

where M is optionally substituted with one or two substituents selected from halo, alkyl, alkoxy, hydroxyl and haloalkyl;

R¹ is phenyl, pyridyl, pyrazolyl, cyclohexyl, or tetrahydropyranyl ring, which is optionally substituted with 1 or 2 substituents Q³ or Q⁴, wherein Q³ and Q⁴ is independently selected from halo, cyano, hydroxy, C₁-C₄alkyl, amino(C₁-C₄)alkyl, C₁-C₄alkyloxy, halo(C₁-C₄)alkyloxyl, hydroxy(C₁-C₄)alkyl, C₁-C₄alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOR^{2a}, —COR^{3a}, or —CH₂R^{4a};

R^{2a} is C₁-C₄ alkyl,

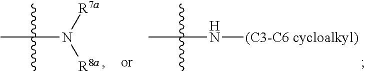

R^{3a} is selected from amino, hydroxy,

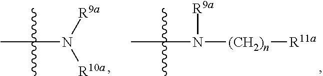

or 4 to 10, 4 to 9, 4 to 8 or 4 to 7 member heterocyclyl which may be substituted with halogen, hydroxy, cyano, oxo, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, —OC(J)R^{u}N(R¹⁴)(R¹⁵), —R^{u}C(J)R^{x}, —R^{u}S(O)ₜR^{w}, —R^{u}C(J)R^{u}N(R¹⁴)(R¹⁵), —OP(O)(OH)₂, —R^{u}N(R¹⁴)(R¹⁵), or a 4 to 6 member heterocyclyl group;

R^{4a} is hydroxy,

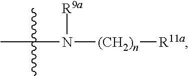

or 4 to 7 member heterocyclyl group,

which may be substituted with halogen, hydroxy, cyano, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, or a 4 to 6 member heterocyclyl group;

R², R^{7a} and R^{8a} are independently C₁-C₄ alkyl, deutero C₁-C₄ alkyl, or C₃-C₆ cycloalkyl;

R³ is C₁-C₄ alkyl, deutero C₁-C₄ alkyl, C₃-C₆ cycloalkyl or SO₂R¹⁹;

R⁴ is hydrogen or C₁-C₄ alkyl;

R^{9a} and R^{10a} are independently selected from hydrogen, hydroxy, C₁-C₄ alkyl, hydroxy(C₁-C₄)alkyl, C₁-C₄ acyl, C₁-C₄ alkyloxy(C₁-C₄)alkyl, C₁-C₄ alkenyl, or, 4 to 7 member heterocyclyl group,

which may be substituted with halogen, hydroxy, cyano, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, or a 4 to 6 member heterocyclyl group;

R^{11a} is

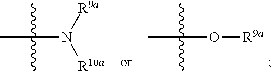

each R^{u} is independently alkylene or a direct bond;

R¹⁴ and R¹⁵ are each independently (i) or (ii) below:

(i) R¹⁴ and R¹⁵ are each independently hydrogen or alkyl; or

(ii) R¹⁴ and R¹⁵, together with the nitrogen atom to which they are attached, form a heterocyclyl or heteroaryl, optionally substituted hydroxyl or alkyl; and

R¹⁹ is alkyl, alkenyl, alkynyl, haloalkyl, cycloalkyl, aryl or heteroaryl;

n is a natural number from 1 to 3.

In certain embodiments, M is optionally substituted with one, two or three groups selected from fluoro, chloro, methyl, methoxy and ethoxy.

In certain embodiments, the compounds provided herein are of formula IB, wherein

E is CO, or SO₂;

M is

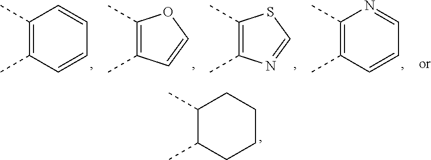

where M is optionally substituted with one or two substituents selected from halo, alkyl, alkoxy, hydroxyl and haloalkyl;

R² and R³ are each C₁-C₄ alkyl or deutero C₁-C₄ alkyl;

R⁴ is hydrogen or C₁-C₄ alkyl;

R¹ is phenyl, pyridyl, pyrazolyl, cyclohexyl, or tetrahydropyranyl ring, which is optionally substituted with 1 or 2 substituents Q^{3a} or Q^{4a}, wherein each of Q^{3a} and Q^{4a} is independently selected from halo, cyano, hydroxy, C₁-C₄ alkyl, amino(C₁-C₄)alkyl, C₁-C₄ alkyloxy, halo(C₁-C₄)alkyloxyl, hydroxy(C₁-C₄)alkyl, C₁-C₄ alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOR^{2a}, —COR^{3a}, and —CH₂R^{4a};

R^{2a} is alkyl C₁-C₄ alkyl,

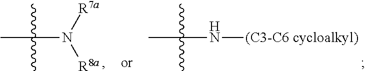

R^{3a} is selected from amino, hydroxy,

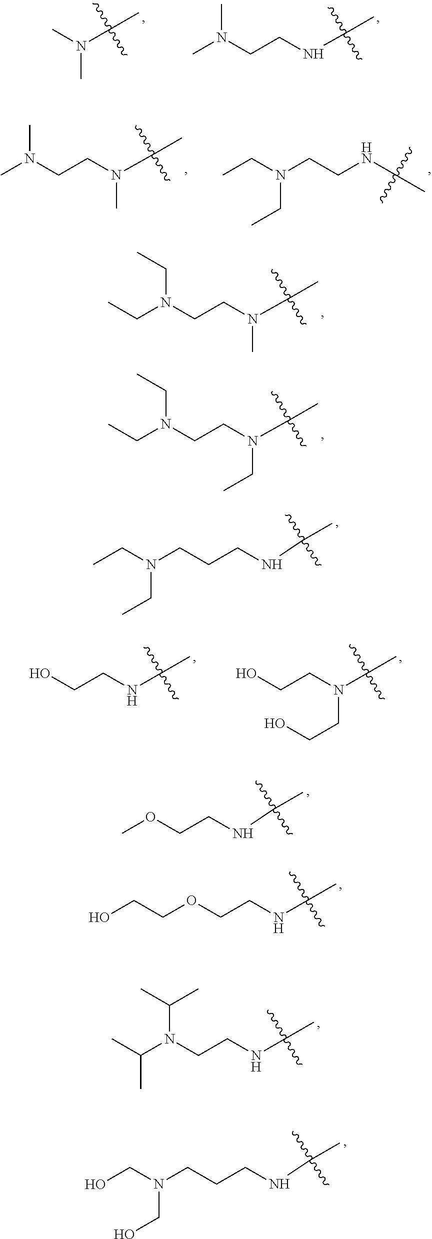

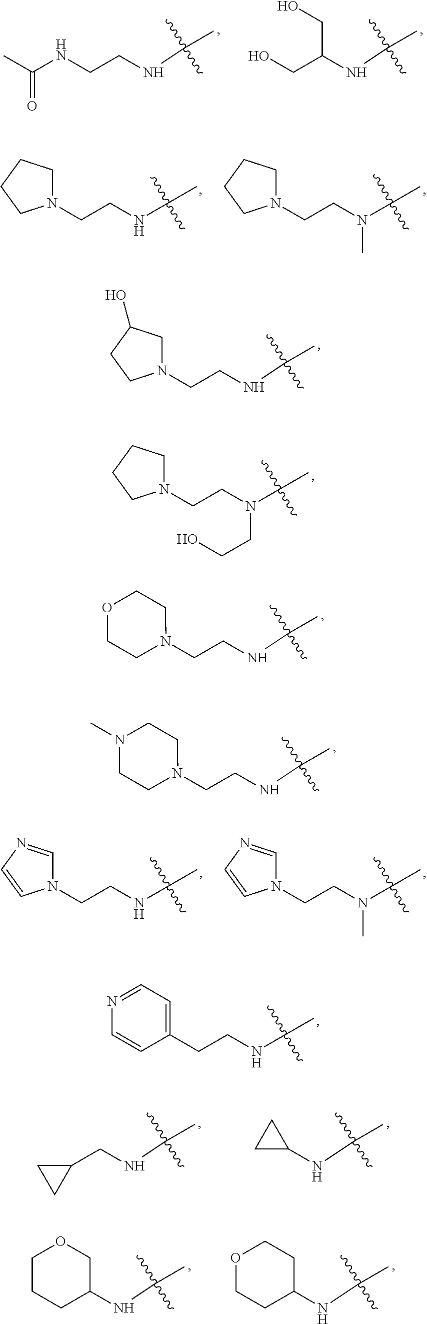

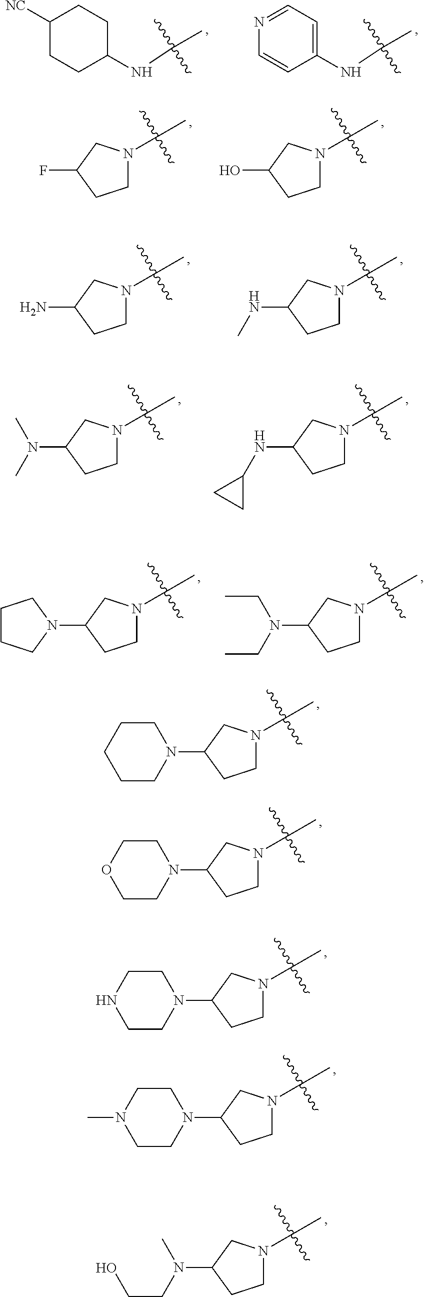

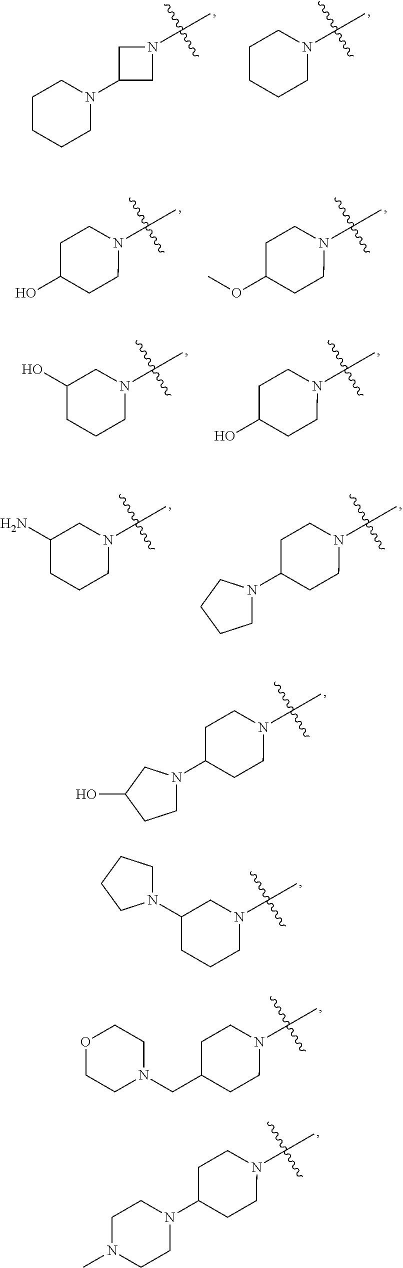

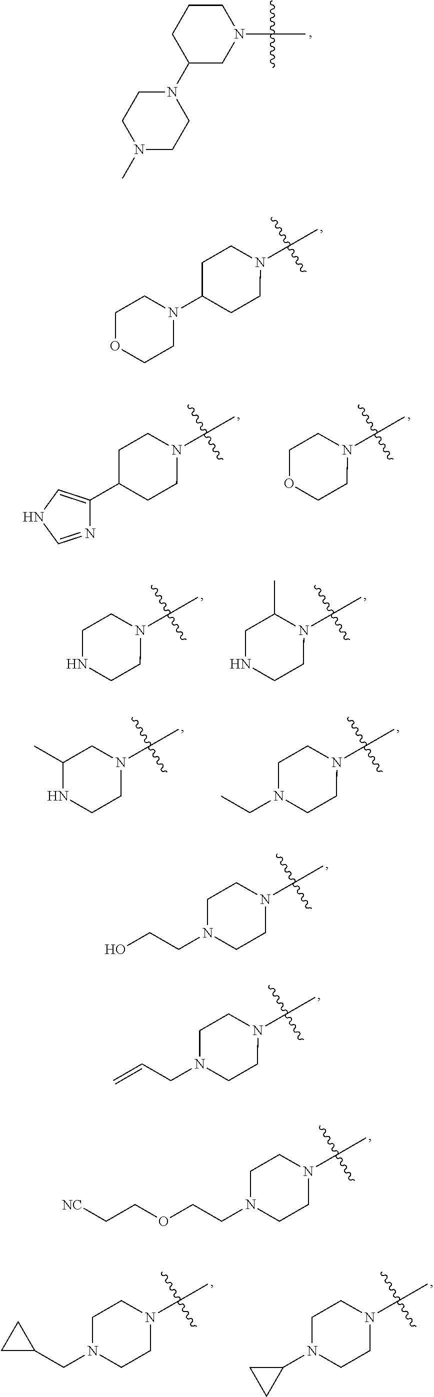

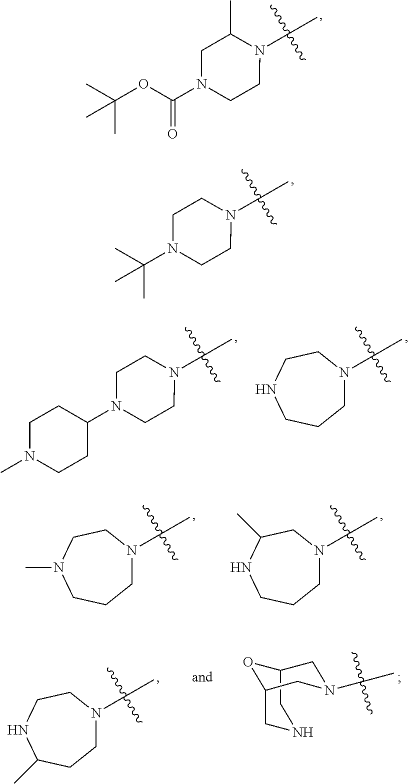

R^{4a} is hydroxy,

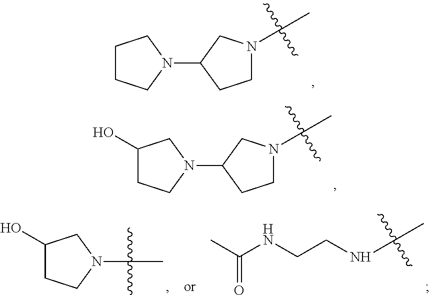

R^{7a} and R^{8a} are independently C₁-C₄ alkyl or C₃-C₆ cycloalkyl; R^{9a} and R^{10a} are independently selected from hydrogen, hydroxy, C₁-C₄ alkyl, hydroxy(C₁-C₄)alkyl, C₁-C₄ acyl, C₁-C₄ alkyloxy(C₁-C₄)alkyl, C₁-C₄ alkenyl, or, 4 to 7 member heterocyclyl group,

which may be substituted with halogen, hydroxy, cyano, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, or a 4 to 6 member heterocyclyl group;

R^{11a} is or

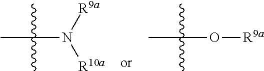

n is a natural number from 1 to 3.

In certain embodiments, the compounds provided herein are of formula IB, wherein E is CO;

M is

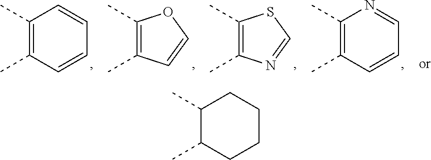

where M is optionally substituted with one or two substituents selected from halo, alkyl, alkoxy, hydroxyl and haloalkyl;

R¹ is phenyl, which is optionally substituted with 1 or 2 substituents Q^{3a}, wherein Q^{3a} is selected from halo, cyano, hydroxy, C₁-C₄ alkyl, amino(C₁-C₄)alkyl, C₁-C₄ alkyloxy, halo(C₁-C₄)alkyloxyl, hydroxy(C₁-C₄)alkyl, C₁-C₄ alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOCH₃, —COR^{3a}, and —CH₂R^{4a}; and the remaining variables are as described therein.

In certain embodiments, the compounds provided herein are of formula IC:

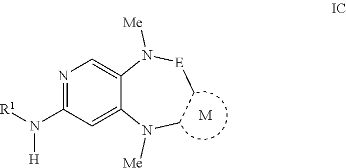

or a pharmaceutically acceptable salt thereof, wherein

E is CO, or SO₂;

M is

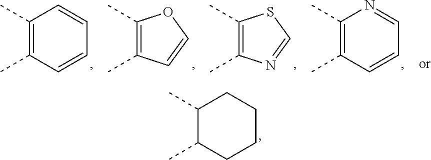

where M is optionally substituted with one or two substituents selected from halo, alkyl, alkoxy, hydroxyl and haloalkyl;

R¹ is phenyl, pyridyl, pyrazolyl, cyclohexyl, cyclopentyl, cyclobutyl, or tetrahydropyranyl ring, which is optionally substituted with 1 or 2 substituents Q^{3a} or Q^{4a}, wherein each of Q^{3a} and Q^{4a} is independently selected from halo, cyano, hydroxy, C₁-C₄ alkyl, amino(C₁-C₄)alkyl, C₁-C₄ alkyloxy, halo(C₁-C₄)alkyloxyl, hydroxy(C₁-C₄)alkyl, C₁-C₄ alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOR^{2a}, —COR^{3a}, and —CH₂R^{4a};

R^{2a} is alkyl C₁-C₄ alkyl,

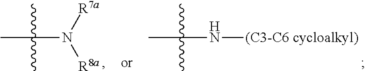

R^{3a} is selected from amino, hydroxy,

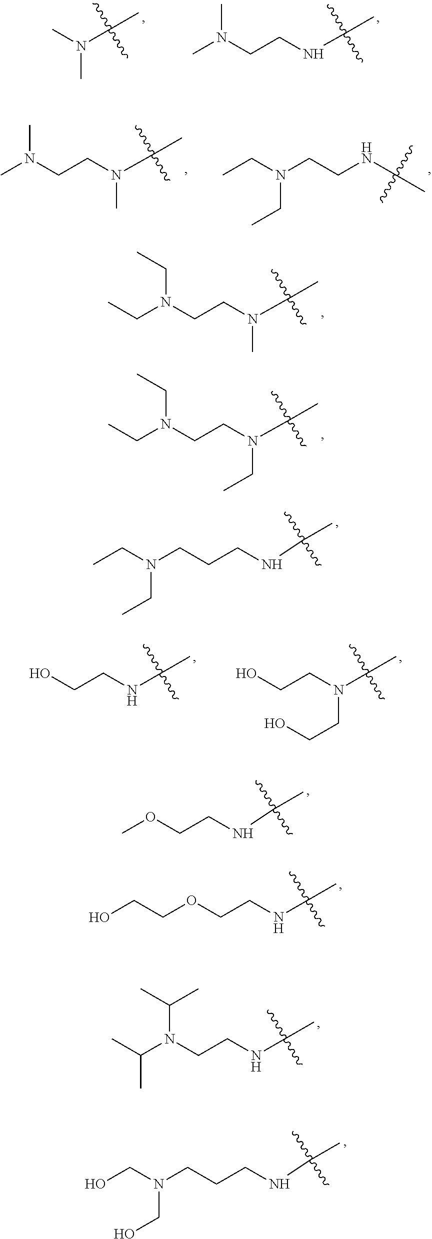

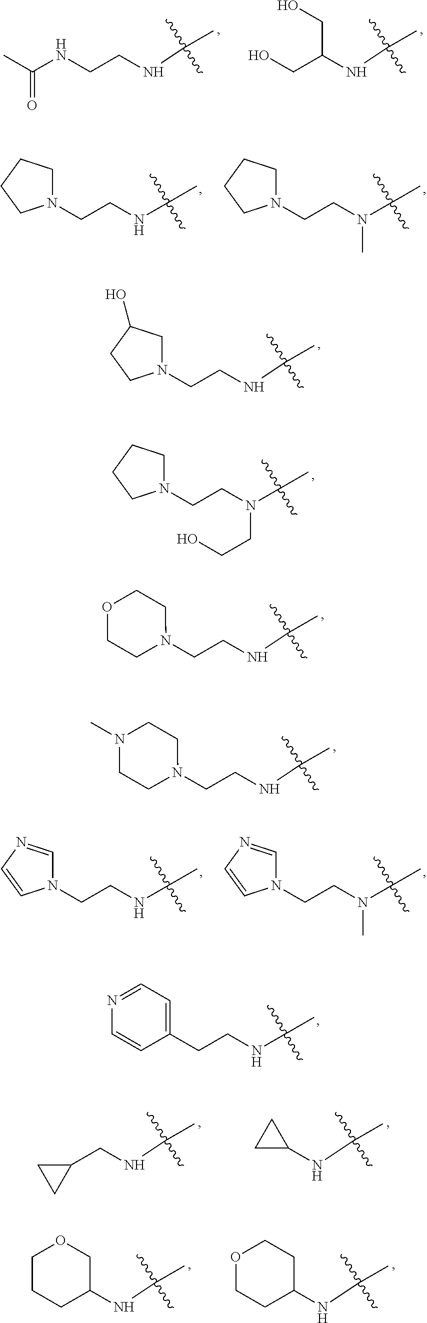

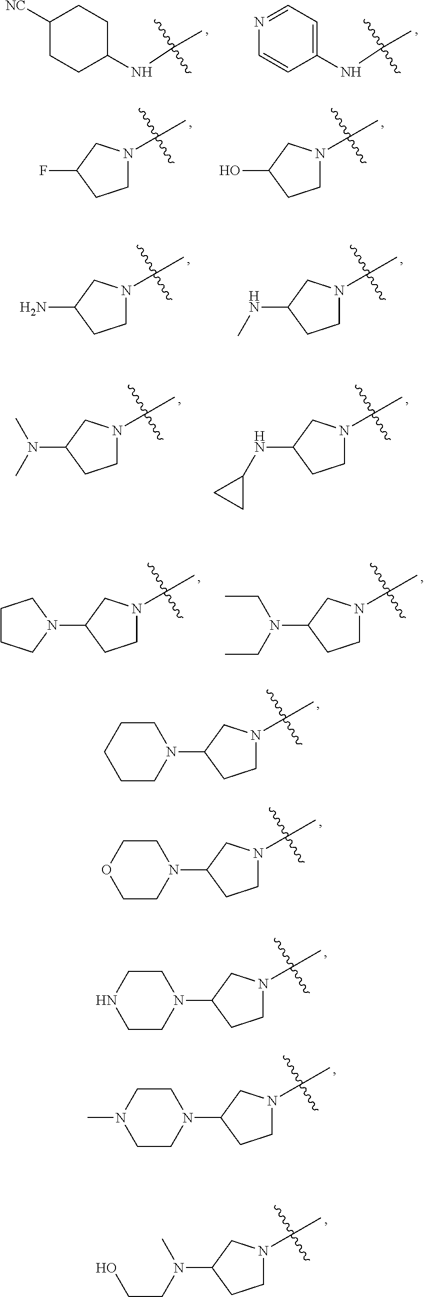

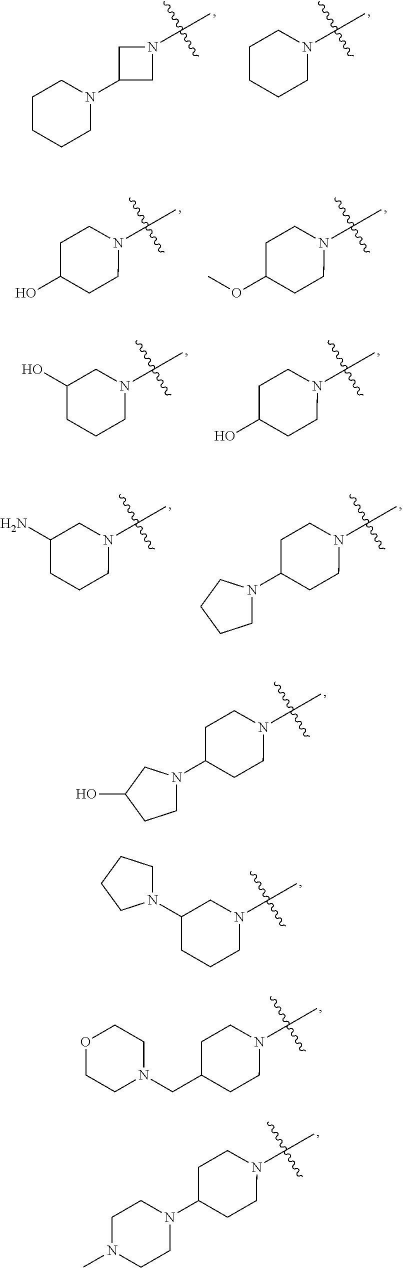

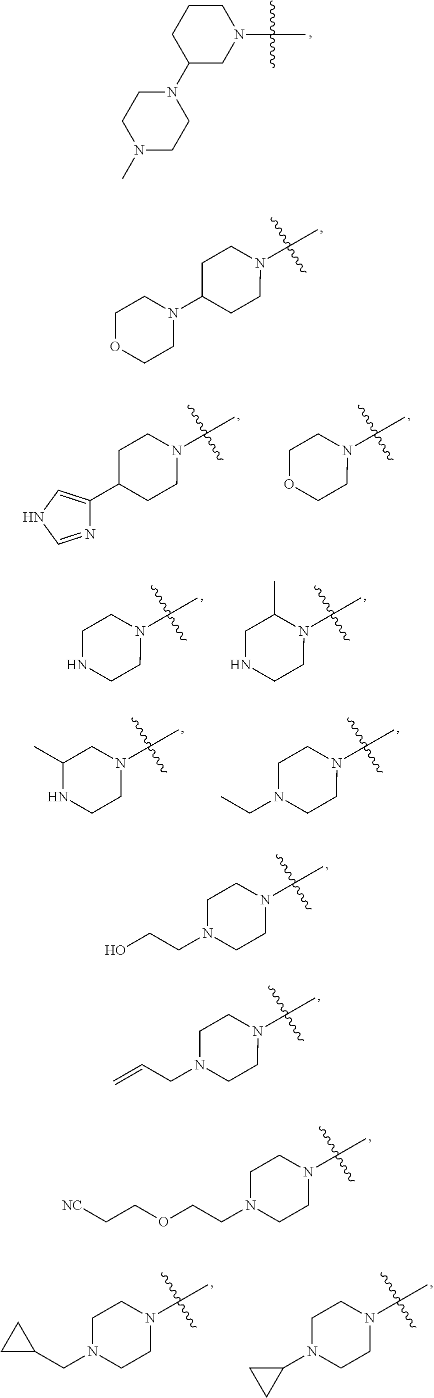

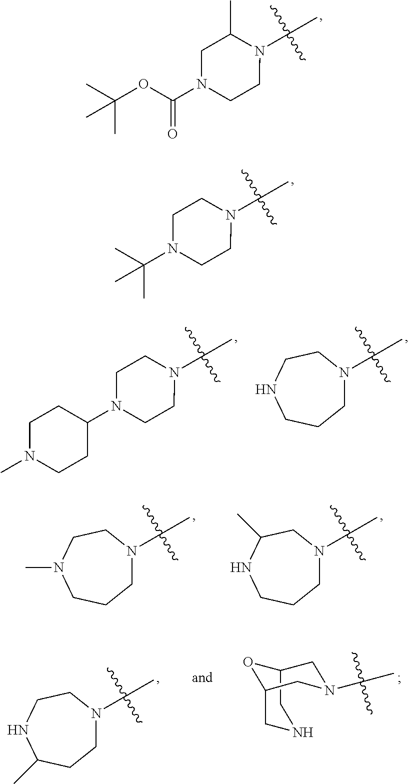

R^{4a} is hydroxy,

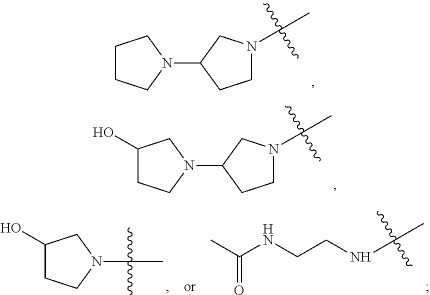

R^{7a} and R^{8a} are independently C₁-C₄ alkyl or C₃-C₆ cycloalkyl; R^{9a} and R^{10a} are independently selected from hydrogen, hydroxy, C₁-C₄ alkyl, hydroxy(C₁-C₄)alkyl, C₁-C₄ acyl, C₁-C₄ alkyloxy(C₁-C₄)alkyl, C₁-C₄ alkenyl, or, 4 to 7 member heterocyclyl group,

which may be substituted with halogen, hydroxy, cyano, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, or a 4 to 6 member heterocyclyl group;

R^{11a} is

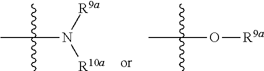

n is a natural number from 1 to 3.

In certain embodiments, the compounds provided herein are of formula ID:

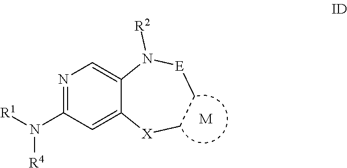

or a pharmaceutically acceptable salt thereof, wherein ring M is aryl, cycloalkyl, heterocyclyl or heteroaryl ring, where ring M is optionally substituted with one or two substituents selected from halo, alkyl, alkoxy, hydroxyl and haloalkyl; and the other variables are as described else wherein. In certain embodiments, M is optionally substituted with one, two or three groups selected from fluoro, chloro, methyl, methoxy and ethoxy.

In certain embodiments, the compounds provided herein are of formula ID, wherein

X is NR³, O, S(O)₀₋₂, or CR^{a}R^{b};

R^{a} and R^{b} are selected as follows:

(i) R^{a} and R^{b} are each independently selected from hydrogen, C₁₋₄alkyl, C₂₋₄alkenyl, C₂₋₄alkynyl, halo C₁₋₄alkyl, C₃₋₆cycloalkyl, aryl, heterocyclyl and heteroaryl; or

(ii) R^{a} and R^{b} together form ═O;

R³ is C₁₋₄alkyl, deutero C₁₋₄alkyl, C₂₋₄alkenyl, C₂₋₄alkynyl, halo C₁₋₄alkyl, C₃₋₆cycloalkyl, SO₂R¹⁹, COR², or —SO₂N(R¹⁴)(R¹⁵);

E is CO, SO₂ or CHCF₃;

ring M is

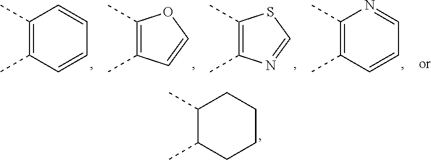

where ring M is optionally substituted with one or two substituents selected from halo, alkyl, alkoxy, hydroxyl and haloalkyl;

R² is C₁-C₄ alkyl or deutero C₁-C₄ alkyl;

R⁴ is hydrogen or C₁-C₄ alkyl;

R¹⁹ is C₁-C₄ alkyl;

R¹ is phenyl, pyridyl, pyrazolyl, cyclohexyl, cyclopentyl, cyclobutyl, or tetrahydropyranyl ring, which is optionally substituted with 1 or 2 substituents Q^{3a} or Q^{4a} wherein each of Q^{3a} and Q^{4a} is independently selected from halo, cyano, hydroxy, C₁-C₄ alkyl,

amino(C₁-C₄)alkyl, C₁-C₄ alkyloxy, halo(C₁-C₄)alkyloxyl, hydroxy(C₁-C₄)alkyl, C₁-C₄ alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOR^{2a}, —COR^{3a}, and —CH₂R^{4a};

R^{2a} is alkyl C₁-C₄ alkyl,

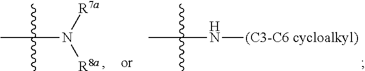

R^{3a} is selected from amino, hydroxy,

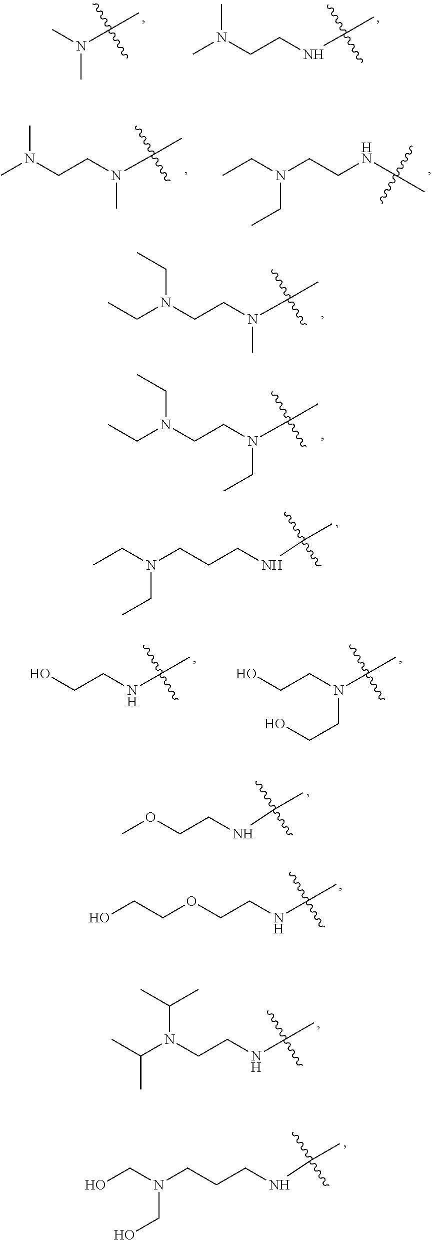

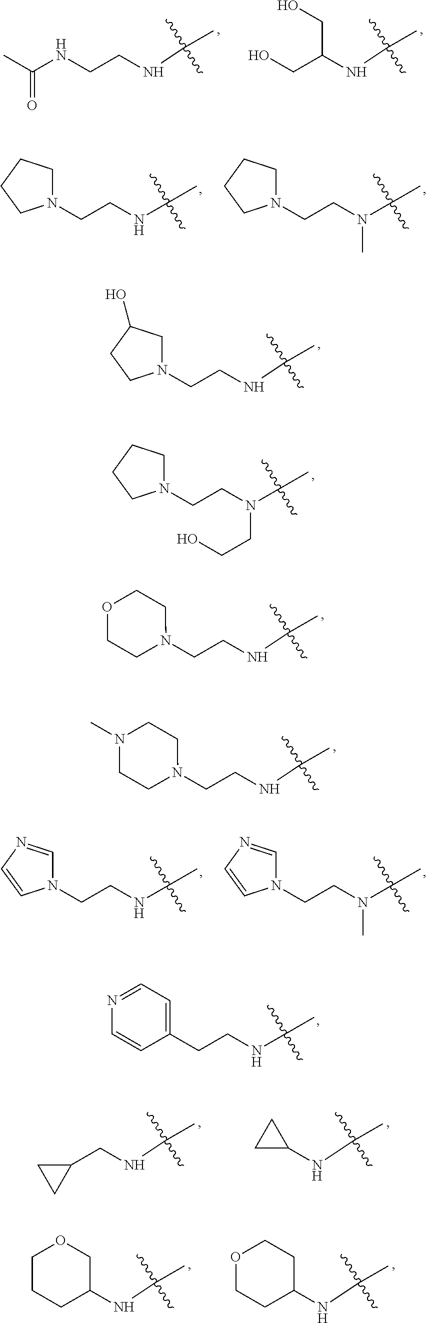

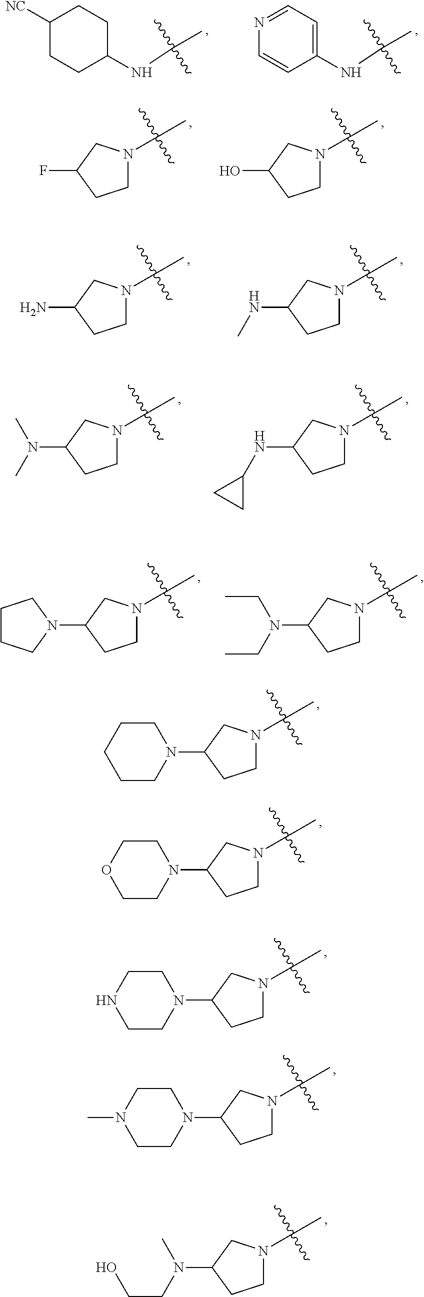

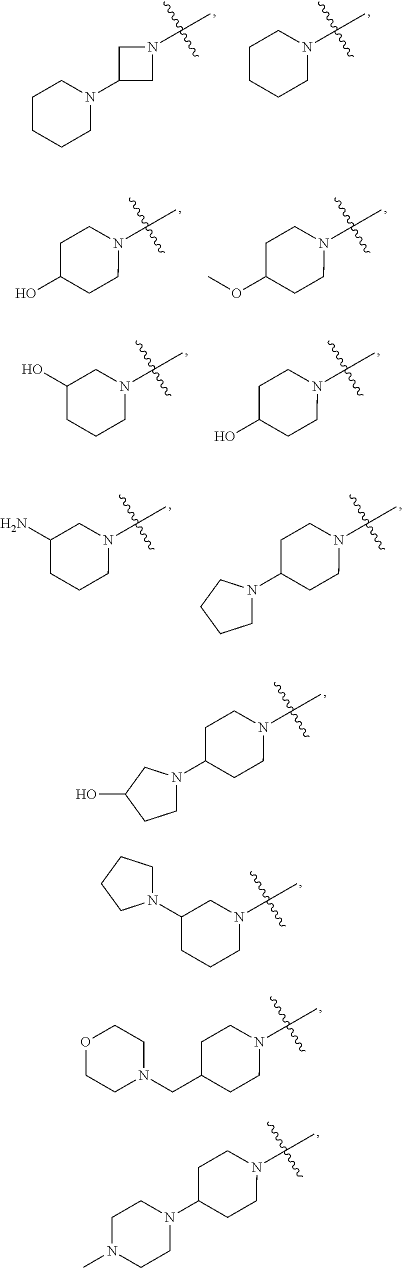

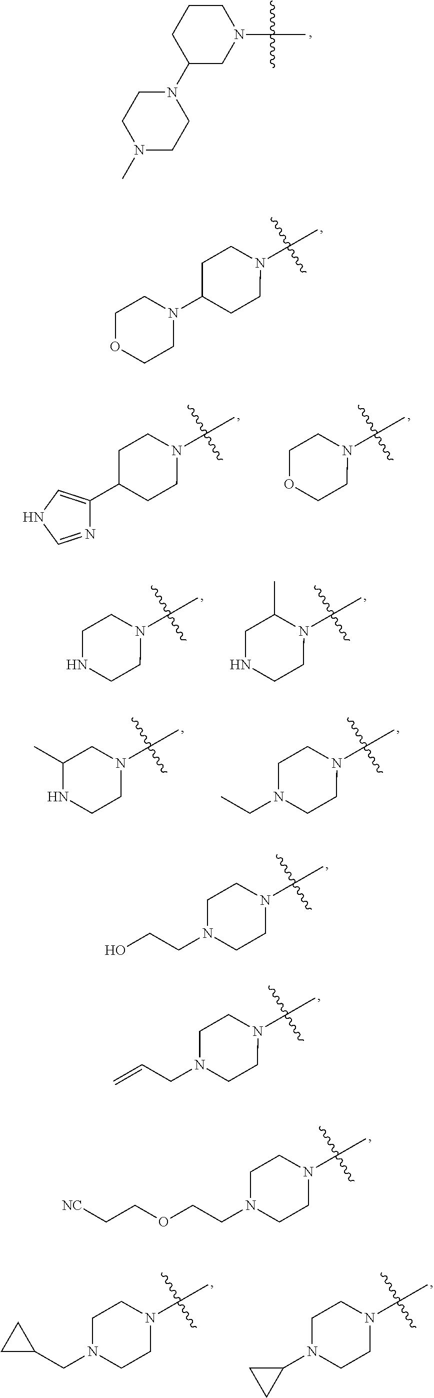

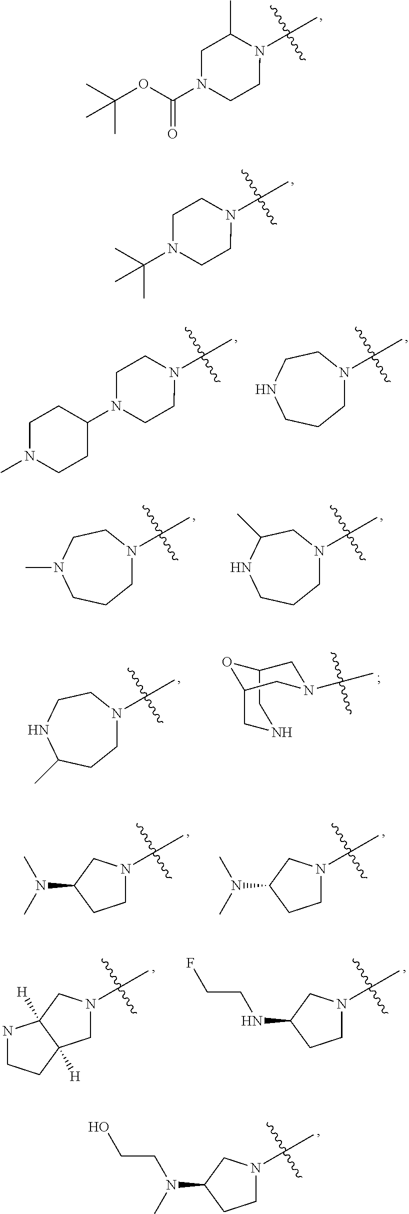

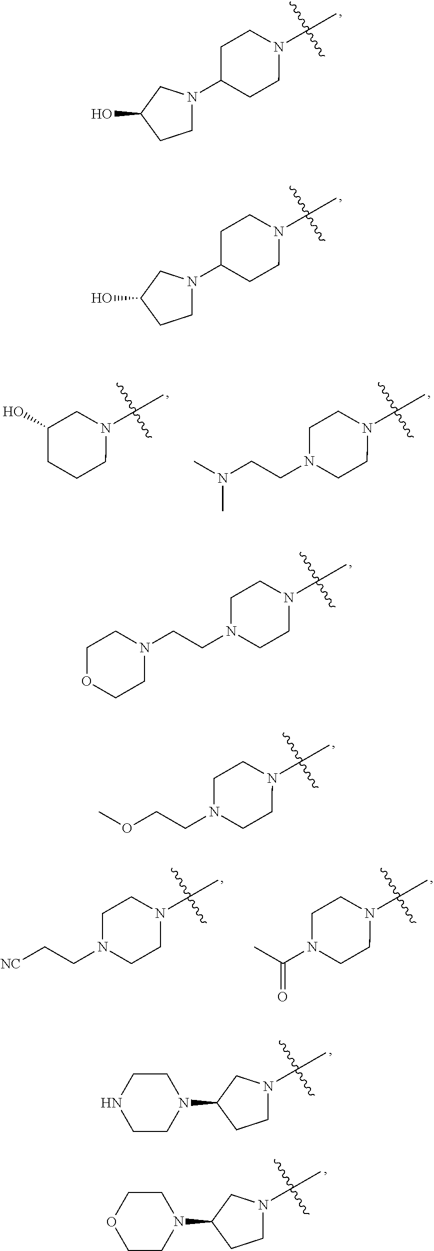

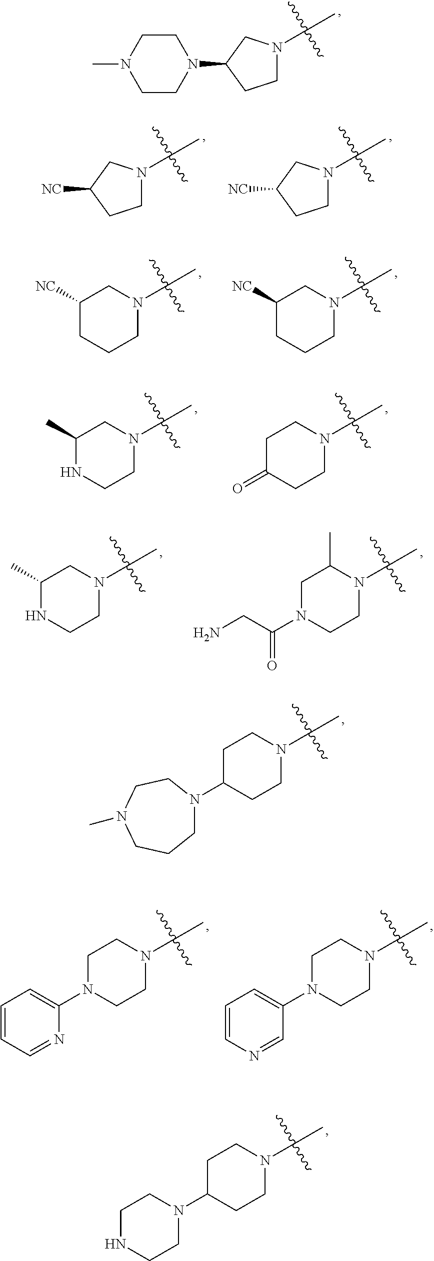

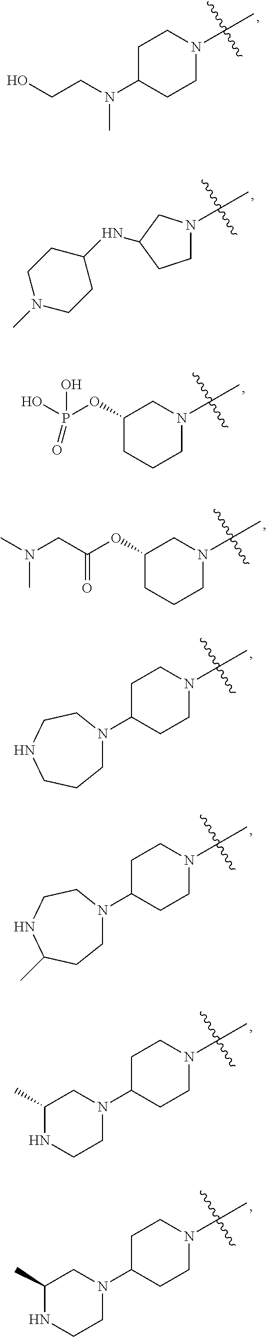

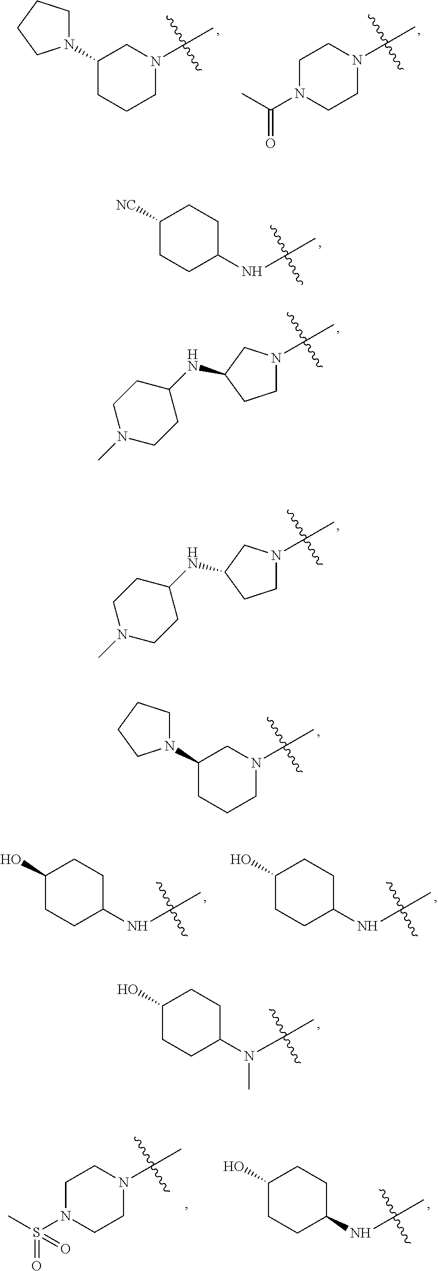

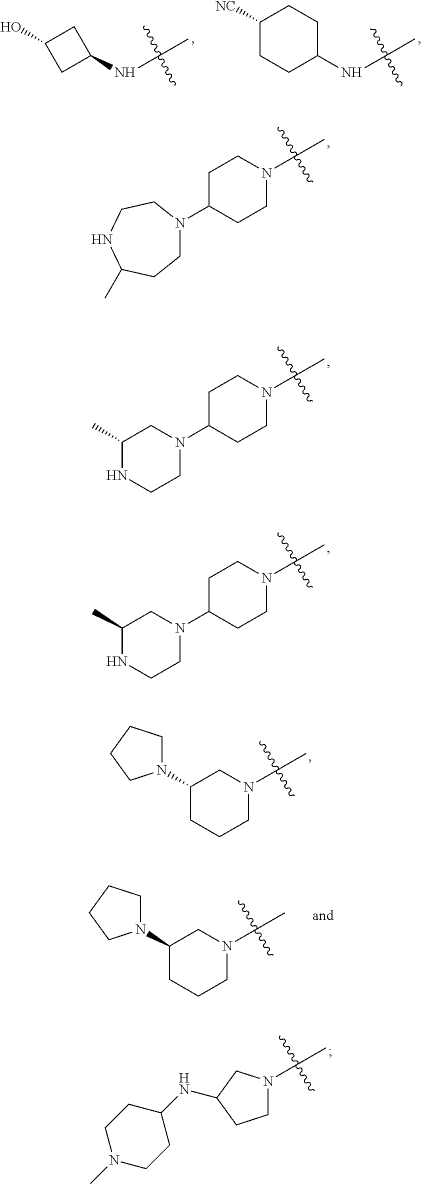

R^{4a} is hydroxy,

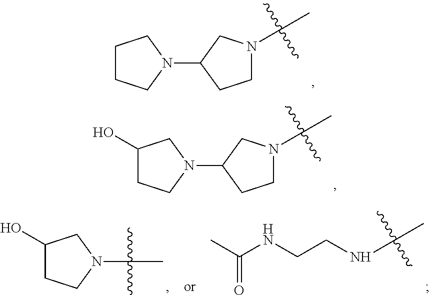

R^{7a} and R^{8a} are independently C₁-C₄ alkyl or C₃-C₆ cycloalkyl; R^{9a} and R^{10a} are independently selected from hydrogen, hydroxy, C₁-C₄ alkyl, hydroxy(C₁-C₄)alkyl, C₁-C₄ acyl, C₁-C₄ alkyloxy(C₁-C₄)alkyl, C₁-C₄ alkenyl, or, 4 to 7 member heterocyclyl group,

which may be substituted with halogen, hydroxy, cyano, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, or a 4 to 6 member heterocyclyl group;

R^{11a} is

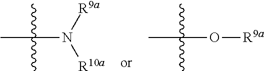

R¹⁴ and R¹⁵ are each independently hydrogen, C₁-C₄ alkyl, halo C₁-C₄ alkyl, hydroxy C₁-C₄ alkyl, C₁-C₄ alkoxy C₁-C₄ alkyl, C₂-C₄ alkenyl, C₂-C₄ alkynyl, C₃-C₆ cycloalkyl, where R¹⁴ and R¹⁵ are each optionally substituted with one, two or three Q⁵ groups; and

n is a natural number from 1 to 3.

In certain embodiments, the compounds provided herein are of formula IB, IC, or ID wherein E is CO;

M is

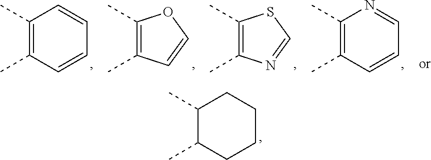

where M is optionally substituted with one or two substituents selected from halo, alkyl, alkoxy, hydroxyl and haloalkyl;

R¹ is phenyl, which is optionally substituted with 1 or 2 substituents Q^{3a}, wherein Q^{3a} is selected from halo, cyano, hydroxy, alkyl, aminoalkyl, alkyloxy, haloalkyloxyl, hydroxyalkyl, alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOCH₃, —COR^{3a} and —CH₂R^{4a}; and the remaining variables are as described elsewhere herein.

In certain embodiments, the compounds provided herein are of formula IB, IC, or ID, wherein E is CO;

M is

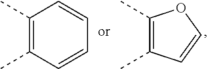

where M is optionally substituted with one or two substituents selected from halo, alkyl, alkoxy, hydroxyl and haloalkyl;

R¹ is phenyl, which is optionally substituted with 1 or 2 substituents Q^{3a}, wherein Q^{3a} is selected from halo, cyano, hydroxy, alkyl, aminoalkyl, alkyloxy, haloalkyloxyl, hydroxyalkyl, alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOCH₃, —COR^{3a}, and —CH₂R^{4a}; R² is CH₃; and the remaining variables are as described elsewhere herein.

In certain embodiments, the compounds provided herein are of formula IB, IC, or ID wherein E is CO;

M is,

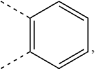

where M is optionally substituted with one or two substituents selected from halo, alkyl, alkoxy, hydroxyl and haloalkyl;

R¹ is cyclohexyl, cyclopentyl or cyclobutyl, which is optionally substituted with 1 or 2 substituents Q^{3a}, wherein Q^{3a} is selected from halo, cyano, hydroxy, alkyl, aminoalkyl, alkyloxy, haloalkyloxyl, hydroxyalkyl, alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOCH₃, —COR^{3a}, and —CH₂R^{4a}; and the remaining variables are as described elsewhere herein.

In certain embodiments, the compounds provided herein are of formula IB, IC, or ID, wherein R¹ is phenyl, pyridyl, pyrazolyl, cyclohexyl, or tetrahydropyranyl ring, which is optionally substituted with 1 or 2 substituents Q^{3a} or Q^{4a}, wherein Q^{3a} is alkyloxy, and Q^{4a} is independently selected from halo, cyano, hydroxy, alkyl, aminoalkyl, alkyloxy, haloalkyloxyl, hydroxyalkyl, alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOCH₃, —COR^{3a} and —CH₂R^{4a}, and the other variables are as described else wherein.

In certain embodiments, the compounds provided herein are of formula IB, IC, or ID, wherein R¹ is:

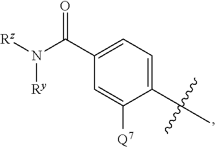

where Q⁷ is hydrogen, hydroxyl, halo, alkyl, alkoxy or haloalkoxy; and R^{y} and R^{z} are each independently selected from (i) or (ii) below:

(i) R^{y} is selected from hydrogen and alkyl; and R^{z} is hydrogen, alkyl, aminoalkyl, hydroxyalkyl, alkoxyalkyl, cycloalkyl, cycloalkylalkyl, heterocyclyl, heterocyclylalkyl, aryl, heteroaryl or heteroarylalkyl, where R^{z} is optionally substituted with one or two alkyl, hydroxyl, alkoxy, —COOH or amino groups; or

(ii) R^{y} and R^{z}, together with the nitrogen atom to which they are attached, form a 5 to 7 membered heterocyclyl or heteroaryl, optionally substituted with one, two or three Q⁵ groups; each Q⁵ is independently selected from halo, hydroxy, amino, cyano, oxo, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —R^{u}N(R^{x})C(J)OR^{x}, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups;

each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O;

each R^{u} is independently alkylene or a direct bond;

R^{w} is alkyl;

each R^{x} is independently hydrogen, alkyl, hydroxyalkyl or alkoxyalkyl; and

t is an integer from 0-2.

In certain embodiments, the compounds provided herein are of formula IB, IC, or ID, wherein R¹ is:

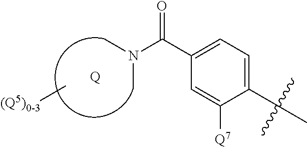

where Q⁷ is hydrogen, hydroxyl, halo, alkyl or alkoxy;

ring Q is a 5 to 7 membered heterocyclyl or heteroaryl ring;

each Q⁵ is independently selected from halo, hydroxy, amino, cyano, oxo, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}N(R¹⁴)(R¹⁵), —R^{u}C(J)R^{x}, —R^{u}C(J)OR^{x}, —R^{u}N(R^{x})C(J)OR^{x}, —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups;

each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O;

each R^{u} is independently alkylene or a direct bond;

R^{w} is alkyl;

each R^{x} is independently hydrogen, alkyl, hydroxyalkyl or alkoxyalkyl; and

t is an integer from 0-2.

In certain embodiments, the compounds provided herein are of formula IB, IC, or ID, wherein R¹ is:

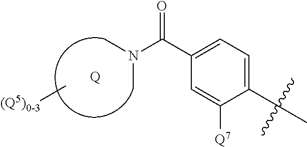

where Q⁷ is hydrogen, hydroxyl, halo, alkyl or alkoxy;

ring Q is a 5 to 7 membered heterocyclyl or heteroaryl ring;

each Q⁵ is independently selected from halo, hydroxy, amino, cyano, oxo, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R¹⁴)(R¹⁵), —R^{u}N(R^{x})C(J)OR^{x}, —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups;

each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

J is O;

each R^{u} is independently alkylene or a direct bond;

R^{w} is alkyl;

each R^{x} is independently hydrogen, alkyl, hydroxyalkyl or alkoxyalkyl; and

t is an integer from 0-2.

In one embodiment, R¹ is:

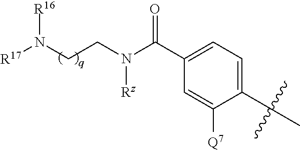

where Q⁷ is alkyl or alkoxy;

R^{z}, R¹⁶ and R¹⁷ are selected as follows:

(i) R^{z}, R¹⁶ and R¹⁷ are each independently hydrogen, alkyl, cycloalkyl or cycloalkylalkyl;

(ii) R^{z} is selected from hydrogen and alkyl; and R¹⁶ and R¹⁷ together with the nitrogen atom on which they are substituted form an optionally substituted 5-7 membered heterocyclyl or heteroaryl ring; where the substituents when present are selected from alkyl, cycloalkyl, cycloalkylalkyl, aminoalkyl, alkoxy, amino and hydroxyl;

(iii) R¹⁶ is selected from hydrogen and alkyl; and R^{z} and R¹⁷ together with the atoms on which they are substituted form an optionally substituted 5-7 membered heterocyclyl ring; where the substituents when present are selected from one, two or three Q⁵ groups;

each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}C(J)OR^{x}, —R^{u}N(R^{x})C(J)OR^{x}, R^{u}C(J)N(R¹⁴)(R¹⁵), and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;

J is O;

each R^{u} is independently alkylene or a direct bond;

R^{w} is alkyl;

each R^{x} is independently hydrogen, alkyl, hydroxyalkyl or alkoxyalkyl;

t is an integer from 0-2; and

q is 1 or 2.

In one embodiment, R¹ is:

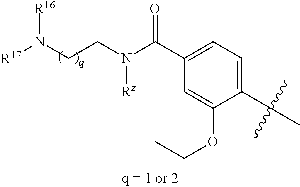

where the variables are as described elsewhere herein. In one embodiment, R^{z}, R¹⁶ and R¹⁷ are selected as follows:

(i) R^{z}, R¹⁶ and R¹⁷ are each independently hydrogen, alkyl, cycloalkyl or cycloalkylalkyl;

(ii) R^{z} is selected from hydrogen and alkyl; and R¹⁶ and R¹⁷ together with the nitrogen atom on which they are substituted form an optionally substituted 5-7 membered heterocyclyl or heteroaryl ring; where the substituents when present are selected from alkyl, cycloalkyl, cycloalkylalkyl, aminoalkyl, alkoxy, amino and hydroxyl;

(iii) R¹⁶ is selected from hydrogen and alkyl; and R^{z} and R¹⁷ together with the atoms on which they are substituted form an optionally substituted 5-7 membered heterocyclyl ring; where the substituents when present are selected from one or two Q⁵ groups;

each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, alkoxy, —COOH, —R^{u}OR^{x}, —R^{u}N(R^{x})C(J)OR^{x}, R^{u}C(J)N(R¹⁴)(R¹⁵), and —R^{u}N(R^{x})S(O)ₜR^{w}, where when Q⁵ is amino, alkyl, cycloalkyl, aryl, heteroaryl or heterocyclyl, Q⁵ is optionally substituted with one or two Q⁶ groups selected from alkyl, alkenyl, alkynyl, cycloalkyl, halo, hydroxyl and amino;

J is O;

each R^{u} is independently alkylene or a direct bond;

R^{w} is alkyl;

each R^{x} is independently hydrogen, alkyl, hydroxyalkyl or alkoxyalkyl;

t is an integer from 0-2; and

q is 1 or 2.

In one embodiment, R¹ is:

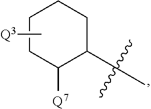

where Q⁷ is hydrogen, hydroxyl, halo, alkyl or alkoxy;

Q³ is —R^{u}OR^{x}, —R^{u}N(R^{y})(R^{z}), —R^{u}C(J)N(R^{y})(R^{z}), —R^{u}N(R^{x})C(J)R^{x} or —R^{u}S(O)ₜN(R^{y})(R^{z});

each R^{u} is independently alkylene or a direct bond;

each R^{x} is independently hydrogen or alkyl;

R^{y} and R^{z} are each independently selected from (i) and (ii) below:

(i) R^{y} is hydrogen; and R^{z} is hydrogen, alkyl, heterocyclyl or heteroaryl; where R^{y} and R^{z} are each optionally substituted with one, two or three Q⁵ groups; or

(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups;

each Q⁵ is independently selected from halo, hydroxy, amino, alkoxy and alkyl; and

J is O.

In one embodiment, the compound provided herein is of Formula I, IA, IB, IC or ID or a pharmaceutically acceptable salt thereof, where R² and R³ are each C₁-C₆alkyl;

E is CO or SO₂;

R¹ is phenyl, pyridyl, pyrazolyl, cyclohexyl, or tetrahydropyranyl ring;

R¹ is optionally substituted with 1 or 2 substituents Q³, each Q³ is independently selected from halo, cyano, hydroxyl, alkyl, alkyloxy, haloalkyloxyl, hydroxyalkyl, alkylthio, 4,5-dihydrooxazol-2-yl amino, pyrimidin-2-amino, piperidin-1-yl, 1-methylpiperidin-4-yl, pyrrolidin-1-yl, —COOH, —R^{u}N(R^{y})(R^{z}), —R^{u}C(J)N(R^{y})(R^{z}), and —R^{u}N(R^{x})S(O)ₜR^{w}, where the alkyl, haloalkyl, aminoalkyl, alkenyl, alkynyl, cycloalkyl, cycloalkenyl, aryl, heteroaryl, and heterocyclyl groups are optionally substituted with one to three Q⁴ groups, each Q⁴ is independently selected from halo, hydroxyl, amino, alkyl, cycloalkyl, haloalkyl and hydroxyalkyl;
each R^{u} is independently alkylene or a direct bond;
R^{w} is alkyl;
each R^{x} is independently hydrogen, alkyl or hydroxyalkyl;
R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R¹⁴)(R¹⁵), —R^{u}N(R^{x})C(J)OR^{x}, —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, and amino;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one or more, in one embodiment, one, two or three Q⁸ groups;
each Q⁸ is independently selected from halo, hydroxy, alkyl, alkoxy, and haloalkyl;
each of R¹⁴ and R¹⁵ is optionally substituted with one or two halo, hydroxy, alkyl, alkoxy or haloalkyl;
J is O; and
t is an integer from 0-2.

In one embodiment, the compound provided herein is of Formula II or II-1:

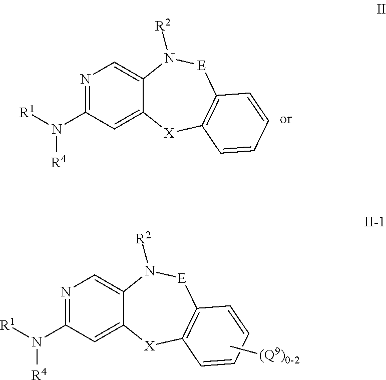

or a pharmaceutically acceptable salt thereof, where X is O or S(O)₀₋₂; each Q⁹ is independently halo, alkyl, haloalkyl, hydroxyl or alkoxy; and the other variables are as described elsewhere herein. In certain embodiment, each Q⁹ is independently selected from chloro, fluoro, methyl, methoxy or ethoxy.

In one embodiment, the compound provided herein is of Formula IIA or IIA-1:

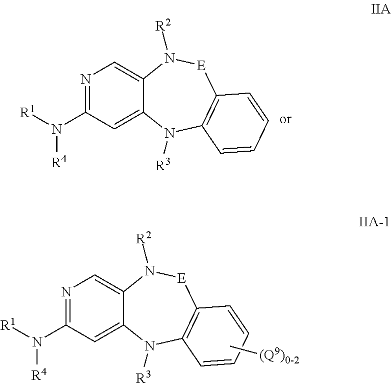

or a pharmaceutically acceptable salt thereof, where the variables are as described elsewhere herein.

In one embodiment, the compound provided herein is of Formula II, II-1, IIA, IIA-1 or a pharmaceutically acceptable salt thereof, where

R² is alkyl or deuteroalkyl;

R³ is alkyl, deuteroalkyl, cycloalkyl or SO₂R¹⁹;

R⁴ hydrogen or alkyl;

E is CO or SO₂;

R¹⁹ is alkyl;

R¹ is aryl, heteroaryl, heterocyclyl or cycloalkyl;

R¹ is optionally substituted with 1 or 2 substituents Q³, each Q³ is independently selected from halo, cyano, alkyl, haloalkyl, alkenyl, alkynyl, cycloalkyl, cycloalkenylalkyl, heteroaryl, heterocyclyl, heterocyclylalkyl, —COOH, —R^{u}OR^{x}, —R^{u}N(R^{y})(R^{z}), —R^{u}C(J)N(R^{y})(R^{z}), —R^{u}S(O)ₜN(R^{y})(R^{z}), and —R^{u}N(R^{x})S(O)ₜR^{w}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;
each R^{u} is independently alkylene or a direct bond;
R^{w} is alkyl or amino;
each R^{x} is independently hydrogen, alkyl or hydroxyalkyl;
R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups;
each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R^{x})C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one or more, in one embodiment, one, two or three Q⁸ groups;
each Q⁸ is independently selected from halo, hydroxy, alkyl, alkoxy, and haloalkyl;
each Q⁹ is independently halo, alkyl, haloalkyl, hydroxyl or alkoxy;
each of R¹⁴ and R¹⁵ is optionally substituted with one or two halo, hydroxy, alkyl, alkoxy or haloalkyl;
J is O; and
t is an integer from 0-2.

In one embodiment, the compound provided herein is of Formula III:

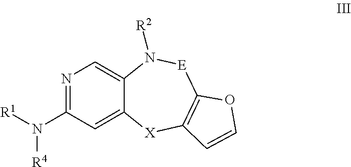

or a pharmaceutically acceptable salt thereof, where X is O or S(O)₀₋₂, and the other variables are as described elsewhere herein.

In one embodiment, the compound provided herein is of Formula IIIA:

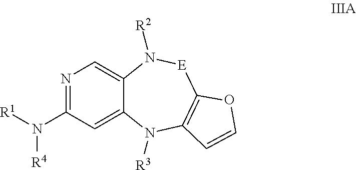

or a pharmaceutically acceptable salt thereof, where the variables are as described elsewhere herein.

In one embodiment, the compound provided herein is of Formula III, IIIA or a pharmaceutically acceptable salt thereof, where R² and R³ are alkyl or deuteroalkyl;

R⁴ hydrogen or alkyl;

E is CO or SO₂;

R¹ is aryl, heteroaryl, heterocyclyl or cycloalkyl;

R¹ is optionally substituted with 1 or 2 substituents Q³, each Q³ is independently selected from halo, cyano, alkyl, haloalkyl, alkenyl, alkynyl, cycloalkyl, cycloalkenylalkyl, heteroaryl, heterocyclyl, heterocyclylalkyl, —COOH, —R^{u}OR^{x}, —R^{u}N(R^{y})(R^{z}), —R^{u}C(J)N(R^{y})(R^{z}), —R^{u}S(O)ₜN(R^{y})(R^{z}) and —R^{u}N(R^{x})S(O)ₜR^{w}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;
each R^{u} is independently alkylene or a direct bond;
R^{w} is alkyl or amino;
each R^{x} is independently hydrogen, alkyl or hydroxyalkyl;
R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R^{x})C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;
each of R¹⁴ and R¹⁵ is optionally substituted with one or two halo, hydroxy, alkyl, alkoxy or haloalkyl;
J is O; and
t is an integer from 0-2.

In one embodiment, the compound provided herein is of Formula III or IIIA: or a pharmaceutically acceptable salt thereof, where R² and R³ are alkyl or deuteroalkyl;

R⁴ hydrogen or alkyl;

E is CO;

R¹ is aryl or cycloalkyl;

R¹ is optionally substituted with 1 or 2 substituents Q³, each Q³ is independently selected from alkyl, —R^{u}OR^{x}, —R^{u}N(R^{y})(R^{z}) and —R^{u}C(J)N(R^{y})(R^{z});
each R^{u} is independently alkylene or a direct bond;
each R^{x} is independently hydrogen, alkyl or hydroxyalkyl;
R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl, optionally substituted with one or more, in one embodiment, one, two or three Q⁵ groups; each Q⁵ is independently selected from amino and heterocyclyl, where each Q⁵ is optionally substituted with one or two alkyl groups; and
J is O.

In one embodiment, the compound provided herein is of Formula III or IIIA: or a pharmaceutically acceptable salt thereof, where R² and R³ are alkyl or deuteroalkyl;

R⁴ hydrogen or alkyl;

E is CO;

R¹ is phenyl, cyclohexyl, cyclopentyl or cyclobutyl;

R¹ is optionally substituted with 1 or 2 substituents Q³, each Q³ is independently selected from alkyl, —R^{u}OR^{x}, —R^{u}N(R^{y})(R^{z}) and —R^{u}C(J)N(R^{y})(R^{z});
each R^{u} is independently alkylene or a direct bond;
each R^{x} is independently hydrogen, alkyl or hydroxyalkyl;
R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl, optionally substituted with one or more, in one embodiment, one, two or three Q⁵ groups; each Q⁵ is independently selected from amino and heterocyclyl, where each Q⁵ is optionally substituted with one or two alkyl groups; and
J is O.

In one embodiment, the compound provided herein is of Formula IV:

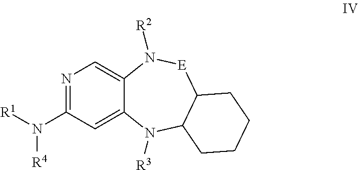

or a pharmaceutically acceptable salt thereof, where the variables are as described elsewhere herein.

In one embodiment, the compound provided herein is of Formula IVA:

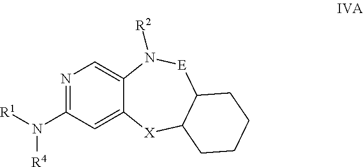

or a pharmaceutically acceptable salt thereof, where X is O or S(O)₀₋₂, and the other variables are as described elsewhere herein.

In one embodiment, the compound provided herein is of Formula IV, IVA or a pharmaceutically acceptable salt thereof, where R² and R³ are alkyl; R⁴ hydrogen or alkyl;

E is CO;

R¹ is aryl or cycloalkyl;

R¹ is optionally substituted with 1 or 2 substituents Q³, each Q³ is independently selected from alkyl, —R^{u}OR^{x}, —R^{u}N(R^{y})(R^{z}) and —R^{u}C(J)N(R^{y})(R^{z});
each R^{u} is independently alkylene or a direct bond;
each R^{x} is independently hydrogen or alkyl;
R^{y} and R^{z} are each independently selected from (i) or (ii) below:
(i) R^{y} is hydrogen or alkyl; and R^{z} is hydrogen or alkyl; or
(ii) R^{y} and R^{z}, together with the nitrogen atom to which they are attached, form a 5 to 7 membered heterocyclyl, optionally substituted with one or two Q⁵ groups; each Q⁵ is independently selected from amino and heterocyclyl, where each Q⁵ is optionally substituted with one or two alkyl groups.

In one embodiment, the compound provided herein is of Formula V:

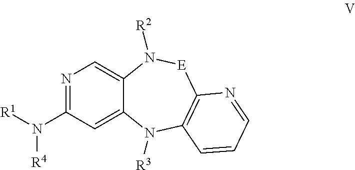

or a pharmaceutically acceptable salt thereof, where the variables are as described elsewhere herein.

In one embodiment, the compound provided herein is of Formula VA:

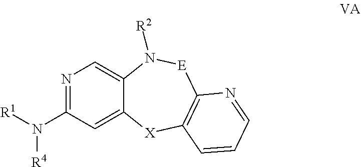

or a pharmaceutically acceptable salt thereof, where X is O or S(O)₀₋₂, and the other variables are as described elsewhere herein.

In one embodiment, the compound provided herein is of Formula V or a pharmaceutically acceptable salt thereof, where R² and R³ are alkyl; R⁴ hydrogen or alkyl;

E is CO;

R¹ is aryl;

R¹ is optionally substituted with 1 or 2 substituents Q³, each Q³ is independently selected from alkyl, —R^{u}OR^{x}, and —R^{u}C(J)N(R^{y})(R^{z});
each R^{u} is independently alkylene or a direct bond;
each R^{x} is independently hydrogen or alkyl;
R^{y} and R^{z}, together with the nitrogen atom to which they are attached, form a 5 to 7 membered heterocyclyl, optionally substituted with one or two Q⁵ groups; each Q⁵ is independently selected from amino and heterocyclyl, where each Q⁵ is optionally substituted with one or two alkyl groups.

In one embodiment, the compound provided herein is of Formula VI or VI-1:

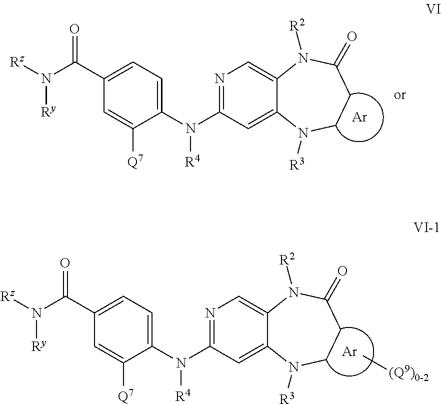

or a pharmaceutically acceptable salt thereof, where ring Ar is 5 or 6 membered aryl or heteroaryl ring; each Q⁹ is independently halo, alkyl, haloalkyl, hydroxyl or alkoxy; and the other variables are as described elsewhere herein. In certain embodiment, each Q⁹ is independently selected from chloro, fluoro, methyl, methoxy or ethoxy.

In one embodiment, the compound provided herein is of Formula VII or VII-1:

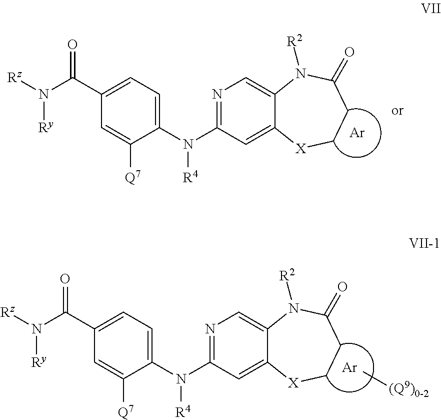

or a pharmaceutically acceptable salt thereof, where ring Ar is 5 or 6 membered aryl or heteroaryl ring; each Q⁹ is independently halo, alkyl, haloalkyl, hydroxyl or alkoxy; X is O or S(O)₀₋₂; and the other variables are as described elsewhere herein. In certain embodiment, each Q⁹ is independently selected from chloro, fluoro, methyl, methoxy or ethoxy.

In one embodiment, the compound provided herein is of Formula VI, VI-1, VII or VII-1 or a pharmaceutically acceptable salt thereof, where ring Ar is 5 or 6 membered aryl or heteroaryl ring;

R² is alkyl or deuteroalkyl;

R³ is alkyl, deuteroalkyl, cycloalkyl or SO₂R¹⁹;

R¹⁹ is alkyl;

Q⁷ is hydrogen, alkyl or alkoxy;

R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R^{x})C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;
each of R¹⁴ and R¹⁵ is optionally substituted with one or two halo, hydroxy, alkyl, alkoxy or haloalkyl;
each Q⁹ is independently halo, alkyl, or alkoxy;
J is O;
each R^{u} is independently alkylene or a direct bond;
R^{w} is alkyl;
each R^{x} is independently hydrogen, alkyl or hydroxyalkyl; and
t is an integer from 0-2.

In one embodiment, the compound provided herein is of Formula VI, VI-1, VII or VII-1 or a pharmaceutically acceptable salt thereof, where ring Ar is 5 or 6 membered aryl or heteroaryl ring;

R² is alkyl or deuteroalkyl;

R³ is alkyl or dueteroalkyl;

Q⁷ is hydrogen, alkyl or alkoxy;

R^{y} and R^{z} are each independently selected from (i) or (ii) below:

(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R^{x})C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

each Q⁹ is independently halo, alkyl, or alkoxy;

J is O;

each R^{u} is independently alkylene or a direct bond;

R^{w} is alkyl;

each R^{x} is independently hydrogen, alkyl or hydroxyalkyl; and

t is an integer from 0-2.

In one embodiment, the compound provided herein is selected from formula VIII-XI:

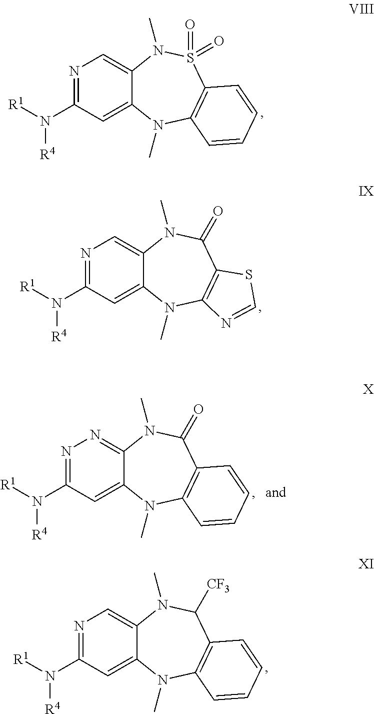

where R⁴ is hydrogen or alkyl;

R¹ is aryl, heteroaryl, heterocyclyl or cycloalkyl;

R¹ is optionally substituted with 1 or 2 substituents Q³, each Q³ is independently selected from halo, cyano, alkyl, haloalkyl, alkenyl, alkynyl, cycloalkyl, cycloalkenylalkyl, heteroaryl, heterocyclyl, heterocyclylalkyl, —COOH, —R^{u}OR^{x}, —R^{u}N(R^{y})(R^{z}), —R^{u}C(J)N(R^{y})(R^{z}), —R^{u}S(O)ₜN(R^{y})(R^{z}) and —R^{u}N(R^{x})S(O)ₜR^{w}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;
each R^{u} is independently alkylene or a direct bond;
R^{w} is alkyl;
each R^{x} is independently hydrogen, alkyl or hydroxyalkyl;
R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R^{x})C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;
each of R¹⁴ and R¹⁵ is optionally substituted with one or two halo, hydroxy, alkyl, alkoxy or haloalkyl;
J is O; and
t is an integer from 0-2.

In one embodiment, the compound provided herein is of formula XII or XII-1

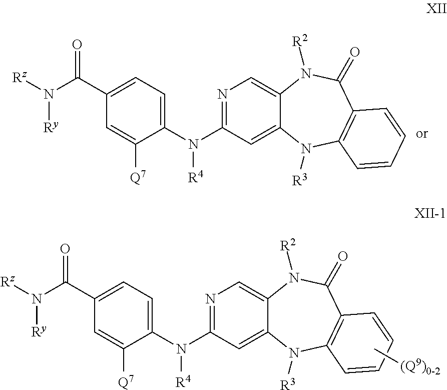

or a pharmaceutically acceptable salt thereof, where Q⁷ is alkyl or alkoxy; each Q⁹ is independently selected from halo, hydroxy, alkyl, alkoxy, and haloalkyl; and the other variables are as described elsewhere herein. In certain embodiment, each Q⁹ is independently selected from chloro, fluoro, methyl, methoxy or ethoxy.

In one embodiment, the compound provided herein is of formula XIII

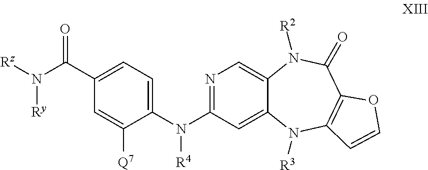

or a pharmaceutically acceptable salt thereof, where Q⁷ is alkyl or alkoxy, and the other variables are as described elsewhere herein.

In one embodiment, the compound provided herein is of formula XII, XII-1 or XIII,

where R² is C₁-C₃alkyl

R³ is C₁-C₃alkyl, C₃-C₆cycloalkyl or SO₂R¹⁹;

R⁴ is hydrogen or C₁-C₃alkyl;

R¹⁹ is C₁-C₃alkyl;

Q⁷ is alkyl or alkoxy;

E is CO or SO₂;

R^{y} and R^{z} are each independently selected from (i) or (ii) below:

(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R^{x})C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;

each R^{u} is independently alkylene or a direct bond;

R^{w} is alkyl;

each R^{x} is independently hydrogen, alkyl or hydroxyalkyl;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

each Q⁹ is independently selected from halo, hydroxy, alkyl, alkoxy, and haloalkyl; J is O; and

t is an integer from 0-2.

In one embodiment, the compound provided herein is of formula XII, XII-1 or XIII

where R² and R³ are each C₁-C₃alkyl or deutero C₁-C₄ alkyl;

R⁴ is hydrogen or C₁-C₃alkyl;

Q⁷ is alkyl or alkoxy;

E is CO or SO₂;

R¹ is phenyl, pyridyl, pyrazolyl, cyclohexyl, or tetrahydropyranyl ring;

R¹ is optionally substituted with 1 or 2 substituents Q³, each Q³ is independently selected from halo, cyano, alkyl, haloalkyl, alkenyl, alkynyl, cycloalkyl, cycloalkenylalkyl, heteroaryl, heterocyclyl, heterocyclylalkyl, —COOH, —R^{u}OR^{x}, —R^{u}N(R^{y})(R^{z}), —R^{u}C(J)N(R^{y})(R^{z}), —R^{u}S(O)ₜN(R^{y})(R^{z}) and —R^{u}N(R^{x})S(O)ₜR^{w}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;

each R^{u} is independently alkylene or a direct bond;

R^{w} is alkyl;

each R^{x} is independently hydrogen, alkyl or hydroxyalkyl;

R^{y} and R^{z} are each independently selected from (i) or (ii) below:

(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or

(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R^{x})C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;

R¹⁴ and R¹⁵ are each independently (i) or (ii) below:

(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or

(ii) R¹⁴ and R¹⁵, together with the nitrogen atom to which they are attached, form a heterocyclyl or heteroaryl, optionally substituted with one or more, in one embodiment, one, two or three Q⁸ groups; each Q⁸ is independently selected from halo, hydroxy, alkyl, alkoxy, and haloalkyl;

each of R¹⁴ and R¹⁵ is optionally substituted with one or two halo, hydroxy, alkyl, alkoxy or haloalkyl;

each Q⁹ is independently selected from halo, alkyl and alkoxy;

J is O; and

t is an integer from 0-2.

In one embodiment, the compound provided herein is of formula XIV

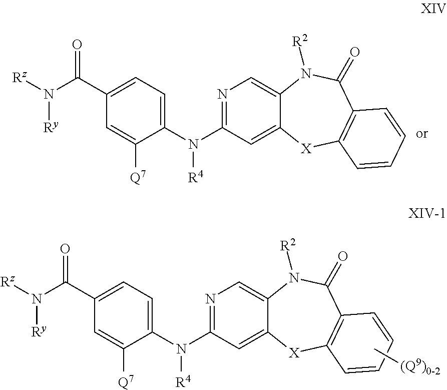

or a pharmaceutically acceptable salt thereof, where X is O or S(O)₀₋₂; each Q⁹ is independently selected from halo, alkyl and alkoxy; Q⁷ is alkyl or alkoxy, and the other variables are as described elsewhere herein. In certain embodiment, each Q⁹ is independently selected from chloro, fluoro, methyl, methoxy or ethoxy.

In one embodiment, the compound provided herein is of formula XIV or XIV-1, where

X is O or S(O)₀₋₂;

R² is C₁-C₃alkyl;

R⁴ is hydrogen or C₁-C₃alkyl;

Q⁷ is alkoxy;

each Q⁹ is independently selected from halo, alkyl, and alkoxy;

E is CO or SO₂; and

R^{y} and R^{z}, together with the nitrogen atom to which they are attached, form a 5 to 7 membered heterocyclyl.

In one embodiment, the compound provided herein is of Formula XV:

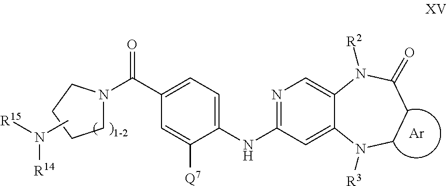

or a pharmaceutically acceptable salt thereof, where ring Ar is 5 or 6 membered aryl or heteroaryl ring, optionally substituted with one, two or three groups selected from halo, alkyl, and alkoxy; and the other variables are as described elsewhere herein.

In one embodiment, the compound provided herein is of Formula XV or a pharmaceutically acceptable salt thereof, where ring Ar is

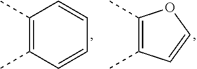

optionally substituted with one, two or three groups selected from halo, alkyl and alkoxy;

R² is alkyl or deuteroalkyl;

R³ is alkyl or deuteroalkyl;

Q⁷ is hydrogen, alkyl or alkoxy;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl; cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one or two Q⁸ groups; each Q⁸ is independently selected from halo, hydroxy, alkyl, alkoxy, and haloalkyl; and
R¹⁴ and R¹⁵ are each independently, optionally substituted with one or two halo, hydroxy, alkyl, alkoxy or haloalkyl.

In one embodiment, the compound is selected from Tables 1, and 1A or a pharmaceutically acceptable salt thereof.

Also provided herein are isotopically enriched analogs of the compounds provided herein. Isotopic enrichment (for example, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); and Wade D, Chem. Biol. Interact. 117: 191 (1999).

Isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g, Foster et al., Adv. Drug Res., vol. 14, pp. 1-36 (1985); Kushner et al., Can. J. Physiol. Pharmacol., vol. 77, pp. 79-88 (1999)).

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, will provide a similar kinetic isotope effects.

Preparation of Compounds

The compounds provided herein can be prepared by methods known to one of skill in the art and following procedures similar to those described in the Examples section herein and routine modifications thereof.

Certain exemplary reaction schemes for the preparation of compounds are illustrated below. In Schemes 1-6 below, the variables are as defined elsewhere herein. For example, in certain embodiments, ring M is aryl, cycloalkyl, heterocyclyl or heteroaryl ring; and R is alkyl or aryl.

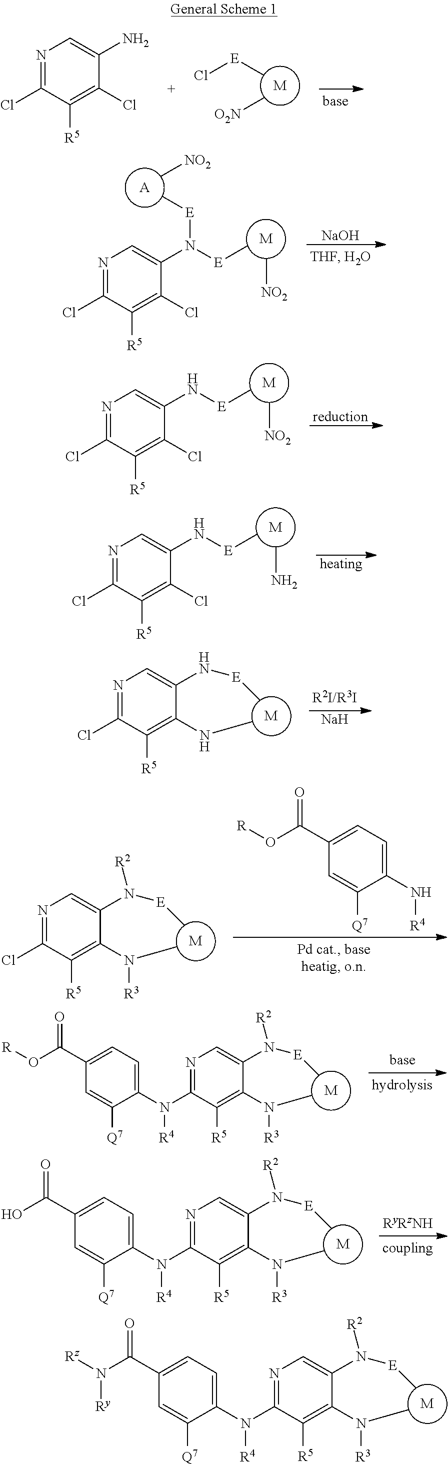

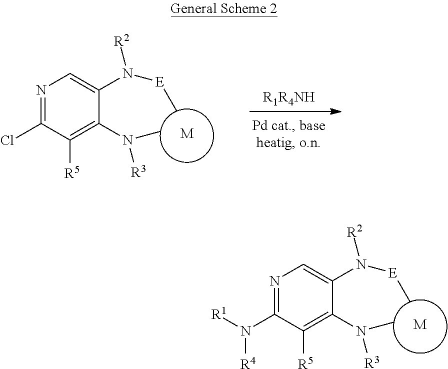

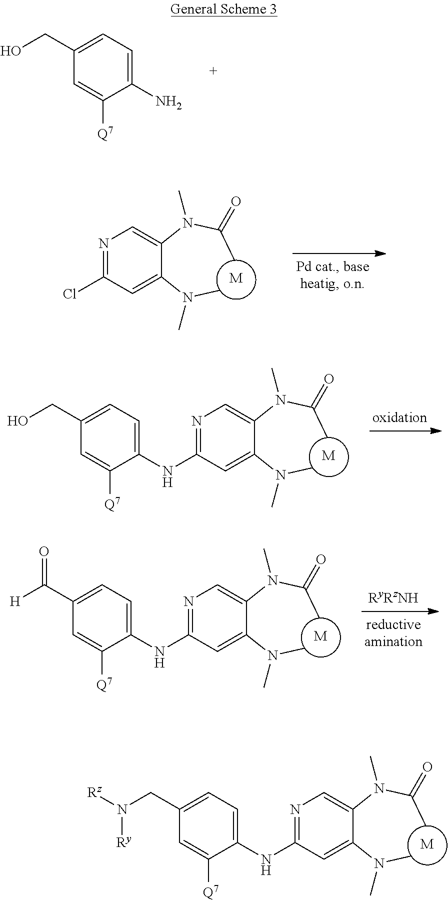

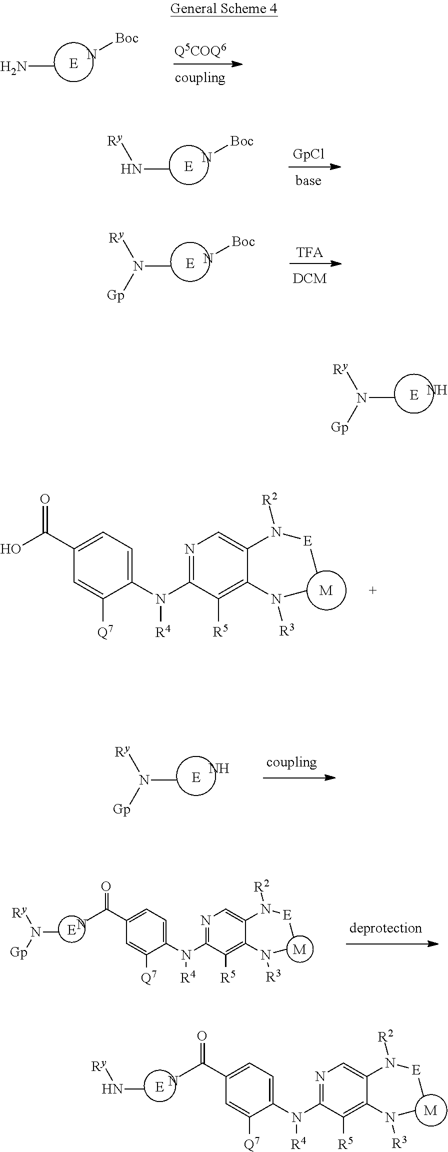

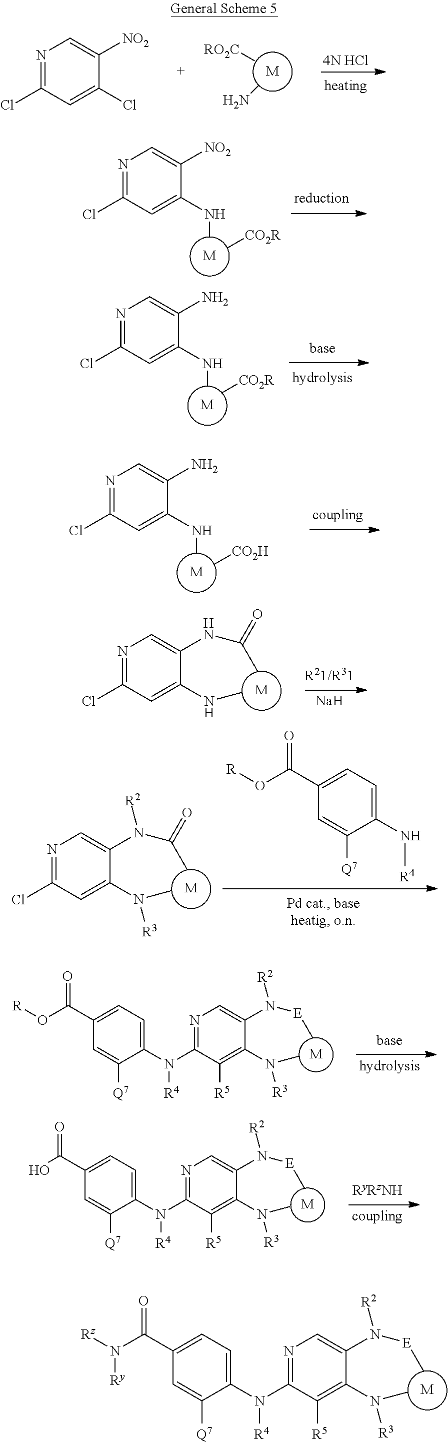

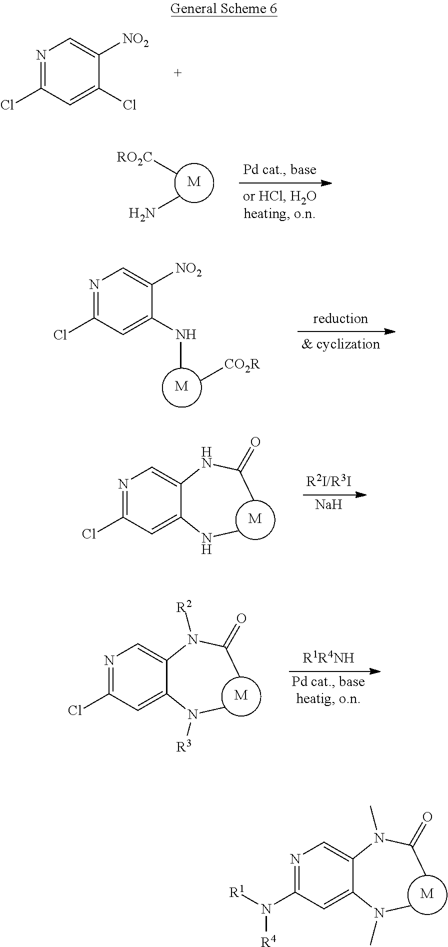

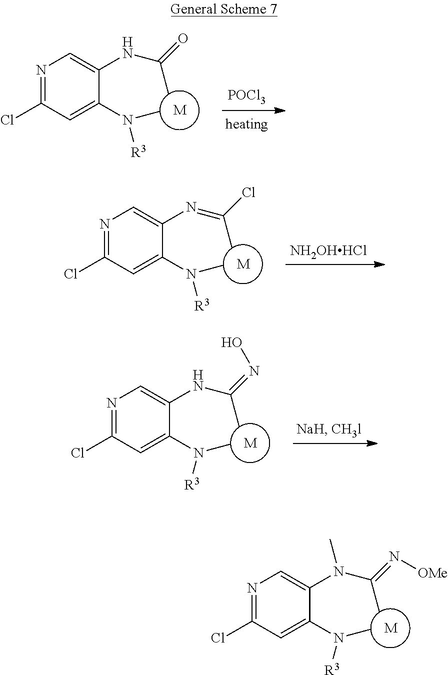

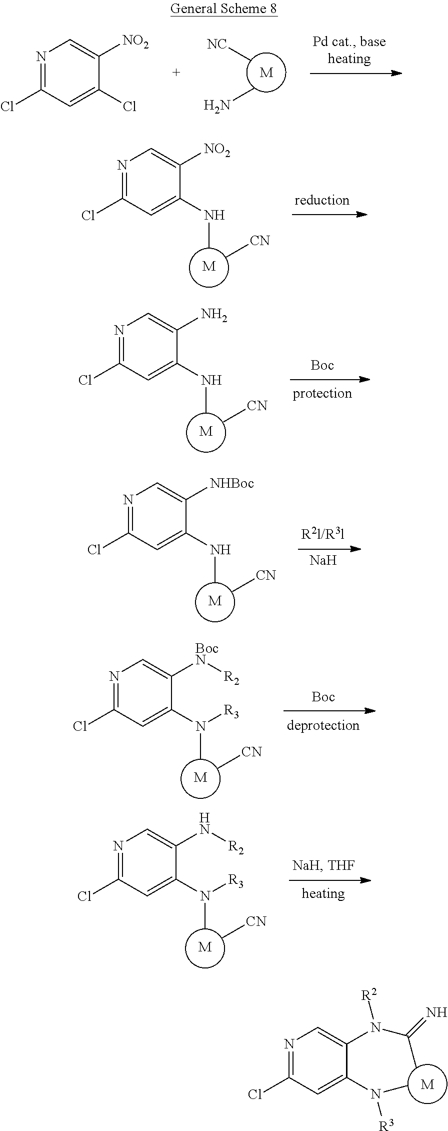

Formulation of Pharmaceutical Compositions

In one embodiment, the pharmaceutical compositions provided herein contain therapeutically effective amounts of one or more of compounds provided herein that are useful in the prevention, treatment, or amelioration of one or more of the symptoms and/or progression of ERK5-mediated diseases and/or diseases mediated by one or more BET family proteins, including BRD2, BRD3, BRD4 and BRDT.

The compositions contain one or more compounds provided herein. The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. The concentrations of the compounds in the compositions are effective for delivery of an amount, upon administration, that treats, prevents, or ameliorates one or more of the symptoms and/or progression of ERK5-mediated diseases and/or diseases mediated by one or more BET family proteins, including BRD2, BRD3, BRD4 and BRDT.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of ERK5-mediated diseases and/or diseases mediated by one or more BET family proteins, including BRD2, BRD3, BRD4 and BRDT.

In certain embodiments, a therapeutically effective dosage should produce a serum concentration of active ingredient of from about 0.1 ng/ml to about 50-100 μg/ml. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing ERK5-mediated diseases and/or diseases mediated by one or more BET family proteins, including BRD2, BRD3, BRD4 and BRDT. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% or about 75-95%.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as ERK5-mediated diseases and/or diseases mediated by one or more BET family proteins, including BRD2, BRD3, BRD4 and BRDT. It is to be understood that such combination therapy constitutes a further aspect of the compositions and methods of treatment provided herein.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

1.1.1 Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

1.1.2 Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

1.1.3 Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

1.1.4 Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

1.1.5 Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

1.1.6 Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and U.S. Pat. Nos. 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

1.1.7 Targeted Formulations

The compounds provided herein, or pharmaceutically acceptable salts thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of disease associated with ERK5 activity and/or activity of one or more BET family proteins, including BRD2, BRD3, BRD4 and BRDT, and a label that indicates that the compound or pharmaceutically acceptable salt thereof is used for treatment, prevention or amelioration of one or more symptoms or progression of ERK5-mediated diseases and/or diseases mediated by one or more BET family proteins, including BRD2, BRD3, BRD4 and BRDT.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

Evaluation of the Activity of the Compounds

Standard physiological, pharmacological and biochemical procedures are available for testing the compounds to identify those that possess a desired biological activity. The inhibitory activity of the compounds provided herein against ERK5 and one or more BET family proteins, including BRD2, BRD3, BRD4 and BRDT, can be readily detected using the assays described herein, as well as assays generally known to those of ordinary skill in the art. Such assays include, but are not limited to assays to determine effect the compounds provided herein on: modulation of cytokines produced by human CD4+ T cells stimulated with PMA/ionomycin, inhibition of cytokine response by primary cynomolgus monkey PBMCs stimulated with LPS, inhibition of cytokine response by primary human PBMCs stimulated with PMA/ionomycin or LPS, inhibition of cytokine response by in vitro-polarized human and murine Th17 cells stimulated with PMA/ionomycin, inhibition of TGF-β-induced fibrotic response in primary human lung fibroblasts, inhibition of pro-inflammatory cytokine response by primary diseased human lung fibroblasts stimulated with IL-17A or IL-17F, inhibition of pro-inflammatory cytokine response by primary human keratinocytes stimulated with IL-17A, inhibition of pro-inflammatory cytokine response by primary human synovial fibroblasts stimulated with IL-17A, TNF-α, or both, inhibition of pro-inflammatory cytokine response to TLR2 or TLR4 agonism in primary human umbilical vein endothelial cells, and inhibition of pro-inflammatory cytokine response by primary human corneal epithelial cells stimulated with IFNγ, IL-1β or IL-17A.

Anti-cancer activity of the compounds provided herein, either alone or in combination with standard of care chemotherapy, such as Ara-C, can be determined, for example, in the MV-4-11 cell proliferation assay.

Anti-inflammatory activity of the compounds can be determined, for example, in DNFB-induced contact hypersensitivity ear inflammation mouse model, imiquimod (IMQ, Aldara™)-induced acute model of psoriasis in mouse, and collagen-induced arthritis in mouse model.

Exemplary methods are described in the Examples section.

Methods of Use of the Compounds and Compositions

Methods of use of the compounds and compositions are also provided. The methods involve both in vitro and in vivo uses of the compounds and compositions. In one embodiment, provided herein are methods of treating a disease in a subject comprising administering to the subject a compound of formula I or pharmaceutically acceptable salt of the compound of formula I. In one embodiment, the disease is mediated by a ERK5 kinase and/or by one or more BET family proteins, including BRD2, BRD3, BRD4 and BRDT. In one embodiment, the disease is modulated by a cytokine, including but not limited to, IL-17, IL-6, and GCSF.

In certain embodiments, the compounds provided herein are useful in treating inflammatory diseases in the airways, such as nonspecific bronchial hyper-reactivity, chronic bronchitis, cystic fibrosis, and acute respiratory distress syndrome (ARDS).

As known to one of skill in the art, increased IL-17A and IL-17F levels correlate with clinical asthma severity. (Al-Ramli W, et al. J Allergy Clin Immunol. 2009; 123: 1185-1187; and Chakir J, et al. J Allergy Clin Immunol. 2003; 111:1293-1298). IL-17 is believed to be involved in the development of respiratory diseases such as asthma and COPD. The IL-17-mediated recruitment and activation of neutrophils in the airways is further thought to mediate other inflammatory diseases in the airways, such as nonspecific bronchial hyperreactivity, chronic bronchitis, cystic fibrosis, and ARDS. (Linden A, et al. Eur Respir J. 2005; 25: 159-172; Halwani R, et al. Chest 2013; 143:494-501). As reported by Yamauchi K. et al., Allergol Int. 2007; 56:321-9), airway remodeling (in large part mediated by myofibroblasts) can lead to irreversible airflow limitation and an increase of airway hyperresponsiveness. In murine studies, McKinley L et al., J Immunol 2008; 181:4089-97, demonstrated that while both Th2 and Th17 cells are able to induce airway hyperresponsiveness (AHR), Th17 cell-mediated airway inflammation and AHR are steroid resistant, indicating a potential role for Th17 cells in steroid-resistant asthma. In certain embodiments, the compounds provided herein inhibit IL-17A- and IL-17F-mediated cytokine response.

In certain embodiments, the compounds provided herein are useful in treating asthma and idiopathic lung fibrosis or idiopathic pulmonary fibrosis (IPF), pulmonary fibrosis, and interstitial lung disease. As known to one of skill in the art, the differentiation of fibroblasts into cell types called myofibroblasts occurs during wound healing, when the cells contribute to the deposition of extracellular matrix (ECM) in the transient process of wound repair. In chronic inflammatory diseases such as asthma, pathological tissue remodeling often occurs, and is mediated by the functions of increased numbers of myofibroblasts in the diseased tissue, see Hinz, B. et al. Am J Pathol. 2007; 170: 1807-1816. In certain embodiments, the compounds provided herein prevent or reduce TGF-β-induced myofibroblast differentiation, as measured by the expression of alpha smooth muscle actin (α-SMA), a hallmark of myofibroblast differentiation (Serini, G. and Gabbiani, G. 1999; Exp. Cell Res. 250: 273-283).

In certain embodiments, the compounds provided herein are useful in treating psoriasis, chronic plaque psoriasis, psoriatic arthritis, acanthosis, atopic dermatitis, various forms of eczema, contact dermatitis (includes allergic dermatitis), systemic sclerosis (scleroderma), wound healing, and drug eruption.

In certain embodiments, the compounds provided herein are useful in treating arthritis and osteoarthritis.

In certain embodiments, the compounds provided herein are useful in treating dry eye syndrome (or keratoconjunctivitis sicca (KCS)).

In certain embodiments, the compounds provided herein are useful as reversible male contraceptives.

In certain embodiments, the compounds provided herein are useful in treating oncological disorders. In another embodiment, the disease is cancer or a proliferation disease. In a further embodiment, the disease is lung, colon, breast, prostate, liver, pancreas, brain, kidney, ovaries, stomach, skin, and bone cancers, gastric, breast, pancreatic cancer, glioma, and hepatocellular carcinoma, papillary renal carcinoma, head and neck squamous cell carcinoma, leukemias, lymphomas, myelomas, and solid tumors. In certain embodiments, the compounds provided herein are useful in treating various forms of leukemia, including acute myeloid leukemia (AML) and chronic lymphocytic leukemia.

In certain embodiments, the compounds provided herein are useful in treating neuropathic and nociceptive pain, chronic or acute, such as, without limitation, allodynia, inflammatory pain, inflammatory hyperalgesia, post herpetic neuralgia, neuropathies, neuralgia, diabetic neuropathy, HIV-related neuropathy, nerve injury, rheumatoid arthritic pain, osteoarthritic pain, burns, back pain, ocular pain, visceral pain, cancer pain, dental pain, headache, migraine, carpal tunnel syndrome, fibromyalgia, neuritis, sciatica, pelvic hypersensitivity, pelvic pain, post operative pain, post stroke pain, and menstrual pain.

In certain embodiments, the compounds provided herein are useful in treating Alzheimer's disease (AD), mild cognitive impairment (MCI), age-associated memory impairment (AAMI), multiple sclerosis, Parkinson's disease, vascular dementia, senile dementia, AIDS dementia, Pick's disease, dementia caused by cerebrovascular disorders, corticobasal degeneration, amyotrophic lateral sclerosis (ALS), Huntington's disease, diminished CNS function associated with traumatic brain injury.

In one embodiment, the disease is inflammation, arthritis, rheumatoid arthritis, spondylarthropathies, gouty arthritis, osteoarthritis, juvenile arthritis, and other arthritic conditions, systemic lupus erthematosus (SLE), skin-related conditions, psoriasis, eczema, Sjögren's_syndrome, burns, dermatitis, neuroinflammation, allergy pain, autoimmune myositis, neuropathic pain, fever, pulmonary disorders, lung inflammation, adult respiratory distress syndrome, pulmonary sarcoisosis, asthma, silicosis, chronic pulmonary inflammatory disease, and chronic obstructive pulmonary disease (COPD), cardiovascular disease, arteriosclerosis, myocardial infarction (including post-myocardial infarction indications), thrombosis, congestive heart failure, cardiac reperfusion injury, as well as complications associated with hypertension and/or heart failure such as vascular organ damage, restenosis, cardiomyopathy, stroke including ischemic and hemorrhagic stroke, reperfusion injury, renal reperfusion injury, ischemia including stroke and brain ischemia, and ischemia resulting from cardiac/coronary bypass, neurodegenerative disorders, liver disease and nephritis, gastrointestinal conditions, inflammatory bowel disease, Crohn's disease, gastritis, irritable bowel syndrome, ulcerative colitis, ulcerative diseases, gastric ulcers, viral and bacterial infections, sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, herpes virus, myalgias due to infection, influenza, autoimmune disease, graft vs. host reaction and allograft rejections, treatment of bone resorption diseases, osteoporosis, multiple sclerosis, cancer, leukemia, lymphoma, colorectal cancer, brain cancer, bone cancer, epithelial call-derived neoplasia (epithelial carcinoma), basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovarian cancer, cervical cancer, lung cancer, breast cancer, skin cancer, squamus cell and/or basal cell cancers, prostate cancer, renal cell carcinoma, and other known cancers that affect epithelial cells throughout the body, chronic myelogenous leukemia (CML), acute myeloid leukemia (AML) and acute promyelocyte leukemia (APL), chronic lymphocytic leukemia (CCL), angiogenesis including neoplasia, metastasis, central nervous system disorders, central nervous system disorders having an inflammatory or apoptotic component, Alzheimer's disease, Parkinson's disease, Huntington's disease, amyotrophic lateral sclerosis, spinal cord injury, and peripheral neuropathy, Canine B-Cell Lymphoma. In a further embodiment, the disease is inflammation, arthritis, rheumatoid arthritis, spondylarthropathies, gouty arthritis, osteoarthritis, juvenile arthritis, and other arthritic conditions, systemic lupus erthematosus (SLE), skin-related conditions, psoriasis, eczema, dermatitis, pain, pulmonary disorders, lung inflammation, adult respiratory distress syndrome, pulmonary sarcoidosis, asthma, chronic pulmonary inflammatory disease, and chronic obstructive pulmonary disease (COPD), cardiovascular disease, arteriosclerosis, myocardial infarction (including post-myocardial infarction indications), congestive heart failure, cardiac reperfusion injury, inflammatory bowel disease, Crohn's disease, gastritis, irritable bowel syndrome, leukemia, lymphoma. In another aspect, the invention provides a method of treating a kinase mediated disorder in a subject comprising: administering to the subject identified as in need thereof a compound, pharmaceutically acceptable salt, ester or prodrug of formula I.

In one embodiment, the compounds provided herein are useful in treating autoimmune and inflammatory diseases or conditions, including but not limited to, rheumatoid arthritis, osteoarthritis, acute gout, psoriasis, systemic lupus erythematosus, multiple sclerosis, inflammatory bowel disease (Crohn's disease and Ulcerative colitis), asthma, chronic obstructive airways disease, pneumonitis, myocarditis, pericarditis, myositis, eczema, dermatitis, alopecia, vitiligo, bullous skin diseases, nephritis, vasculitis, atherosclerosis, Alzheimer's disease, depression, retinitis, uveitis, scleritis, hepatitis, pancreatitis, primary biliary cirrhosis, sclerosing cholangitis, Addison's disease, hypophysitis, thyroiditis, type I diabetes and acute rejection of transplanted organs. In certain embodiments, the autoimmune and inflammatory diseases or conditions include acute inflammatory conditions such as acute gout, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, vasculitis with organ involvement and acute rejection of transplanted organs.

In certain embodiments, autoimmune and inflammatory diseases or conditions include diseases or conditions which involve inflammatory responses to infections with bacteria, viruses, fungi, parasites or their toxins, such as sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, postsurgical syndromes, sarcoidosis, Herxheimer reactions, encephalitis, myelitis, meningitis, malaria, SIRS associated with viral infections such as influenza, herpes zoster, herpes simplex, coronavirus.

In one embodiment, the disease or condition is associated with systemic inflammatory response syndrome, such as sepsis, burns, pancreatitis, major trauma, haemorrhage and ischaemia. In this embodiment, the compound is administered at the point of diagnosis to reduce the incidence of: SIRS, the onset of shock, multi-organ dysfunction syndrome, which includes the onset of acute lung injury, ARDS, acute renal, hepatic, cardiac and gastro-intestinal injury and mortality. In another embodiment, the compound is administered prior to surgical or other procedures associated with a high risk of sepsis, haemorrhage, extensive tissue damage, SIRS or MODS (multiple organ dysfunction syndrome). In one embodiment, disease is sepsis, sepsis syndrome, septic shock or endotoxaemia. In one embodiment, disease is acute or chronic pancreatitis.

In one embodiment, the compounds provided herein are useful in a method of contraception in a male subject.

Combination Therapy

The compounds provided herein may be administered as the sole active ingredient or in combination with other active ingredients. Other active ingredients that may be used in combination with the compounds provided herein include but are not limited to, compounds known to treat ERK5-mediated diseases and/or diseases mediated by one or more BET family proteins, including BRD2, BRD3, BRD4 and BRDT.

In certain embodiment, the compounds herein are administered in combination with other kinase inhibitors. Exemplary kinase inhibitors are known in the art, and include, but are not limited to commercially available compounds AS703026 and SB203580. In one embodiment, the compounds herein are administered in combination with anti-cancer agents or anti-inflammatory agents or DMARD (Disease-Modifying Antirheumatic Drug). In one embodiment, the compounds herein are administered in combination with Ara-C.

It will be appreciated that every suitable combination of the compounds provided herein with one or more of the aforementioned compounds and optionally one or more further pharmacologically active substances is contemplated herein.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

The compounds provided herein are prepared by the synthetic procedures known in the art and described herein. Synthetic procedures for exemplary compounds are described in Examples 1-11.

Compound Y1 was purchased from Shanghai IS Chemical Technology. Compounds F1 and F2 were purchased from Thonson Technology in P. R. China. All reagents and solvents were obtained from commercial sources, unless otherwise indicated.

Proton nuclear magnetic resonance (¹H NMR) spectra were recorded on a Bruker 400 MHz NMR spectrometer in deuterated solvents using the residual ¹H solvent peak as the internal standard. LC/MS (ES) analysis was performed with an Agilent 1260 Infinity Series LC/MSD using ChemStation software equipped with a C₁₈ reverse phase column (Phenomenex Kinetex 5 m XB-C18 50×2.10 mm column, or Agilent Poreshell 120 EC-C18 3.0×50 mm column), or Agilent 1100 Series LC/MSD using ChemStation software equipped with a C₁₈ reverse phase column (Onyx, monolithic C18 column, 50×2.0 mm; Phenomenex; Torrance, Calif.), and using a binary system of water and acetonitrile with 0.1% trifluoroacetic acid as a modifier. Flash silica gel column chromatography was carried out on a CombiFlash R_{f} system (by Teledyne ISCO) or a Biotage SP-4 automated purification system using pre-packed silica gel cartridges. HPLC purification was performed by using an Agilent 1200 Series with a C₁₈ reverse phase column (Luna 5 u C18 (2) 100 A, 150×21.2 mm, 5 micron; Phenomenex; Torrance, Calif.) and using a binary system of water and acetonitrile with 0.1% acetic acid as a modifier.

### Example 1

### Preparation of 3-(2-Ethoxy-4-(4-(pyrrolidin-1-yl)piperidine-1-carbonyl)phenylamino)-5,11-dimethyl-5H-benzo[e]pyrido[3,4-b][1,4]diazepin-10(11H)-one (Compound 46)

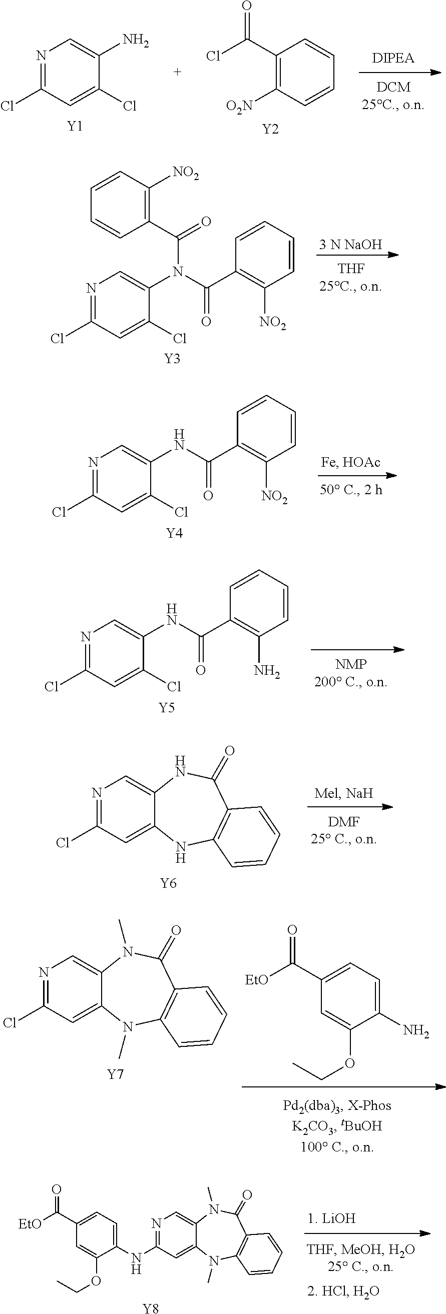

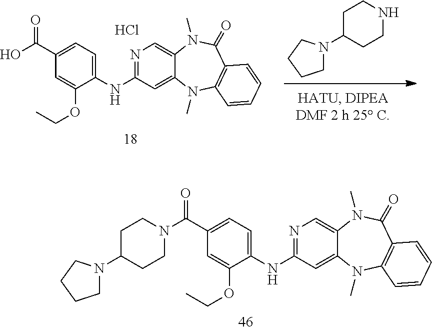

### Step I. N-(4, 6-Dichloropyridin-3-yl)-2-nitro-N-(2-nitrobenzoyl)benzamide (Y3)

2-nitrobenzoyl chloride (Y2, 11.10 mL, 84.0 mmol) was added slowly to a solution of 5-amino-2, 4-dichloropyridine (Y1, 6.520 g, 40.0 mmol) and DIPEA (27.9 mL, 160 mmol) in DCM (100 mL) at 0° C. under N₂. The mixture was then stirred at room temperature for 1.5 h. The reaction mixture was concentrated using a rotavapor. The residue Y3 (brown syrup) was used for the next step without further purification. Some reaction mixture was washed with H₂O. The aqueous phase was extracted once with DCM. The combined organic phase was further washed with sat. NaHCO₃ aqueous solution and sat. NaCl aqueous solution then dried over Na₂SO₄. The DCM phase was filtered and concentrated. The residue was rinsed with small amount of DCM. The precipitate was dried under vacuum to provide compound Y3 as white solid. ¹H NMR (400 MHz, DMSO) δ 8.63 (s, 1H), 8.24 (d, J=8.3, 2H), 8.08 (d, J=1.0, 1H), 7.97-7.80 (m, 4H), 7.75 (t, J=7.8, 2H); ESMS m/z: 461.0 [M+H⁺], 483.0 [M+Na⁺].

### Step II: N-(4, 6-Dichloropyridin-3-yl)-2-nitrobenzamide (Y4)

A suspension of above crude Y3 (˜40.0 mmol) in THF (90 mL) and NaOH aqueous solution (˜3.5 N, 72 mL, ˜252 mmol) was stirred vigorously at room temperature overnight. The reaction mixture was diluted with sat. NaCl solution (˜72 mL). The aqueous phase was extracted with EtOAc (200 mL and ˜100 mL×2). The combined organic phase was washed with sat. NaHCO₃ (˜50 mL×2) and sat. NaCl solution (˜50 mL×2), then dried over NaSO₄. Filtration and concentration under vacuum provided compound Y4 (11.24 g, 90% for two steps) as pale white solid. ¹H NMR (400 MHz, CDCl₃) δ 9.42 (br s, 1H), 8.19 (dd, J=1.0, 8.2, 1H), 7.74 (m, 4H), 7.45 (s, 1H); ESMS m/z: 312.0, 314.0 [M+H⁺], 334.0, 336.0 [M+Na⁺].

### Step III: 2-Amino-N-(4, 6-dichloropyridin-3-yl)benzamide (Y5)

A suspension of compound Y4 (12.48 g, 35.80 mmol) and Fe powder (4.47 g, 80.0 mmol) in HOAc (or HOAc/MeOH, 1:1 v/v, 80 mL) was heated at 50° C. with rigorous stirring under N₂ for 2 h. Additional Fe powder (1.12 g, 10 mmol) was added twice during 2 h. The reaction mixture was stirred at 50° C. for additional 1 h. At 25° C., the extra Fe was removed with a magnetic stir bar. The reaction mixture was quenched with 1 N NaOH aqueous solution and the aqueous solution was saturated with NaCl. The product was extracted by EtOAc. The combined EtOAc phase was washed with sat. NaHCO₃ solution (3×). The combined NaHCO₃ solution was extracted once with EtOAc. The combined EtOAc phase was then washed with sat. NaCl solution and dried over Na₂SO₄. Filtration and concentration under vacuum provided compound Y5 (10.26 g, 91%) as pale white solid. ¹H NMR (400 MHz, DMSO) δ 10.07 (br s, 1H), 8.56 (s, 1H), 7.94 (t, J=1.6, 1H), 7.74 (dd, J=1.4, 8.0, 1H), 7.25 (ddd, J=1.5, 7.1, 8.4, 1H), 6.78 (dd, J=0.9, 8.3, 1H), 6.67-6.58 (m, 1H), 6.53 (br s, 2H); ESMS m/z: 282.1, 284.0 [M+H⁺], 304.0, 306.0 [M+Na⁺].

### Step IV: 3-Chloro-5H-benzo[e]pyrido[3,4-b][1,4]diazepin-10(11H)-one (Y6)

A suspension of compound Y5 (10.263 g, 36.38 mmol) in NMP (80.0 mL) was heated at 200° C. under N₂ for 4 h. At 25° C., 0.33 N HCl aqueous solution (240 mL) was added. The generated suspension was stirred at room temperature for 1 h. The precipitates were filtered and washed with H₂O, then dried under vacuum to provide compound Y6 (8.623 g, 96%) as yellow solid. ¹H NMR (400 MHz, DMSO) δ 10.05 (s, 1H), 8.76 (s, 1H), 7.85 (s, 1H), 7.80-7.68 (m, 1H), 7.47-7.32 (m, 1H), 6.96 (m, 3H); ESMS m/z: 246.1, 248.1 [M+H⁺], 268.0 [M+Na⁺].

### Step V: 3-Chloro-5,11-dimethyl-5H-benzo[e]pyrido[3,4-b][1,4]diazepin-10(11H)-one (Y7)

NaH (60%, 2.15 g, 53.9 mmol) was added portionwise to a suspension of compound Y6 (5.513 g, 22.4 mmol) and MeI (3.36 mL, 53.9 mmol) in anhydrous DMF (67.3 mL) at 0° C. under N₂. The reaction mixture was then stirred at room temperature under N₂ overnight. At 0° C., a 0.25 N HCl aqueous solution (˜200 mL) was added slowly to the generated suspension reaction mixture. The original precipitates (probably NaI) were dissolved, then new precipitates were formed quickly. The mixture was stirred at room temperature for 1 h. Hexanes (50 mL) was added and the mixture was stirred at room temperature for additional 1 h. Filtration and the precipitates were washed with H₂O, then dried under vacuum to provide compound Y7 (5.145 g, 84%) as yellow solid. ¹H NMR (400 MHz, DMSO) δ 8.36 (s, 1H), 7.65 (dd, J=1.7, 7.7, 1H), 7.55-7.44 (m, 1H), 7.34 (s, 1H), 7.23-7.11 (m, 2H), 3.45 (s, 3H), 3.31 (s, 3H); ESMS m/z: 274.1, 276.1 [M+H⁺], 296.1, 298.0 [M+Na⁺].

### Step VI: Ethyl 4-(5,11-dimethyl-10-oxo-10,11-dihydro-5H-benzo[e]pyrido[3,4-b][1,4]diazepin-3-ylamino)-3-ethoxybenzoate (Y8)

A mixture of compound Y7 (4.927 g, 18.0 mmol), ethyl 4-amino-3-ethoxybenzoate (4.520 g, 21.6 mmol), X-Phos (755.1 mg, 1.58 mmol), and K₂CO₃ (14.93 g, 108.0 mmol) in ^{t}BuOH (90 mL) was bubbled with N₂ for 30 sec. Pd₂(dba)₃ (494.5 mg, 0.540 mmol) was added and the mixture was bubbled with N₂ for additional 1 minute. The suspension was then heated at 100° C. (flushed with condenser) under N₂ for 23 h. The reaction mixture was diluted with EtOAc at 25° C. The suspension was filtered through a Celite filter column. The precipitates were washed with EtOAc. The filtrate (˜350 mL) was washed with 0.5 N HCl aqueous solution (˜35 mL×2), and sat. NaCl aqueous solution (˜20 mL×2). The combined aqueous phase was extracted once with EtOAc (˜150 mL). The EtOAc phase was then washed with sat. NaCl aqueous solution (˜25 mL×2), and dried over Na₂SO₄. Filtration and concentrated with a rotavapor. The residue was diluted with CH₃CN. The tiny amount of insoluble yellow solid was filtered and the CH₃CN filtrate was concentrated and dried under vacuum to provide the crude compound Y8 as yellow solid. The crude Y8 was used for next step reaction without further purification. ESMS m/z: 447.2 [M+H⁺], 469.1 [M+Na⁺].

### Step VII: 4-(5,11-Dimethyl-10-oxo-10,11-dihydro-5H-benzo[e]pyrido[3,4-b][1,4]diazepin-3-ylamino)-3-ethoxybenzoic acid (18)

The crude compound Y8 (˜78.5% pure, 10.000 g, ˜17.6 mmol) was dissolved in THF (52.7 mL), MeOH (17.6 mL) and H₂O (17.6 mL). LiOH monohydrate (2.066 g, 49.2 mmol) was added and the mixture was stirred at room temperature for 4 h. Additional LiOH monohydrate (1.033 g, 24.6 mmol) and H₂O (10 mL) were added and the mixture was stirred at room temperature for additional 1.5 h. The reaction mixture was concentrated by rotavapor. The residue was diluted with 0.5 N NaOH aqueous solution (˜200 mL). The basic aqueous phase was washed with ether (˜75 mL×2). The basic aqueous solution was acidified with 3 N HCl solution (˜60 mL) (pH˜1, too acidic). The generated precipitates were filtered and washed with H₂O several times, and rinsed with small amount of EtOAc, then dried under vacuum to provide compound 18 (HCl salt, 6.071 g, 76% for two steps) as tan solid. The above EtOAc rinse solution was mixed with the aqueous filtrate (˜500 mL) and stored at room temperature for 1 week. More precipitates were formed and filtered. The precipitates were washed with H₂O several times then dried under vacuum to provide additional compound 18 (871.0 mg, 11%) as brown solid. ¹H NMR (400 MHz, DMSO) δ 9.65-9.27 (br s, 1H), 8.14 (s, 1H), 8.00 (br s, 1H), 7.68 (dd, J=1.7, 7.7, 1H), 7.64-7.44 (m, 3H), 7.29 (d, J=8.1, 1H), 7.20 (t, J=7.5, 1H), 7.05 (s, 1H), 4.17 (q, J=7.0, 2H), 3.45 (s, 3H), 3.33 (s, 3H), 1.33 (t, J=6.9, 3H); ESMS m/z: 419.1 [M+H⁺].

### Step VIII: 3-(2-Ethoxy-4-(4-(pyrrolidin-1-yl)piperidine-1-carbonyl)phenylamino)-5,11-dimethyl-5H-benzo[e]pyrido[3,4-b][1,4]diazepin-10(11H)-one (46)

To a solution of compound 18 (2.40 g, 5.74 mmol) and 4-pyrrolidin-1-ylpiperidine (1.062 g, 6.88 mmol) in DMF (30 mL) were add DIPEA (4.00 mL, 22.9 mmol) and HATU (3.053 g, 8.03 mmol) at 25° C. The reaction mixture was stirred at room temperature for two hours and then concentrated by a lyophilizer. 200˜250 mL of water was added to the reaction mixture and the precipitates were formed. The mixture was stirred at room temperature for 30 minutes and then filtered. The precipitates were dried in lyophilizer overnight. The residue was combined with crude from another 400 mg scale reaction and then purified by Prep HPLC. Lyophilization of the pure product fractions provided compound 46 (2.15 g, 58%) as white powder. ¹H NMR (400 MHz, CDCl₃) δ 8.11-8.01 (m, 2H), 7.83 (dd, J=1.7, 7.7, 1H), 7.45-7.36 (m, 1H), 7.12 (dd, J=3.1, 10.7, 2H), 7.06-6.96 (m, 3H), 6.53 (s, 1H), 4.13 (q, J=7.0, 2H), 3.57 (s, 3H), 3.31 (s, 3H), 2.93 (s, 2H), 2.75 (s, 4H), 2.47 (s, 1H), 2.02 (s, 1H), 2.02-1.92 (m, 2H), 1.87 (s, 4H), 1.65 (s, 2H), 1.47 (t, J=7.0, 3H); ESMS m/z: 555.3 [M+H⁺], 1131.6 [2M+Na⁺].

### Example 2

### Preparation of 5,11-dimethyl-3-(2-methyl-4-(pyrrolidin-1-ylmethyl)phenylamino)-5H-benzo[e]pyrido[3,4-b][1,4]diazepin-10(11H)-one (Compound 106)

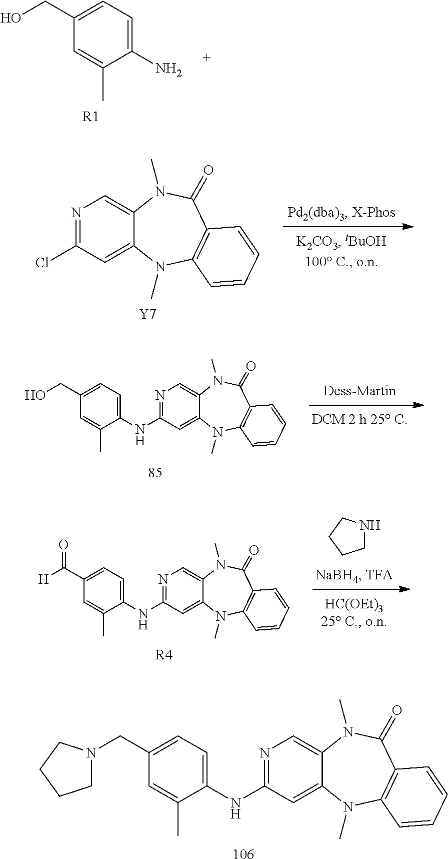

### Step I. 3-(4-(hydroxymethyl)-2-methylphenylamino)-5,11-dimethyl-5H-benzo[e]pyrido[3,4-b][1,4]diazepin-10(11H)-one (85)

A mixture of (4-amino-3-methyl-phenyl)methanol (R1, 89.2 mg, 0.650 mmol), 3-Chloro-5,11-dimethyl-5H-benzo[e]pyrido[3,4-b][1,4]diazepin-10(11H)-one (Y7, 136.8 mg, 0.500 mmol), X-Phos (209.7 mg, 0.044 mmol), and K₂CO₃ (414.6 mg, 3.00 mmol) in tBuOH (5.0 mL) was bubbled with N₂ for 15 sec. Pd₂(dba)₃ (27.5 mg, 0.0300 mmol) was added and the mixture was bubbled with N₂ again for additional 10 sec. The mixture was then heated in a sealed vial at 100° C. under N₂ overnight. At room temperature, the reaction mixture was diluted with DMF, then filtered through a small round filter (PTFE 0.45 μm). The reaction vial and the filter were washed with DMF (3×1 mL). The combined filtrate was purified by HPLC to provide compound 85 (40.5 mg, 22%) as pale white solid. 1H NMR (400 MHz, CDCl₃) δ 7.98 (s, 1H), 7.84 (dd, J=1.7, 7.8, 1H), 7.44-7.34 (m, 2H), 7.30 (s, 1H), 7.25 (d, J=8.2, 1H), 7.17-7.09 (m, 1H), 6.97 (d, J=8.3, 1H), 6.59 (s, 1H), 6.34 (s, 1H), 4.70 (s, 2H), 3.55 (d, J=12.8, 3H), 3.20 (s, 3H), 2.29 (s, 3H); ESMS m/z: 375.2 [M+H⁺], 395.2 [M+Na⁺].

### Step II: 4-(5,11-dimethyl-10-oxo-10,11-dihydro-5H-benzo[e]pyrido[3,4-b][1,4]diazepin-3-ylamino)-3-methylbenzaldehyde (R4)

The Dess-Martin periodinane (37.3 mg, 0.088 mmol) was added to a solution of compound 85 (30.0 mg, 0.080 mmol) in DCM (0.80 mL) at room temperature. The mixture was stirred at room temperature for 2 h. The reaction was quenched with 1 N NaOH (˜0.1 mL) and water (˜0.5 mL). The mixture was extracted with EtOAc. The combined EtOAc phase was washed with sat. NaHCO₃ and sat NaCl aqueous solutions, then dried over Na2SO4. Filtration, concentration, followed by HPLC purification provided compound R4 (8.2 mg, 28%) as light yellow solid. 1H NMR (400 MHz, CDCl₃) δ 9.90 (s, 1H), 8.12 (s, 1H), 7.89-7.80 (m, 2H), 7.76 (s, 2H), 7.43 (s, 1H), 7.15 (s, 1H), 7.03 (d, J=8.1, 1H), 6.68 (s, 1H), 6.62 (s, 1H), 3.60 (s, 3H), 3.32 (s, 3H), 2.38 (s, 3H) (YH-002-51P); ESMS m/z: 373.2 [M+H⁺], 395.1 [M+Na⁺], 767.3 [2M+Na⁺].

### Step III: 5,11-dimethyl-3-(2-methyl-4-(pyrrolidin-1-ylmethyl)phenylamino)-5H-benzo[e]pyrido[3,4-b][1,4]diazepin-10(11H)-one (106)

NaBH₄ (2.8 mg, 0.075 mmol) was added to a solution of compound R4 (4.7 mg, 0.012 mmol), pyrrolidine (6.2 μL, 0.075 mmol), and TFA (9.3 μL, 0.12 mmol) in HC(OEt)3 (0.50 mL) at room temperature. The mixture was stirred at room temperature overnight. The reaction was quenched with diluted HCl aqueous solution, then diluted with DMF. Filtration through a small round filter and the filtrate was purified by HPLC to provide 106 (3.4 mg, 64%) as white solid. 1H NMR (400 MHz, CDCl3) δ 8.00 (s, 1H), 7.83 (s, 1H), 7.37 (s, 2H), 7.27 (s, 1H), 7.22 (d, J=8.1, 1H), 7.13 (s, 1H), 6.99 (s, 1H), 6.36 (s, 2H), 3.71 (s, 2H), 3.57 (s, 3H), 3.21 (s, 3H), 2.70 (br s, 4H), 2.28 (s, 3H), 1.89 (br s, 4H); ESMS m/z: 428.3 [M+H⁺].

### Example 3

### Preparation of (4-((5,11-dimethyl-10,10-dioxido-5,11-dihydrobenzo[f]pyrido[3,4-c][1,2,5]thiadiazepin-3-yl)amino)-3-ethoxyphenyl)(4-(pyrrolidin-1-yl)piperidin-1-yl)methanone (S1)

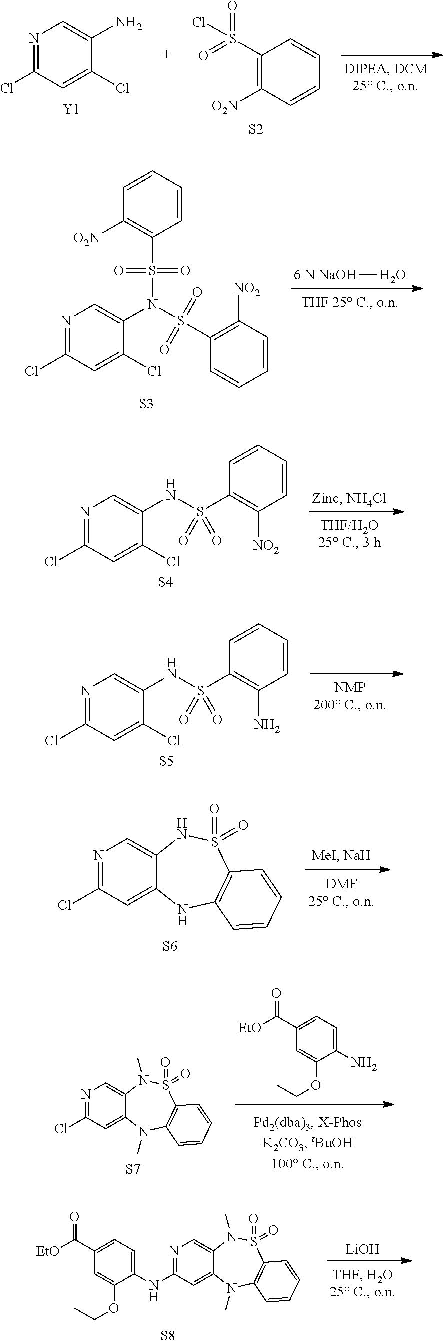

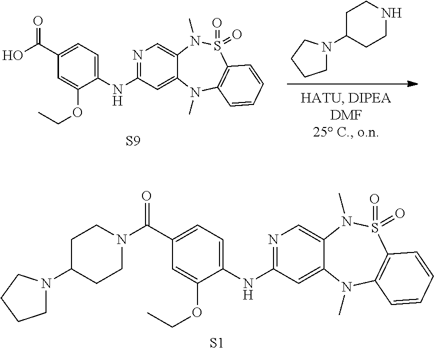

### Step I: N-(4,6-dichloropyridin-3-yl)-2-nitro-N-(2-nitrophenylsulfonyl)-benzenesulfonamide (S3)

2-nitrobenzenesulfonyl chloride (1.773 g, 8.00 mmol) was added to a solution of 5-amino-2,4-dichloropyridine (652.0 mg, 4.0 mmol) and DIPEA (4.18 mL, 24.0 mmol) in DCM (16 mL) at 25° C. The mixture was stirred at 25° C. overnight. The LCMS indicated that the major product was compound S3 and the minor peak was S4. The reaction mixture was concentrated using a rotavapor and the residue used for the next reaction without further purification. ESMS m/z: 555.0 [M+Na⁺].

### Step II: N-(4,6-dichloropyridin-3-yl)-2-nitrobenzenesulfonamide (S4)

To a solution of N-(4,6-dichloropyridin-3-yl)-2-nitro-N-((2-nitrophenyl)sulfonyl)benzenesulfonamide, compound S3 (4 mmol) in THF (9.4 mL) was added 6 N NaOH (3 mL) and the mixture was stirred at room temperature overnight. After the reaction went to completion THF was removed using a rotavapor and the aqueous layer was extracted with ethyl acetate. The combined organic phase was washed with saturated sodium chloride aqueous solution, dried over anhydrous sodium sulfate, filtered, and concentrated by rotary evaporation to provide compound S4 (1.1 g, 79% for two steps). ¹H NMR (400 MHz, DMSO) δ 7.87 (s, 1H), 7.85-7.81 (m, 1H), 7.68-7.64 (m, 1H), 7.63-7.55 (m, 2H), 7.40-7.38 (m, 1H), 7.36 (s, 1H). ESMS m/z: 348.0 [M+H⁺], 369.9 [M+Na⁺].

### Step III: 2-amino-N-(4,6-dichloropyridin-3-yl)benzenesulfonamide (S5)

Compound S4 (1.1 g, 3.2 mmol) in a mixed solution of tetrahydrofuran (10 mL) and water (5 mL) was treated with zinc (1 g, 16 mmol) and NH₄Cl (0.86 g, 16 mmol) at room temperature for 3 hours. Zinc was filtered off and THF was concentrated by rotary evaporation. The remaining water was lyophilized which provided the product S5 (0.9 g, 90%). ¹H NMR (400 MHz, CDCl₃) δ 8.15 (s, 1H), 7.71 (s, 1H), 7.37 (d, J=8.3, 1H), 7.26-7.19 (m, 1H), 6.75 (d, J=8.4, 1H), 6.53 (t, J=7.5, 1H). ESMS m/z: 318.0 [M+H⁺], 340.0 [M+Na⁺].

### Step IV: 3-chloro-5,11-dihydrobenzo[f]pyrido[3,4-c][1,2,5]thiadiazepine 10,10-dioxide (S6)

Compound S5 (0.9 g, 3 mmol) in N-methylpyrrolidinone (0.3 M) was heated at 200° C. overnight. When cooled to room temperature water (˜38 mL) was added with vigorous stirring which generated a precipitate that was washed with cold water then dried under vacuum to provide compound S6 (0.25 g, 31%) as a dark brown solid. ¹H NMR (400 MHz, DMSO) δ 10.24 (s, 1H), 9.74 (s, 1H), 7.89 (s, 1H), 7.72 (d, J=7.9, 1H), 7.59-7.51 (m, 1H), 7.25 (d, J=8.3, 1H), 7.09-7.01 (m, 2H). ESMS m/z: 381.8 [M+H⁺].

### Step V: 3-chloro-5,11-dimethyl-5,11-dihydrobenzo[f]pyrido[3,4-c][1,2,5]thiadiazepine 10,10-dioxide (S7)

Compound S6 (0.25 g, 0.8875 mmol) and MeI (0.29 g, 2.04 mmol) was dissolved in anhydrous DMF (0.2 M). This mixture was cooled to 0° C. under N₂. To this mixture NaH (0.049 g, 2.04 mmol) was added slowly and purge with N₂. The reaction was stirred at 0° C. for 30 minutes, allowed to warm up to room temperature and was left stirring at 25° C. overnight. The reaction mixture was cooled down to 0° C. and then quenched with water (13 mL). The mixture was stirred at room temperature for 30 minutes. The precipitate was filtered and washed with water, then dried under vacuum to provide compound S7 (0.2 g, 73%). ¹H NMR (400 MHz, DMSO) δ 8.18 (s, 1H), 7.83-7.74 (m, 2H), 7.57 (dd, J=0.8, 8.3, 1H), 7.36 (td, J=1.0, 7.6, 1H), 7.19 (s, 1H), 3.53 (s, 3H), 2.84 (s, 3H). ESMS m/z: 310.0 [M+H⁺], 332.0 [M+Na⁺].

### Step VI: Ethyl4-((5,11-dimethyl-10,10-dioxido-5,11-dihydrobenzo[f]pyrido[3,4-c][1,2,5]thiadiazepin-3-yl)amino)-3-ethoxybenzoate (S8)

A mixture of compound S7 (0.20 g, 0.64 mmol), ethyl 4-amino-3-ethoxybenzoate (0.1486 g, 0.71 mmol), X-Phos (0.02708 g, 0.057 mmol), K₂CO₃ (0.2677 g, 1.94 mmol) in t-butanol (8 mL, 0.08M) was bubbled with N₂ for 30 sec. Pd₂(dba)₃ (0.03547 g, 0.039 mmol) was added and the mixture was bubbled with N₂ for additional 1 minute. The suspension was then heated at 100° C. under N₂ for 23 h. The suspension was filtered through a Celite filter column. The precipitate was washed with methanol. The combined filtrate was concentrated using a rotavapor and dried under vacuum to provide the crude compound S8 as a dark gum, which was used in the next reaction without further purification. ESMS m/z: 483.1 [M+H⁺], 505.1 [M+Na⁺].

### Step VII: 4-((5,11-dimethyl-10,10-dioxido-5,11-dihydrobenzo[f]pyrido[3,4-c][1,2,5]thiadiazepin-3-yl)amino)-3-ethoxybenzoic acid (59)

The crude compound S8 (˜0.170 g, ˜0.352 mmol) was dissolved in THF (6.8 mL). A solution of LiOH (0.0422 g, 1.76 mmol) in water (1.7 mL) was added. The reaction mixture was stirred at room temperature overnight. The THF was removed using a rotavapor. 6 N HCl (3 mL) was added to adjust the aqueous phase to pH˜1-2. The resulting precipitate was filtered, washed with water, then dried under vacuum to provide compound S9 (0.170 g, 54% for two steps) as a yellow solid. An analytical sample of compound S9 was purified by HPLC for characterization. ¹H NMR (400 MHz, DMSO) δ 8.33 (s, 2H), 8.05 (s, 1H), 7.78 (d, J=6.1, 1H), 7.69-7.65 (m, 1H), 7.55-7.48 (m, 3H), 7.30-7.26 (m, 1H), 7.02 (s, 1H), 4.19-4.18 (m, 2H), 3.50 (s, 3H), 2.93 (s, 3H), 1.42 (t, J=7.0, 3H). ESMS m/z: 455.1 [M+H⁺].

### Step VIII: (4-((5,11-dimethyl-10,10-dioxido-5,11-dihydrobenzo[f]pyrido[3,4-c][1,2,5]thiadiazepin-3-yl)amino)-3-ethoxyphenyl)(4-(pyrrolidin-1-yl)piperidin-1-yl)methanone (S1)

To a solution of compound S9 (25 mg, 0.055 mmol) and 4-pyrrolidin-1-ylpiperidine (10 mg, 0.066 mmol) in DMF (0.275 mL) were add DIPEA (0.0287 mL, 0.165 mmol) and HATU (25 mg, 0.066 mmol) at 25° C. The reaction mixture was stirred at 25° C. overnight, concentrated using a rotavapor and the residue was purified by HPLC. Lyophilization of the pure product fractions provided S1 (0.95 mg, 2.9%) as a white powder. ¹H NMR (400 MHz, DMSO) δ 8.34-8.24 (m, 2H), 8.02 (s, 1H), 7.81-7.76 (m, 1H), 7.72-7.66 (m, 1H), 7.55-7.50 (m, 1H), 7.29-7.23 (m, 1H), 6.99 (s, 2H), 6.95-6.89 (m, 1H), 4.16 (d, J=6.9, 2H), 3.50 (s, 3H), 3.5-3.3 (m, 6H), 3.08-2.97 (m, 2H), 2.93 (s, 3H), 2.27-2.16 (m, 1H), 1.91-1.81 (m, 2H), 1.7-1.67 (m, 4H), 1.40-1.3 (m, 5H). ESMS m/z: 591.2 [M+H⁺], 613.3 [M+Na]⁺.

### Example 4

### (R)-3-((2-ethoxy-4-(3-(isopropylamino)pyrrolidine-1-carbonyl)phenyl)amino)-5,11dimethyl-5H-benzo[e]pyrido[3,4-b][1,4]diazepin-10(11H)-one (P5)

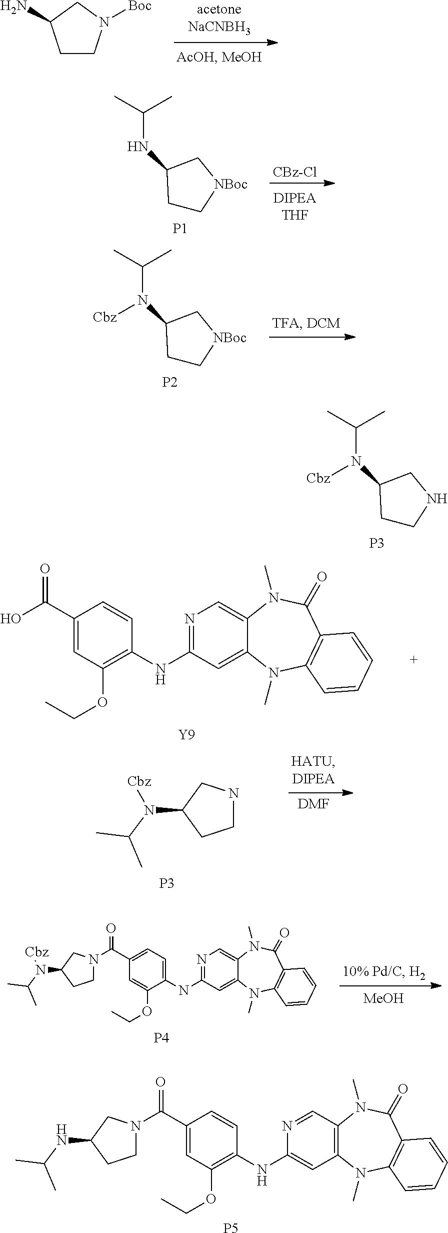

### Step I: (R)-tert-butyl 3-(isopropylamino)pyrrolidine-1-carboxylate (P1)

To a solution of (R)-tert-butyl 3-aminopyrrolidine-1-carboxylate (1.5 g, 8.1 mmol) and acetone (5.6 mL) in MeOH (27 mL), added NaCNBH₃ (1.0 g, 16 mmol), AcOH (0.72 mL) and reacted at room temperature overnight. The reaction mixture was concentrated and quenched with saturated aqueous NaHCO₃ and extracted with DCM. The organic layer was dried over anhydrous Na₂SO₄, filtered and concentrated to give an orange oil which was purified by ISCO CombiFlash chromatography (silica, 40% EtOAc/hexane) to provide the title compound as a pale yellow oil (423 mg, 24%). ESMS m/z: 229.2 [M+H⁺].

### Step II: (R)-tert-butyl 3-(((benzyloxy)carbonyl)(isopropyl)amino)pyrrolidine-1-carboxylate (P2)

To P1 (423 mg, 1.85 mmol) added THF (5.8 mL) and the reaction mixture was cooled to 0° C. To this solution added DIPEA (0.81 mL, 4.63 mmol) and finally benzyl chloroformate (0.31 mL, 2.22 mmol). The reaction mixture was stirred from 0° C. to room temperature for 3.5 h. The reaction mixture was concentrated, diluted with water and extracted with EtOAc. The organic layer was dried over anhydrous Na₂SO₄, filtered and concentrated to give a yellow oil which was purified by ISCO CombiFlash chromatography (silica, 20% EtOAc/hexane) to provide the title compound as a clear oil (574 mg, 86%). ¹H NMR DMSO-d₆ δ: 7.39-7.30 (m, 5H), 5.09 (s, 2H), 4.19-4.08 (m, 2H), 3.40-3.28 (m, 3H), 3.22-3.11 (m, 1H), 2.27-2.17 (m, 1H), 1.89-1.81 (m, 1H), 1.39 (s, 9H), 1.14-1.09 (m, 6H). ESMS m/z: 385.3 [M+Na⁺].

### Step III: (R)-benzylisopropyl(pyrrolidin-3-yl)carbamate (P3)

P2 (418.6 mg, 1.15 mmol) was treated with DCM (3.46 mL), cooled in an ice bath, added TFA (1.62 mL) and reacted from 0° C. to room temperature for 25 min. The reaction mixture was concentrated and the residue was dissolved in 1M Na₂CO₃ until basic and extracted with DCM to obtain the title compound as a pale yellow oil in quantitative yield. ¹H NMR DMSO-d₆ δ: 8.34 (bs, 1H), 7.41-7.33 (m, 5H), 5.12 (s, 2H), 4.23-4.14 (m, 2H), 3.41-3.18 (m, 3H), 3.05-2.97 (m, 1H), 2.11-2.00 (m, 2H), 1.13-1.09 (m, 6H). ESMS m/z: 263 [M+H⁺].

### Step IV: (R)-benzyl(1-(4-((5,11-dimethyl-10-oxo-10,11-dihydro-5H-benzo[e]pyrido[3,4-b][1,4]diazepin-3-yl)amino)-3-ethoxybenzoyl)pyrrolidin-3-yl)(isopropyl)carbamate (P4)

To a DMF solution (3 mL) in a round bottle flask was added 40 mg (0.095 mmol) of compound Y9 and 30 mg of P3 (0.11 mmol) and stirred for 10 min. followed by the addition of 44 mg of HATU (0.11 mmol) and 0.05 mL of DIPEA (0.3 mmol). Stirring was continued at room temperature overnight. The reaction progress was monitored by LC/MS. The solvent was removed by rotary evaporation. The remaining material was subjected to HPLC purification. The appropriate fractions were collected and dried to give 27 mg of P4 (42% yield). ESMS m/z: 663.3 [M+H⁺].

### Step V: (R)-3-((2-ethoxy-4-(3-(isopropylamino)pyrrolidine-1-carbonyl)phenyl)amino)-5,11dimethyl-5H-benzo[e]pyrido[3,4-b][1,4]diazepin-10(11H)-one (P5)

To a MeOH solution (2 mL) in a round bottle flask was added 27 mg (0.04 mmol) of P4, followed by adding 1.2 mg of Pd/C (10%) under bubbled hydrogen gas, and stir at room temperature overnight. The Pd was removed by filtration. The filtrate was collected and concentrated. The residue was purified by preparative HPLC. Fractions containing the title compound were collected and dried to give 15.4 mg of P5 (72% yield)¹H NMR DMSO-d₆ d (d, 1H), 8.22 (s, 1H) 8.16 (s, 1H), 7.65 (d, 1H), 7.49, (t, 1H), 7.24 (d, 1H), 7.22 (m, 2H), 7.12 (m, 2H), 4.17 (q, 2H), 3.65-3.55 (m, 2H), 3.45 (s, 3H), 3.28 (s, 1H), 2.8 (m, 1H), 2.6 (m, 1H), 2.11 (m, 1H), 1.65 (m, 1H), 1.43 (t, 3H), 1.00 (t, 3H), 0.92 (q, 3H). ESMS m/z: 529.3 [M+H⁺].

### Example 5

### Synthesis of 6-((2-Ethoxy-4-(4-(pyrrolidin-1-yl)piperidine-1-carbonyl)phenyl)amino)-4,9-dimethyl-4H-furo[3,2-e]pyrido[3,4-b][1,4]diazepin-10(9H)-one (91)

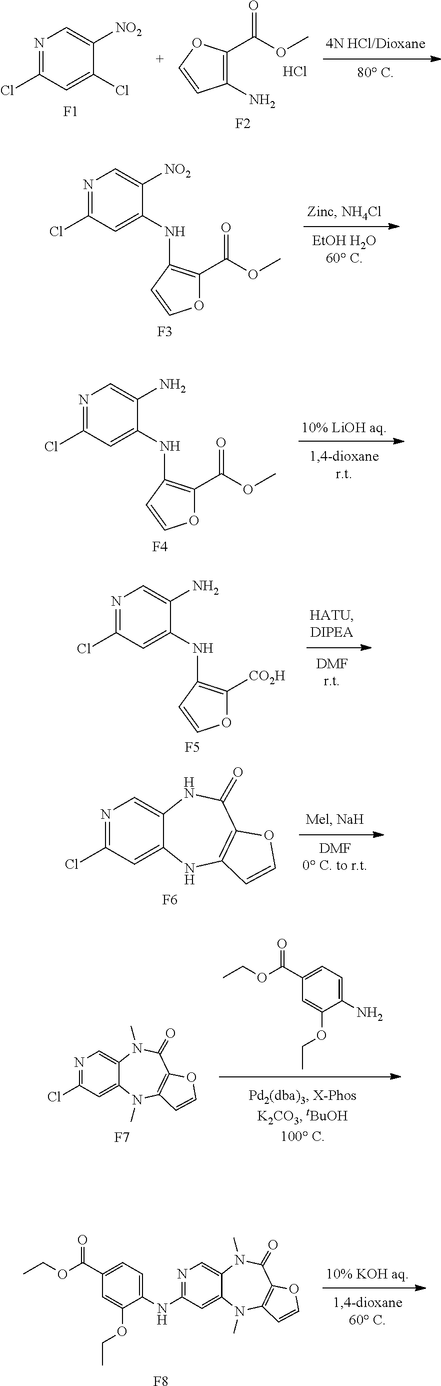

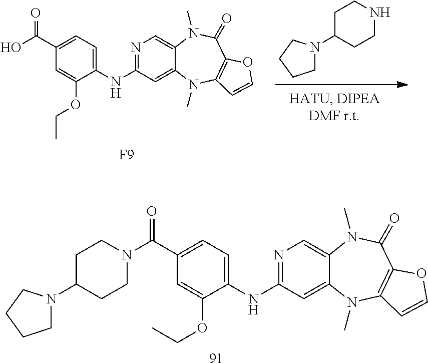

### Step I: Methyl 3-((2-chloro-5-nitropyridin-4-yl)amino)furan-2-carboxylate (F3)

To a 250 mL round bottom flask was added 2,4-dichloro-5-nitropyridine (F1, 5 g, 25.9 mmol) and methyl 3-aminofuran-2-carboxylate hydrochloride salt (F2, 5.52 g, 31.1 mmol). 40 mL of 4N HCl in 1,4-dioxane and 40 mL of 1,4-dioxane was used to dissolve the starting materials. The mixture was stirred at 80° C. for 4 days. After the reaction mixture was cooled down, it was added slowly to 450 mL of water. The precipitate was collected and dried to give F3 (6.18 g, 80% yield). It was used in the next step without further purification. ESMS m/z: 298.1 [M+H⁺], 320.1 [M+Na⁺]

### Step II: Methyl 3-((5-amino-2-chloropyridin-4-yl)amino)furan-2-carboxylate (F4)

To a 500 mL round bottom flask was added F3 (10.5 g, 35.3 mmol), zinc (9.23 g, 141 mmol) and ammonium chloride (7.55 g, 141 mmol) then 150 mL of ethanol and 100 mL of water were added. The reaction mixture was stirred at 60° C. for 16 h. The reaction mixture was concentrated using a rotavapor to remove ethanol. More water was added to the mixture resulting in a cloudy solution which was filtered. To the cake 2.5 N HCL was added forming a cloudy solution. To the clear filtrate 2.5 N HCl solution was added. In both cases the pH was adjusted to ˜1. Both solutions were stirred at room temperature for 1 hour and filtered. To the filtrates, 2.5 N NaOH solution was used to adjust the pH to ˜4.5, resulting in a precipitation which was filtered. Both cakes were combined and dried to provide compound F4 (6.11 g, 64% yield) which was used in the next step without further purification. ESMS m/z: 268.0 [M+H⁺], 290.0 [M+Na⁺]

### Step III: 3-((5-Amino-2-chloropyridin-4-yl)amino)furan-2-carboxylic acid (F5)

To compound F4 (3.76 g, 14 mmol) was added 5 mL of 1, 4-dioxane and 35 mL of 10% LiOH aqueous solution. 30 mL of water was added to dilute the sticky solid. The mixture was stirred at room temperature for 3 hours. 2.5 N HCl solution was used to acidify the reaction mixture to ˜pH 4.5. The resulting cloudy solution was filtered, the precipitate was collected, and dried to provide compound F5 (1.85 g, 52% yield). It was used in next step without further purification. ESMS m/z: 254.0 [M+H⁺]

### Step IV: 6-Chloro-4H-furo[3,2-e]pyrido[3,4-b][1,4]diazepin-10(9H)-one (F6)

To compound F5 (2.34 g, 9.23 mmol) was added DMF (20 mL) and DIPEA (4.82 mL, 27.7 mmol). The mixture was stirred at room temperature for 10 minutes before HATU (5.26 g, 13.8 mmol) was added and stirred for two hours. The mixture was then slowly added into 300 mL of water. The resulting precipitate was collected and dried to provide compound F6 (2.02 g, 93% yield). It was used in the next step without further purification. ESMS m/z: 236.1 [M+H⁺], 258.0 [M+Na⁺]; ¹H NMR (400 MHz, d6-DMSO) δ 9.14 (s, 1H), 8.95 (s, 1H), 7.69 (d, J=13.6, 2H), 6.60 (s, 1H), 6.19 (s, 1H).

### Step V: 6-Chloro-4,9-dimethyl-4H-furo[3,2-e]pyrido[3,4-b][1,4]diazepin-10(9H)-one (F7)

To compound F6 (0.98 g, 4.16 mmol) was added anhydrous DMF (15 mL) and MeI (1.04 mL, 16.64 mmol). The mixture was then stirred in an ice bath for 10 minutes before 60% NaH in mineral oil (0.58 g, 14.56 mmol) was slowly added. The reaction was stirred and the ice bath was allowed to gradually warm to room temperature over 1.5 hours then stirred an additional 1.5 hours. The mixture was concentrated under vacuum. Brine was added followed by an extraction with EtOAc. The organic phase was collected and dried over Na₂SO₄, filtered, and concentrated using a rotavapor. Hexanes (20 mL) was added to the mixture and stirred at room temperature for 30 minutes. The mixture was filtered and the insoluble material was collected and dried under vacuum to provide compound F7 (0.568 g, 52% yield). ESMS m/z: 264.1, 266.1 [M+H⁺], 286.1, 288.1 [M+Na⁺]; ¹H NMR (400 MHz, CDCl₃) δ 8.04 (d, J=16.9, 1H), 7.50 (t, J=8.2, 1H), 6.85 (s, 1H), 6.25 (d, J=2.0, 1H), 3.45-3.36 (m, 3H), 3.25 (d, J=18.8, 3H)

### Step VI: Ethyl 4-((4,9-dimethyl-10-oxo-9,10-dihydro-4H-furo[3,2-e]pyrido[3,4-b][1,4]diazepin-6-yl)amino)-3-ethoxybenzoate (F8)

A mixture of compound F7 (230 mg, 0.87 mmol), ethyl 4-amino-3-ethoxybenzoate (237 mg, 1.13 mmol), Pd₂(dba)₃ (48 mg, 0.052 mmol), X-Phos (36.6 mg, 0.077 mmol), and K₂CO₃ (361.7 mg, 2.62 mmol) in ^{t}BuOH (5 mL) was bubbled with N₂ for 2 minutes. The N₂-purged mixture was then heated at 100° C. for 5 hours. The mixture was concentrated using a rotavapor before it was extracted with DCM and water. The organic phase was collected and dried over Na₂SO₄, filtered, and concentrated using a rotavapor. The brown solid containing F8 was used in the next step without further purification. ESMS m/z: 437.1 [M+H⁺]

### Step VII: 4-((4,9-Dimethyl-10-oxo-9,10-dihydro-4H-furo[3,2-e]pyrido[3,4-b][1,4]diazepin-6-yl)amino)-3-ethoxybenzoic acid (F9)

The crude compound F8 (˜70% pure, 536 mg, ˜0.87 mmol) was dissolved in 1,4-dioxane (4 mL). Then 10% KOH aqueous solution (5 mL) was added to the mixture and stirred at 60° C. for 2 hours. More water was added to dilute the reaction mixture and it was acidified using a 2.5 N HCl solution. The resulting precipitate was collected and dried under vacuum to provide compound F9 (226 mg, 63% yield for last two steps). It was used in the next step without future purification. ESMS m/z: 409.2 [M+H]; ¹H NMR (400 MHz, CDCl₃) δ 8.19 (d, J=8.5, 1H), 8.04 (s, 1H), 7.77 (dd, J=1.8, 8.5, 1H), 7.58 (d, J=1.8, 1H), 7.47 (d, J=2.0, 1H), 7.39 (s, 1H), 6.45 (s, 1H), 6.25 (d, J=2.0, 1H), 4.22 (q, J=7.0, 2H), 3.44 (s, 3H), 3.29 (s, 3H), 1.52 (t, J=7.0, 3H)

### Step VIII: 6-((2-Ethoxy-4-(4-(pyrrolidin-1-yl)piperidine-1-carbonyl)phenyl)amino)-4,9-dimethyl-4H-furo[3,2-e]pyrido[3,4-b][1,4]diazepin-10(9H)-one (91)

To a mixture of compound F9 (35 mg, 0.0857 mmol) and 4-(pyrrolidin-1-yl)piperidine (19.8 mg, 0.129 mmol) was added anhydrous DMF (3 mL) and DIPEA (0.0702 mL, 0.403 mmol). The mixture was stirred at room temperature for 10 minutes before HATU (48.9 mg, 0.129 mmol) was added. The reaction was stirred for an additional 2 hours. The reaction mixture was purified using HPLC to provide compound 91 (13.5 mg, 29% yield). ESMS m/z: 545.3 [M+H]; ¹H NMR (400 MHz, CDCl₃) δ 8.08 (d, J=8.0, 1H), 8.00 (s, 1H), 7.46 (d, J=2.0, 1H), 7.11 (s, 1H), 7.04-6.96 (m, 2H), 6.39 (s, 1H), 6.25 (d, J=2.0, 1H), 4.15 (q, J=7.0, 2H), 3.42 (s, 3H), 3.27 (s, 3H), 3.02 (s, 6H), 2.86-2.71 (m, 2H), 2.06 (s, 1H), 2.05 (s, 2H), 2.01 (s, 5H), 1.85 (s, 2H), 1.48 (t, J=7.0, 3H).

### Example 6

### Synthesis of 7-Chloro-5H-dipyrido[3,4-b:3′,2′-e][1,4]diazepin-11(10H)-one (C2)

### 7-Chloro-5H-dipyrido[3,4-b:3′,2′-e][1,4]diazepin-11(10H)-one (C2)

Ethyl 3-((2-chloro-5-nitropyridin-4-yl)amino)picolinate (C1, 400 mg, 1.24 mmol) was dissolved in THF/H₂O (7.6 mL: 2.4 mL) and zinc (405 mg, 6.19 mmol), ammonium chloride (331 mg, 6.19 mmol) were added. The reaction mixture was stirred at room temperature for 2 h. The reaction mixture was filtered and the solid was washed with EtOAc and MeOH multiple times. The filtrate was evaporated and lyophilized over night to obtain C2 as a yellow solid (269 mg, 87%) ESMS m/z: 247.1 [M+H⁺], 269.0 [M+Na⁺].

Tables 1 and 1A below provide further examples prepared using procedures similar to those described in Examples 1-5, and routine modifications thereof. The electrospray mass spectrometry characterization data for the compounds is provided in Tables 1 and 1A.

### Example 7

### Preparation of 3-((1r,4r)-4-hydroxycyclohexylamino)-5,11-dimethyl-5H-benzo[e]pyrido[3,4-b][1,4]diazepin-10(11H)-one (33)

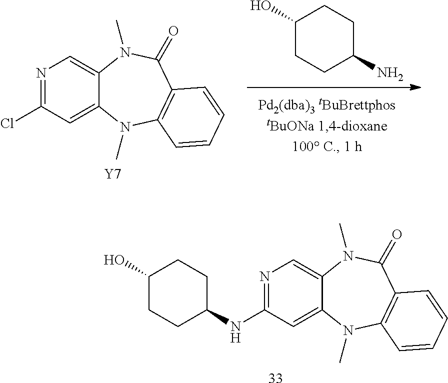

### Step I: 3-((1r,4r)-4-hydroxycyclohexylamino)-5,11-dimethyl-5H-benzo[e]pyrido[3,4-b][1,4]diazepin-10(11H)-one (33)

A mixture of 3-chloro-5,11-dimethyl-5H-benzo[e]pyrido[3,4-b][1,4]diazepin-10(11H)-one (4.13 g, 15.1 mmol), trans-4-aminocyclohexanol (2.09 g, 18.1 mmol), Pd₂(dba)₃ (691 mg, 0.755 mmol), ^{t}BuBrettPhos (732 mg, 1.51 mmol), and ^{t}BuONa (5.08 g, 52.9 mmol) in 1,4-dioxane (150 mL) was stirred at 100° C. for 1 h. After cooling to room temperature, the reaction mixture was filtered through a pad of Celite pad, and then the solvent was removed under reduced pressure. After the purification by silica gel column chromatography (Biotage Ultra 100 g, toluene to 15% ethanol-toluene), the residue was suspended with ethyl acetate. The precipitate was collected by filtration and then dried in vacuo to afford the title compound 33 (2.30 g, 43%) as a pale yellow solid. ¹H NMR (400 MHz, DMSO-d₆) δ 1.11-1.20 (4H, m), 1.80-1.88 (4H, s), 3.17 (3H, s), 3.37 (3H, s), 3.38 (1H, brs), 3.57-3.58 (1H, m), 4.50 (1H, d, J=4.4 Hz), 6.18 (1H, s), 6.34 (1H, d, J=8.0 Hz), 7.11 (1H, t, J=8.0 Hz), 7.17 (1H, d, J=8.0 Hz), 7.45 (1H, t, J=8.0 Hz), 7.60 (1H, d, J=8.0 Hz), 7.92 (1H, s); HRESIMS (+) calcd. for C₂₀H₂₅N₄O₂ 353.19775, found 353.19729.

### Example 8

### Preparation of 3-(((1r,4r)-4-hydroxycyclohexyl)amino)-11-methyl-5-(methylsulfonyl)-5H-benzo[e]pyrido[3,4-b][1,4]diazepin-10(11H)-one (T86)

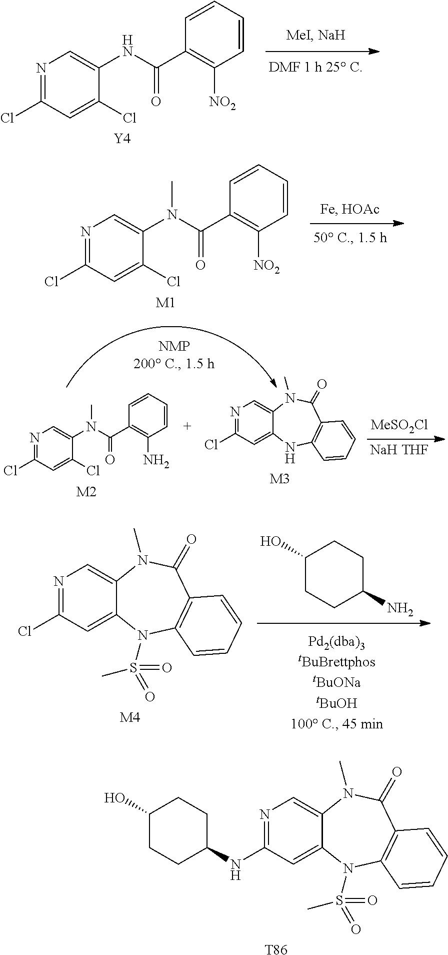

### Step I: N-(4,6-dichloropyridin-3-yl)-N-methyl-2-nitrobenzamide (M1)

NaH (60%, 54.6 mg, 1.37 mmol) was added portionwise to a mixture of Y4 (328.3 g, 1.052 mmol) and MeI (78.8 μL, 1.26 mmol) in anhydrous DMF (3.16 mL) at 25° C. under N₂. The reaction mixture was then stirred at 25° C. under N₂ for 1 h. The reaction was quenched with 0.5 N HCl aqueous solution. After having been stirred at 25° C. for ˜15 min, the generated brown gum was separated from the reaction solution and re-dissolved in DCM. The DCM solution was washed with sat. NaCl aqueous solution, then dried over Na₂SO₄. The dried DCM phase was filtered and concentrated in vacuo to provide M1 (235.9 mg, 69%) as a brown gum. ¹H NMR (400 MHz, CDCl₃) δ 8.37 (s, 1H), 8.07 (dd, J=1.5, 7.9, 1H), 7.56-7.44 (m, 2H), 7.41 (m, 1H), 7.33 (dd, J=1.6, 7.5, 1H), 3.48 (s, 3H); ESMS found m/z 326.0 ([M+H⁺], C₁₃H⁹Cl₂N₃O₃ requires 325.0021).

### Step II: 3-chloro-11-methyl-5H-benzo[e]pyrido[3,4-b][1,4]diazepin-10(11H)-one (M3)

A suspension of compound M1 (235.9 mg, 0.72 mmol) and Fe (133.3 mg, 2.39 mmol) in HOAc (0.72 mL) and MeOH (0.72 mL) was heated at 50° C. with rigorous stirring under N₂ for 1.5 h. The reaction was quenched with 1 N NaOH aqueous solution. The aqueous phase was extracted with EtOAc. The combined EtOAc phase was washed with sat. NaHCO₃ and sat. NaCl aqueous solution, then dried over Na₂SO₄. The dried organic phase was filtered and concentrated in vacuo to provide the crude M2 (205.1 mg, ˜96%) as a yellow solid. ESMS found m/z 296.0 ([M+H⁺], C₁₃H₁₁Cl₂N³⁰ requires 295.0279). The crude M2 (contains small amount of M3) was used for next step cyclization reaction without further purification.

A solution of the crude M2 (88.9 mg, 0.30 mmol) in NMP (0.51 mL) was heated at 200° C. under N₂ for 1.5 h. H₂O was added and the mixture was stirred at 25° C. for 10 min. The generated precipitates were filtered and washed with H₂O, then dried in vacuo to provide M3 (60.5 mg, 74% for two steps) as a tan solid. ¹H NMR (400 MHz, DMSO-d₆) δ 8.68 (s, 1H), 8.28 (s, 1H), 7.72 (dd, J=1.6, 8.1, 1H), 7.48-7.36 (m, 1H), 7.13 (s, 1H), 7.08-6.99 (m, 2H), 3.41 (s, 3H); ESMS found m/z 260.0 ([M+H⁺], C₁₃H₁₀ClN₃O requires 259.0512).

### Step III: 3-chloro-11-methyl-5-(methylsulfonyl)-5H-benzo[e]pyrido[3,4-b][1,4]diazepin-10(11H)-one (M4)

To a solution of compound M3 (80.0 mg, 0.308 mmol) in THF (1.54 mL) was added sodium hydride (22 mg, 0.924 mmol) at 0° C. Then the mixture was stirred at room temperature for 30 minutes. The mixture was cooled to 0° C. again and methanesulfonyl chloride (105 mg, 0.924 mmol) was added slowly. The ice bath was removed and the reaction was left at room temperature overnight. At 0° C., the reaction was quenched with water. The reaction mixture was concentrated by rotavapor and the residue was purified by HPLC to provide M4 (86.6 mg, 83%) as a white powder. ¹H NMR (400 MHz, DMSO-d₆) δ 8.68 (s, 1H), 7.83-7.80 (m, 2H), 7.7-7.6 (m, 1H), 7.57-7.53 (m, 2H), 3.56 (s, 3H), 3.31 (m, 3H); ESMS m/z: 338.0 [M+Na⁺], 360.0 [M+Na⁺].

### Step IV: 3-(((1r,4r)-4-hydroxycyclohexyl)amino)-11-methyl-5-(methylsulfonyl)-5H-benzo[e]pyrido[3,4-b][1,4]diazepin-10(11H)-one (T86)

A mixture of compound M4 (50 mg, 0.148 mmol), ^{t}BuBrettphos (7.5 mg, 0.015 mmol), NaO^{t}Bu (49 mg, 0.581 mmol) and (1r,4r)-4-aminocyclohexanol (22 mg, 0.192 mmol) in tert-butanol (1.85 mL) was purged by nitrogen for 10 sec. Pd₂(dba)₃ (6.7 mgs, 0.007 mmol) was added and the mixture was purged with nitrogen again for 15 sec. Then the mixture was heated at 100° C. for 45 minutes. The reaction mixture was filtered through a short pad of celite and the solid was washed with methanol. The filtrate was concentrated by rotavapor and the residue was purified by HPLC to provide T86 (25 mg, 41%) as a white powder. ¹H NMR (400 MHz, DMSO-d₆) δ 8.18 (s, 1H), 7.76-7.74 (m, 1H), 7.62-7.59 (m, 1H), 7.52-7.44 (m, 2H), 6.75-6.73 (d, 1H), 6.55 (s, 1H), 4.55 (s, 1H), 3.57 (s, 2H), 3.46 (s, 3H), 3.13 (s, 3H), 1.93-1.81 (m, 4H), 1.23-1.18 (m, 4H); ESMS m/z: 417.1 [M+H⁺], 439.1 [M+Na⁺].

### Example 9

### Preparation of 3-((2-ethoxy-4-(morpholine-4-carbonyl)phenyl)amino)-11-methylbenzo[f]pyrido[4,3-b][1,4]oxazepin-10(11H)-one

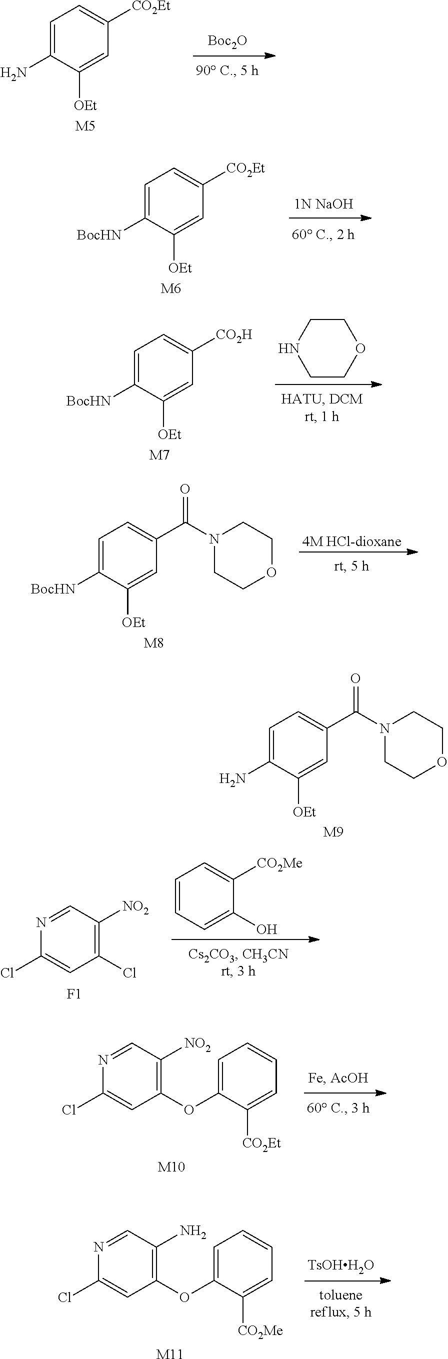

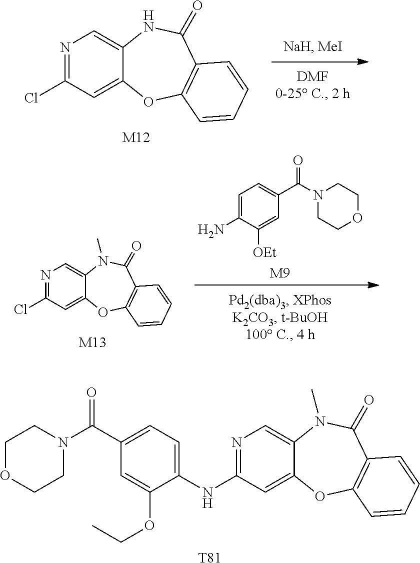

### Step I: ethyl 4-((tert-butoxycarbonyl)amino)-3-ethoxybenzoate (M6)

A mixture of M5 (ethyl 4-amino-3-ethoxybenzoate, 3.14 g, 15.0 mmol) and di-tert-butyl dicarbonate (16.5 g, 75.6 mmol) was stirred at 90° C. for 5 h. The reaction mixture was purified by silica gel column chromatography (hexane to 20% ethyl acetate-hexane) to provide the title compound M6 (6.30 g, quant.) as a colorless solid. LRMS (ESI): 310 [M+H⁺].

### Step II: 4-((tert-butoxycarbonyl)amino)-3-ethoxybenzoic acid (M7)

To a stirred solution of M6 (4.60 g, 14.9 mmol) in methanol (60 mL) and tetrahydrofuran (120 mL) was added 1 N NaOH aqueous solution (30 mL) and the mixture was stirred at 60° C. for 2 h. The reaction mixture was concentrated in vacuo, and then added water and 2 N HCl to adjust the pH to 3. The precipitate was collected and dried to give the title compound M7 (4.31 g, quant.) as a colorless solid. LRMS (ESI) 280 [M−H⁺].

### Step III: tert-butyl (2-ethoxy-4-(morpholine-4-carbonyl)phenyl)carbamate (M8)

To a mixture of M7 (4-((tert-butoxycarbonyl)amino)-3-ethoxybenzoic acid, 1.41 g, 5.01 mmol), morpholine (520 μL, 6.01 mmol), N,N-diisopropylethylamine (3.50 mL, 20.1 mmol) in CH₂Cl₂ (25 mL) was added HATU (2.30 g, 6.05 mmol) at 0° C. and the mixture was stirred at room temperature for 1 h. The reaction mixture was concentrated in vacuo and then purified by silica gel column chromatography (ethyl acetate) to provide the title compound M8 (1.80 g, quant.) as a colorless amorphous. LRMS (ESI) 351 [M+H⁺].

### Step IV: (4-amino-3-ethoxyphenyl)(morpholino)methanone (M9)

To a stirred solution of M8 (tert-butyl (2-ethoxy-4-(morpholine-4-carbonyl)phenyl)carbamate, 1.79 g, 5.11 mmol) in ethanol (6 mL) was added 4 N HCl-dioxane (12 mL), and the mixture was stirred at room temperature for 5 h. The reaction mixture was concentrated in vacuo and then added saturated aqueous NaHCO₃ to adjust pH to 10 and the mixture was extracted with ethyl acetate. The organic layer was dried over anhydrous Na₂SO₄ and then the solvent was removed in vacuo. The resulting material was purified by silica gel column chromatography to provide the title compound M9 (1.30 g, quant.) as a pale yellow oil. LRMS (ESI) 251 [M+H⁺].

### Step V: methyl 2-((2-chloro-5-nitropyridin-4-yl)oxy)benzoate (M10)

To a stirred solution of F1 (2,4-dichloro-5-nitropyridine, 96.5 mg, 0.500 mmol) and methyl salicylate (77.0 mg, 0.506 mmol) in acetonitrile (2.5 mL) was added cesium carbonate (245 mg, 0.752 mmol) and the mixture was stirred at room temperature for 3 h. Water was added to the reaction mixture and then extracted with ethyl acetate. The organic layer was dried over anhydrous Na₂SO₄ and the solvent was removed in vacuo. The residue was purified by silica gel column chromatography (20% ethyl acetate-hexane) to provide the title compound M10 (134 mg, 87%) as a colorless solid. LRMS (ESI) 309 [M+H⁺].

### Step VI: methyl 2-((5-amino-2-chloropyridin-4-yl)oxy)benzoate (M11)

A mixture of M10 (methyl 2-((2-chloro-5-nitropyridin-4-yl)oxy)benzoate, 391 mg, 1.27 mmol), iron powder (346 mg, 6.20 mmol) in acetic acid (13 mL) was heated at 60° C. for 3 h. The reaction mixture was filtered through a pad of Celite and eluted with hot ethyl acetate. To the mixture was added saturated aqueous NaHCO₃ to adjust pH to 10 and the mixture was extracted with ethyl acetate. The organic layer was dried over anhydrous Na₂SO₄ and then the solvent was removed in vacuo. The resulting material was purified by silica gel column chromatography (50% ethyl acetate-hexane) to provide the title compound M11 (345 mg, 97%) as a colorless solid. LRMS (ESI) 279 [M+H⁺].

### Step VII: 3-chlorobenzo[f]pyrido[4,3-b][1,4]oxazepin-10(11H)-one (M12)

A mixture of M11 (methyl 2-((5-amino-2-chloropyridin-4-yl)oxy)benzoate, 100 mg, 0.359 mmol) and p-toluenesulfonic acid monohydrate (137 mg, 0.720 mmol) in toluene (18 mL) was heated to reflux for 5 h. The mixture was concentrated in vacuo and then ethanol (5 mL) was added to the residue. The precipitate was collected and dried to give the title compound M12 (83.3 mg, 94%) as a colorless solid. LRMS (ESI) 247 [M+H⁺].

### Step VIII: 3-chloro-11-methylbenzo[f]pyrido[4,3-b][1,4]oxazepin-10(11H)-one (M13)

To a stirred solution of M12 (3-chlorobenzo[f]pyrido[4,3-b][1,4]oxazepin-10(11H)-one, 100 mg, 0.405 mmol) and iodomethane (40 μL, 0.64 mmol) in N,N-dimethylformamide (1.2 mL) was added sodium hydride (20 mg, 0.50 mmol, 60% oil suspension) at 0° C. The reaction mixture was stirred at 0° C. for 1 h and then at room temperature for 1 h. The reaction was quenched by ice-water and the solid precipitated. The precipitate was collected and dried to give the title compound M13 (80.5 mg, 76%) as a colorless solid. LRMS (ESI) 261 [M+H⁺].

### Step IX: 3-((2-ethoxy-4-(morpholine-4-carbonyl)phenyl)amino)-11-methylbenzo[f]pyrido[4,3-b][1,4]oxazepin-10(11H)-one (T81)

A mixture of M13 (3-chloro-11-methylbenzo[f]pyrido[4,3-b][1,4]oxazepin-10(11H)-one, 40.0 mg, 0.162 mmol), (4-amino-3-ethoxyphenyl)(morpholino)methanone (85.0 mg, 0.243 mmol), XPhos (4.0 mg, 8.4 μmol), Pd₂(dba)₃ (25.0 mg, 27.3 μmol), potassium carbonate (134 mg, 0.970 mmol) in tert-butyl alcohol (1 mL) was heated at 100° C. for 4 h. The reaction mixture was diluted with ethyl acetate and then filtered through a pad of Celite. The mixture was dried over anhydrous Na₂SO₄ and then the solvent was removed in vacuo. The resulting material was purified by silica gel column chromatography (ethyl acetate to 10% methanol-ethyl acetate) to provide the title compound T81 (29.9 mg, 39%) as a yellow foam. LRMS (ESI) 475 [M+H⁺].

### Example 10

### Preparation of 3-((2-ethoxy-4-(morpholine-4-carbonyl)phenyl)amino)-11-methylbenzo[f]pyrido[4,3-b][1,4]thiazepin-10(11H)-one (T82)

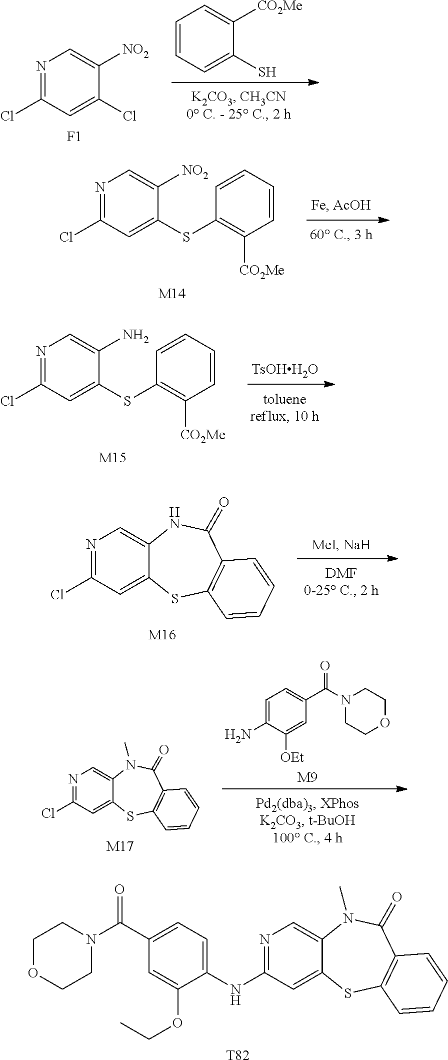

### Step I: methyl 2-((2-chloro-5-nitropyridin-4-yl)thio)benzoate (M14)

To a stirred suspension of F1 (290 mg, 1.50 mmol) and potassium carbonate (312 mg, 2.26 mmol) in acetonitrile (5.5 mL) was added a solution of methyl thiosalicylate (253 mg, 1.50 mmol) in acetonitrile (2 mL) at 0° C. The mixture was stirred 0° C. for 1 h and then at room temperature for 1 h. Water was added to the reaction mixture and then extracted with ethyl acetate. The organic layer was dried over anhydrous Na₂SO₄ and then the solvent was removed in vacuo. The residue was purified by silica gel column chromatography (25% ethyl acetate-hexane) to provide the title compound M14 (473 mg, 97%) as a yellow solid. LRMS (ESI) 325 [M+H⁺].

### Step II: methyl 2-((5-amino-2-chloropyridin-4-yl)thio)benzoate (M15)

A mixture of M14 (methyl 2-((2-chloro-5-nitropyridin-4-yl)thio)benzoate, 438 mg, 1.35 mmol) and iron powder (377 mg, 6.75 mmol) in acetic acid (13 mL) was heated at 60° C. for 3 h. The reaction mixture was filtered through a pad of Celite and eluted with hot ethyl acetate. To the mixture was added saturated aqueous NaHCO₃ to adjust pH to 10 and the mixture was extracted with ethyl acetate. The organic layer was dried over anhydrous Na₂SO₄ and then the solvent was removed in vacuo. The resulting material was purified by silica gel column chromatography (33% ethyl acetate-hexane) to provide the title compound M15 (367 mg, 92%) as a pale yellow solid. LRMS (ESI) 295 [M+H⁺].

### Step III: 3-chlorobenzo[f]pyrido[4,3-b][1,4]thiazepin-10(11H)-one (M16)

A mixture of M15 (methyl 2-((5-amino-2-chloropyridin-4-yl)thio)benzoate (200 mg, 0.679 mmol) and p-toluene sulfonic acid monohydrate (260 mg, 1.37 mmol) in toluene (34 mL) was heated to reflux for 10 h. The mixture was concentrated in vacuo and then ethanol (5 mL) was added to the residue. The precipitate was collected and dried to give the title compound M16 (178 mg, 100%) as a pale yellow solid. LRMS (ESI) 263 [M+H⁺].

### Step IV: 3-chloro-11-methylbenzo[f]pyrido[4,3-b][1,4]thiazepin-10(11H)-one (M17)

To a stirred solution of M16 (3-chlorobenzo[f]pyrido[4,3-b][1,4]thiazepin-10(11H)-one, 235 mg, 0.895 mmol) and iodomethane (90 μL, 1.5 mmol) in N,N-dimethylformamide (2.5 mL) was added sodium hydride (45.0 mg, 1.13 mmol, 60% oil suspension) at 0° C. The reaction mixture was stirred at 0° C. for 1 h and then at room temperature for 1 h. The reaction was quenched by ice-water and the solid precipitated. The precipitate was collected and dried to give the title compound M17 (191 mg, 77%) as a pale yellow solid. LRMS (ESI) 277 [M+H⁺].

### Step V: 3-((2-ethoxy-4-(morpholine-4-carbonyl)phenyl)amino)-11-methylbenzo[f]pyrido[4,3-b][1,4]thiazepin-10(11H)-one (T82)

A mixture of M17 (3-chloro-11-methylbenzo[f]pyrido[4,3-b][1,4]thiazepin-10(11H)-one, 36.0 mg, 0.130 mmol), (4-amino-3-ethoxyphenyl)(morpholino)methanone (68.5 mg, 0.195 mmol), XPhos (3.2 mg, 6.7 μmol), Pd₂(dba)₃ (20.0 mg, 21.8 μmol), potassium carbonate (108 mg, 0.781 mmol) in tert-butyl alcohol (1 mL) was heated at 100° C. for 4 h. The reaction mixture was diluted with ethyl acetate and then filtered through a pad of Celite. The mixture was dried over anhydrous Na₂SO₄ and then the solvent was removed in vacuo. The resulting material was purified by silica gel column chromatography (ethyl acetate) to provide the title compound T82 (23.0 mg, 36%) as a yellow foam. LRMS (ESI) 491 [M+H⁺].

### Example 11

### Preparation of 3-((2-ethoxy-4-(morpholine-4-carbonyl)phenyl)amino)-11-methylbenzo[f]pyrido[4,3-b][1,4]thiazepin-10(11H)-one 5-oxide (T83) and 3-((2-ethoxy-4-(morpholine-4-carbonyl)phenyl)amino)-11-methylbenzo[f]pyrido[4,3-b][1,4]thiazepin-10(11H)-one 5-oxide (T84)

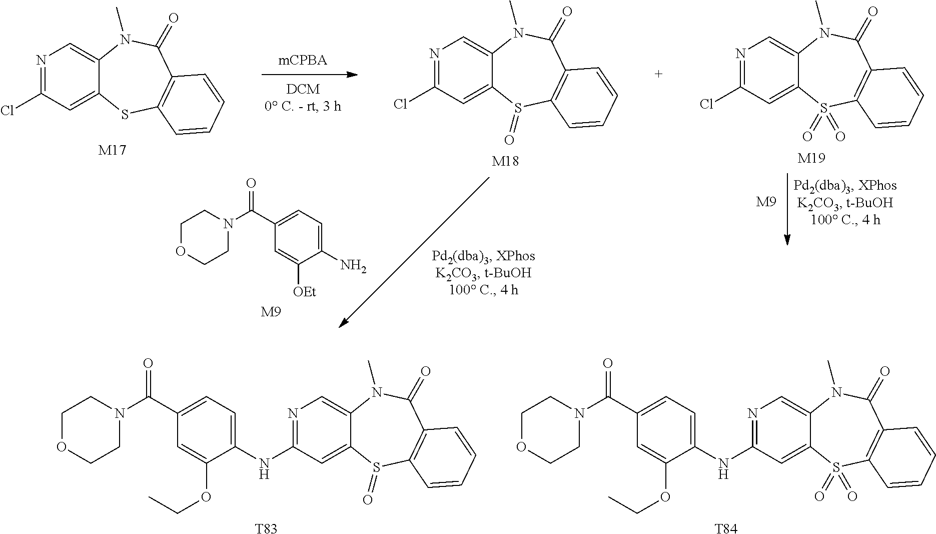

### Step I: 3-chloro-11-methylbenzo[f]pyrido[4,3-b][1,4]thiazepin-10(11H)-one 5-oxide (M18) and 3-chloro-11-methylbenzo[f]pyrido[4,3-b][1,4]thiazepin-10(11H)-one 5,5-dioxide (M19)

To a solution of M17 (3-chloro-11-methylbenzo[f]pyrido[4,3-b][1,4]thiazepin-10(11H)-one, 700 mg, 2.53 mmol) in dichloromethane (26 mL) was added 3-chloroperbenzoic acid (880 mg, 5.10 mmol, 70% purity) at 0° C. The reaction mixture was stirred at room temperature for 3 h. Saturated aqueous Na₂S₂O₃ was added to the mixture at 0° C. and the mixture was extracted with ethyl acetate. The organic layer was dried over anhydrous Na₂SO₄ and then the solvent was removed in vacuo. The resulting material was purified by silica gel column chromatography (25% ethyl acetate-hexane) to provide the sulfoxide M18 (339 mg, 46%) as a colorless solid and the sulfone M19 (393 mg, 50%) as a colorless solid. For M18, LRMS (ESI) 293 [M+H⁺]; for M19, LRMS (ESI) 309 [M+H⁺].

### Step IIa: 3-((2-ethoxy-4-(morpholine-4-carbonyl)phenyl)amino)-11-methylbenzo[f]pyrido[4,3-b][1,4]thiazepin-10(11H)-one 5-oxide (T83)

A mixture of M18 (3-chloro-11-methylbenzo[f]pyrido[4,3-b][1,4]thiazepin-10(11H)-one 5-oxide, 33.0 mg, 0.113 mmol), M9 (60.0 mg, 0.171 mmol), XPhos (2.8 mg, 5.9 μmol), Pd₂(dba)₃ (18.0 mg, 19.7 μmol), potassium carbonate (94.0 mg, 0.680 mmol) in tert-butyl alcohol (1 mL) was heated at 100° C. for 4 h. The reaction mixture was diluted with ethyl acetate and then filtered through a pad of Celite. The mixture was dried over anhydrous Na₂SO₄ and then the solvent was removed in vacuo. The resulting material was purified by silica gel column chromatography (ethyl acetate) to provide the title compound T83 (22.0 mg, 38%) as a pale yellow solid. LRMS (ESI) 507 [M+H⁺].

### Step IIb: 3-((2-ethoxy-4-(morpholine-4-carbonyl)phenyl)amino)-11-methylbenzo[f]pyrido[4,3-b][1,4]thiazepin-10(11H)-one 5-oxide (T84)

A mixture of M19 (3-chloro-11-methylbenzo[f]pyrido[4,3-b][1,4]thiazepin-10(11H)-one 5,5-dioxide, 30.0 mg, 97.2 μmol), M9 (51.0 mg, 0.146 mmol), XPhos (2.5 mg, 5.2 μmol), Pd₂(dba)₃ (16.0 mg, 17.5 μmol), potassium carbonate (81.0 mg, 0.586 mmol) in tert-butyl alcohol (1 mL) was heated at 100° C. for 4 h. The reaction mixture was diluted with ethyl acetate and then filtered through a pad of Celite. The mixture was dried over anhydrous Na₂SO₄ and then the solvent was removed in vacuo. The resulting material was purified by silica gel column chromatography (ethyl acetate) to provide the title compound T84 (18.5 mg, 34%) as a yellow solid. LRMS (ESI) 523 [M+H+].

### Example 11

### Kinase Profiling and Determination of Inhibitor IC₅₀ Values

In this example, potency of the compounds as ERK5 inhibitors was assessed, and the compounds were screened for activity against several kinases in Jurkat cells.

Lysate Preparation for Probe-Based Inhibitor Kinase Profiling:

Jurkat cell pellets were resuspended in four volumes of lysis buffer (50 mM HEPES pH 7.5, 150 mM NaCl, 0.1% Triton-X-100, 1% v/v phosphatase, inhibitor cocktail II [EMD/Calbiochem, #524625]), sonicated using a tip, sonicator, and Dounce homogenized. Lysate was cleared by centrifugation, at 16,000 g for 15 min., was gel filtered (BioRad 10DG) and MnCl₂ was then added to a final concentration of 20 mM before treatment with the test compound and probe labeling. Final test compound concentrations used for IC₅₀ determinations were 10 or 1 or 0.1 μM. All compound treatments were performed at room temperature.

Probe Labeling:

Acyl phosphate ATP probe (AX9989) labeling reactions were performed at room temperature with a final probe concentration of 5 μM after a 15 min pre-incubation of lysate with a test compound. All reactions were performed in duplicate, using 445 μl/sample at 5 mg/ml. Probe-labeled lysates were denatured and reduced (6 M urea, 10 mM DTT, 65° C., 15 min), alkylated (40 mM iodoacetamide, 37° C., 30 min, and gel filtered (Sephadex G25) into 10 mM ammonium bicarbonate, 2 M urea, 5 mM methionine. The desalted protein mixture was digested with trypsin (0.015 mg/ml) for 1 hr at 37° C., and desthiobiotinylated peptides were captured using a 12.5 ml high-capacity streptavidin resin (Thermo Scientific). Captured peptides were then washed extensively using 150 μl/wash with three different wash buffers: (A) 3 times with 1% triton, 0.5% tergitol, 1 mM EDTA in PBS; (B) 18 times with PBS; and (C) 8 times with HPLC grade water. Peptides were eluted from the streptavidin beads using two 35-μL washes of a 50% CH₃CN/water mixture containing 0.1% TFA at room temperature.

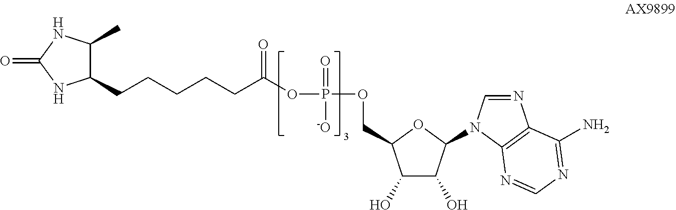

Determination of Percent Inhibition of AX9989 Labeling:

Kinase active site peptides were identified and quantified using LC/MS. Percent inhibition was calculated as the normalized decrease in the ESI-MS fragment intensities of probe-labeled peptides in samples incubated with the test compound compared to those without. For selected kinases, including ERK5, % inhibition by exemplary test compounds is provided in Table 1.

ERK5 IC₅₀ values were calculated from the percent inhibition and the screening concentration. The compounds provided herein were found to have activity as shown in Table 1, and Table 1A.

The compounds in Table 1 were also tested against the kinases listed below. These kinases were not significantly inhibited (i.e. <35%) by any of the test compounds listed in Table 1, at the indicated screening concentrations. The kinases as follows: PIK3C2Pβ, CDK2, PIP5K3, CaMK2γ, p386, p38γ, RSK1, CaMK2δ, BRAF, CaMK1δ, CaMK4, CDC2, CDK11, CDK8, CDK5, CDK6, CHK1, CHK2, CSK, DNAPK, εEF2K, ERK1, FER, FRAP, GCK, GSK3β, IKKα, ILK, IRAK4, IRE1, JAK1, JNK1, JNK2, JNK3, KHS1, LATS1, LKB1, LOK, MAP2K1, MAP2K2, MAP2K3, MAP2K4, MAP2K6, MAP3K2, MAP3K3, MAP3K4, MARK2, MARK3, MARK4, MAST3, MASTL, MLK3, MLKL, MSK1, MSK2, MST2, MST3, MST4, YSK1, NDR1, NDR2, NEK1, NEK6, NEK7, NEK9, p38α, p70S6K, p70S6K, p70S6Kβ, PCTAIRE2, PEK, PHKγ2, PI4Kα, PI4KαP2, PI4Kρ, PIP4K2α, PITSLRE, PKCι, PKR, PRPK, ROCK1, RSK1, RSK2, RSK3, SGK3, SLK, SMG1, TAO1, TAO3, TLK1, TLK2, Wnk1, Wnk2, Wnk3, ZAK, ZAP70, ZC1/HGK, ZC2/TNIK, and ZC3/MINK.

In Table 1, the IC₅₀ (nM) for ERK5 are represented as follows:
A is ≦50; B is 50-100; C is 101-500; and D is >500
Percent inhibition of ERK5, AurA, AurB or AurC, JAK1, AMPKa1 or AMPKa2, TAO2, ACK, ABL or ARG, is represented as follows:
A>90%; B is >75% to ≦90%; C is >50% to ≦75%; D is >35% to ≦50%; E is ≦35%; and n.d. is not determined.

TABLE 1
Exemplary compounds and their activity

| | | | | | Kinase | | | | | | | |
|---|---|---|---|---|---|---|---|---|---|---|---|---|
| | | | | | [Inhibition % at indicated screening concentration] | | | | | | | |
| | | | | | | | | | AMPK | | | |
| | | | | kinase | | | AurB | | a1 or | | | ABL |
| | | ESI-MS | ERK5 IC₅₀ | profiling screening | | | or | | AMPK | | | or |
| No. | structure | m/z (m + H)⁺ | (nM) | concentration (μM) | ERK5 | AurA | AurC | JAK1 | a2 | TAO2 | ACK | ARG |
| 1 | 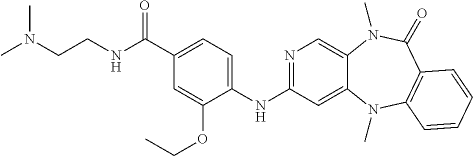 [SMILES: CCOc1cc(C(=O)NCCN(C)C)ccc1Nc1cc2c(cn1)N(C)C(=O)c1ccccc1N2C] | 489.3 | C | 1.0 | B | n.d. | n.d. | n.d. | n.d. | n.d. | n.d. | n.d. |
| 2 | 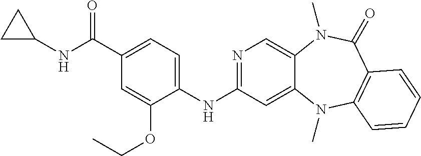 [SMILES: CCOc1cc(C(=O)NC2CC2)ccc1Nc1cc2c(cn1)N(C)C(=O)c1ccccc1N2C] | 458.3 | D | 1.0 | E | n.d. | n.d. | n.d. | n.d. | n.d. | n.d. | n.d. |
| 3 | 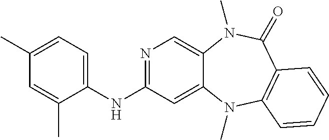 [SMILES: Cc1ccc(Nc2cc3c(cn2)N(C)C(=O)c2ccccc2N3C)c(C)c1] | 359.2 | D | 1.0 | D | n.d. | n.d. | n.d. | n.d. | n.d. | n.d. | n.d. |
| 4 | 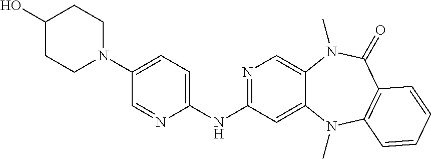 [SMILES: CN1C(=O)c2ccccc2N(C)c2cc(Nc3ccc(N4CCC(O)CC4)cn3)ncc21] | 431.3 | D | 10.0 | B | A | A | C | C | C | B | C |
| 5 | 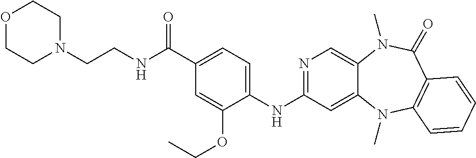 [SMILES: CCOc1cc(C(=O)NCCN2CCOCC2)ccc1Nc1cc2c(cn1)N(C)C(=O)c1ccccc1N2C] | 531.3 | C | 1.0 | C | E | E | E | E | E | E | D |
| 6 | 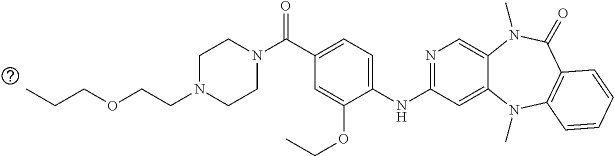 [SMILES: CCCOCCN1CCN(C(=O)c2ccc(Nc3cc4c(cn3)N(C)C(=O)c3ccccc3N4C)c(OCC)c2)CC1] | 575.3 | A | 1.0 | A | E | E | E | E | E | E | E |
| 7 | 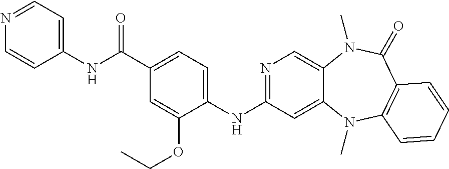 [SMILES: CCOc1cc(C(=O)Nc2ccncc2)ccc1Nc1cc2c(cn1)N(C)C(=O)c1ccccc1N2C] | 495.3 | D | 1.0 | D | E | E | E | E | E | E | E |
| 8 | 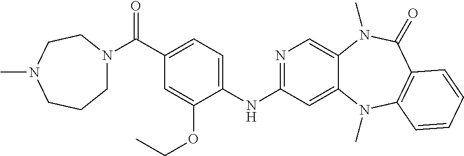 [SMILES: CCOc1cc(C(=O)N2CCCN(C)CC2)ccc1Nc1cc2c(cn1)N(C)C(=O)c1ccccc1N2C] | 515.3 | C | 1.0 | A | E | E | E | E | E | E | E |
| 9 | 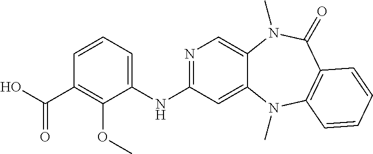 [SMILES: COc1c(Nc2cc3c(cn2)N(C)C(=O)c2ccccc2N3C)cccc1C(=O)O] | 405.2 | D | 1.0 | E | E | E | E | E | E | E | E |
| 10 | 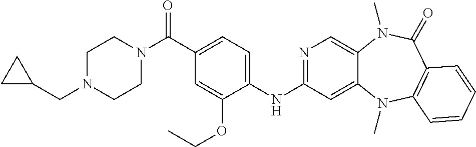 [SMILES: CCOc1cc(C(=O)N2CCN(CC3CC3)CC2)ccc1Nc1cc2c(cn1)N(C)C(=O)c1ccccc1N2C] | 541.3 | A | 0.1 | B | E | E | E | E | E | E | E |
| 11 | 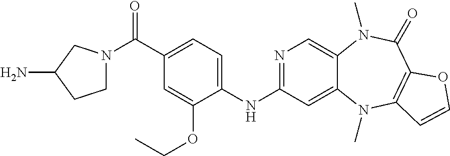 [SMILES: CCOc1cc(C(=O)N2CCC(N)C2)ccc1Nc1cc2c(cn1)N(C)C(=O)c1occc1N2C] | 477.3 | A | 0.1 | A | E | E | E | E | E | E | E |
| 12 | 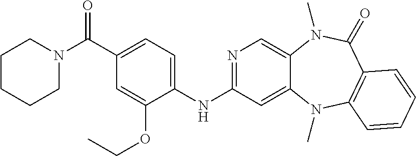 [SMILES: CCOc1cc(C(=O)N2CCCCC2)ccc1Nc1cc2c(cn1)N(C)C(=O)c1ccccc1N2C] | 486.3 | C | 1.0 | B | E | E | E | E | E | E | E |
| 13 | 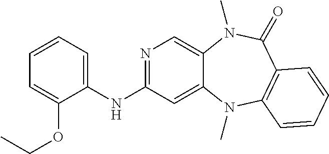 [SMILES: CCOc1ccccc1Nc1cc2c(cn1)N(C)C(=O)c1ccccc1N2C] | 375.3 | D | 1.0 | C | E | E | E | E | E | E | E |
| 14 | 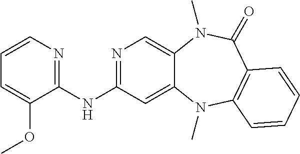 [SMILES: COc1cccnc1Nc1cc2c(cn1)N(C)C(=O)c1ccccc1N2C] | 362.2 | D | 1.0 | A | E | E | E | E | E | E | E |
| 15 | 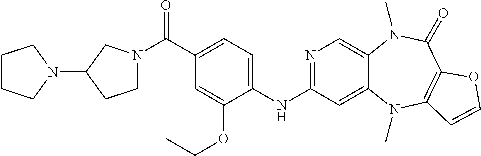 [SMILES: CCOc1cc(C(=O)N2CCC(N3CCCC3)C2)ccc1Nc1cc2c(cn1)N(C)C(=O)c1occc1N2C] | 531.3 | A | 0.1 | B | E | E | E | E | E | E | E |
| 16 | 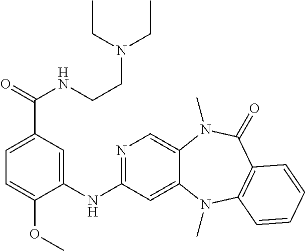 [SMILES: CCN(CC)CCNC(=O)c1ccc(OC)c(Nc2cc3c(cn2)N(C)C(=O)c2ccccc2N3C)c1] | 503.4 | C | 1.0 | B | E | E | E | E | E | E | E |
| 17 | 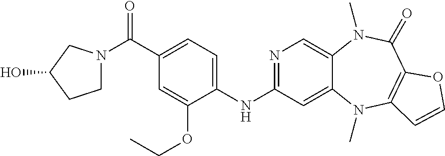 [SMILES: CCOc1cc(C(=O)N2CC[C@H](O)C2)ccc1Nc1cc2c(cn1)N(C)C(=O)c1occc1N2C] | 478.3 | A | 0.1 | B | E | E | E | E | E | E | E |
| 18 | 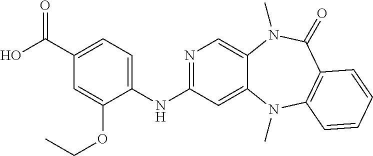 [SMILES: CCOc1cc(C(=O)O)ccc1Nc1cc2c(cn1)N(C)C(=O)c1ccccc1N2C] | 419.2 | D | 1.0 | C | E | E | E | E | E | E | E |
| 19 | 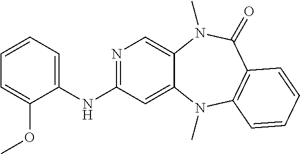 [SMILES: COc1ccccc1Nc1cc2c(cn1)N(C)C(=O)c1ccccc1N2C] | 361.2 | D | 1.0 | C | E | E | E | E | E | E | E |
| 20 | 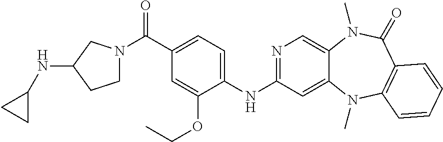 [SMILES: CCOc1cc(C(=O)N2CCC(NC3CC3)C2)ccc1Nc1cc2c(cn1)N(C)C(=O)c1ccccc1N2C] | 527.3 | A | 0.1 | B | E | E | E | E | E | E | E |
| 21 | 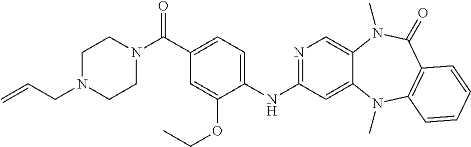 [SMILES: C=CCN1CCN(C(=O)c2ccc(Nc3cc4c(cn3)N(C)C(=O)c3ccccc3N4C)c(OCC)c2)CC1] | 527.3 | A | 0.1 | B | E | E | E | E | E | E | E |
| 22 | 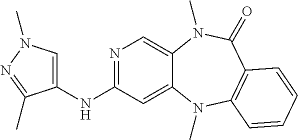 [SMILES: Cc1nn(C)cc1Nc1cc2c(cn1)N(C)C(=O)c1ccccc1N2C] | 349.2 | C | 1.0 | B | E | E | E | E | E | E | E |
| 23 | 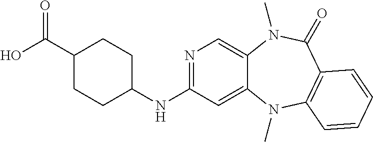 [SMILES: CN1C(=O)c2ccccc2N(C)c2cc(NC3CCC(C(=O)O)CC3)ncc21] | 381.3 | D | 1.0 | D | E | E | E | E | E | E | E |
| 24 | 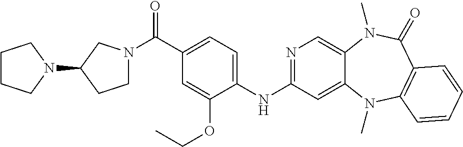 [SMILES: CCOc1cc(C(=O)N2CC[C@@H](N3CCCC3)C2)ccc1Nc1cc2c(cn1)N(C)C(=O)c1ccccc1N2C] | 541.3 | A | 0.1 | A | E | E | E | E | E | E | E |
| 25 | 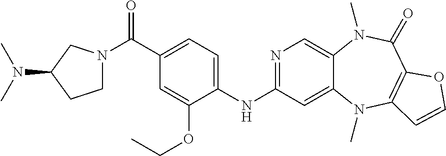 [SMILES: CCOc1cc(C(=O)N2CC[C@@H](N(C)C)C2)ccc1Nc1cc2c(cn1)N(C)C(=O)c1occc1N2C] | 505.3 | A | 0.1 | A | E | E | E | E | E | E | E |
| 26 | 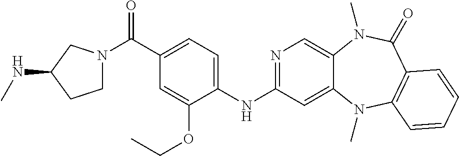 [SMILES: CCOc1cc(C(=O)N2CC[C@@H](NC)C2)ccc1Nc1cc2c(cn1)N(C)C(=O)c1ccccc1N2C] | 501.3 | A | 0.1 | B | E | E | E | E | E | E | E |
| 27 | 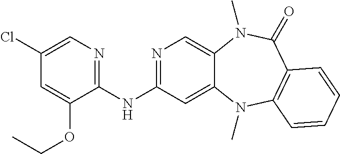 [SMILES: CCOc1cc(Cl)cnc1Nc1cc2c(cn1)N(C)C(=O)c1ccccc1N2C] | 396.2 | D | 1.0 | E | E | E | E | E | E | E | E |
| 28 | 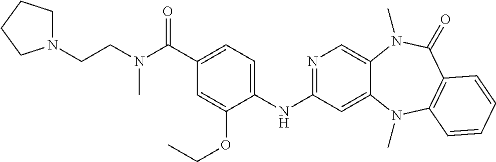 [SMILES: CCOc1cc(C(=O)N(C)CCN2CCCC2)ccc1Nc1cc2c(cn1)N(C)C(=O)c1ccccc1N2C] | 529.3 | B | 0.1 | A | E | E | E | E | E | E | E |
| 29 | 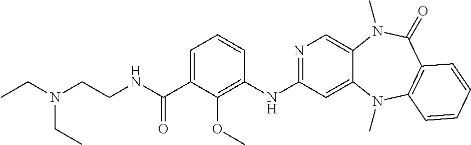 [SMILES: CCN(CC)CCNC(=O)c1cccc(Nc2cc3c(cn2)N(C)C(=O)c2ccccc2N3C)c1OC] | 503.3 | C | 1.0 | C | E | E | E | E | E | E | E |
| 30 | 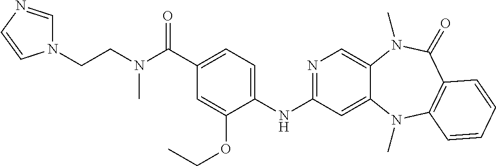 [SMILES: CCOc1cc(C(=O)N(C)CCn2ccnc2)ccc1Nc1cc2c(cn1)N(C)C(=O)c1ccccc1N2C] | 526.3 | A | 0.1 | B | E | E | E | E | E | E | E |
| 31 | 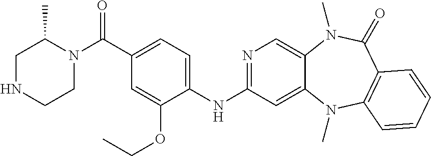 [SMILES: CCOc1cc(C(=O)N2CCNC[C@@H]2C)ccc1Nc1cc2c(cn1)N(C)C(=O)c1ccccc1N2C] | 501.3 | A | 0.1 | B | E | E | E | E | E | E | E |
| 32 | 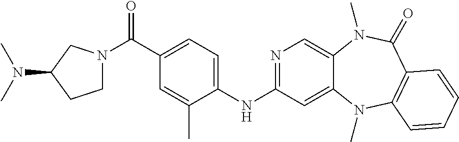 [SMILES: Cc1cc(C(=O)N2CC[C@@H](N(C)C)C2)ccc1Nc1cc2c(cn1)N(C)C(=O)c1ccccc1N2C] | 485.3 | A | 0.1 | B | E | E | E | E | E | E | E |
| 33 | 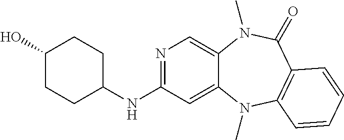 | 353.3 | C | 1.0 | B | E | E | E | E | E | E | E |
| 34 | 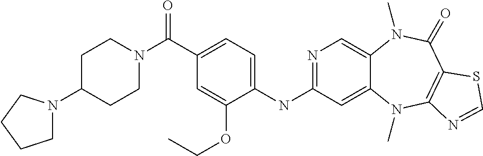 [SMILES: CCOc1cc(C(=O)N2CCC(N3CCCC3)CC2)ccc1Nc1cc2c(cn1)N(C)C(=O)c1scnc1N2C] | 562.2 | n.d. | 1 | A | E | E | E | E | E | E | E |
| 35 | 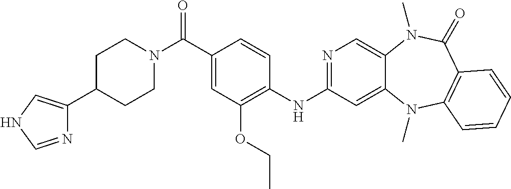 [SMILES: CCOc1cc(C(=O)N2CCC(c3c[nH]cn3)CC2)ccc1Nc1cc2c(cn1)N(C)C(=O)c1ccccc1N2C] | 552.3 | C | 0.1 | D | E | E | E | E | E | E | E |
| 36 | 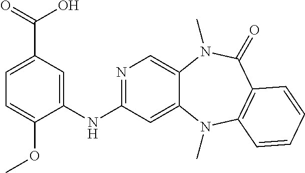 [SMILES: COc1ccc(C(=O)O)cc1Nc1cc2c(cn1)N(C)C(=O)c1ccccc1N2C] | 405.2 | D | 1.0 | D | E | E | E | E | E | E | E |
| 37 | 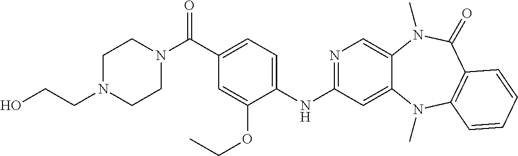 [SMILES: CCOc1cc(C(=O)N2CCN(CCO)CC2)ccc1Nc1cc2c(cn1)N(C)C(=O)c1ccccc1N2C] | 531.3 | A | 0.1 | B | E | E | E | E | E | E | E |
| 38 | 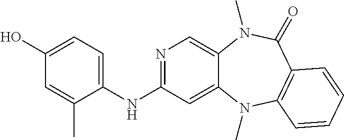 [SMILES: Cc1cc(O)ccc1Nc1cc2c(cn1)N(C)C(=O)c1ccccc1N2C] | 361.2 | C | 1.0 | C | E | E | E | E | E | E | E |
| 39 | 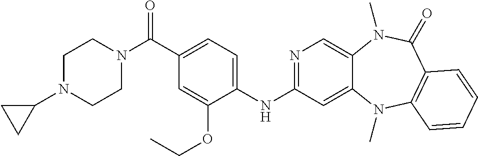 [SMILES: CCOc1cc(C(=O)N2CCN(C3CC3)CC2)ccc1Nc1cc2c(cn1)N(C)C(=O)c1ccccc1N2C] | 527.3 | A | 0.1 | C | E | E | E | E | E | E | E |
| 40 | 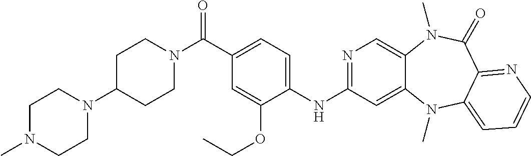 [SMILES: CCOc1cc(C(=O)N2CCC(N3CCN(C)CC3)CC2)ccc1Nc1cc2c(cn1)N(C)C(=O)c1ncccc1N2C] | 585.4 | A | 1.0 | A | E | E | E | E | E | E | E |
| 41 | 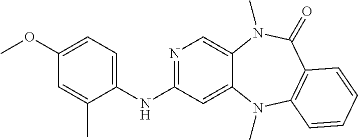 [SMILES: COc1ccc(Nc2cc3c(cn2)N(C)C(=O)c2ccccc2N3C)c(C)c1] | 375.3 | C | 1.0 | B | E | E | E | E | E | E | E |
| 42 | 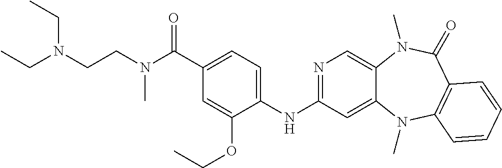 [SMILES: CCOc1cc(C(=O)N(C)CCN(CC)CC)ccc1Nc1cc2c(cn1)N(C)C(=O)c1ccccc1N2C] | 531.3 | A | 1.0 | A | E | E | E | E | E | E | E |
| 43 | 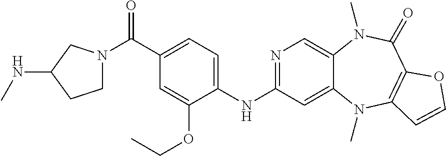 [SMILES: CCOc1cc(C(=O)N2CCC(NC)C2)ccc1Nc1cc2c(cn1)N(C)C(=O)c1occc1N2C] | 491.3 | A | 0.1 | A | E | E | E | E | E | E | E |
| 44 | 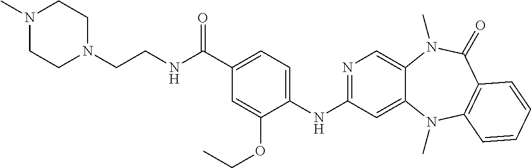 [SMILES: CCOc1cc(C(=O)NCCN2CCN(C)CC2)ccc1Nc1cc2c(cn1)N(C)C(=O)c1ccccc1N2C] | 544.3 | C | 1.0 | B | E | E | E | E | E | E | E |
| 45 | 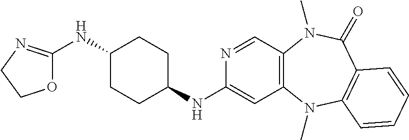 [SMILES: CN1C(=O)c2ccccc2N(C)c2cc(N[C@H]3CC[C@H](NC4=NCCO4)CC3)ncc21] | 421.3 | C | 1.0 | B | E | E | E | E | E | E | E |
| 46 | 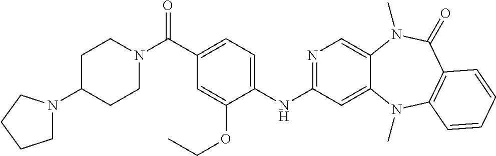 [SMILES: CCOc1cc(C(=O)N2CCC(N3CCCC3)CC2)ccc1Nc1cc2c(cn1)N(C)C(=O)c1ccccc1N2C] | 555.3 | A | 1.0 | A | E | E | E | E | E | E | E |
| 47 | 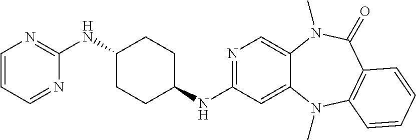 [SMILES: CN1C(=O)c2ccccc2N(C)c2cc(N[C@H]3CC[C@H](Nc4ncccn4)CC3)ncc21] | 430.3 | D | 1.0 | D | E | E | E | E | E | E | E |
| 48 | 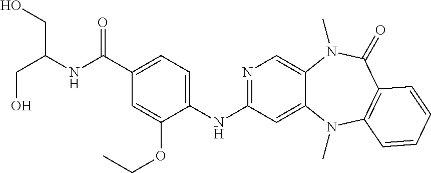 [SMILES: CCOc1cc(C(=O)NC(CO)CO)ccc1Nc1cc2c(cn1)N(C)C(=O)c1ccccc1N2C] | 492.2 | C | 1.0 | C | E | E | E | E | E | E | E |
| 49 | 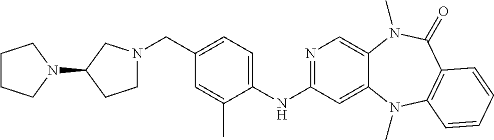 [SMILES: Cc1cc(CN2CC[C@@H](N3CCCC3)C2)ccc1Nc1cc2c(cn1)N(C)C(=O)c1ccccc1N2C] | 497.3 | A | 0.1 | A | E | E | E | E | E | E | E |
| 50 | 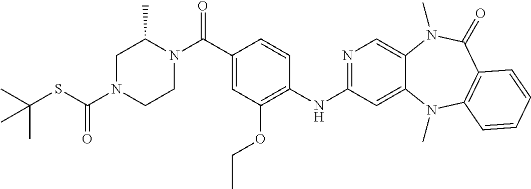 [SMILES: CCOc1cc(C(=O)N2CCN(C(=O)SC(C)(C)C)C[C@@H]2C)ccc1Nc1cc2c(cn1)N(C)C(=O)c1ccccc1N2C] | 601.3 | C | 0.1 | D | E | E | E | E | E | E | E |
| 51 | 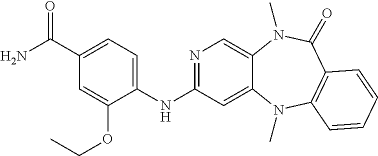 [SMILES: CCOc1cc(C(N)=O)ccc1Nc1cc2c(cn1)N(C)C(=O)c1ccccc1N2C] | 418.3 | C | 0.1 | D | E | E | E | E | E | E | E |
| 52 | 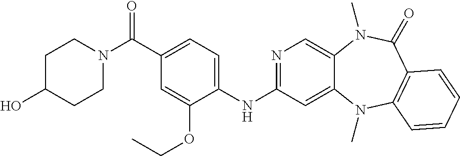 [SMILES: CCOc1cc(C(=O)N2CCC(O)CC2)ccc1Nc1cc2c(cn1)N(C)C(=O)c1ccccc1N2C] | 502.3 | B | 1.0 | A | E | E | E | E | E | E | E |
| 53 | 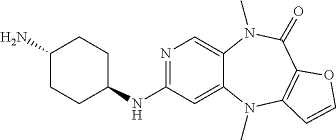 [SMILES: CN1C(=O)c2occc2N(C)c2cc(N[C@H]3CC[C@H](N)CC3)ncc21] | 342.2 | C | 1.0 | B | E | E | E | E | E | E | E |
| 54 | 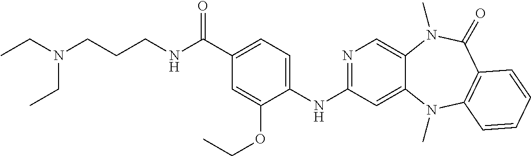 [SMILES: CCOc1cc(C(=O)NCCCN(CC)CC)ccc1Nc1cc2c(cn1)N(C)C(=O)c1ccccc1N2C] | 531.3 | C | 1.0 | B | E | E | E | E | E | E | E |
| 55 | 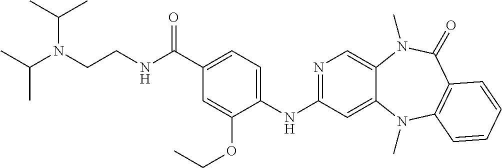 [SMILES: CCOc1cc(C(=O)NCCN(C(C)C)C(C)C)ccc1Nc1cc2c(cn1)N(C)C(=O)c1ccccc1N2C] | 545.3 | A | 0.1 | C | E | E | E | E | E | E | E |
| 56 | 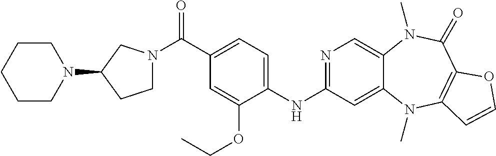 [SMILES: CCOc1cc(C(=O)N2CC[C@@H](N3CCCCC3)C2)ccc1Nc1cc2c(cn1)N(C)C(=O)c1occc1N2C] | 545.3 | A | 0.1 | A | E | E | E | E | E | E | E |
| 57 | 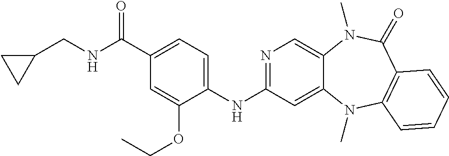 [SMILES: CCOc1cc(C(=O)NCC2CC2)ccc1Nc1cc2c(cn1)N(C)C(=O)c1ccccc1N2C] | 472.3 | C | 1.0 | C | E | E | E | E | E | E | E |
| 58 | 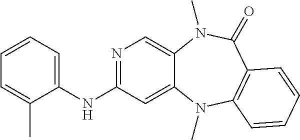 [SMILES: Cc1ccccc1Nc1cc2c(cn1)N(C)C(=O)c1ccccc1N2C] | 345.2 | D | 1.0 | C | E | E | E | E | E | E | E |
| 59 | 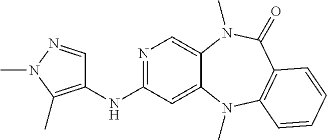 [SMILES: Cc1c(Nc2cc3c(cn2)N(C)C(=O)c2ccccc2N3C)cnn1C] | 349.2 | C | 1.0 | B | E | E | E | E | E | E | E |
| 60 | 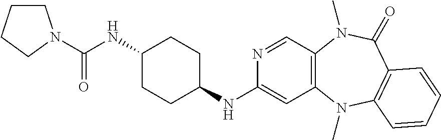 [SMILES: CN1C(=O)c2ccccc2N(C)c2cc(N[C@H]3CC[C@H](NC(=O)N4CCCC4)CC3)ncc21] | 449.4 | C | 1.0 | B | E | E | E | E | E | E | E |
| 61 | 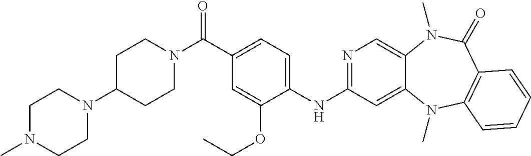 [SMILES: CCOc1cc(C(=O)N2CCC(N3CCN(C)CC3)CC2)ccc1Nc1cc2c(cn1)N(C)C(=O)c1ccccc1N2C] | 584.3 | B | 1.0 | A | E | E | E | E | E | E | E |
| 62 | 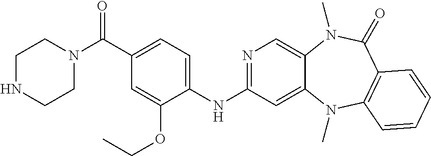 [SMILES: CCOc1cc(C(=O)N2CCNCC2)ccc1Nc1cc2c(cn1)N(C)C(=O)c1ccccc1N2C] | 487.3 | A | 1.0 | A | E | E | E | E | E | E | E |
| 63 | 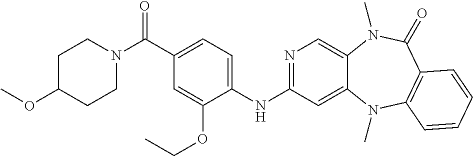 [SMILES: CCOc1cc(C(=O)N2CCC(OC)CC2)ccc1Nc1cc2c(cn1)N(C)C(=O)c1ccccc1N2C] | 516.2 | B | 1.0 | A | E | E | E | E | E | E | E |
| 64 | 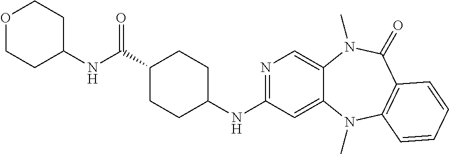 | 464.3 | C | 1.0 | B | E | E | E | E | E | E | E |
| 65 | 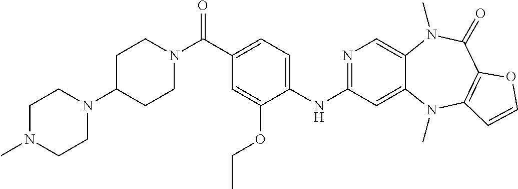 [SMILES: CCOc1cc(C(=O)N2CCC(N3CCN(C)CC3)CC2)ccc1Nc1cc2c(cn1)N(C)C(=O)c1occc1N2C] | 574.3 | A | 0.1 | A | E | E | E | E | E | E | E |
| 66 | 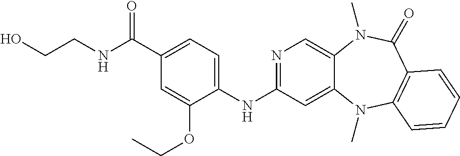 [SMILES: CCOc1cc(C(=O)NCCO)ccc1Nc1cc2c(cn1)N(C)C(=O)c1ccccc1N2C] | 462.3 | D | 1.0 | C | E | E | E | E | E | E | E |
| 67 | 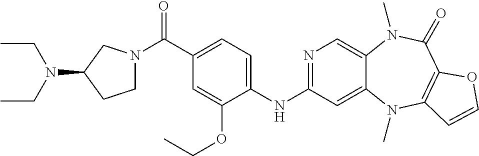 [SMILES: CCOc1cc(C(=O)N2CC[C@@H](N(CC)CC)C2)ccc1Nc1cc2c(cn1)N(C)C(=O)c1occc1N2C] | 533.3 | A | 0.1 | A | E | E | E | E | E | E | E |
| 68 | 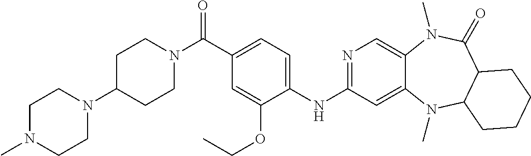 [SMILES: CCOc1cc(C(=O)N2CCC(N3CCN(C)CC3)CC2)ccc1Nc1cc2c(cn1)N(C)C(=O)C1CCCCC1N2C] | 590.4 | D | 1.0 | E | E | E | E | E | E | E | E |
| 69 | 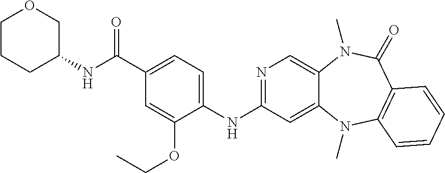 [SMILES: CCOc1cc(C(=O)N[C@@H]2CCCOC2)ccc1Nc1cc2c(cn1)N(C)C(=O)c1ccccc1N2C] | 502.3 | C | 1.0 | C | E | E | E | E | E | E | E |
| 70 | 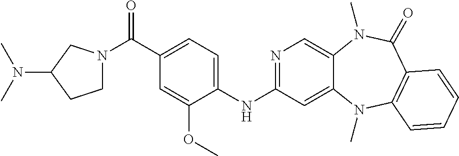 [SMILES: COc1cc(C(=O)N2CCC(N(C)C)C2)ccc1Nc1cc2c(cn1)N(C)C(=O)c1ccccc1N2C] | 501.2 | A | 0.1 | C | E | E | E | E | E | E | E |
| 71 | 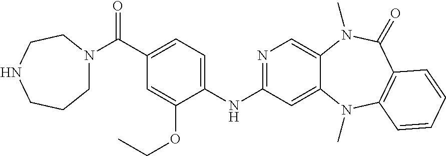 [SMILES: CCOc1cc(C(=O)N2CCCNCC2)ccc1Nc1cc2c(cn1)N(C)C(=O)c1ccccc1N2C] | 501.3 | A | 1.0 | A | E | E | E | E | E | E | E |
| 72 | 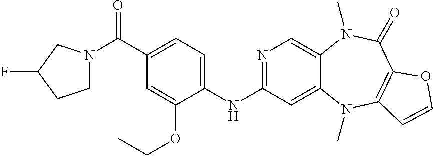 [SMILES: CCOc1cc(C(=O)N2CCC(F)C2)ccc1Nc1cc2c(cn1)N(C)C(=O)c1occc1N2C] | 480.3 | A | 0.1 | B | E | E | E | E | E | E | E |
| 73 | 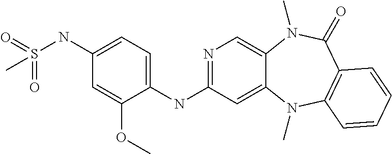 [SMILES: COc1cc(NS(C)(=O)=O)ccc1Nc1cc2c(cn1)N(C)C(=O)c1ccccc1N2C] | 454.2 | C | 1.0 | B | E | E | E | E | E | E | E |
| 74 | 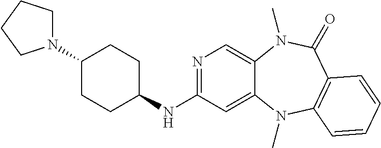 [SMILES: CN1C(=O)c2ccccc2N(C)c2cc(N[C@H]3CC[C@H](N4CCCC4)CC3)ncc21] | 406.3 | C | 1.0 | C | E | E | E | E | E | E | E |
| 75 | 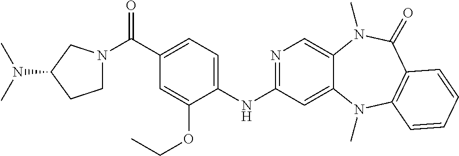 [SMILES: CCOc1cc(C(=O)N2CC[C@H](N(C)C)C2)ccc1Nc1cc2c(cn1)N(C)C(=O)c1ccccc1N2C] | 515.3 | A | 1.0 | A | E | E | E | E | E | E | E |
| 76 | 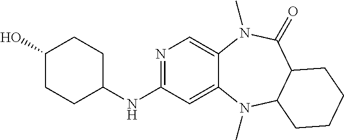 | 359.3 | D | 1.0 | E | E | E | E | E | E | E | E |
| 77 | 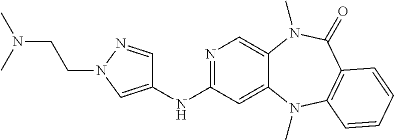 [SMILES: CN(C)CCn1cc(Nc2cc3c(cn2)N(C)C(=O)c2ccccc2N3C)cn1] | 392.3 | C | 1.0 | B | C | B | E | E | D | E | E |
| 78 | 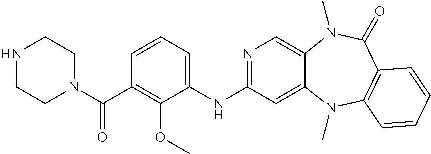 [SMILES: COc1c(Nc2cc3c(cn2)N(C)C(=O)c2ccccc2N3C)cccc1C(=O)N1CCNCC1] | 473.3 | D | 1.0 | C | E | E | E | E | E | E | E |
| 79 | 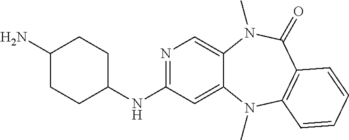 [SMILES: CN1C(=O)c2ccccc2N(C)c2cc(NC3CCC(N)CC3)ncc21] | 352.3 | C | 1.0 | B | E | E | E | E | E | E | E |
| 80 | 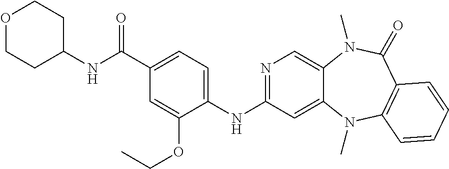 [SMILES: CCOc1cc(C(=O)NC2CCOCC2)ccc1Nc1cc2c(cn1)N(C)C(=O)c1ccccc1N2C] | 502.3 | C | 1.0 | C | E | E | E | E | E | E | E |
| 81 | 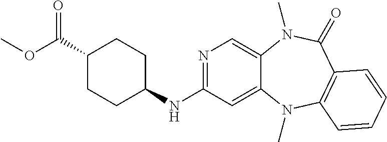 [SMILES: COC(=O)[C@H]1CC[C@H](Nc2cc3c(cn2)N(C)C(=O)c2ccccc2N3C)CC1] | 395.2 | D | 1.0 | C | E | E | E | E | E | E | E |
| 82 | 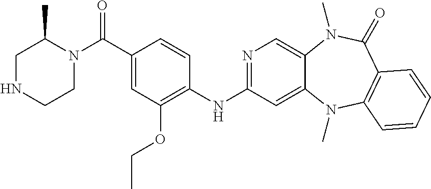 [SMILES: CCOc1cc(C(=O)N2CCNC[C@H]2C)ccc1Nc1cc2c(cn1)N(C)C(=O)c1ccccc1N2C] | 501.3 | A | 0.1 | B | E | E | E | E | E | E | E |
| 83 | 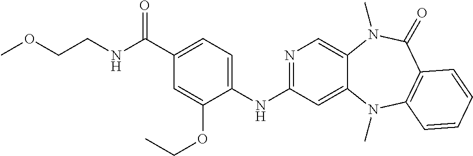 [SMILES: CCOc1cc(C(=O)NCCOC)ccc1Nc1cc2c(cn1)N(C)C(=O)c1ccccc1N2C] | 476.3 | C | 1.0 | B | E | E | E | E | E | E | E |
| 84 | 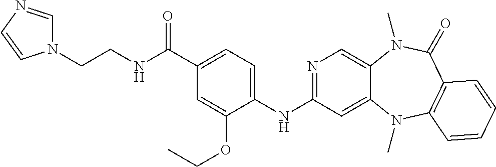 [SMILES: CCOc1cc(C(=O)NCCn2ccnc2)ccc1Nc1cc2c(cn1)N(C)C(=O)c1ccccc1N2C] | 512.3 | B | 1.0 | A | E | E | E | E | E | E | E |
| 85 | 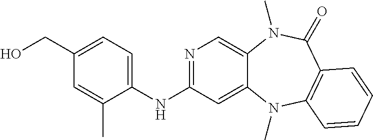 [SMILES: Cc1cc(CO)ccc1Nc1cc2c(cn1)N(C)C(=O)c1ccccc1N2C] | 375.2 | C | 1.0 | B | E | E | E | E | E | E | E |
| 86 | 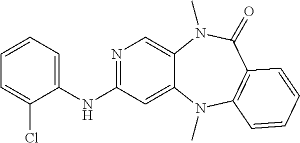 [SMILES: CN1C(=O)c2ccccc2N(C)c2cc(Nc3ccccc3Cl)ncc21] | 365.2 | D | 1.0 | C | E | E | E | E | E | E | E |
| 87 | 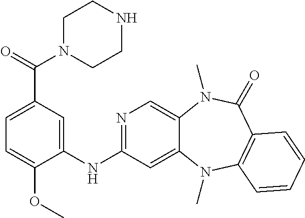 [SMILES: COc1ccc(C(=O)N2CCNCC2)cc1Nc1cc2c(cn1)N(C)C(=O)c1ccccc1N2C] | 473.3 | C | 1.0 | C | E | E | E | E | E | E | E |
| 88 | 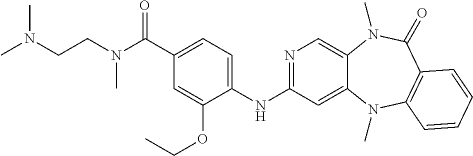 [SMILES: CCOc1cc(C(=O)N(C)CCN(C)C)ccc1Nc1cc2c(cn1)N(C)C(=O)c1ccccc1N2C] | 503.3 | A | 0.1 | A | E | E | E | E | E | E | E |
| 89 | 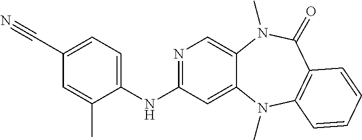 [SMILES: Cc1cc(C#N)ccc1Nc1cc2c(cn1)N(C)C(=O)c1ccccc1N2C] | 370.1 | C | 1.0 | C | E | E | E | E | E | E | E |
| 90 | 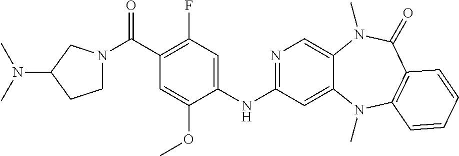 [SMILES: COc1cc(C(=O)N2CCC(N(C)C)C2)c(F)cc1Nc1cc2c(cn1)N(C)C(=O)c1ccccc1N2C] | 519.2 | C | 0.1 | E | E | E | E | E | E | E | E |
| 91 | 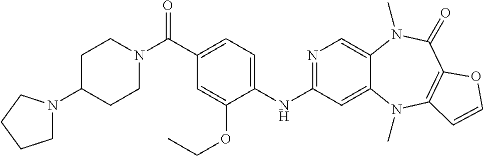 [SMILES: CCOc1cc(C(=O)N2CCC(N3CCCC3)CC2)ccc1Nc1cc2c(cn1)N(C)C(=O)c1occc1N2C] | 545.3 | A | 0.1 | A | E | E | E | E | E | E | E |
| 92 | 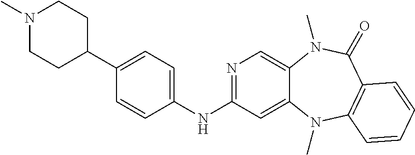 [SMILES: CN1CCC(c2ccc(Nc3cc4c(cn3)N(C)C(=O)c3ccccc3N4C)cc2)CC1] | 428.2 | A | 1.0 | A | E | C | D | E | C | E | E |
| 93 | 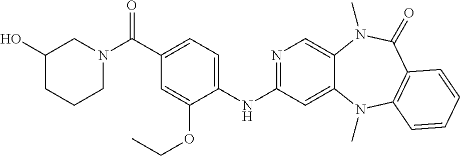 [SMILES: CCOc1cc(C(=O)N2CCCC(O)C2)ccc1Nc1cc2c(cn1)N(C)C(=O)c1ccccc1N2C] | 502.3 | B | 1.0 | A | E | E | E | E | E | E | E |
| 94 | 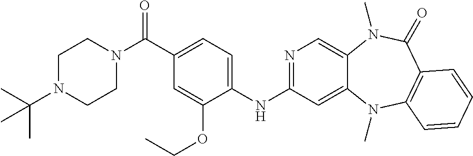 [SMILES: CCOc1cc(C(=O)N2CCN(C(C)(C)C)CC2)ccc1Nc1cc2c(cn1)N(C)C(=O)c1ccccc1N2C] | 543.3 | A | 0.1 | B | E | E | E | E | E | E | E |
| 95 | 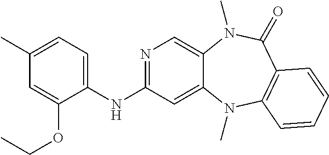 [SMILES: CCOc1cc(C)ccc1Nc1cc2c(cn1)N(C)C(=O)c1ccccc1N2C] | 389.3 | D | 1.0 | E | E | E | E | E | E | E | E |
| 96 | 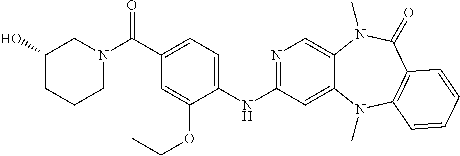 [SMILES: CCOc1cc(C(=O)N2CCC[C@H](O)C2)ccc1Nc1cc2c(cn1)N(C)C(=O)c1ccccc1N2C] | 502.2 | A | 0.1 | A | E | E | E | E | E | E | E |
| 97 | 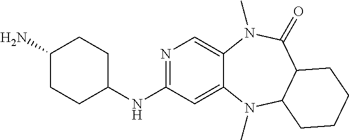 | 358.3 | D | 1.0 | E | E | E | E | E | E | E | E |
| 98 | 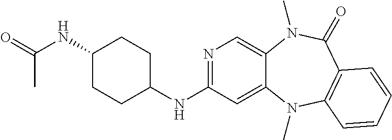 | 394.3 | D | 1.0 | C | E | E | E | E | E | E | E |
| 99 | 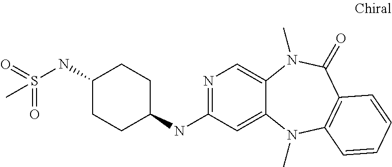 [SMILES: CN1C(=O)c2ccccc2N(C)c2cc(N[C@H]3CC[C@H](NS(C)(=O)=O)CC3)ncc21] | 430.2 | C | 1.0 | B | E | E | E | E | E | E | E |
| 100 | 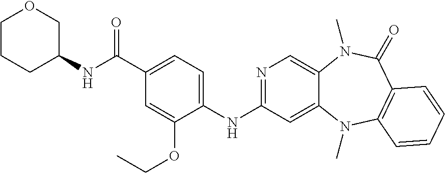 [SMILES: CCOc1cc(C(=O)N[C@H]2CCCOC2)ccc1Nc1cc2c(cn1)N(C)C(=O)c1ccccc1N2C] | 502.2 | D | 1.0 | C | E | E | E | E | E | E | E |
| 101 | 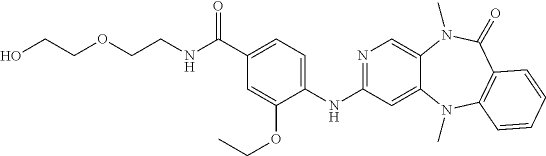 [SMILES: CCOc1cc(C(=O)NCCOCCO)ccc1Nc1cc2c(cn1)N(C)C(=O)c1ccccc1N2C] | 506.3 | C | 1.0 | B | E | E | E | E | E | E | E |
| 102 | 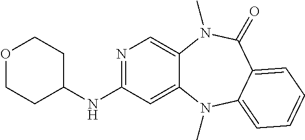 [SMILES: CN1C(=O)c2ccccc2N(C)c2cc(NC3CCOCC3)ncc21] | 339.2 | C | 1.0 | B | E | E | E | E | E | E | E |
| 103 | 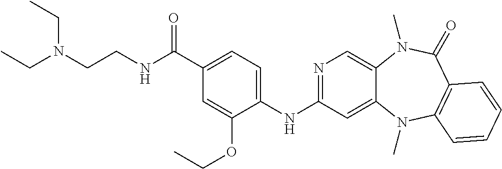 [SMILES: CCOc1cc(C(=O)NCCN(CC)CC)ccc1Nc1cc2c(cn1)N(C)C(=O)c1ccccc1N2C] | 517.3 | A | 0.1 | A | E | E | E | E | E | E | E |
| 104 | 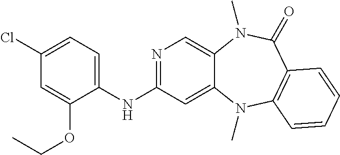 [SMILES: CCOc1cc(Cl)ccc1Nc1cc2c(cn1)N(C)C(=O)c1ccccc1N2C] | 409.2 | D | 1.0 | D | E | E | E | E | E | E | E |
| 105 | 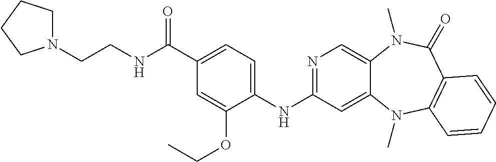 [SMILES: CCOc1cc(C(=O)NCCN2CCCC2)ccc1Nc1cc2c(cn1)N(C)C(=O)c1ccccc1N2C] | 515.3 | C | 1.0 | B | E | E | E | E | E | E | E |
| 106 | 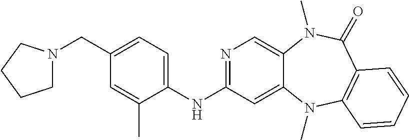 [SMILES: Cc1cc(CN2CCCC2)ccc1Nc1cc2c(cn1)N(C)C(=O)c1ccccc1N2C] | 428.3 | A | 1.0 | A | E | E | E | E | C | E | E |
| 107 | 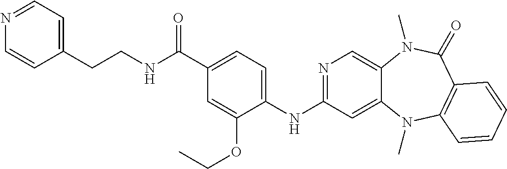 [SMILES: CCOc1cc(C(=O)NCCc2ccncc2)ccc1Nc1cc2c(cn1)N(C)C(=O)c1ccccc1N2C] | 523.3 | D | 1.0 | C | E | E | E | E | E | E | E |
| 108 | 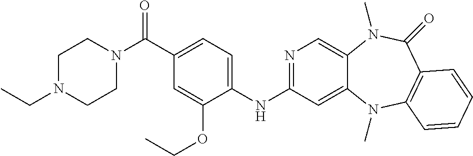 [SMILES: CCOc1cc(C(=O)N2CCN(CC)CC2)ccc1Nc1cc2c(cn1)N(C)C(=O)c1ccccc1N2C] | 515.3 | A | 1.0 | A | E | E | E | E | E | E | E |
| 109 | 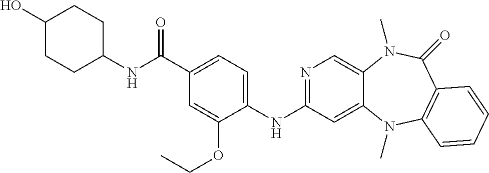 [SMILES: CCOc1cc(C(=O)NC2CCC(O)CC2)ccc1Nc1cc2c(cn1)N(C)C(=O)c1ccccc1N2C] | 516.3 | D | 1.0 | D | E | E | E | E | E | E | E |
| 110 | 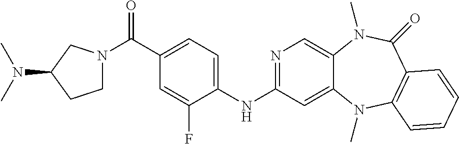 [SMILES: CN1C(=O)c2ccccc2N(C)c2cc(Nc3ccc(C(=O)N4CC[C@@H](N(C)C)C4)cc3F)ncc21] | 489.3 | A | 10.0 | A | D | C | E | E | C | E | E |
| 111 | 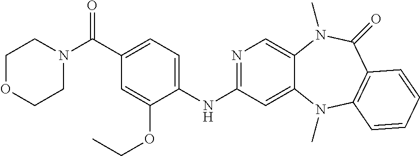 [SMILES: CCOc1cc(C(=O)N2CCOCC2)ccc1Nc1cc2c(cn1)N(C)C(=O)c1ccccc1N2C] | 488.2 | A | 1.0 | E | E | E | E | E | E | E | E |
| 112 | 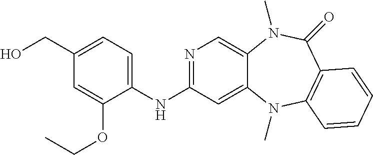 [SMILES: CCOc1cc(CO)ccc1Nc1cc2c(cn1)N(C)C(=O)c1ccccc1N2C] | 405.2 | C | 0.1 | D | E | E | E | E | E | E | E |
| 113 | 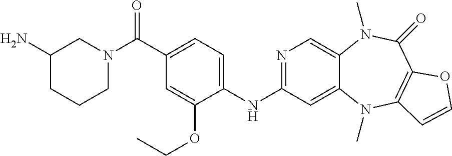 [SMILES: CCOc1cc(C(=O)N2CCCC(N)C2)ccc1Nc1cc2c(cn1)N(C)C(=O)c1occc1N2C] | 491.1 | A | 0.1 | A | E | E | E | E | E | E | E |
| 114 | 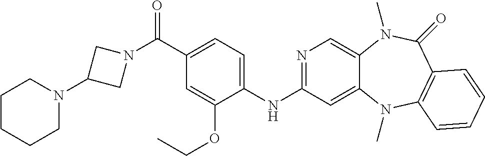 [SMILES: CCOc1cc(C(=O)N2CC(N3CCCCC3)C2)ccc1Nc1cc2c(cn1)N(C)C(=O)c1ccccc1N2C] | 541.3 | A | 0.1 | A | E | E | E | E | E | E | E |
| 115 | 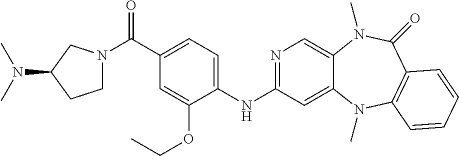 [SMILES: CCOc1cc(C(=O)N2CC[C@@H](N(C)C)C2)ccc1Nc1cc2c(cn1)N(C)C(=O)c1ccccc1N2C] | 515.3 | A | 1.0 | A | E | E | E | E | E | E | E |
| 116 | 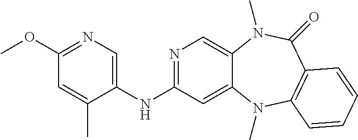 [SMILES: COc1cc(C)c(Nc2cc3c(cn2)N(C)C(=O)c2ccccc2N3C)cn1] | 376.3 | D | 1.0 | C | E | E | E | E | E | E | E |
| 117 | 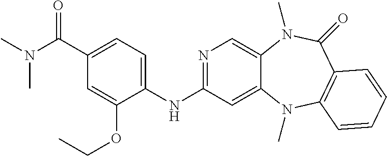 [SMILES: CCOc1cc(C(=O)N(C)C)ccc1Nc1cc2c(cn1)N(C)C(=O)c1ccccc1N2C] | 446.3 | n.d. | n.d. | n.d. | n.d. | n.d. | n.d. | n.d. | n.d. | n.d. | n.d. |
| 118 | 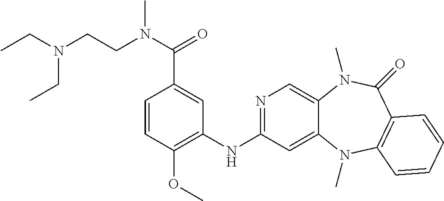 [SMILES: CCN(CC)CCN(C)C(=O)c1ccc(OC)c(Nc2cc3c(cn2)N(C)C(=O)c2ccccc2N3C)c1] | 517.3 | n.d. | n.d. | n.d. | n.d. | n.d. | n.d. | n.d. | n.d. | n.d. | n.d. |
| 119 | 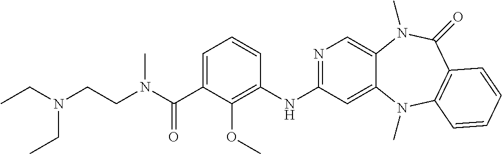 [SMILES: CCN(CC)CCN(C)C(=O)c1cccc(Nc2cc3c(cn2)N(C)C(=O)c2ccccc2N3C)c1OC] | 517.3 | n.d. | n.d. | n.d. | n.d. | n.d. | n.d. | n.d. | n.d. | n.d. | n.d. |
| 120 | 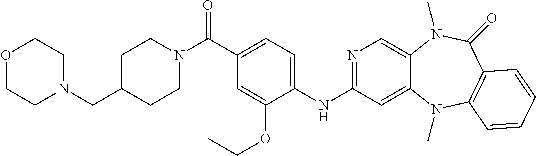 [SMILES: CCOc1cc(C(=O)N2CCC(CN3CCOCC3)CC2)ccc1Nc1cc2c(cn1)N(C)C(=O)c1ccccc1N2C] | 585.3 | A | 1.0 | A | E | E | E | E | E | E | E |
| 121 | 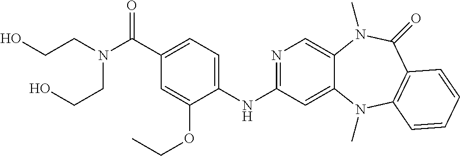 [SMILES: CCOc1cc(C(=O)N(CCO)CCO)ccc1Nc1cc2c(cn1)N(C)C(=O)c1ccccc1N2C] | 506.1 | n.d. | n.d. | n.d. | n.d. | n.d. | n.d. | n.d. | n.d. | n.d. | n.d. |
| 122 | 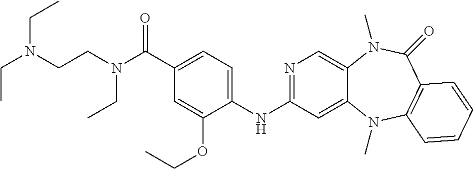 [SMILES: CCOc1cc(C(=O)N(CC)CCN(CC)CC)ccc1Nc1cc2c(cn1)N(C)C(=O)c1ccccc1N2C] | 545.3 | A | 1.0 | A | E | E | E | E | E | E | E |
| T1 | 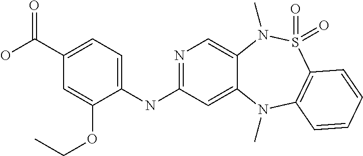 [SMILES: CCOc1cc(C(=O)O)ccc1Nc1cc2c(cn1)N(C)S(=O)(=O)c1ccccc1N2C] | 455.0 | D | 10 | E | E | E | E | E | E | E | E |
| T2 | 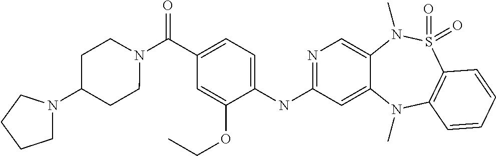 [SMILES: CCOc1cc(C(=O)N2CCC(N3CCCC3)CC2)ccc1Nc1cc2c(cn1)N(C)S(=O)(=O)c1ccccc1N2C] | 591.3 | D | 10 | D | E | E | E | E | E | E | E |
| T3 | 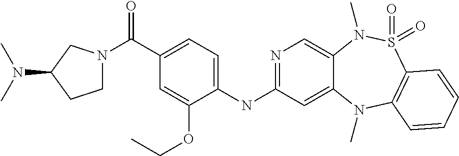 [SMILES: CCOc1cc(C(=O)N2CC[C@@H](N(C)C)C2)ccc1Nc1cc2c(cn1)N(C)S(=O)(=O)c1ccccc1N2C] | 551.3 | D | 10 | E | E | E | E | E | E | E | E |
| T4 | 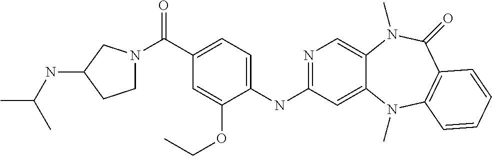 [SMILES: CCOc1cc(C(=O)N2CCC(NC(C)C)C2)ccc1Nc1cc2c(cn1)N(C)C(=O)c1ccccc1N2C] | 529.3 | A | 1 | A | E | E | E | E | E | E | E |
| T5 | 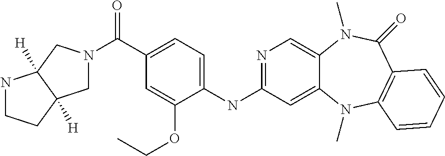 [SMILES: CCOc1cc(C(=O)N2C[C@H]3CCN[C@H]3C2)ccc1Nc1cc2c(cn1)N(C)C(=O)c1ccccc1N2C] | 513.3 | A | 1 | A | E | E | E | E | E | E | E |
| T6 | 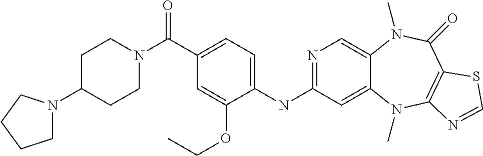 [SMILES: CCOc1cc(C(=O)N2CCC(N3CCCC3)CC2)ccc1Nc1cc2c(cn1)N(C)C(=O)c1scnc1N2C] | 562.3 | A | 1 | A | E | E | E | E | E | E | E |
| T7 | 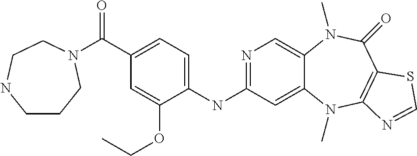 [SMILES: CCOc1cc(C(=O)N2CCCNCC2)ccc1Nc1cc2c(cn1)N(C)C(=O)c1scnc1N2C] | 508.1 | B | 1 | A | E | E | E | E | E | E | E |
| T8 | 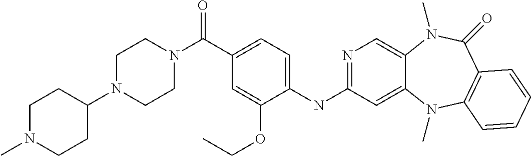 [SMILES: CCOc1cc(C(=O)N2CCN(C3CCN(C)CC3)CC2)ccc1Nc1cc2c(cn1)N(C)C(=O)c1ccccc1N2C] | 584.3 | A | 1 | A | E | E | E | E | E | E | E |
| T9 | 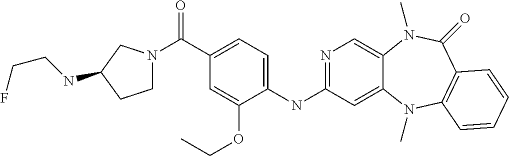 [SMILES: CCOc1cc(C(=O)N2CC[C@@H](NCCF)C2)ccc1Nc1cc2c(cn1)N(C)C(=O)c1ccccc1N2C] | 533.2 | A | 1 | A | E | E | E | E | E | E | E |
| T10 | 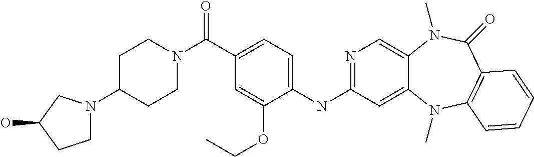 [SMILES: CCOc1cc(C(=O)N2CCC(N3CC[C@@H](O)C3)CC2)ccc1Nc1cc2c(cn1)N(C)C(=O)c1ccccc1N2C] | 571.2 | A | 1 | A | E | E | E | E | E | E | E |
| T11 | 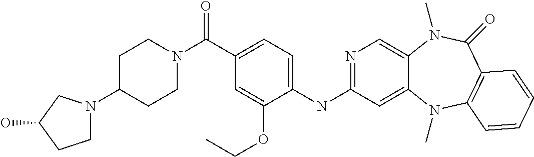 [SMILES: CCOc1cc(C(=O)N2CCC(N3CC[C@H](O)C3)CC2)ccc1Nc1cc2c(cn1)N(C)C(=O)c1ccccc1N2C] | 571.3 | A | 1 | A | E | E | E | E | E | E | E |
| T14 | 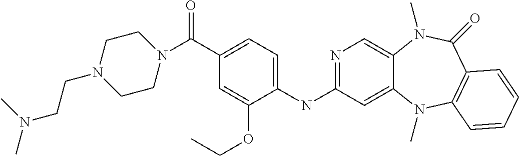 [SMILES: CCOc1cc(C(=O)N2CCN(CCN(C)C)CC2)ccc1Nc1cc2c(cn1)N(C)C(=O)c1ccccc1N2C] | 558.3 | A | 1 | A | E | E | E | E | E | E | E |
| T15 | 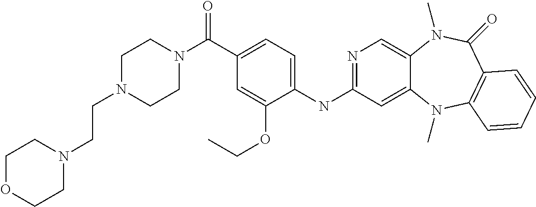 [SMILES: CCOc1cc(C(=O)N2CCN(CCN3CCOCC3)CC2)ccc1Nc1cc2c(cn1)N(C)C(=O)c1ccccc1N2C] | 600.2 | A | 1 | A | E | E | E | E | E | E | E |
| T18 | 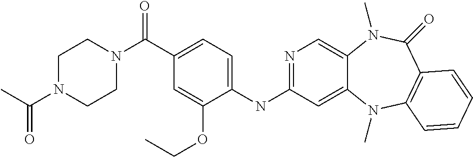 [SMILES: CCOc1cc(C(=O)N2CCN(C(C)=O)CC2)ccc1Nc1cc2c(cn1)N(C)C(=O)c1ccccc1N2C] | 529.3 | A | 1 | A | E | E | E | E | E | E | E |
| T20 | 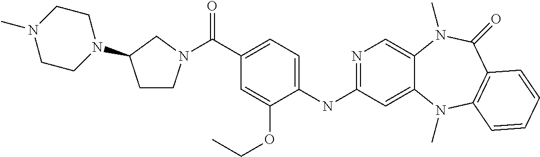 [SMILES: CCOc1cc(C(=O)N2CC[C@@H](N3CCN(C)CC3)C2)ccc1Nc1cc2c(cn1)N(C)C(=O)c1ccccc1N2C] | 570.3 | A | 1 | A | E | E | E | E | E | E | E |
| T22 | 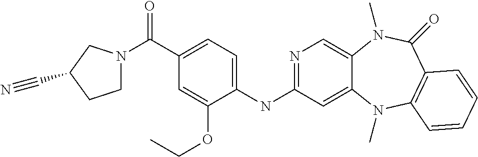 [SMILES: CCOc1cc(C(=O)N2CC[C@H](C#N)C2)ccc1Nc1cc2c(cn1)N(C)C(=O)c1ccccc1N2C] | 497.2 | A | 1 | A | E | E | E | E | E | E | E |
| T31 | 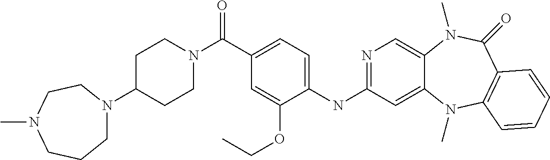 [SMILES: CCOc1cc(C(=O)N2CCC(N3CCCN(C)CC3)CC2)ccc1Nc1cc2c(cn1)N(C)C(=O)c1ccccc1N2C] | 598.3 | A | 1 | A | E | E | E | E | E | E | E |
| T34 | 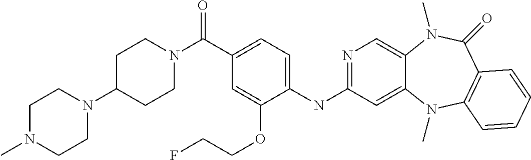 [SMILES: CN1CCN(C2CCN(C(=O)c3ccc(Nc4cc5c(cn4)N(C)C(=O)c4ccccc4N5C)c(OCCF)c3)CC2)CC1] | 602.2 | A | 1 | A | E | E | E | E | E | E | E |
| T42 | 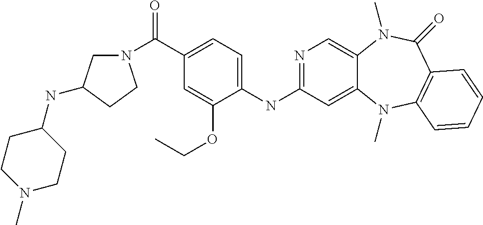 [SMILES: CCOc1cc(C(=O)N2CCC(NC3CCN(C)CC3)C2)ccc1Nc1cc2c(cn1)N(C)C(=O)c1ccccc1N2C] | 584.3 | B | 1 | A | E | E | E | E | E | E | E |
| T47 | 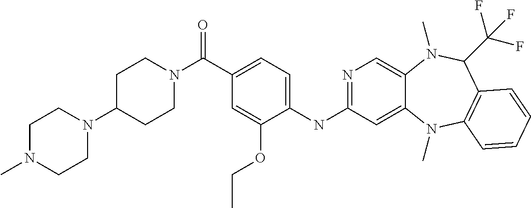 [SMILES: CCOc1cc(C(=O)N2CCC(N3CCN(C)CC3)CC2)ccc1Nc1cc2c(cn1)N(C)C(C(F)(F)F)c1ccccc1N2C] | 638.3 | D | 10 | C | E | E | E | E | E | E | E |
| T48 | 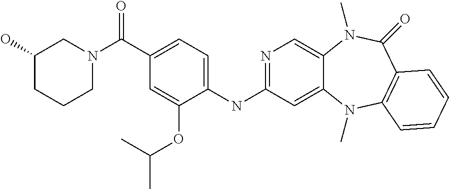 [SMILES: CC(C)Oc1cc(C(=O)N2CCC[C@H](O)C2)ccc1Nc1cc2c(cn1)N(C)C(=O)c1ccccc1N2C] | 516.2 | C | 10 | A | E | E | E | E | E | E | E |
| T49 | 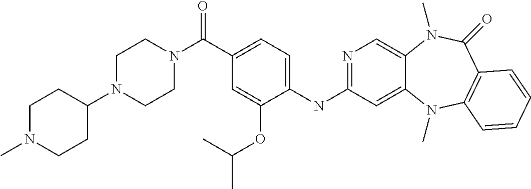 [SMILES: CC(C)Oc1cc(C(=O)N2CCN(C3CCN(C)CC3)CC2)ccc1Nc1cc2c(cn1)N(C)C(=O)c1ccccc1N2C] | 597.3 | B | 1 | A | E | E | E | E | E | E | E |
| T50 | 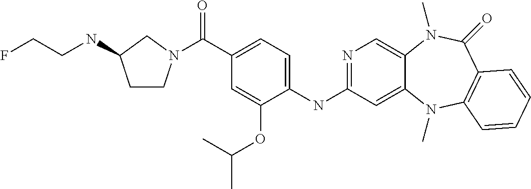 [SMILES: CC(C)Oc1cc(C(=O)N2CC[C@@H](NCCF)C2)ccc1Nc1cc2c(cn1)N(C)C(=O)c1ccccc1N2C] | 547.3 | C | 1 | B | E | E | E | E | E | E | E |
| T54 | 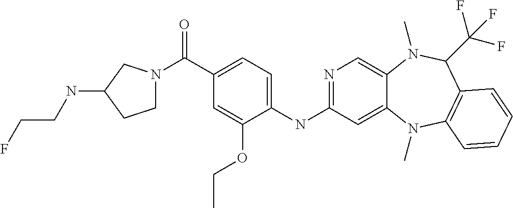 [SMILES: CCOc1cc(C(=O)N2CCC(NCCF)C2)ccc1Nc1cc2c(cn1)N(C)C(C(F)(F)F)c1ccccc1N2C] | 587.3 | D | 10 | C | E | E | E | E | E | E | E |
| T56 | 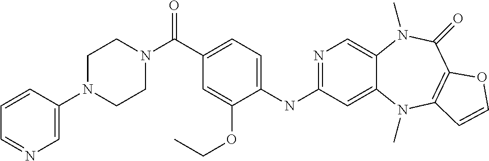 [SMILES: CCOc1cc(C(=O)N2CCN(c3cccnc3)CC2)ccc1Nc1cc2c(cn1)N(C)C(=O)c1occc1N2C] | 554.2 | A | 1 | A | E | E | E | E | E | E | E |
| T59 | 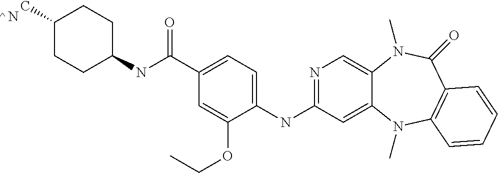 | 525.2 | C | 1 | C | E | E | E | E | E | E | E |
| T62 | 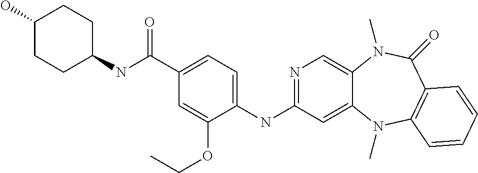 [SMILES: CCOc1cc(C(=O)N[C@H]2CC[C@H](O)CC2)ccc1Nc1cc2c(cn1)N(C)C(=O)c1ccccc1N2C] | 516.2 | C | 1 | B | E | E | E | E | E | E | E |
| T63 | 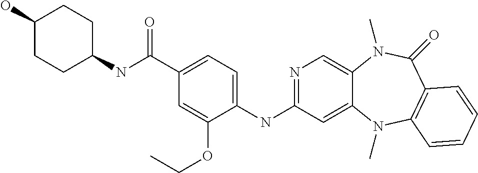 [SMILES: CCOc1cc(C(=O)N[C@H]2CC[C@@H](O)CC2)ccc1Nc1cc2c(cn1)N(C)C(=O)c1ccccc1N2C] | 516.2 | C | 1 | B | E | E | E | E | E | E | E |
| T64 | 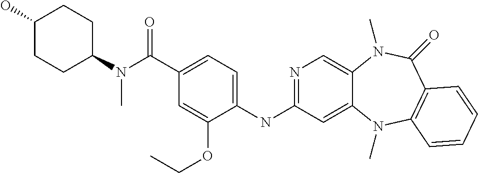 [SMILES: CCOc1cc(C(=O)N(C)[C@H]2CC[C@H](O)CC2)ccc1Nc1cc2c(cn1)N(C)C(=O)c1ccccc1N2C] | 530.3 | B | 1 | A | E | E | E | E | E | E | E |
| T65 | 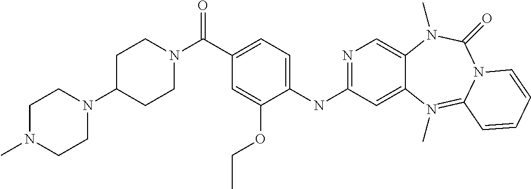 | 571.5 | D | 10 | D | E | E | E | E | E | E | E |
| T66 | 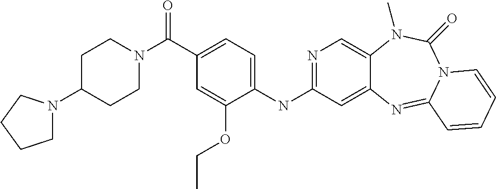 [SMILES: CCOc1cc(C(=O)N2CCC(N3CCCC3)CC2)ccc1Nc1cc2c(cn1)N(C)C(=O)N1C=CC=CC1=N2] | 542.4 | D | 10 | D | E | E | E | E | E | C | E |
| T67 | 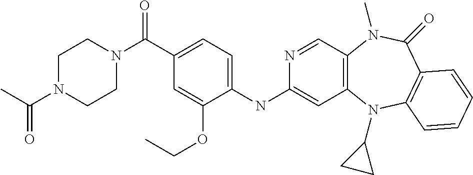 [SMILES: CCOc1cc(C(=O)N2CCN(C(C)=O)CC2)ccc1Nc1cc2c(cn1)N(C)C(=O)c1ccccc1N2C1CC1] | 555.2 | A | 1 | A | E | E | E | E | E | D | E |
| T68 | 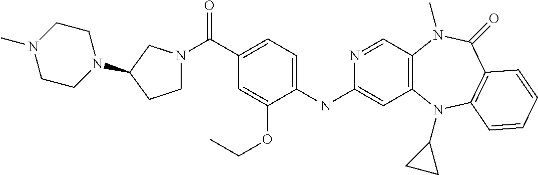 [SMILES: CCOc1cc(C(=O)N2CC[C@@H](N3CCN(C)CC3)C2)ccc1Nc1cc2c(cn1)N(C)C(=O)c1ccccc1N2C1CC1] | 596.3 | A | 1 | A | E | E | E | E | E | E | E |
| T69 | 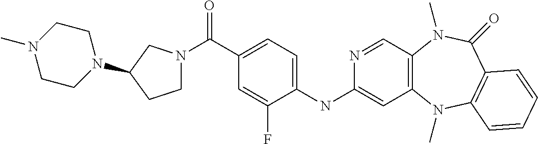 [SMILES: CN1CCN([C@@H]2CCN(C(=O)c3ccc(Nc4cc5c(cn4)N(C)C(=O)c4ccccc4N5C)c(F)c3)C2)CC1] | 544.3 | A | 1 | A | E | E | E | E | E | D | E |
| T70 | 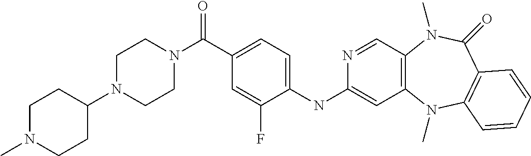 [SMILES: CN1CCC(N2CCN(C(=O)c3ccc(Nc4cc5c(cn4)N(C)C(=O)c4ccccc4N5C)c(F)c3)CC2)CC1] | 558.3 | A | 1 | A | E | E | E | E | E | E | E |
| T71 | 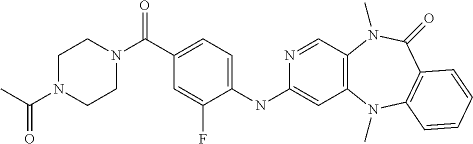 [SMILES: CC(=O)N1CCN(C(=O)c2ccc(Nc3cc4c(cn3)N(C)C(=O)c3ccccc3N4C)c(F)c2)CC1] | 503.2 | A | 1 | A | E | E | E | E | E | E | E |
| T72 | 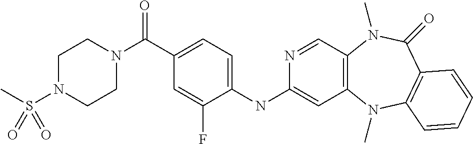 [SMILES: CN1C(=O)c2ccccc2N(C)c2cc(Nc3ccc(C(=O)N4CCN(S(C)(=O)=O)CC4)cc3F)ncc21] | 539.2 | A | 1 | A | E | D | E | E | E | E | E |
| T73 | 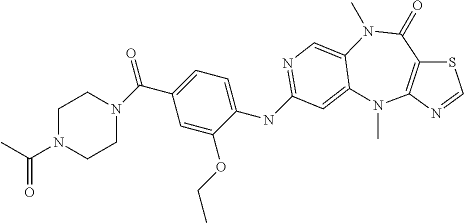 [SMILES: CCOc1cc(C(=O)N2CCN(C(C)=O)CC2)ccc1Nc1cc2c(cn1)N(C)C(=O)c1scnc1N2C] | 536.2 | D | 1 | D | E | E | E | E | E | E | E |
| T74 | 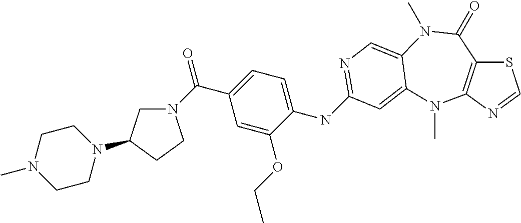 [SMILES: CCOc1cc(C(=O)N2CC[C@@H](N3CCN(C)CC3)C2)ccc1Nc1cc2c(cn1)N(C)C(=O)c1scnc1N2C] | 577.2 | D | 1 | C | E | E | E | E | E | E | E |
| T75 | 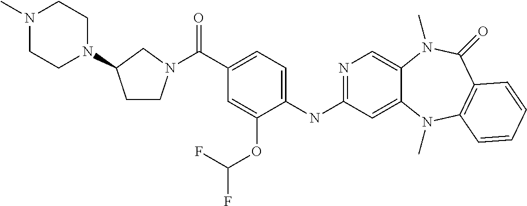 [SMILES: CN1CCN([C@@H]2CCN(C(=O)c3ccc(Nc4cc5c(cn4)N(C)C(=O)c4ccccc4N5C)c(OC(F)F)c3)C2)CC1] | 592.3 | A | 1 | A | E | E | E | E | E | E | E |
| T76 | 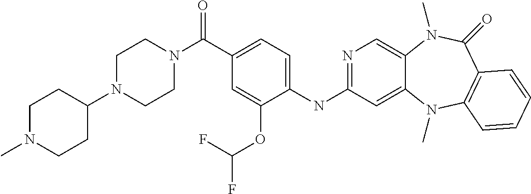 [SMILES: CN1CCC(N2CCN(C(=O)c3ccc(Nc4cc5c(cn4)N(C)C(=O)c4ccccc4N5C)c(OC(F)F)c3)CC2)CC1] | 606.3 | A | 1 | A | E | E | E | E | E | E | E |
| T77 | 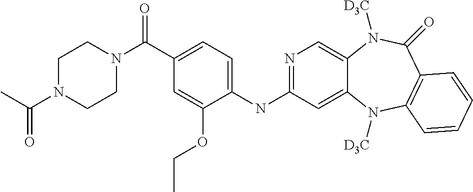 [SMILES: [2H]C([2H])([2H])N1C(=O)c2ccccc2N(C([2H])([2H])[2H])c2cc(Nc3ccc(C(=O)N4CCN(C(C)=O)CC4)cc3OCC)ncc21] | 535.2 | B | 1 | A | E | E | E | E | E | E | E |
| T79 | 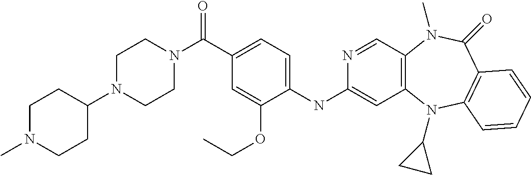 [SMILES: CCOc1cc(C(=O)N2CCN(C3CCN(C)CC3)CC2)ccc1Nc1cc2c(cn1)N(C)C(=O)c1ccccc1N2C1CC1] | 610.3 | A | 1 | A | E | E | E | E | E | E | E |
| T80 | 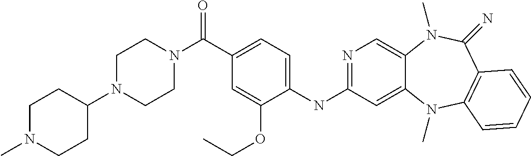 [SMILES: CCOc1cc(C(=O)N2CCN(C3CCN(C)CC3)CC2)ccc1Nc1cc2c(cn1)N(C)C(=N)c1ccccc1N2C] | 583.4 | D | 10 | E | E | E | E | E | E | E | E |
| T81 | 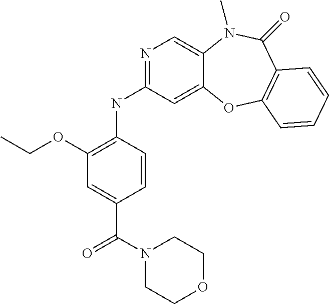 [SMILES: CCOc1cc(C(=O)N2CCOCC2)ccc1Nc1cc2c(cn1)N(C)C(=O)c1ccccc1O2] | 475.2 | C | 1 | B | E | E | E | E | E | E | E |
| T82 | 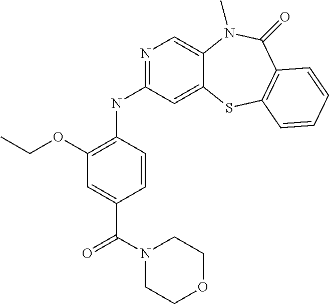 [SMILES: CCOc1cc(C(=O)N2CCOCC2)ccc1Nc1cc2c(cn1)N(C)C(=O)c1ccccc1S2] | 491.2 | C | 1 | C | E | E | E | E | E | E | E |
| T83 | 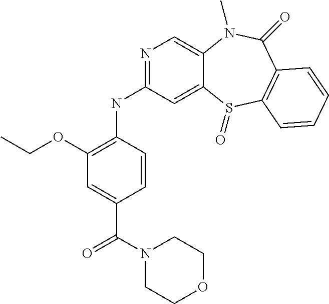 [SMILES: CCOc1cc(C(=O)N2CCOCC2)ccc1Nc1cc2c(cn1)N(C)C(=O)c1ccccc1S2=O] | 507.2 | D | 10 | B | E | E | E | E | E | E | E |
| T84 | 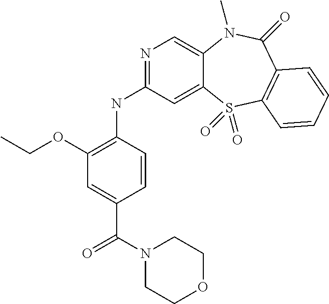 [SMILES: CCOc1cc(C(=O)N2CCOCC2)ccc1Nc1cc2c(cn1)N(C)C(=O)c1ccccc1S2(=O)=O] | 523.2 | D | 10 | B | E | E | E | E | E | E | E |
| T85 | 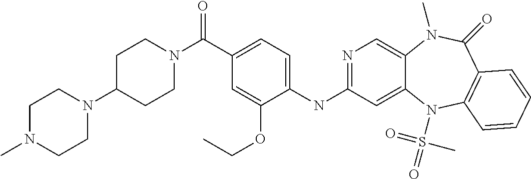 [SMILES: CCOc1cc(C(=O)N2CCC(N3CCN(C)CC3)CC2)ccc1Nc1cc2c(cn1)N(C)C(=O)c1ccccc1N2S(C)(=O)=O] | 648.3 | D | 1 | E | E | E | E | E | E | E | E |
| T86 | 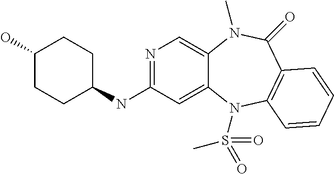 [SMILES: CN1C(=O)c2ccccc2N(S(C)(=O)=O)c2cc(N[C@H]3CC[C@H](O)CC3)ncc21] | 417.1 | D | 1 | D | E | E | E | E | E | E | E |
| T87 | 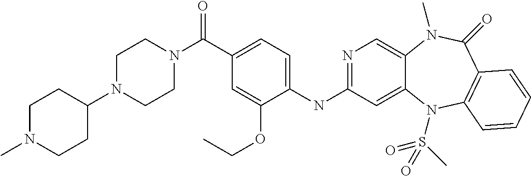 [SMILES: CCOc1cc(C(=O)N2CCN(C3CCN(C)CC3)CC2)ccc1Nc1cc2c(cn1)N(C)C(=O)c1ccccc1N2S(C)(=O)=O] | 648.4 | D | 1 | C | E | E | E | E | E | E | E |
| T88 | 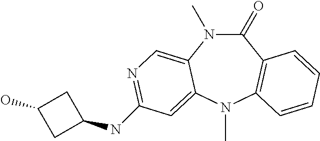 [SMILES: CN1C(=O)c2ccccc2N(C)c2cc(N[C@H]3C[C@H](O)C3)ncc21] | 325.2 | D | 10 | B | C | B | E | E | D | E | E |
| T92 | 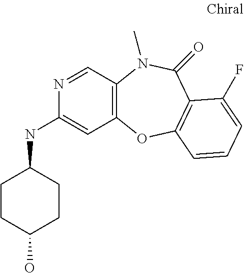 [SMILES: CN1C(=O)c2c(F)cccc2Oc2cc(N[C@H]3CC[C@H](O)CC3)ncc21] | 358.2 | C | 10 | A | E | D | E | E | C | E | E |
| T95 | 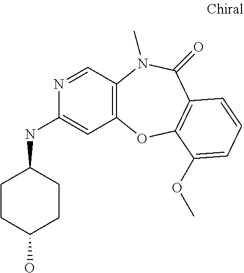 [SMILES: COc1cccc2c1Oc1cc(N[C@H]3CC[C@H](O)CC3)ncc1N(C)C2=O] | 370.2 | D | 10 | A | E | E | E | D | C | E | E |
| T97 | 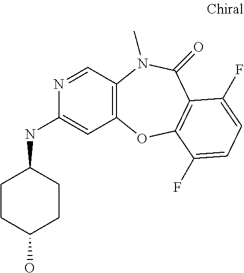 [SMILES: CN1C(=O)c2c(F)ccc(F)c2Oc2cc(N[C@H]3CC[C@H](O)CC3)ncc21] | 376.1 | D | 10 | A | E | E | E | E | C | E | E |
| T98 | 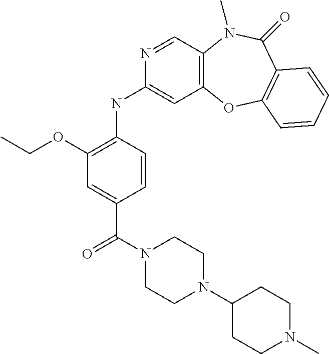 [SMILES: CCOc1cc(C(=O)N2CCN(C3CCN(C)CC3)CC2)ccc1Nc1cc2c(cn1)N(C)C(=O)c1ccccc1O2] | 571.3 | C | 10 | A | E | E | E | E | E | E | E |
| T100 | 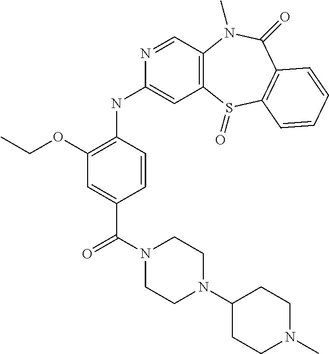 [SMILES: CCOc1cc(C(=O)N2CCN(C3CCN(C)CC3)CC2)ccc1Nc1cc2c(cn1)N(C)C(=O)c1ccccc1S2=O] | 603.3 | D | 10 | B | E | E | E | E | E | E | E |
| T102 | 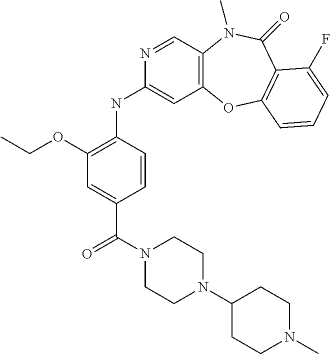 [SMILES: CCOc1cc(C(=O)N2CCN(C3CCN(C)CC3)CC2)ccc1Nc1cc2c(cn1)N(C)C(=O)c1c(F)cccc1O2] | 589.3 | C | 10 | A | E | E | E | E | E | E | E |
| T115 | 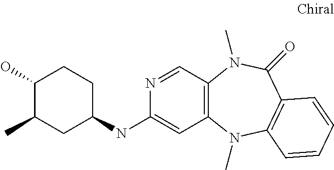 [SMILES: C[C@@H]1C[C@H](Nc2cc3c(cn2)N(C)C(=O)c2ccccc2N3C)CC[C@H]1O] | 367.2 | C | 10 | A | E | E | E | E | E | E | E |

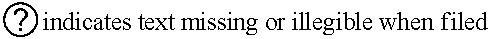

### Example 11A

### Recombinant Human ERK5 Assay

In this example, potency of the compounds in recombinant human ERK5 was assayed.

Recombinant human ERK5 catalytic domain radiometric assays were performed by Reaction Biology, Corp (Malvern, Pa.). The HotSpot radiometric assay is based on conventional filter-binding assays (Nature Biotechnology (2011) 29: 1039-1045). Compounds were tested at 20 μM down by ⅓ in a 10 point serial dilution series. Briefly, [γ-33P]-ATP was used as the tracer along with 10 μM cold ATP to label ERK5 in the presence of 20 μM myelin basic protein substrate.

The compounds tested and their IC₅₀ (μM) for recombinant ERK5 are provided in Table 1A. The IC₅₀ (μM) are represented as follows:

A is <0.02; B is 0.02-0.05; C is >0.05-0.2; D is >0.2, and

ND is no data.

TABLE 1A

| | | ESI − MS | rhERK5 |
|---|---|---|---|
| | | m/z | IC₅₀ |
| No. | Structure | [M + H]⁺ | (μM) |
| T8 | 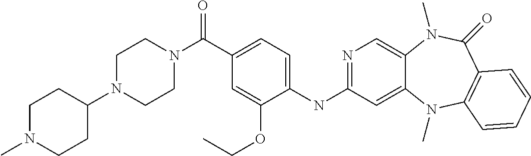 [SMILES: CCOc1cc(C(=O)N2CCN(C3CCN(C)CC3)CC2)ccc1Nc1cc2c(cn1)N(C)C(=O)c1ccccc1N2C] | 584.3 | A |
| T9 | 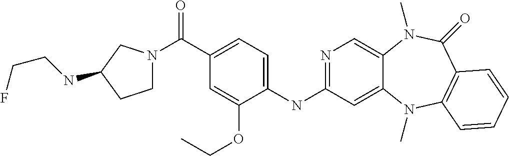 [SMILES: CCOc1cc(C(=O)N2CC[C@@H](NCCF)C2)ccc1Nc1cc2c(cn1)N(C)C(=O)c1ccccc1N2C] | 533.2 | B |
| T10 | 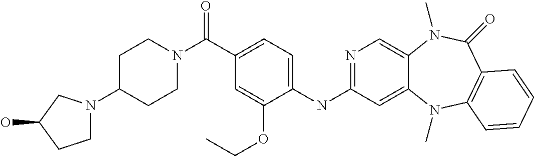 [SMILES: CCOc1cc(C(=O)N2CCC(N3CC[C@@H](O)C3)CC2)ccc1Nc1cc2c(cn1)N(C)C(=O)c1ccccc1N2C] | 571.2 | B |
| T11 | 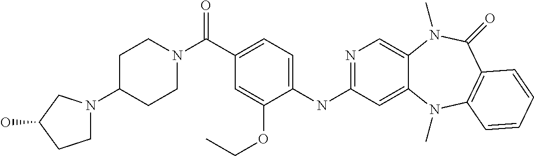 [SMILES: CCOc1cc(C(=O)N2CCC(N3CC[C@H](O)C3)CC2)ccc1Nc1cc2c(cn1)N(C)C(=O)c1ccccc1N2C] | 571.3 | B |
| T12 | 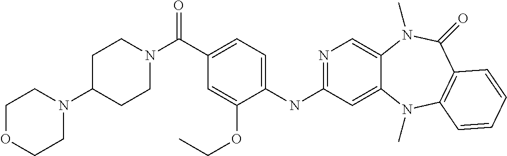 [SMILES: CCOc1cc(C(=O)N2CCC(N3CCOCC3)CC2)ccc1Nc1cc2c(cn1)N(C)C(=O)c1ccccc1N2C] | 571.2 | B |
| T13 | 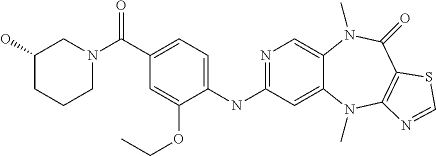 [SMILES: CCOc1cc(C(=O)N2CCC[C@H](O)C2)ccc1Nc1cc2c(cn1)N(C)C(=O)c1scnc1N2C] | 509.1 | C |
| T14 | 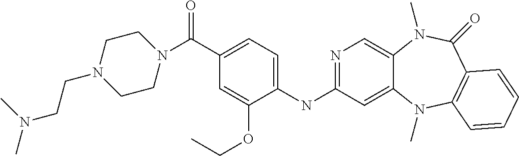 [SMILES: CCOc1cc(C(=O)N2CCN(CCN(C)C)CC2)ccc1Nc1cc2c(cn1)N(C)C(=O)c1ccccc1N2C] | 558.3 | B |
| T15 | 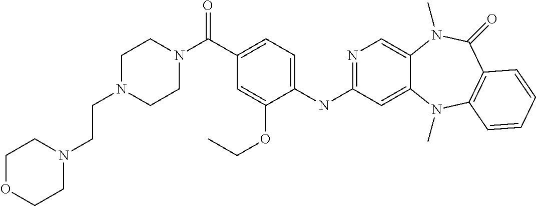 [SMILES: CCOc1cc(C(=O)N2CCN(CCN3CCOCC3)CC2)ccc1Nc1cc2c(cn1)N(C)C(=O)c1ccccc1N2C] | 600.2 | B |
| T16 | 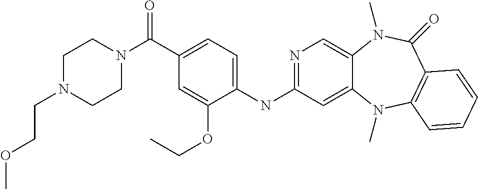 [SMILES: CCOc1cc(C(=O)N2CCN(CCOC)CC2)ccc1Nc1cc2c(cn1)N(C)C(=O)c1ccccc1N2C] | 545.2 | B |
| T17 | 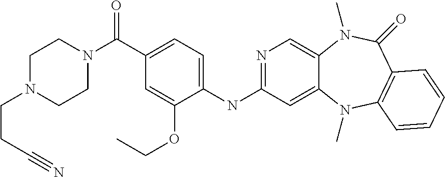 [SMILES: CCOc1cc(C(=O)N2CCN(CCC#N)CC2)ccc1Nc1cc2c(cn1)N(C)C(=O)c1ccccc1N2C] | 540.3 | B |
| T18 | 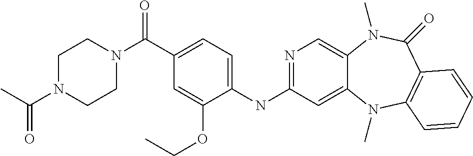 [SMILES: CCOc1cc(C(=O)N2CCN(C(C)=O)CC2)ccc1Nc1cc2c(cn1)N(C)C(=O)c1ccccc1N2C] | 529.3 | B |
| T19 | 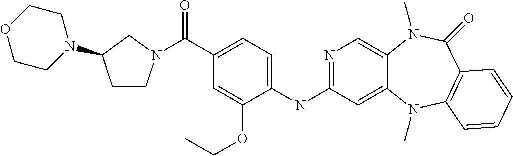 [SMILES: CCOc1cc(C(=O)N2CC[C@@H](N3CCOCC3)C2)ccc1Nc1cc2c(cn1)N(C)C(=O)c1ccccc1N2C] | 557.2 | C |
| T20 | 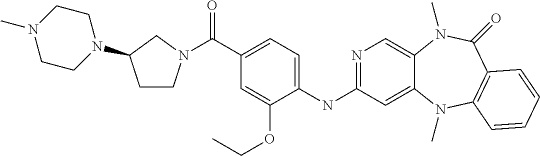 [SMILES: CCOc1cc(C(=O)N2CC[C@@H](N3CCN(C)CC3)C2)ccc1Nc1cc2c(cn1)N(C)C(=O)c1ccccc1N2C] | 570.3 | B |
| T21 | 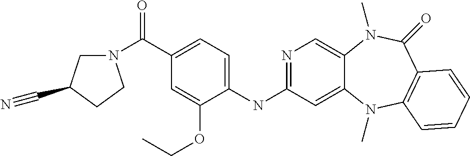 [SMILES: CCOc1cc(C(=O)N2CC[C@@H](C#N)C2)ccc1Nc1cc2c(cn1)N(C)C(=O)c1ccccc1N2C] | 497.2 | A |
| T22 | 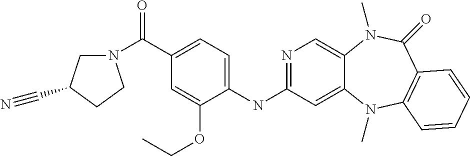 [SMILES: CCOc1cc(C(=O)N2CC[C@H](C#N)C2)ccc1Nc1cc2c(cn1)N(C)C(=O)c1ccccc1N2C] | 497.2 | C |
| T23 | 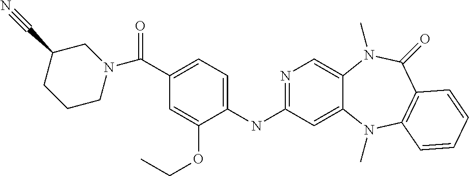 [SMILES: CCOc1cc(C(=O)N2CCC[C@@H](C#N)C2)ccc1Nc1cc2c(cn1)N(C)C(=O)c1ccccc1N2C] | 511.2 | D |
| T24 | 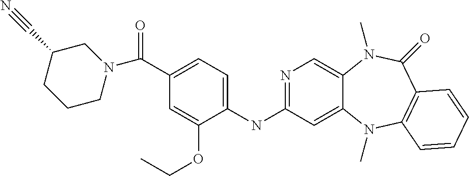 [SMILES: CCOc1cc(C(=O)N2CCC[C@H](C#N)C2)ccc1Nc1cc2c(cn1)N(C)C(=O)c1ccccc1N2C] | 511.1 | D |
| T25 | 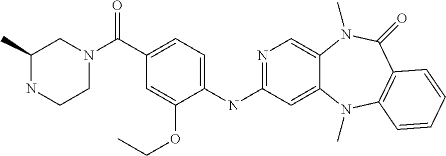 [SMILES: CCOc1cc(C(=O)N2CCN[C@@H](C)C2)ccc1Nc1cc2c(cn1)N(C)C(=O)c1ccccc1N2C] | 501.2 | A |
| T26 | 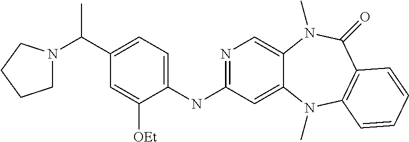 [SMILES: CCOc1cc(C(C)N2CCCC2)ccc1Nc1cc2c(cn1)N(C)C(=O)c1ccccc1N2C] | 472.2 | A |
| T27 | 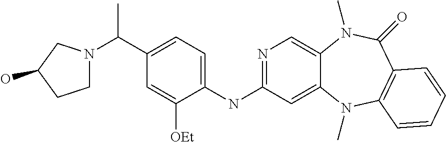 [SMILES: CCOc1cc(C(C)N2CC[C@@H](O)C2)ccc1Nc1cc2c(cn1)N(C)C(=O)c1ccccc1N2C] | 488.2 | A |
| T28 | 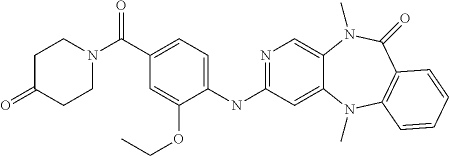 [SMILES: CCOc1cc(C(=O)N2CCC(=O)CC2)ccc1Nc1cc2c(cn1)N(C)C(=O)c1ccccc1N2C] | 500.2 | A |
| T29 | 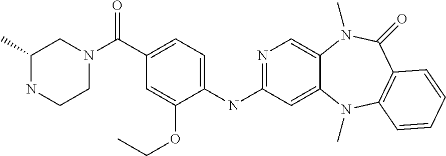 [SMILES: CCOc1cc(C(=O)N2CCN[C@H](C)C2)ccc1Nc1cc2c(cn1)N(C)C(=O)c1ccccc1N2C] | 501.2 | A |
| T30 | 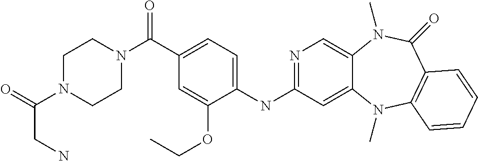 [SMILES: CCOc1cc(C(=O)N2CCN(C(=O)CN)CC2)ccc1Nc1cc2c(cn1)N(C)C(=O)c1ccccc1N2C] | 544.2 | A |
| T31 | 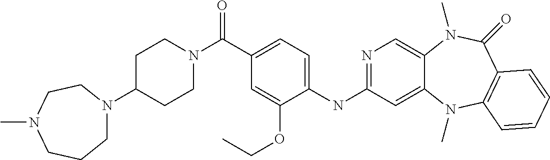 [SMILES: CCOc1cc(C(=O)N2CCC(N3CCCN(C)CC3)CC2)ccc1Nc1cc2c(cn1)N(C)C(=O)c1ccccc1N2C] | 598.3 | A |
| T32 | 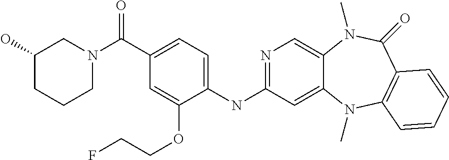 [SMILES: CN1C(=O)c2ccccc2N(C)c2cc(Nc3ccc(C(=O)N4CCC[C@H](O)C4)cc3OCCF)ncc21] | 520.2 | C |
| T33 | 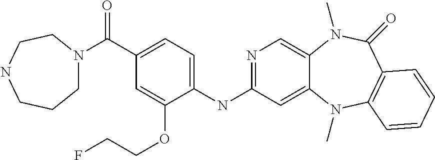 [SMILES: CN1C(=O)c2ccccc2N(C)c2cc(Nc3ccc(C(=O)N4CCCNCC4)cc3OCCF)ncc21] | 519.2 | A |
| T34 | 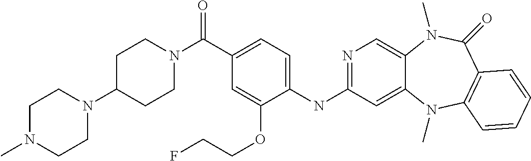 [SMILES: CN1CCN(C2CCN(C(=O)c3ccc(Nc4cc5c(cn4)N(C)C(=O)c4ccccc4N5C)c(OCCF)c3)CC2)CC1] | 602.2 | A |
| T35 | 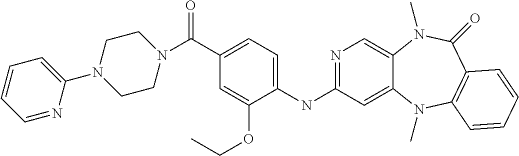 [SMILES: CCOc1cc(C(=O)N2CCN(c3ccccn3)CC2)ccc1Nc1cc2c(cn1)N(C)C(=O)c1ccccc1N2C] | 564.2 | B |
| T36 | 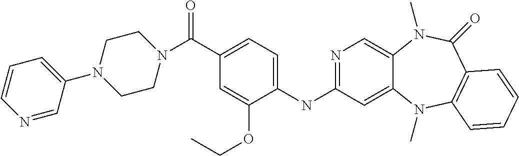 [SMILES: CCOc1cc(C(=O)N2CCN(c3cccnc3)CC2)ccc1Nc1cc2c(cn1)N(C)C(=O)c1ccccc1N2C] | 564.3 | A |
| T37 | 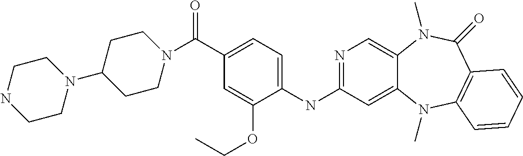 [SMILES: CCOc1cc(C(=O)N2CCC(N3CCNCC3)CC2)ccc1Nc1cc2c(cn1)N(C)C(=O)c1ccccc1N2C] | 670.3 | B |
| T38 | 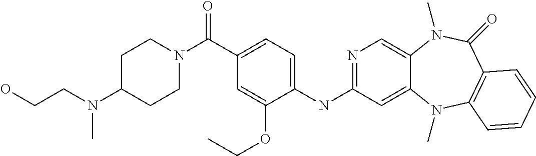 [SMILES: CCOc1cc(C(=O)N2CCC(N(C)CCO)CC2)ccc1Nc1cc2c(cn1)N(C)C(=O)c1ccccc1N2C] | 559.3 | B |
| T39 | 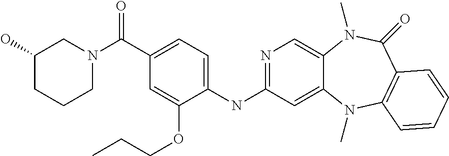 [SMILES: CCCOc1cc(C(=O)N2CCC[C@H](O)C2)ccc1Nc1cc2c(cn1)N(C)C(=O)c1ccccc1N2C] | 516.3 | D |
| T40 | 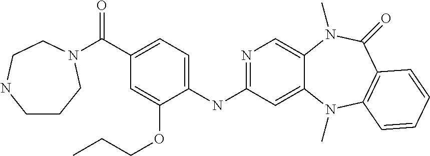 [SMILES: CCCOc1cc(C(=O)N2CCCNCC2)ccc1Nc1cc2c(cn1)N(C)C(=O)c1ccccc1N2C] | 515.3 | B |
| T41 | 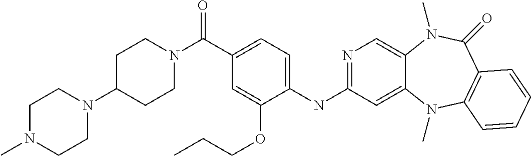 [SMILES: CCCOc1cc(C(=O)N2CCC(N3CCN(C)CC3)CC2)ccc1Nc1cc2c(cn1)N(C)C(=O)c1ccccc1N2C] | 598.3 | B |
| T42 | 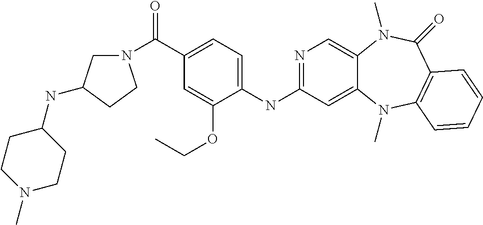 [SMILES: CCOc1cc(C(=O)N2CCC(NC3CCN(C)CC3)C2)ccc1Nc1cc2c(cn1)N(C)C(=O)c1ccccc1N2C] | 584.3 | C |
| T43 | 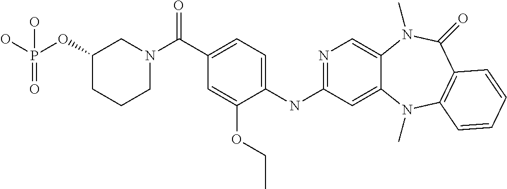 [SMILES: CCOc1cc(C(=O)N2CCC[C@H](OP(=O)(O)O)C2)ccc1Nc1cc2c(cn1)N(C)C(=O)c1ccccc1N2C] | 582.2 | B |
| T44 | 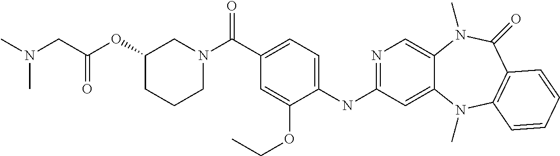 [SMILES: CCOc1cc(C(=O)N2CCC[C@H](OC(=O)CN(C)C)C2)ccc1Nc1cc2c(cn1)N(C)C(=O)c1ccccc1N2C] | 587.3 | B |
| T45 | 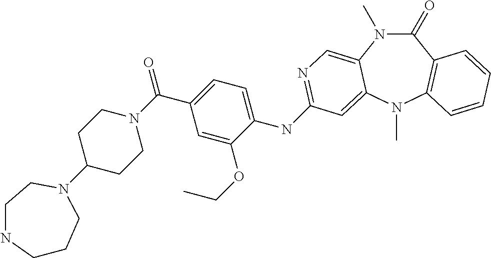 [SMILES: CCOc1cc(C(=O)N2CCC(N3CCCNCC3)CC2)ccc1Nc1cc2c(cn1)N(C)C(=O)c1ccccc1N2C] | 584.3 | A |
| T46 | 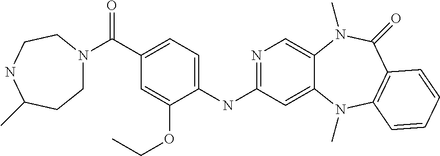 [SMILES: CCOc1cc(C(=O)N2CCNC(C)CC2)ccc1Nc1cc2c(cn1)N(C)C(=O)c1ccccc1N2C] | 515.3 | A |
| T47 | 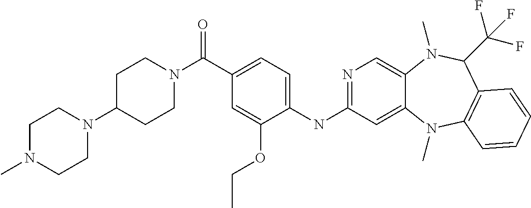 [SMILES: CCOc1cc(C(=O)N2CCC(N3CCN(C)CC3)CC2)ccc1Nc1cc2c(cn1)N(C)C(C(F)(F)F)c1ccccc1N2C] | 638.3 | D |
| T48 | 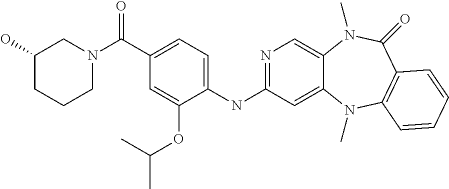 [SMILES: CC(C)Oc1cc(C(=O)N2CCC[C@H](O)C2)ccc1Nc1cc2c(cn1)N(C)C(=O)c1ccccc1N2C] | 516.2 | C |
| T49 | 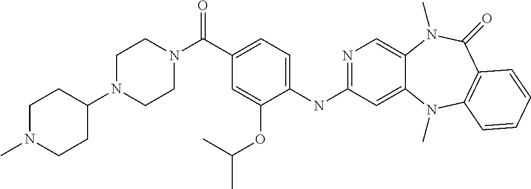 [SMILES: CC(C)Oc1cc(C(=O)N2CCN(C3CCN(C)CC3)CC2)ccc1Nc1cc2c(cn1)N(C)C(=O)c1ccccc1N2C] | 597.3 | C |
| T50 | 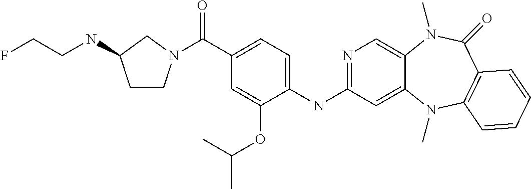 [SMILES: CC(C)Oc1cc(C(=O)N2CC[C@@H](NCCF)C2)ccc1Nc1cc2c(cn1)N(C)C(=O)c1ccccc1N2C] | 547.3 | C |
| T51 | 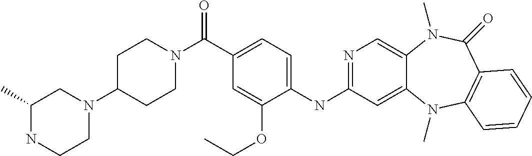 [SMILES: CCOc1cc(C(=O)N2CCC(N3CCN[C@H](C)C3)CC2)ccc1Nc1cc2c(cn1)N(C)C(=O)c1ccccc1N2C] | 584.3 | B |
| T52 | 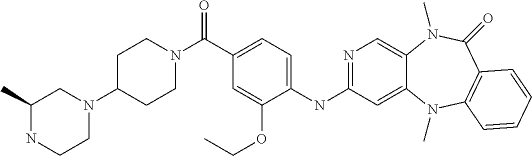 [SMILES: CCOc1cc(C(=O)N2CCC(N3CCN[C@@H](C)C3)CC2)ccc1Nc1cc2c(cn1)N(C)C(=O)c1ccccc1N2C] | 584.3 | B |
| T53 | 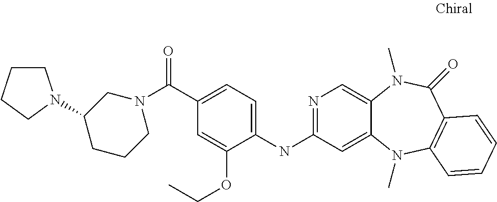 [SMILES: CCOc1cc(C(=O)N2CCC[C@H](N3CCCC3)C2)ccc1Nc1cc2c(cn1)N(C)C(=O)c1ccccc1N2C] | 555.3 | C |
| T54 | 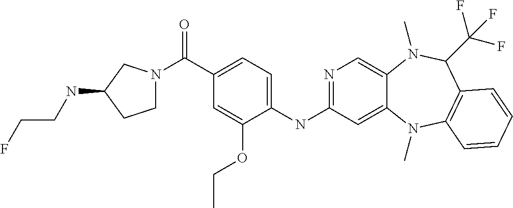 [SMILES: CCOc1cc(C(=O)N2CC[C@@H](NCCF)C2)ccc1Nc1cc2c(cn1)N(C)C(C(F)(F)F)c1ccccc1N2C] | 587.3 | D |
| T55 | 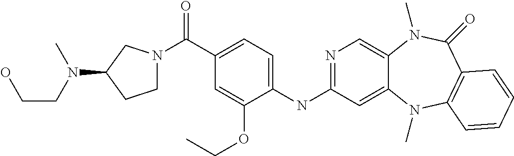 [SMILES: CCOc1cc(C(=O)N2CC[C@@H](N(C)CCO)C2)ccc1Nc1cc2c(cn1)N(C)C(=O)c1ccccc1N2C] | 545.1 | B |
| T56 | 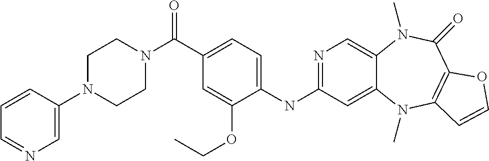 [SMILES: CCOc1cc(C(=O)N2CCN(c3cccnc3)CC2)ccc1Nc1cc2c(cn1)N(C)C(=O)c1occc1N2C] | 554.2 | A |
| T57 | 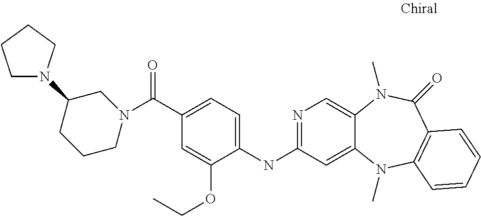 [SMILES: CCOc1cc(C(=O)N2CCC[C@@H](N3CCCC3)C2)ccc1Nc1cc2c(cn1)N(C)C(=O)c1ccccc1N2C] | 555.3 | C |
| T58 | 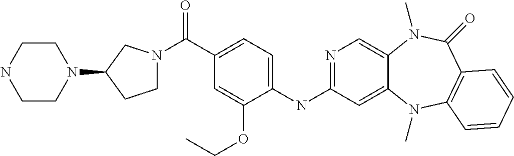 [SMILES: CCOc1cc(C(=O)N2CC[C@@H](N3CCNCC3)C2)ccc1Nc1cc2c(cn1)N(C)C(=O)c1ccccc1N2C] | 556.1 | B |
| T60 | 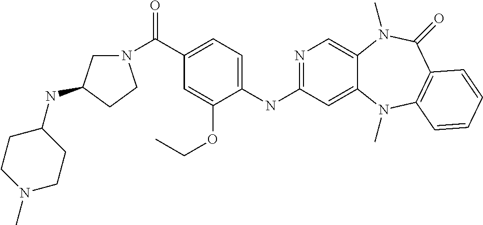 [SMILES: CCOc1cc(C(=O)N2CC[C@@H](NC3CCN(C)CC3)C2)ccc1Nc1cc2c(cn1)N(C)C(=O)c1ccccc1N2C] | 584.3 | ND |
| T61 | 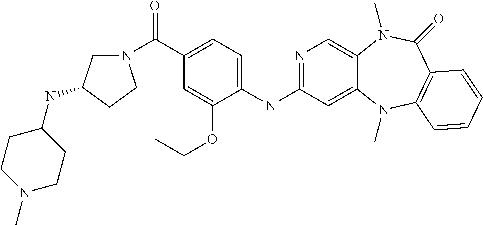 [SMILES: CCOc1cc(C(=O)N2CC[C@H](NC3CCN(C)CC3)C2)ccc1Nc1cc2c(cn1)N(C)C(=O)c1ccccc1N2C] | 584.3 | ND |
| T62 | 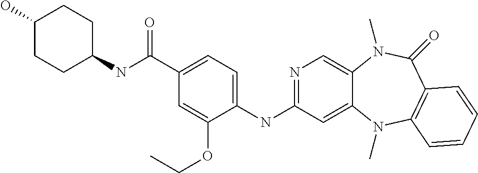 [SMILES: CCOc1cc(C(=O)N[C@H]2CC[C@H](O)CC2)ccc1Nc1cc2c(cn1)N(C)C(=O)c1ccccc1N2C] | 516.2 | D |
| T63 | 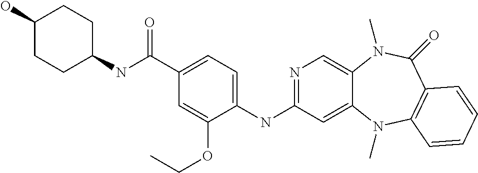 [SMILES: CCOc1cc(C(=O)N[C@H]2CC[C@@H](O)CC2)ccc1Nc1cc2c(cn1)N(C)C(=O)c1ccccc1N2C] | 516.2 | D |
| T78 | 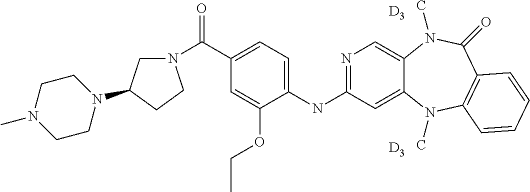 | 576.2 | ND |
| T89 | 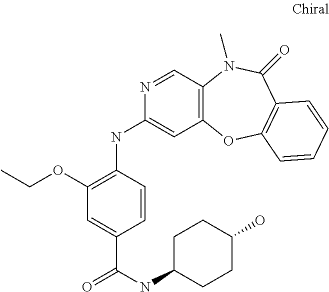 [SMILES: CCOc1cc(C(=O)N[C@H]2CC[C@H](O)CC2)ccc1Nc1cc2c(cn1)N(C)C(=O)c1ccccc1O2] | 503.2 | C |
| T90 | 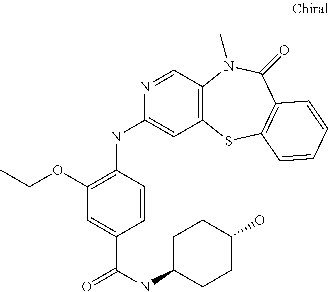 [SMILES: CCOc1cc(C(=O)N[C@H]2CC[C@H](O)CC2)ccc1Nc1cc2c(cn1)N(C)C(=O)c1ccccc1S2] | 519.2 | ND |
| T91 | 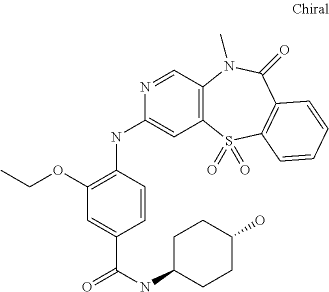 [SMILES: CCOc1cc(C(=O)N[C@H]2CC[C@H](O)CC2)ccc1Nc1cc2c(cn1)N(C)C(=O)c1ccccc1S2(=O)=O] | 551.2 | ND |
| T92 | 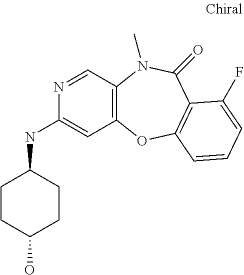 [SMILES: CN1C(=O)c2c(F)cccc2Oc2cc(N[C@H]3CC[C@H](O)CC3)ncc21] | 358.2 | C |
| T93 | 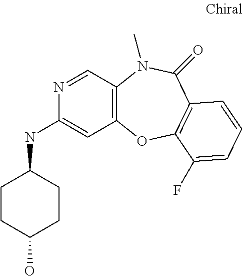 [SMILES: CN1C(=O)c2cccc(F)c2Oc2cc(N[C@H]3CC[C@H](O)CC3)ncc21] | 358.2 | D |
| T94 | 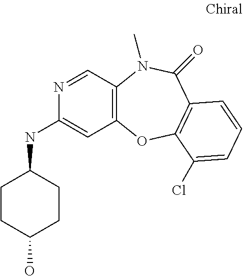 [SMILES: CN1C(=O)c2cccc(Cl)c2Oc2cc(N[C@H]3CC[C@H](O)CC3)ncc21] | 374.1 | D |
| T95 | 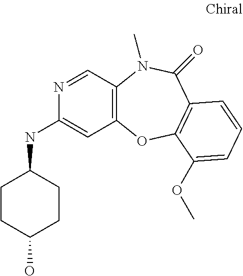 [SMILES: COc1cccc2c1Oc1cc(N[C@H]3CC[C@H](O)CC3)ncc1N(C)C2=O] | 370.2 | D |
| T96 | 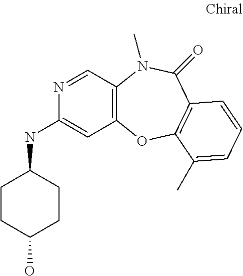 [SMILES: Cc1cccc2c1Oc1cc(N[C@H]3CC[C@H](O)CC3)ncc1N(C)C2=O] | 354.2 | D |
| T97 | 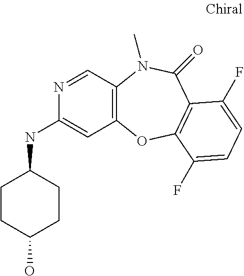 [SMILES: CN1C(=O)c2c(F)ccc(F)c2Oc2cc(N[C@H]3CC[C@H](O)CC3)ncc21] | 376.1 | D |
| T98 | 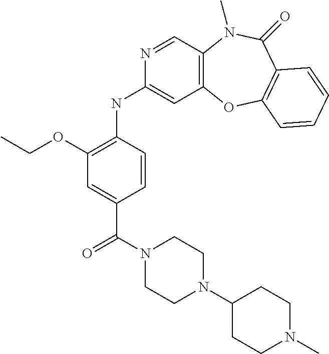 [SMILES: CCOc1cc(C(=O)N2CCN(C3CCN(C)CC3)CC2)ccc1Nc1cc2c(cn1)N(C)C(=O)c1ccccc1O2] | 571.3 | B |
| T99 | 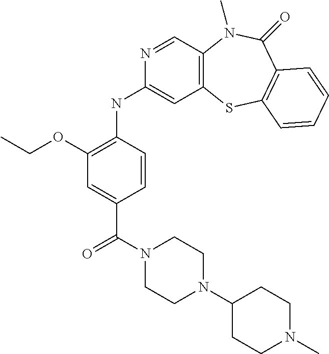 [SMILES: CCOc1cc(C(=O)N2CCN(C3CCN(C)CC3)CC2)ccc1Nc1cc2c(cn1)N(C)C(=O)c1ccccc1S2] | 587.3 | C |
| T101 | 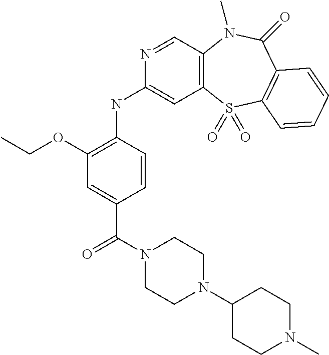 [SMILES: CCOc1cc(C(=O)N2CCN(C3CCN(C)CC3)CC2)ccc1Nc1cc2c(cn1)N(C)C(=O)c1ccccc1S2(=O)=O] | 619.3 | ND |
| T102 | 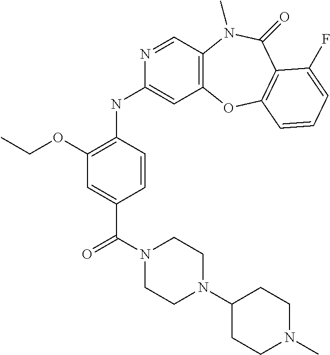 [SMILES: CCOc1cc(C(=O)N2CCN(C3CCN(C)CC3)CC2)ccc1Nc1cc2c(cn1)N(C)C(=O)c1c(F)cccc1O2] | 589.3 | B |
| T103 | 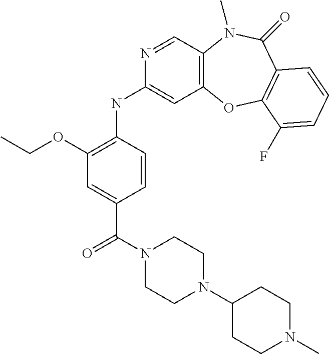 [SMILES: CCOc1cc(C(=O)N2CCN(C3CCN(C)CC3)CC2)ccc1Nc1cc2c(cn1)N(C)C(=O)c1cccc(F)c1O2] | 589.3 | C |
| T104 | 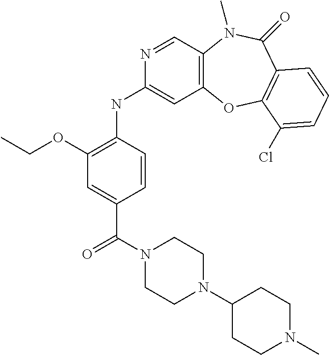 [SMILES: CCOc1cc(C(=O)N2CCN(C3CCN(C)CC3)CC2)ccc1Nc1cc2c(cn1)N(C)C(=O)c1cccc(Cl)c1O2] | 605.3 | C |
| T105 | 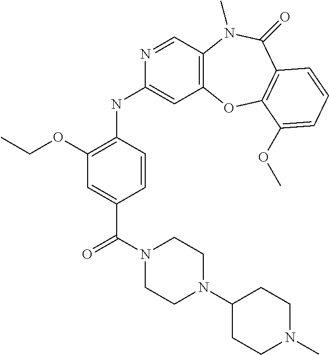 [SMILES: CCOc1cc(C(=O)N2CCN(C3CCN(C)CC3)CC2)ccc1Nc1cc2c(cn1)N(C)C(=O)c1cccc(OC)c1O2] | 601.3 | C |
| T106 | 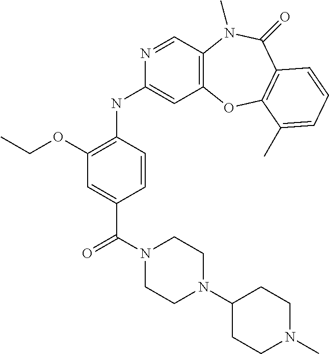 [SMILES: CCOc1cc(C(=O)N2CCN(C3CCN(C)CC3)CC2)ccc1Nc1cc2c(cn1)N(C)C(=O)c1cccc(C)c1O2] | 585.3 | C |
| T107 | 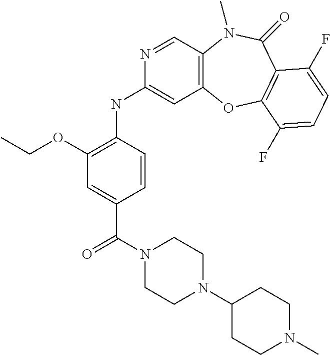 [SMILES: CCOc1cc(C(=O)N2CCN(C3CCN(C)CC3)CC2)ccc1Nc1cc2c(cn1)N(C)C(=O)c1c(F)ccc(F)c1O2] | 607.3 | B |
| T108 | 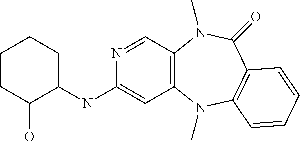 [SMILES: CN1C(=O)c2ccccc2N(C)c2cc(NC3CCCCC3O)ncc21] | 353.2 | ND |
| T109 | 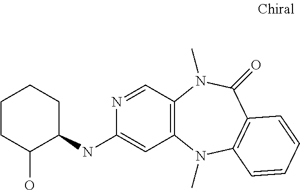 [SMILES: CN1C(=O)c2ccccc2N(C)c2cc(N[C@@H]3CCCCC3O)ncc21] | 353.2 | ND |
| T110 | 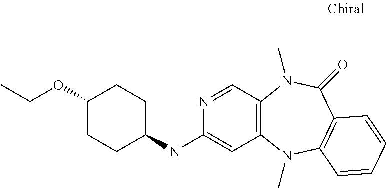 [SMILES: CCO[C@H]1CC[C@H](Nc2cc3c(cn2)N(C)C(=O)c2ccccc2N3C)CC1] | 381.2 | C |
| T111 | 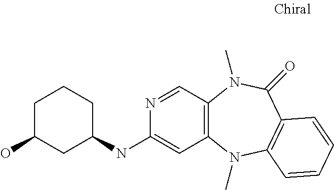 [SMILES: CN1C(=O)c2ccccc2N(C)c2cc(N[C@@H]3CCC[C@H](O)C3)ncc21] | 353.2 | ND |
| T112 | 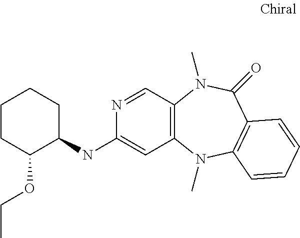 [SMILES: CCO[C@@H]1CCCC[C@H]1Nc1cc2c(cn1)N(C)C(=O)c1ccccc1N2C] | 381.2 | ND |
| T113 | 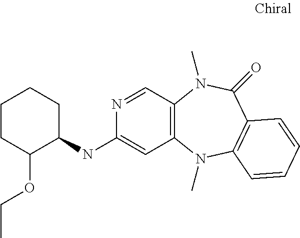 [SMILES: CCOC1CCCC[C@H]1Nc1cc2c(cn1)N(C)C(=O)c1ccccc1N2C] | 381.2 | D |
| T114 | 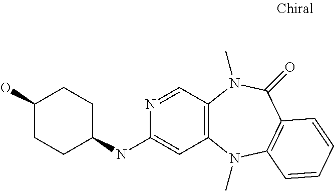 [SMILES: CN1C(=O)c2ccccc2N(C)c2cc(N[C@H]3CC[C@@H](O)CC3)ncc21] | 353.2 | D |
| T115 | 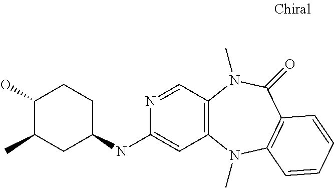 [SMILES: C[C@@H]1C[C@H](Nc2cc3c(cn2)N(C)C(=O)c2ccccc2N3C)CC[C@H]1O] | 367.2 | D |
| T116 | 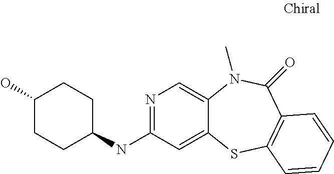 [SMILES: CN1C(=O)c2ccccc2Sc2cc(N[C@H]3CC[C@H](O)CC3)ncc21] | 356.1 | D |
| T117 | 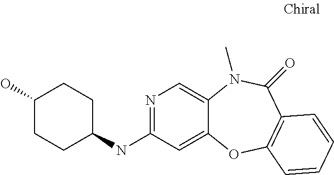 [SMILES: CN1C(=O)c2ccccc2Oc2cc(N[C@H]3CC[C@H](O)CC3)ncc21] | 340.2 | D |
| T118 | 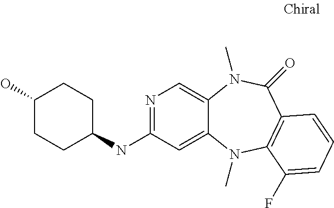 [SMILES: CN1C(=O)c2cccc(F)c2N(C)c2cc(N[C@H]3CC[C@H](O)CC3)ncc21] | 371.2 | D |
| T119 | 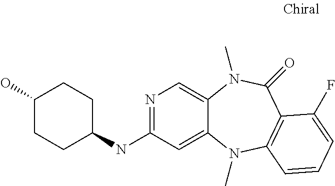 [SMILES: CN1C(=O)c2c(F)cccc2N(C)c2cc(N[C@H]3CC[C@H](O)CC3)ncc21] | 371.2 | C |
| T120 | 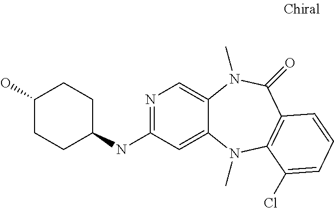 [SMILES: CN1C(=O)c2cccc(Cl)c2N(C)c2cc(N[C@H]3CC[C@H](O)CC3)ncc21] | 387.2 | D |
| T121 | 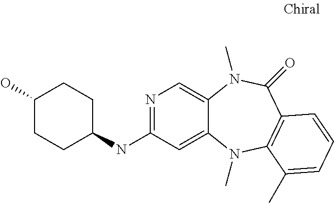 [SMILES: Cc1cccc2c1N(C)c1cc(N[C@H]3CC[C@H](O)CC3)ncc1N(C)C2=O] | 367.2 | ND |
| T122 | 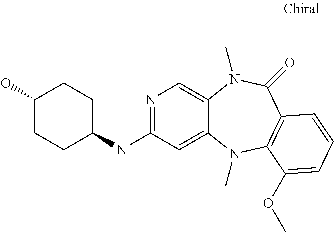 [SMILES: COc1cccc2c1N(C)c1cc(N[C@H]3CC[C@H](O)CC3)ncc1N(C)C2=O] | 383.2 | C |
| T123 | 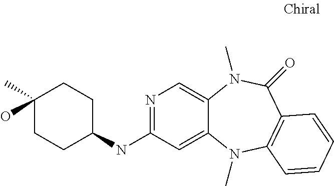 [SMILES: CN1C(=O)c2ccccc2N(C)c2cc(N[C@H]3CC[C@@](C)(O)CC3)ncc21] | 367.2 | ND |
| T124 | 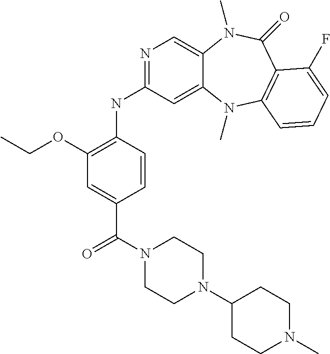 [SMILES: CCOc1cc(C(=O)N2CCN(C3CCN(C)CC3)CC2)ccc1Nc1cc2c(cn1)N(C)C(=O)c1c(F)cccc1N2C] | 602.3 | B |
| T125 | 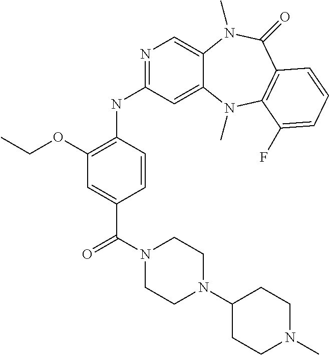 [SMILES: CCOc1cc(C(=O)N2CCN(C3CCN(C)CC3)CC2)ccc1Nc1cc2c(cn1)N(C)C(=O)c1cccc(F)c1N2C] | 602.3 | C |
| T126 | 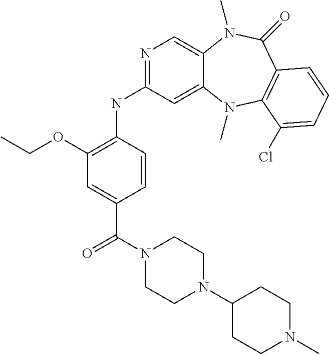 [SMILES: CCOc1cc(C(=O)N2CCN(C3CCN(C)CC3)CC2)ccc1Nc1cc2c(cn1)N(C)C(=O)c1cccc(Cl)c1N2C] | 618.3 | C |
| T127 | 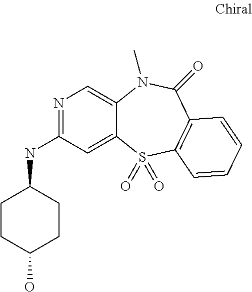 [SMILES: CN1C(=O)c2ccccc2S(=O)(=O)c2cc(N[C@H]3CC[C@H](O)CC3)ncc21] | 388.1 | ND |
| T128 | 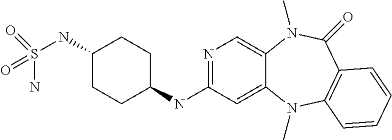 [SMILES: CN1C(=O)c2ccccc2N(C)c2cc(N[C@H]3CC[C@H](NS(N)(=O)=O)CC3)ncc21] | 431.2 | C |
| T129 | 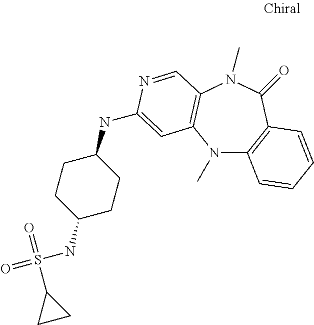 [SMILES: CN1C(=O)c2ccccc2N(C)c2cc(N[C@H]3CC[C@H](NS(=O)(=O)C4CC4)CC3)ncc21] | 456.2 | C |
| T130 | 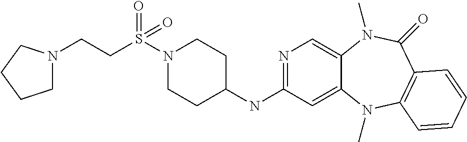 [SMILES: CN1C(=O)c2ccccc2N(C)c2cc(NC3CCN(S(=O)(=O)CCN4CCCC4)CC3)ncc21] | 499.2 | ND |
| T131 | 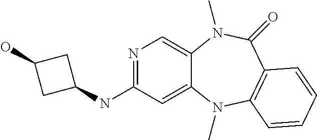 [SMILES: CN1C(=O)c2ccccc2N(C)c2cc(N[C@H]3C[C@@H](O)C3)ncc21] | 325.2 | ND |
| T132 | 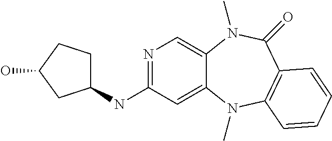 [SMILES: CN1C(=O)c2ccccc2N(C)c2cc(N[C@@H]3CC[C@@H](O)C3)ncc21] | 339.2 | ND |
| T133 | 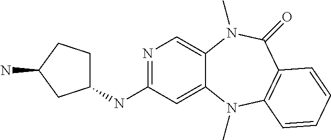 [SMILES: CN1C(=O)c2ccccc2N(C)c2cc(N[C@H]3CC[C@H](N)C3)ncc21] | 338.2 | ND |
| T134 | 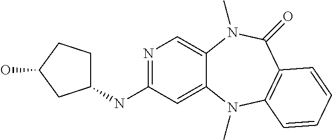 [SMILES: CN1C(=O)c2ccccc2N(C)c2cc(N[C@H]3CC[C@@H](O)C3)ncc21] | 339.2 | ND |
| T135 | 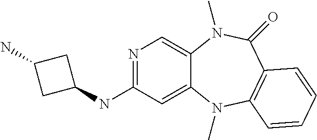 [SMILES: CN1C(=O)c2ccccc2N(C)c2cc(N[C@H]3C[C@H](N)C3)ncc21] | 324.2 | ND |
| T136 | 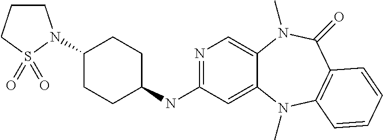 [SMILES: CN1C(=O)c2ccccc2N(C)c2cc(N[C@H]3CC[C@H](N4CCCS4(=O)=O)CC3)ncc21] | 456.2 | C |
| T137 | 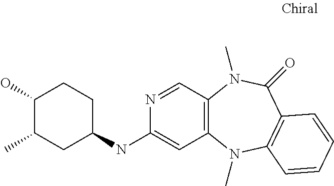 [SMILES: C[C@H]1C[C@H](Nc2cc3c(cn2)N(C)C(=O)c2ccccc2N3C)CC[C@H]1O] | 367.2 | C |

### Example 12

### Effect on Modulation of Cytokines Produced by Human CD4+T Cells

Purification of Human CD4+ T Cells (Protocol A):

Freshly isolated human peripheral blood mononuclear cells (PBMCs) were purchased from Astarte Biologics (Redmond, Wash.). CD4+ T cells were isolated from the PBMCs using the EasySep Negative Selection Human CD4+ T Cell Enrichment Kit (Stemcell Technologies, Vancouver, Canada) according to manufacturer's instructions briefly as follows: Unwanted cells were specifically labeled with a cocktail of bispecific tetrameric antibody complexes (TAC) against dextran and cell surface antigens (CD8, CD14, CD16, CD19, CD20, CD36, CD56, CD66b, CD123, TCRγ/δ, glycophorin A). Labeled cells were then targeted for removal by incubation with dextran-coated magnetic particles, leaving the desired CD4+ T cells.

Inhibition of Cytokine Response by Primary Human CD4+ T Cells Stimulated with PMA/Ionomycin.

Human CD4+ T cells, isolated as shown in protocol A, were seeded at 2e6 cells/ml in RPMI 1640 containing 10% (v/v) charcoal-dextran treated, heat inactivated FBS (Omega Scientific, Tarzana, Calif.), 0.05 mM 2-Mercaptoethanol (Sigma-Aldrich, St. Louis, Mo.), and 1×Pen/Strep Amphotericin B (Lonza, Allendale, N.J.). Cells were pre-treated with test compounds (10 μM) for 1 h at 37° C. in a humidified atmosphere at 5% CO₂. Cells were then stimulated with 50 ng/ml phorbol 12-myristate 13-acetate (PMA)(Sigma-Aldrich, St. Louis, Mo.)+1 μg/ml ionomycin (Life Technologies, Grand Island, N.Y.) for 20 h at 37° C. in a humidified atmosphere at 5% CO₂. Supernatant was collected and analyzed using Bio-Plex Pro Human Th17 Cytokine 15-Plex Panel (Bio-Rad Laboratories, Hercules, Calif.) as per manufacturer's protocols. Magnetic beads were read on the Bio-Plex MAGPIX multiplex reader instrument using the accompanying xPONENT 4.2 acquisition software (Bio-Rad Laboratories, Hercules, Calif.). Data were analyzed via the Bio-Plex Manager software v6.1 (Bio-Rad Laboratories).

TABLE 2
Compound-mediated modulation of cytokines produced by human
CD4+ T cells stimulated with PMA/ionomycin

| | Percent inhibition at 10 μM (relative to DMSO control) | | | | | |
|---|---|---|---|---|---|---|
| | com- | com- | com- | com- | com- | com- |
| | pound | pound | pound | pound | pound | pound |
| Analyte | 61 | 57 | 27 | 62 | 46 | 65 |
| G-CSF | 25.3 | 34.5 | 34.6 | 27.6 | 26.4 | 36.1 |
| GM-CSF | 96.6 | 95.9 | 92.8 | 95.4 | 96.7 | 89.5 |
| IFNγ | −11.8 | 50.9 | 37.8 | 15.6 | 18.3 | 12.3 |
| IL-4 | −7.7 | 31.4 | 30.1 | −4.8 | 11.7 | 10.1 |
| IL-5 | 21.9 | 42.8 | 26.3 | 29.7 | 49.1 | 47.7 |
| IL-6 | 85.8 | 91.1 | 86.5 | 86.9 | 89.3 | 87.7 |
| IL-8 | −257.7 | −44.1 | −23.4 | −178.9 | −108.8 | −227.7 |
| IL-10 | 97.6 | 97.7 | 94.4 | 97.2 | 97.3 | 98.0 |
| IL-13 | 0.6 | 55.6 | 28.9 | 19.8 | 32.1 | 18.3 |
| IL-17A | 45.4 | 78.8 | 60.6 | 49.7 | 52.2 | 39.3 |
| MCP-1 | 65.5 | 70.6 | 65.7 | 66.1 | 67.6 | 64.2 |
| MIP-1β | 59.2 | 56.2 | 35.0 | 66.9 | 63.9 | 55.8 |
| TNFα | 50.9 | 58.3 | 34.5 | 70.1 | 64.0 | 56.9 |

The values in Table 2 represent the average % inhibition by 10 μM test compound relative to the 0.1% DMSO control. IL-1β, IL-2, IL-7, and IL-12 (p70) were below the limit of detection under conditions used. The concentrations of the observed analytes of the DMSO control, from top to bottom as listed, are as follows: 31, 585, 24178, 22, 48, 91, 728, 94, 156, 705, 15, 1045, 5501 pg/ml.

### Example 13

### Inhibition of Cytokine Response by Primary Cynomolgus Monkey PBMCs Stimulated with LPS

Freshly isolated cynomolgus monkey (M. fascicularis) peripheral blood mononuclear cells (PBMCs) were purchased from SNBL (Everett, Wash.). Cells were seeded at 1.8e6 cells/ml in RPMI 1640 (Life Technologies, Grand Island, N.Y.) containing 10% (v/v) charcoal-dextran treated, heat inactivated FBS (Omega Scientific, Tarzana, Calif.), 0.05 mM 2-Mercaptoethanol (Sigma-Aldrich, St. Louis, Mo.), and 1×Pen/Strep Amphotericin B (Lonza, Allendale, N.J.). Cells were pre-treated with 0.1% DMSO or with a test compound for 1 h at 37° C. in a humidified atmosphere at 5% CO₂. Compounds were tested at 10 μM down by ⅕ in a 4-point serial dilution series. Cells were then stimulated with 100 ng/ml LPS from E. coli 0111:B4 (EMD Millipore, Billerica, Mass.) for 20 h at 37° C. in a humidified atmosphere at 5% CO₂. Supernatant was collected and analyzed using the Milliplex Map Non-human Primate Cytokine Magnetic Bead Panel Kit (EMD Millipore) as per manufacturer's protocols. Magnetic beads were read on the Bio-Plex MAGPIX multiplex reader instrument using the accompanying xPONENT 4.2 acquisition software (Bio-Rad Laboratories, Hercules, Calif.). Data were analyzed via the Bio-Plex Manager software v6.1 (Bio-Rad Laboratories). EC₅₀ values were determined using GraphPad Prism software v5.04 (La Jolla, Calif.), with cytokine levels from cells treated with DMSO+stimulation typically normalized as 100% and cytokine levels from cells treated with DMSO+no Stimulation typically normalized as 0%. Results are reported in Table 3.

TABLE 3
Inhibitory EC₅₀ values of compounds 8 and 46 for cytokines
released by cynomolgus monkey PBMCs stimulated with LPS

| | |
|---|---|
| Cynomolgus | EC₅₀ (μM) under LPS stimulation |

| monkey cytokine | compound 8 | compound 46 |
|---|---|---|
| G-CSF | 0.58 | 0.26 |
| GM-CSF | 0.50 | 0.18 |
| IFN-γ | 0.55 | 0.28 |
| IL-10 | 0.52 | 0.31 |
| IL-12/IL-23 (p40) | 0.75 | 0.36 |
| IL-1β | 0.47 | 0.20 |
| IL-1ra | 0.71 | 0.32 |
| IL-4 | 1.29 | 0.48 |
| IL-6 | 1.42 | 0.60 |
| IL-8 | 0.98 | 0.35 |
| MCP-1 | 0.32 | 0.13 |
| MIP-1α | 1.63 | 0.78 |
| MIP-1β | 4.45 | 2.20 |
| sCD40L | 1.14 | 0.41 |
| TNF-α | 0.63 | 0.24 |
| VEGF | 0.90 | 0.45 |

### Example 14

### Inhibition of Cytokine Response by Primary Human PBMCs Stimulated with PMA/Ionomycin or LPS

Freshly isolated human PBMCs were purchased from Astarte Biologics (Redmond, Wash.). Cells were seeded at 2e6 cells/ml in RPMI 1640 (Life Technologies, Grand Island, N.Y.) containing 10% (v/v) charcoal-dextran treated, heat inactivated FBS (Omega Scientific, Tarzana, Calif.), 0.05 mM 2-Mercaptoethanol (Sigma-Aldrich, St. Louis, Mo.), and 1×Pen/Strep Amphotericin B (Lonza, Allendale, N.J.). Cells were pre-treated with 0.1% DMSO or with test compounds for 1 h at 37° C. in a humidified atmosphere at 5% CO₂. Compounds were tested at 10 μM down by ⅕ in a 4-point serial dilution series. Cells were then stimulated with either 50 ng/ml phorbol 12-myristate 13-acetate (PMA)(Sigma-Aldrich)+1 μg/ml ionomycin (Life Technologies) or with 100 ng/ml LPS from E. coli 0111:B4 (EMD Millipore, Billerica, Mass.) for 20 h at 37° C. in a humidified atmosphere at 5% CO₂. Supernatant was collected and analyzed using the Bio-Plex Pro Human Cytokine 17-Plex Panel (Bio-Rad Laboratories, Hercules, Calif.) as per manufacturer's protocols. Magnetic beads were read on the Bio-Plex MAGPIX multiplex reader instrument using the accompanying xPONENT 4.2 acquisition software (Bio-Rad Laboratories). Data were analyzed via the Bio-Plex Manager software v6.1 (Bio-Rad Laboratories). EC₅₀ values for cytokines in range were determined using GraphPad Prism software v5.04 (La Jolla, Calif.), with cytokine levels from cells treated with DMSO+Stimulation normalized as 100% and cytokine levels from cells treated with DMSO+No Stimulation normalized as 0%.

TABLE 4
Inhibitory EC₅₀ values of test compounds for cytokines released
by human PBMCs stimulated with PMA/ionomycin or LPS

| | EC₅₀ (μM) under PMA/ | | EC₅₀ (μM) under LPS | |
|---|---|---|---|---|
| | ionomycin stimulation | | stimulation | |
| Cytokine | Compound 8 | Compound 46 | Compound 8 | Compound 46 |
| G-CSF | n/c | n/c | 4.95 | 2.52 |
| GM-CSF | 4.67 | 3.27 | 4.04 | 2.14 |
| IL-1β | n/c | n/c | 3.92 | 1.65 |
| IL-2 | n/c | n/c | 7.93 | 3.12 |
| IL-4 | >10 | >10 | 10.9 | 5.15 |
| IL-5 | 3.88 | 3.16 | n/c | n/c |
| IL-6 | 2.35 | 0.90 | 4.49 | 2.07 |
| IL-7 | >10 | >10 | 9.89 | 4.21 |
| IL-10 | 0.627 | 0.181 | 1.09 | 0.467 |
| IL-12 | >10 | >10 | 1.95 | 0.713 |
| IL-13 | 4.22 | 4.77 | 6.90 | 3.12 |
| IL-17A | >10 | >10 | >10 | >10 |
| IFNγ | n/c | n/c | 1.67 | 0.630 |
| MCP-1 | 0.401 | 0.143 | 0.237 | 0.104 |
| MIP-1β | 4.28 | 8.07 | n/c | n/c |
| TNFα | 8.17 | 4.14 | 0.304 | 0.124 |

n/c: EC₅₀ values were not calculated

### Example 15

### Inhibition of Cytokine Response by In Vitro-Polarized Human Th17 Cells Stimulated with PMA/Ionomycin

In vitro differentiation of human Th17 cells: Human CD4+ T cells (isolated as described in protocol A) were polarized into Th17 cells as follows: Tissue culture-treated 100 mm plates (Corning, Corning, N.Y.) were coated with 10 μg/ml anti-human CD3 clone OKT3 antibody overnight at 4° C. Polarization was initiated by seeding approximately 2-3 e7 human CD4+ T cells onto the anti-CD3 plates in Polarization media consisting of: RPMI 1640 medium (Lonza, Allendale, N.J.) supplemented with 10% (v/v) charcoal-dextran treated, heat inactivated FBS (Omega Scientific, Tarzana, Calif.), 0.05 mM 2-Mercaptoethanol (Sigma-Aldrich, St. Louis, Mo.), and 1×Pen/Strep Amphotericin B (Lonza), with added recombinant human IL-23 (40 ng/ml) and anti-CD28 clone CD28.2 antibody (2 μg/ml); all recombinant human cytokines and anti-human neutralizing antibodies were purchased from eBioscience (San Diego, Calif.). Cells were incubated in the above Polarization media for 3 days at 37° C. in a humidified atmosphere at 5% CO₂. After 3 days, the Polarization media was replaced with Maintenance media, consisting of RPMI 1640/FBS/2-Mercaptoethanol/Pen/Strep Amphotericin B supplemented with recombinant human IL-23 (40 ng/ml) alone, and the cells were re-plated onto regular tissue-culture-treated plates (not anti-CD3 coated). Cells were incubated in the Maintenance media for an additional 3 days at 37° C. in a humidified atmosphere at 5% CO₂.

Inhibition of PMA/ionomycin-stimulated cytokine response by in vitro-differentiated human Th17 cells: Human Th17 cells, generated as described above, were seeded at 2e6 cells/ml in RPMI 1640 containing 10% (v/v) charcoal-dextran treated, heat inactivated FBS (Omega Scientific, Tarzana, Calif.), 0.05 mM 2-Mercaptoethanol (Sigma-Aldrich, St. Louis, Mo.), and 1×Pen/Strep Amphotericin B (Lonza, Allendale, N.J.). Cells were pre-treated with test compounds (10 μM) for 1 h at 37° C. in a humidified atmosphere at 5% CO₂. Cells were then stimulated with 50 ng/ml phorbol 12-myristate 13-acetate (PMA) (Sigma-Aldrich, St. Louis, Mo.)+1 μg/ml ionomycin (Life Technologies, Grand Island, N.Y.) for 20 h at 37° C. in a humidified atmosphere at 5% CO₂. Supernatant was collected and analyzed using Bio-Plex Pro Human Th17 Cytokine 15-Plex Panel (Bio-Rad Laboratories, Hercules, Calif.) as per manufacturer's protocols. Magnetic beads were read on the Bio-Plex MAGPIX multiplex reader instrument using the accompanying xPONENT 4.2 acquisition software (Bio-Rad Laboratories). Data were analyzed via the Bio-Plex Manager software v6.1 (Bio-Rad Laboratories). Cytokine secretion levels from Th17 cells treated with 0.1% DMSO+Stimulation were normalized to 100%.

TABLE 5
Compound-mediated modulation of cytokines produced by human Th17
cells stimulated with PMA/ionomycin.

| | Percent inhibition at 10 μM (relative to DMSO control) | | | | | |
|---|---|---|---|---|---|---|
| | Com- | Com- | Com- | Com- | Com- | Com- |
| | pound | pound | pound | pound | pound | pound |
| Cytokine | 61 | 57 | 62 | 46 | 65 | 115 |
| IFNγ | 7.6 | 17.4 | 11.7 | 11.9 | −0.6 | 18.4 |
| IL-4 | −19.5 | −11.8 | −17 | −2.8 | 4.6 | 1.5 |
| IL-6 | 91.4 | 93.1 | 87.8 | 90.6 | 87.3 | 92.5 |
| IL-10 | 78.6 | 80 | 80.3 | 79.7 | 78.6 | 80.1 |
| IL-17A | 37.5 | 49.5 | 27.7 | 56.6 | 13.9 | 68.3 |
| IL-17F | 80.9 | 67.4 | 60 | 81.8 | 39.7 | 83.5 |
| IL-21 | 22.4 | 28.6 | 2.5 | −6.1 | 0.8 | −86.1 |
| IL-22 | 65.8 | 76.8 | 66.2 | 74 | 61.7 | 75.7 |
| IL-23 | −2.8 | 29.8 | 15.8 | −58.8 | −79.4 | −30.7 |
| IL-31 | −1.1 | 3 | −1.4 | −3 | −3.2 | 3.4 |
| sCD40L | 19 | 32.3 | 31.9 | 36.3 | 31.6 | 53.7 |
| TNFα | −6.3 | 5.6 | −10.4 | 22 | 11.3 | 57.1 |

Values in Table 5 represent the average % inhibition by 10 μM compound relative to the 0.1% DMSO control. IL-1β, IL-25, and IL-33 were below the limit of detection under conditions used. The concentrations of the observed analytes from top to bottom, as listed, are as follows: 8899, 567, 174, 99, 10380, 511, 239, 548, 81, 966, 504, and 76223 pg/ml.

### Example 16

### Inhibition of Cytokine Response by In Vitro-Polarized Murine Th17 Cells Stimulated with PMA/Ionomycin

Purification of murine CD4+ T cells: To isolate mouse splenocytes, freshly isolated mouse spleens were minced in culture media (RPMI 1640 (Lonza, Allendale, N.J.) containing 10% (v/v) charcoal-dextran filtered, heat inactivated FBS (Omega Scientific, Tarzana, Calif.), 0.05 mM 2-Mercaptoethanol (Sigma-Aldrich, St. Louis, Mo.), and 1×Pen/Strep Amphotericin B (Lonza)), using a sterile razor blade then pushed through 70 and 40 μm cell strainers. Cells were resuspended in ACK Lysing Buffer (Lonza) for 3 minutes at room temperature to lyse the red blood cells. CD4+ T cells were isolated from the mouse splenocytes using the EasySep negative selection mouse CD4+ T cell isolation kit (Stemcell, Vancouver, Canada) according to manufacturer's instructions briefly as follows: Unwanted cells were specifically labeled by incubation with a cocktail of biotinylated antibodies directed against cell surface antigens on mouse cells of hematopoietic origin (CD8a, CD11b, CD11c, CD19, CD45R/B220, CD49b, TCRγ/δ, and TER119). Labeled cells were then magnetically removed using streptavidin-bound magnetic beads, leaving the desired CD4+ T cells.

In vitro mouse Th17 differentiation: Mouse CD4+ T cells isolated as above were polarized into Th17 cells as follows: Tissue culture-treated 100 mm plates (Corning, Corning, N.Y.) were coated with 2 μg/ml purified NA/LE hamster anti-mouse CD3e clone 145-2C11 antibody (BD Biosciences, San Jose, Calif.) overnight at 4° C. Each coated plate was seeded with ˜2-3 e6 mouse CD4+ T cells in polarization media consisting of culture media (RPMI 1640/FBS/2-Mercaptoethanol/Pen/Strep Amphotericin B) supplemented with: recombinant mouse IL-6 (50 ng/ml), IL-13 (50 ng/ml), IL-23 (50 ng/ml)(R&D Systems, Minneapolis, Minn.), recombinant human TGF1β (5 ng/ml) (Cell Signaling Technology, Danvers, Mass.), anti-CD28 clone 37.51 (10 μg/ml)(BD Biosciences, San Jose, Calif.), anti-IL-2 clone JES6-1A12 (1 μg/ml), anti-IL-4 clone 11B11 (10 μg/ml), and anti-IFNγ clone XMG1.2 (10 μg/ml). All recombinant mouse cytokines and anti-mouse neutralizing antibodies were purchased from eBioscience (San Diego, Calif.) unless otherwise specified. Cells were incubated in the above Polarization media for 3 d at 37° C. in a humidified atmosphere at 5% CO₂. After 3 d, cells were re-polarized as above, except for the following cytokine and neutralizing antibody concentration changes: recombinant mouse IL-6 (40 ng/ml), recombinant human TGFβ (1 ng/ml), anti-CD28 clone 37.51 (5 μg/ml), anti-IL-2 clone JES6-1A12 (0.5 μg/ml), anti-IL-4 clone 11B11 (5 μg/ml), and anti-IFNγ clone XMG1.2 (5 μg/ml). Cells were maintained under the above conditions for an additional 3 d at 37° C. in humidified atmosphere at 5% CO₂ for a total of 6 days of polarization.

Inhibition of cytokine response by in vitro-polarized murine Th17 cells stimulated with PMA/ionomycin: Murine Th17 cells, polarized as described above, were seeded at 2e6 cells/ml in culture media. Cells were pre-treated with test compounds (10 μM down by ⅕ in a 4-point serial dilution series) for 1 h at 37° C. in a humidified atmosphere at 5% CO₂. Cells were then stimulated with 50 ng/ml phorbol 12-myristate 13-acetate (PMA)(Sigma-Aldrich, St. Louis, Mo.)+1 μg/ml ionomycin (Life Technologies, Grand Island, N.Y.) for 20 h at 37° C. in a humidified atmosphere at 5% CO₂. Supernatant was collected and analyzed using Bio-Plex Pro Mouse Cytokine Th17 Panel A 6-Plex and the Bio-Plex Pro Mouse Cytokine Th17 Panel B 8-Plex (Bio-Rad Laboratories, Hercules, Calif.) as per manufacturer's protocols. Magnetic beads were read on the Bio-Plex MAGPIX multiplex reader instrument using the accompanying xPONENT 4.2 acquisition software (Bio-Rad Laboratories, Hercules, Calif.). Data were analyzed via the Bio-Plex Manager software v6.1 (Bio-Rad Laboratories). EC₅₀ values were determined using GraphPad Prism software v5.04 (La Jolla, Calif.), with cytokine levels from cells treated with DMSO+Stimulation typically normalized as 100%. EC₅₀s were not calculated for cytokines that were below limits of detection or which did not have consistent and/or significant inhibition (EC₅₀>10 μM).

TABLE 6
Inhibitory EC₅₀ values of compounds on cytokines secreted by in
vitro-polarized, PMA/ionomycin-stimulated, murine Th17 cells

| | EC₅₀ (nM) of cytokine reduction | | | | | | |
|---|---|---|---|---|---|---|---|
| compound | | | | | | | IL- |
| # | IFNγ | IL-6 | IL-10 | IL-17A | IL-21 | IL-22 | 23p19 |
| 61 | >10,000 | 1602 | 1091 | >10,000 | 4240 | 2980 | 800 |
| 57 | 6926 | 2924 | 2154 | >10,000 | 3990 | 2120 | 840 |
| 62 | 8545 | 2476 | 1820 | >10,000 | 4980 | 5010 | 1340 |
| 46 | 4270 | 1736 | 577 | >10,000 | 4850 | 2630 | 480 |
| 65 | >10,000 | 2383 | 979 | >10,000 | 8280 | 5080 | 940 |
| 115 | 1771 | 1166 | 373 | 4985 | 2110 | 880 | 360 |

### Example 17

### Inhibition of Human PBMC TNF-α Response to Endotoxin

Human PBMCs (Astarte Biologics, Redmond, Wash.) were seeded at 2e6 cells/ml in RPMI 1640 containing 10% (v/v) charcoal-dextran treated, heat inactivated FBS (Omega Scientific, Tarzana, Calif.), 0.05 mM 2-Mercaptoethanol (Sigma-Aldrich, St. Louis, Mo.), and 1×Pen/Strep Amphotericin B (Lonza, Allendale, N.J.). Cells were pre-treated with test compounds (10 μM down by ⅕ in a 4-point serial dilution series) for 1 h at 37° C. in a humidified atmosphere at 5% CO₂. Cells were then stimulated with 0.1 μg/ml lipopolysaccharide (LPS) from E. coli 0111:B4 (EMD Millipore, Billerica, Mass.) for 16 h at 37° C. in a humidified atmosphere at 5% CO₂. Supernatant was collected and a 25-fold dilution was analyzed for TNF-α concentration using an ELISA kit from Life Technologies (Grand Island, N.Y.) according to the manufacturer's instructions. Absorbance at 450 nm was read using the Synergy 2 multi-detection microplate reader (BioTek Instruments, Winooski, Vt.). EC₅₀ values were determined using GraphPad Prism software v5.04 (La Jolla, Calif.) and TNF-α levels from cells treated with DMSO+LPS were normalized as 100%.

TABLE 7
Compound EC₅₀ values for the inhibition of
the human PBMC TNF-α response to endotoxin

| Compound | ave. ± S.D. (μM) | total n |
|---|---|---|
| 61 | 0.89 ± 0.29 | 6 |
| 53 | 4.13 | 1 |
| 68 | >10 | 1 |
| 57 | 1.08 | 1 |
| 54 | 0.60 | 1 |
| 88 | 0.60 | 1 |
| 44 | 0.59 | 1 |
| 101 | 0.97 | 1 |
| 83 | 1.48 | 1 |
| 103 | 0.62 ± 0.04 | 2 |
| 80 | 1.29 | 1 |
| 116 | 4.37 | 1 |
| 59 | 5.84 | 1 |
| 41 | 7.61 | 1 |
| 27 | >10 | 1 |
| 14 | 2.85 | 1 |
| 3 | 4.23 | 1 |
| 74 | 4.96 | 1 |
| 64 | 3.17 | 1 |
| 45 | 1.58 | 1 |
| 102 | 2.52 | 1 |
| 40 | >10 | 1 |
| 84 | 1.18 | 1 |
| 105 | 1.10 | 1 |
| 66 | 1.33 | 1 |
| 62 | 0.72 | 1 |
| 52 | 0.82 | 1 |
| 95 | 3.73 | 1 |
| 85 | 1.35 | 1 |
| 107 | 1.31 | 1 |
| 5 | 1.75 | 1 |
| 8 | 0.65 | 1 |
| 46 | 0.47 | 1 |
| 16 | 1.87 | 1 |
| 65 | 0.46 | 1 |
| 118 | 3.32 | 1 |
| 119 | 3.96 | 1 |
| 73 | 1.84 | 1 |
| 91 | 0.71 | 1 |
| 37 | 0.96 | 1 |
| 12 | 0.63 | 1 |
| 111 | 0.54 | 1 |
| 120 | 0.86 | 1 |
| 6 | 0.54 | 1 |
| 71 | 0.78 | 1 |
| 108 | 0.70 | 1 |
| 75 | 0.61 | 1 |
| 121 | 0.95 | 1 |
| 42 | 0.65 | 1 |
| 114 | 0.53 | 1 |
| 115 | 0.84 ± 0.53 | 4 |
| 69 | 1.07 | 1 |
| 100 | 1.02 | 1 |
| 63 | 0.87 | 1 |
| 93 | 0.76 | 1 |
| 96 | 0.57 | 1 |
| 28 | 0.48 | 1 |
| 89 | 1.75 | 1 |
| 25 | 1.17 ± 0.07 | 2 |
| 82 | 1.54 | 1 |
| 35 | 6.00 | 1 |
| 50 | 2.58 | 1 |
| 31 | 1.25 | 1 |
| 67 | 0.92 ± 0.18 | 2 |
| 15 | 0.49 ± 0.03 | 2 |
| 56 | 0.60 ± 0.19 | 2 |
| 72 | 1.23 ± 0.15 | 2 |
| 17 | 2.23 | 1 |
| 11 | 2.52 | 1 |
| 43 | 1.48 | 1 |
| 51 | 2.04 | 1 |
| 117 | 1.49 | 1 |
| 122 | 1.13 ± 0.38 | 2 |
| 30 | 1.26 ± 0.17 | 2 |
| 55 | 1.16 ± 0.45 | 2 |
| 110 | 1.76 ± 0.50 | 2 |
| 32 | 6.36 | 1 |
| 106 | 2.84 | 1 |
| 70 | 2.10 | 1 |
| 49 | 1.51 | 1 |
| 39 | 1.77 | 1 |
| 94 | 0.98 | 1 |
| 24 | 0.91 | 1 |
| 21 | 1.22 | 1 |
| 10 | 1.28 | 1 |
| T88 | 2.53 | 1 |
| T26 | 0.41 | 1 |
| T27 | 0.37 | 1 |
| T21 | 1.28 | 1 |
| T22 | 0.82 ± 0.14 | 2 |
| T28 | 0.48 | 1 |
| T25 | 0.48 | 1 |
| T29 | 0.35 | 1 |
| T71 | 4.20 | 1 |
| T7 | 0.34 | 1 |
| T13 | 0.95 | 1 |
| T24 | 3.27 | 1 |
| T23 | 2.39 | 1 |
| T5 | 0.84 | 1 |
| T40 | 0.49 | 1 |
| T46 | 0.60 | 1 |
| T48 | 0.74 | 1 |
| T62 | 0.49 ± 0.10 | 2 |
| T63 | 0.75 | 1 |
| T39 | 0.92 | 1 |
| T33 | 0.67 | 1 |
| T32 | 0.87 | 1 |
| T59 | 0.72 | 1 |
| T4 | 0.49 | 1 |
| T18 | 0.82 ± 0.10 | 2 |
| T64 | 0.62 | 1 |
| T9 | 0.59 | 1 |
| T77 | 6.33 | 1 |
| T73 | >10 | 1 |
| T72 | 4.25 | 1 |
| T17 | 0.90 | 1 |
| T66 | >10 | 1 |
| T30 | 0.77 | 1 |
| T69 | 3.48 | 1 |
| T55 | 0.84 | 1 |
| T16 | 0.92 | 1 |
| T50 | 0.63 | 1 |
| T56 | 0.30 | 1 |
| T67 | 4.90 | 1 |
| T53 | 0.89 | 1 |
| T57 | 0.65 | 1 |
| T58 | 1.04 | 1 |
| T19 | 0.69 | 1 |
| T14 | 0.72 ± 0.05 | 2 |
| T70 | 2.09 | 1 |
| T38 | 0.32 | 1 |
| T6 | 0.40 | 1 |
| T35 | 0.66 | 1 |
| T36 | 0.11 | 1 |
| T20 | 0.46 ± 0.14 | 2 |
| T10 | 0.65 | 1 |
| T12 | 0.68 | 1 |
| T11 | 0.62 | 1 |
| T65 | >10 | 1 |
| T78 | 6.26 | 1 |
| T74 | 4.82 | 1 |
| T43 | >10 | 1 |
| T80 | >15 | 1 |
| T8 | 0.41 ± 0.12 | 2 |
| T42 | 0.68 ± 0.45 | 2 |
| T45 | 1.48 | 1 |
| T51 | 0.74 | 1 |
| T52 | 1.08 | 1 |
| T60 | 0.95 | 1 |
| T61 | 0.42 | 1 |
| T44 | 0.65 | 1 |
| T54 | 7.14 | 1 |
| T75 | 1.55 ± 1.11 | 3 |
| T68 | 1.40 | 1 |
| T49 | 0.30 ± 0.12 | 2 |
| T31 | 0.49 | 1 |
| T41 | 0.37 | 1 |
| T15 | 0.80 ± 0.27 | 2 |
| T34 | 0.49 ± 0.04 | 2 |
| T76 | 1.11 | 1 |
| T79 | 7.20 | 1 |
| T47 | 3.97 | 1 |
| T85 | >15 | 1 |
| T37 | 0.69 | 1 |
| T135 | 9.20 | 1 |
| T131 | 6.06 | 1 |
| T133 | 9.84 | 1 |
| T132 | 3.28 | 1 |
| T134 | 3.68 | 1 |
| T117 | 2.70 ± 2.72 | 2 |
| T108 | 3.01 | 1 |
| T109 | 2.16 | 1 |
| T111 | 1.92 | 1 |
| T114 | 3.07 ± 3.35 | 2 |
| T96 | 1.10 ± 0.86 | 2 |
| T116 | 1.66 ± 1.53 | 2 |
| T92 | 0.44 ± 0.29 | 2 |
| T93 | 1.72 ± 1.34 | 2 |
| T115 | 0.78 ± 0.69 | 2 |
| T121 | 2.99 | 1 |
| T123 | 2.10 | 1 |
| T137 | 1.11 ± 0.99 | 2 |
| T95 | 1.38 ± 1.71 | 2 |
| T118 | 2.43 ± 1.78 | 2 |
| T119 | 0.98 ± 0.93 | 2 |
| T94 | 1.17 ± 1.12 | 2 |
| T97 | 0.43 ± 0.45 | 2 |
| T110 | 2.74 ± 1.87 | 2 |
| T112 | 2.36 | 1 |
| T113 | 2.95 ± 2.17 | 2 |
| T122 | 3.49 ± 3.00 | 2 |
| T120 | 2.39 ± 2.11 | 2 |
| T127 | 4.60 | 1 |
| T128 | 2.51 ± 1.47 | 2 |
| T129 | 1.79 ± 1.44 | 2 |
| T136 | 2.03 ± 1.91 | 2 |
| T130 | 2.46 | 1 |
| T89 | 1.72 ± 1.51 | 2 |
| T90 | >15 | 1 |
| T91 | >15 | 1 |
| T98 | 0.81 ± 0.92 | 2 |
| T106 | 0.27 ± 0.17 | 2 |
| T99 | 3.22 ± 2.75 | 2 |
| T102 | 0.29 ± 0.28 | 2 |
| T103 | 1.23 ± 1.31 | 2 |
| T105 | 0.43 ± 0.28 | 2 |
| T124 | 0.31 ± 0.22 | 2 |
| T125 | 0.79 ± 0.20 | 2 |
| T100 | >15 | 1 |
| T104 | 0.51 ± 0.24 | 2 |
| T107 | 0.30 ± 0.25 | 2 |
| T126 | 0.97 ± 0.83 | 2 |
| T101 | >15 | 1 |

### Example 18

### Inhibition of Pro-Inflammatory Cytokine Response by Human Bronchial Epithelial Cells Stimulated with IL-17A or IL-17F

The virally immortalized, normal human bronchial epithelial cell line BEAS-2B was purchased from ATCC (Manassas, Va.). Primary bronchial epithelial cells isolated from a patient diagnosed with asthma were obtained from Lonza (Allendale, N.J.). Both cells were maintained in complete Bronchial Epithelial Cell Growth Medium (BEGM) (Lonza). Cells were seeded at 1e5 cells/ml and incubated at 37° C. in a humidified atmosphere at 5% CO₂ overnight to adhere. Cells were pre-treated with test compounds (10 μM down by ⅕ in a 3- or 4-point serial dilution series) for 1 h at 37° C. in a humidified atmosphere at 5% CO₂. DMSO at a final concentration of 0.1% served as the non-inhibited control. Budesonide (Selleck Chemicals, Houston, Tex.), a clinically approved glucocorticoid steroid for the treatment of asthma, was tested as a positive control at 0.25 μM. Cells were stimulated with 50 ng/ml of either human recombinant IL-17A or IL-17F (eBioscience, San Diego, Calif.), for 48 h. Supernatant was then collected and analyzed for cytokine concentrations using the Bio-Plex Pro Human Cytokine 17-Plex Panel (Bio-Rad Laboratories, Hercules, Calif.) according to the manufacturer's instructions. Magnetic beads were measured on the Bio-Plex MAGPIX multiplex reader instrument using the accompanying xPONENT 4.2 acquisition software (Bio-Rad Laboratories). Data were analyzed via the Bio-Plex Manager software v6.1 (Bio-Rad Laboratories). For the primary asthmatic bronchial epithelial cells, five (+IL-17F) to thirteen (+IL-17A) cytokines were in range of detection under the conditions used. EC₅₀ values were determined using GraphPad Prism software v5.04 (La Jolla, Calif.), with cytokine levels from cells treated with DMSO+stimulation typically normalized as 100%. Shown in the data table are the EC₅₀s for the cytokines in the multiplex that exhibited ≧50% inhibition by the highest concentration of compound tested (10 μM). When compounds reduced the cytokine levels to below non-stimulated concentrations, those lower values were used to normalize to 0% for EC₅₀ calculations. Select cytokines were analyzed for comparison using the BEAS-2B cell line.

TABLE 8
EC₅₀ values for compound-mediated inhibition of IL-17A- and
IL-17F-mediated cytokine response by primary asthmatic human
bronchial epithelial cells

| | EC₅₀ values (μM) | | | | | |
|---|---|---|---|---|---|---|
| | +50 ng/ml IL-17A | | | +50 ng/ml IL-17F | | |
| | com- | com- | com- | com- | com- | com- |
| | pound | pound | pound | pound | pound | pound |
| Cytokine | 61 | 65 | 115 | 61 | 65 | 115 |
| G-CSF | 1.66 | 2.68 | 0.66 | 0.91 | 1.77 | 0.21 |
| GM-CSF | 3.38 | 4.06 | 1.39 | BLQ | BLQ | BLQ |
| IFN-γ | >10 | >10 | >10 | 0.60 | 1.98 | 0.34 |
| IL-1β | 1.26 | 1.57 | 0.95 | 0.79 | 1.57 | 0.38 |
| IL-2 | 10.5 | >10 | 5.31 | BLQ | BLQ | BLQ |
| IL-6 | 0.50 | 0.56 | 0.16 | 0.38 | 0.59 | 0.34 |
| IL-8 | 0.51 | 0.63 | 0.15 | 0.52 | 1.27 | 0.28 |
| MCP-1 | 6.25 | >10 | 6.10 | BLQ | BLQ | BLQ |

Budesonide at 0.25 μM had <50% inhibition of measured cytokines (data not shown). N/D: not determined. BLQ: cytokine levels were below limits of quantitation.

TABLE 9
EC₅₀ values for compound-mediated inhibition of IL-17A- and
IL-17F-mediated cytokine response by immortalized normal human
bronchial epithelial BEAS-2B cells

| | EC₅₀ values (μM) | | | | | |
|---|---|---|---|---|---|---|
| | +50 ng/ml IL-17A | | | +50 ng/ml IL-17F | | |
| | com- | com- | com- | com- | com- | com- |
| | pound | pound | pound | pound | pound | pound |
| Cytokine | 61 | 46 | 115 | 61 | 46 | 115 |
| IL-6 | 0.68 | 0.51 | 0.21 | 0.42 | 0.38 | 0.14 |
| IL-8 | 0.42 | 0.40 | 0.42 | 0.51 | 0.32 | 0.13 |

### Example 19

### Inhibition of TGF-β-Induced Fibrotic Response in Primary Human Lung Fibroblasts

Nontransformed, human fetal lung fibroblasts (HFL-1) were obtained from ATCC (Manassas, Va.) and maintained in F12K media containing 10% (v/v) charcoal-dextran treated, heat inactivated FBS (Omega Scientific, Tarzana, Calif.). Primary lung fibroblasts isolated from a patient diagnosed with asthma were obtained from Lonza (Allendale, N.J.) and maintained in FGM-2 fibroblast growth medium (Lonza). Cells were seeded at a density to provide 50-60% confluence after an overnight incubation at 37° C. in a humidified atmosphere at 5% CO₂. Cells were then serum-starved overnight in the respective base media (F12K or FBM Fibroblast Basal Medium) supplemented with 0.5% (w/v) RIA-grade BSA (Sigma-Aldrich, St. Louis, Mo.). Cells were pre-treated with test compounds (10 μM down by ⅕ in a 3- or 4-point serial dilution series) for 1 h at 37° C. in a humidified atmosphere at 5% CO₂. DMSO at a final concentration of 0.1% served as the non-inhibited control. A-83-01 (Tocris Bioscience, R&D Systems, Minneapolis, Minn.), an inhibitor of the TGF-β receptor, was used as the positive control at 4.5 μM. Cells were stimulated with 2 ng/ml of recombinant human TGF-β1 (R&D Systems, Minneapolis, Minn.) for 48 h at 37° C. in a humidified atmosphere at 5% CO₂ to induce differentiation. Cells were rinsed in phosphate-buffered saline, and lysed in 1× Cell Lysis buffer (Cell Signaling Technology, Danvers, Mass.) supplemented with 1 mM PMSF (Sigma). Protein content was determined using the Bio-Rad Dc Protein kit (Bio-Rad Laboratories, Hercules, Calif.), and normalized prior to adding an equal volume of 2× Laemmli sample buffer (Sigma). Proteins in the whole cell lysates were resolved via SDS-PAGE gel electrophoresis, and α-SMA was detected via Western blot analysis using a rabbit anti-human α-SMA polyclonal antibody (Abcam, Cambridge, Mass.). α-SMA signal was normalized to the signal of β-actin as detected using mouse anti-human θ-actin antibody (Cell Signaling Technology). DyLight 680 and 800 conjugated secondary antibodies were used and the infrared signals were detected using the Odyssey Imaging System (LI-COR Biotechnology, Lincoln, Nebr.). EC₅₀ values were determined using GraphPad Prism (La Jolla, Calif.) software v5.04, with signal from cells treated with 0.1% DMSO+TGF-β normalized to 100%.

TABLE 10
EC₅₀ values for compound-mediated inhibition of
fibrotic response as measured by α-SMA expression

Inhibition of α-SMA expression, EC₅₀ (μM)

| Compound | HFL-1 | Asthmatic lung fibroblasts |
|---|---|---|
| compound 61 | 0.75-2.3 (n = 2) | 3.69 |
| compound 115 | N/D | 1.37 |

### Example 20

### Inhibition of Pro-Inflammatory Cytokine Response by Primary Diseased Human Lung Fibroblasts Stimulated with IL-17A or IL-17F

Primary lung fibroblasts isolated from a patient diagnosed with asthma were obtained from Lonza (Allendale, N.J.) and maintained in FGM-2 fibroblast growth medium (Lonza). Cells were seeded at 1e5 cells/ml and incubated at 37° C. in a humidified atmosphere at 5% CO₂ overnight to adhere. Cells were pre-treated with test compounds (10 μM down by ⅕ in a 3- or 4-point serial dilution series) for 1 h at 37° C. in a humidified atmosphere at 5% CO₂. DMSO at a final concentration of 0.1% served as the non-inhibited control. Cells were stimulated with 50 ng/ml of either human recombinant IL-17A or IL-17F (eBioscience, San Diego, Calif.), for 48 h. Supernatant was then collected and analyzed for cytokine concentrations using the Bio-Plex Pro Human Cytokine 17-Plex Panel (Bio-Rad Laboratories, Hercules, Calif.) according to the manufacturer's instructions. Magnetic beads were measured on the Bio-Plex MAGPIX multiplex reader instrument using the accompanying xPONENT 4.2 acquisition software (Bio-Rad Laboratories). Data were analyzed via the Bio-Plex Manager software v6.1 (Bio-Rad Laboratories). Only four (+IL-17F) to six (+IL-17A) cytokines were in range of detection under the conditions used. EC₅₀ values were determined using GraphPad Prism software v5.04 (La Jolla, Calif.), with cytokine levels from cells treated with DMSO+stimulation typically normalized as 100%. While MCP-1 was not increased by IL-17A or IL-17F stimulation over non-stimulated levels, the compounds reduced MCP-1 to below non-stimulated concentrations. Shown in the data table below are the EC₅₀s for the four cytokines from the multiplex that exhibited ≧50% inhibition by the highest concentration of compound tested (10 μM). When compounds reduced the cytokine levels to below non-stimulated concentrations, those lower values were used to normalize to 0% for EC₅₀ calculations.

TABLE 11
EC₅₀ values for compound-mediated inhibition of cytokine response by
diseased primary human lung fibroblasts.

| | EC₅₀ values (μM) | | | | | |
|---|---|---|---|---|---|---|
| | +50 ng/ml IL-17A | | | +50 ng/ml IL-17F | | |
| | Com- | Com- | Com- | Com- | Com- | Com- |
| | pound | pound | pound | pound | pound | pound |
| Cytokine | 61 | 65 | 115 | 61 | 65 | 115 |
| IL-6 | 6.98 | 5.69 | 1.16 | 4.45 | 2.49 | 0.95 |
| IL-8 | 0.54 | 0.48 | <0.08 | 0.45 | 0.52 | <0.08 |
| G-CSF | 1.99 | 3.50 | 0.86 | BLQ | BLQ | BLQ |
| MCP-1 | 1.33 | 1.31 | 0.35 | 1.57 | 1.85 | 0.36 |

G-CSF concentrations were below the limit of detection when cells were stimulated with IL-17F.

### Example 21

### Inhibition of Pro-Inflammatory Cytokine Response by Primary Human Keratinocytes Stimulated with IL-17A

Primary human keratinocytes were obtained from Life Technologies (Grand Island, N.Y.) and maintained in growth supplemented Defined Keratinocyte-SFM Medium (Life Technologies). Cells were seeded at 2.5e4 cells/ml onto collagen-coated plates and incubated at 37° C. in a humidified atmosphere at 5% CO₂ until the cells reached confluence. Cells were pre-treated with test compounds (10 μM down by ⅕ in a 4-point serial dilution series) for 1 h at 37° C. in a humidified atmosphere at 5% CO₂. DMSO at a final concentration of 0.1% served as the non-inhibited control. Cells were then stimulated with 200 ng/ml human recombinant IL-17A (eBioscience, San Diego, Calif.) for 48 h. Supernatant was collected and analyzed for cytokine concentrations using the Human Inflammatory Magnetic 5-Plex Panel from Life Technologies according to the manufacturer's instructions. Magnetic beads were measured on the Bio-Plex MAGPIX multiplex reader instrument using the accompanying xPONENT 4.2 acquisition software (Bio-Rad Laboratories, Hercules, Calif.). Data were analyzed via the Bio-Plex Manager software v6.1 (Bio-Rad Laboratories). EC₅₀ values were determined using GraphPad Prism software v5.04 (La Jolla, Calif.), with cytokine levels from cells treated with DMSO+Stimulation typically normalized as 100% and cytokine levels from cells treated with DMSO+No Stimulation typically normalized as 0%.

TABLE 12
EC₅₀ values for Compound 115-mediated inhibition of IL-17A-
stimulated cytokine response by primary human keratinocytes.

| Cytokine | Inhibition EC₅₀ (μM) |
|---|---|
| IL-1β | 0.50 |
| IL-6 | 0.35 |
| IL-8 | 0.46 |
| GM-CSF | 2.18 |
| TNF-α | 1.00 |

### Example 22

### MV-4-11 Proliferation EC₅₀ Assay

Human biphenotypic B myelomonocytic leukemia MV-4-11 cells (ATCC, Manassas, Va.) were treated with test compounds at 2e5 cells/ml in IMDM medium containing 10% (v/v) FBS (Life Technologies, Grand Island, N.Y.) and 1×Pen/Strep Amphotericin B (Lonza, Allendale, N.J.). Compounds were tested at 25 μM down by ⅓ in a 9 point serial dilution series. Cells were incubated for 48 h at 37° C. in a humidified atmosphere at 5% CO₂. MV-4-11 cell proliferation was then indirectly determined via cellular ATP content using the CellTiter-Glo® Luminescent Cell Viability Assay (Promega, Madison, Wis.) as per the manufacturer's instructions. Briefly, experimental plates were equilibrated at room temperature for 30 minutes. An equal volume of CellTiter-Glo® reagent was added to each well and incubated for 15 minutes at room temperature. Luminescent signal was read using the Wallac 1420 Victor² multilabel microplate reader (Perkin Elmer, Waltham, Mass.). EC₅₀ values were determined using GraphPad Prism software v5.04.

TABLE 13
MV-4-11 proliferation EC₅₀ values

| compound | ave. ± S.D. (μM) | n |
|---|---|---|
| 61 | 0.70 ± 0.05 | 2 |
| 4 | 0.06 | 1 |
| 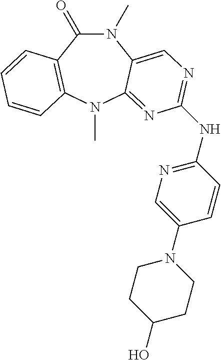 [SMILES: CN1C(=O)c2ccccc2N(C)c2nc(Nc3ccc(N4CCC(O)CC4)cn3)ncc21] | 1.12 | 1 |
| Compound IV-5 in WO 2010/080712 | | |

### Example 23

### Combination Studies with Ara-C

Human biphenotypic B myelomonocytic leukemia MV-4-11 cells (ATCC, Manassas, Va.) treated with test compounds at 2e5 cells/ml in IMDM medium containing 10% (v/v) FBS (Life Technologies, Grand Island, N.Y.) and 1×Pen/Strep Amphotericin B (Lonza, Allendale, N.J.). The final concentration of a test compound was set at a concentration previously determined to be its EC₅₀ and Y3 EC₅₀ values, while the combination test drug (Arabinofuranosyl Cytidine (Ara-C))(Sigma, St. Louis, Mo.) was assayed in serially-diluted 9-point concentrations against this background. Cells were treated for 48 h at 37° C. in a humidified atmosphere at 5% CO₂. Cell viability was then determined indirectly via ATP content using the CellTiter-Glo® luminescent cell viability assay (Promega, Madison, Wis.) as per the manufacturer's instructions as follows: Briefly, experimental plates were equilibrated at room temperature for 30 minutes. An equal volume of CellTiter-Glo reagent was added to each well and incubated for 15 minutes at room temperature. Luminescent signal was read using the Wallac 1420 Victor2 multilabel microplate reader (Perkin Elmer, Waltham, Mass.). EC₅₀ values were determined using GraphPad (La Jolla, Calif.) Prism software v5.04 using the following parameters: The average values for the 0.25% DMSO controls, compound 61 at EC₅₀ and at ⅓ EC₅₀ were normalized to 100% activity for each respective condition.

TABLE 14
EC₅₀ values of Ara-C in combination with Compound 61
MV-4-11 cells

| | EC₅₀ (μM) of | | |
|---|---|---|---|
| | Ara-C in combination with a | | |
| | fixed concentration of compound 61 | | |
| Test drug | not present | at EC₅₀ | at ⅓ EC₅₀ |
| Ara-C | 0.353 | 0.108 | 0.265 |

EC₅₀ values of Ara-C in combination with Compound 61 at its EC₅₀ or at V3 EC₅₀ using the MV-4-11 cell line demonstrate more potent Ara-C EC₅₀ values in the presence of compound 61.

### Example 24

### Inhibition of Pro-Inflammatory Cytokine Response by Primary Human Synovial Fibroblasts Stimulated with IL-17A, TNF-α, or Both

Primary synovial fibroblasts isolated from the knee of a patient diagnosed with rheumatoid arthritis/osteoarthritis were obtained from Asterand (Detroit, Mich.) and maintained in FGM-2 fibroblast growth medium (Lonza, Allendale, N.J.). Cells were seeded at approximately 1.6e4 cells/ml and incubated at 37° C. in a humidified atmosphere at 5% CO₂ until the cells reached ˜90% confluence. Cells were pre-treated with the test compounds (10 μM down by ⅕ in a 4-point serial dilution series) for 1 h at 37° C. in a humidified atmosphere at 5% CO₂. DMSO at a final concentration of 0.1% served as the non-inhibited control. Dexamethasone (Sigma-Aldrich, St. Louis, Mo.) at 0.25 μM was used as a positive control. Cells were stimulated with 10 ng/ml human recombinant IL-17A (eBioscience, San Diego, Calif.), 10 ng/ml TNF-α, (Cell Signaling Technology, Danvers, Mass.) or both, for 48 h. Supernatant was then collected and analyzed for cytokine concentrations using the Bio-Plex Pro Human Cytokine 17-Plex Panel supplemented with RANTES and VEGF Singleplex analytes (Bio-Rad Laboratories, Hercules, Calif.) according to the manufacturer's instructions. Magnetic beads were measured on the Bio-Plex MAGPIX multiplex reader instrument using the accompanying xPONENT 4.2 acquisition software (Bio-Rad Laboratories). Data were analyzed via the Bio-Plex Manager software v6.1 (Bio-Rad Laboratories). EC₅₀ values were determined using GraphPad Prism software v5.04 (La Jolla, Calif.), with cytokine levels from cells treated with DMSO+Stimulation typically normalized as 100% and cytokine levels from cells treated with DMSO+No Stimulation typically normalized as 0%. Shown in the data tables are the cytokines from the multiplex which exhibited ≧50% inhibition by the highest concentration of compound tested (10 μM).

TABLE 15
Stimulation of secreted cytokine levels (pg/ml)

| | Cytokine Concentrations (pg/ml) | | | |
|---|---|---|---|---|
| Cytokine | Nonstimulated | +10 ng/ml IL-17A | +10 ng/ml TNF-α | +Both IL-17A + TNF-α |
| G-CSF | 5.3 ± 0.5 | 280.5 ± 62.3 | 778.8 ± 7.5 | 22,445.0 ± 883.0 |
| IL-6 | 27.1 ± 1.4 | 229.4 ± 8.6 | 13,540.7 ± 601.2 | 42,142.2 ± 1585.8 |
| IL-8 | 47.1 ± 0.6 | 295.7 ± 45.8 | 5,093.8 ± 343.3 | 14,658.0 ± 1551.1 |
| RANTES | 4.8 ± 0.8 | 4.2 ± 0.5 | 808.9 ± 2.3 | 365.4 ± 38.6 |

Representative analysis of a 48 hr culture supernatant from DMSO-treated, diseased primary human synovial fibroblast demonstrated the synergistic stimulation of secreted cytokine levels (pg/ml) by a combination of IL-17A and TNF-α.

TABLE 16
EC₅₀ values for exemplary compounds-mediated inhibition of
cytokine response by diseased primary human synovial fibroblasts

| | EC₅₀ (μM) for | | | EC₅₀ (μM) for | | |
|---|---|---|---|---|---|---|
| | compound 61 | | | compound 115 | | |
| Cytokine | +IL-17A | +TNF-α | +Both | +IL-17A | +TNF-α | +Both |
| G-CSF | 2.59 | 1.27 | >10 | 2.67 | 1.25 | >10 |
| IL-6 | 1.67 | 1.05 | >10 | 1.04 | 1.71 | >10 |
| IL-8 | 0.87 | 4.51 | >10 | 0.39 | >10 | >10 |
| RANTES | >10 | 2.78 | 1.35 | >10 | >10 | 1.88 |

The positive control, dexamethasone (0.25 μM) was able to reduce doubly-stimulated cytokine response of only IL-6 and G-CSF but not IL-8 nor RANTES (data not shown).

### Example 25

### In Vivo Study in DNFB-Induced Ear Inflammation Mouse Model

In vivo T-cell mediated immune response was examined in a mouse model of CHS using the hapten dinitrofluorobenzene (DNFB) as the induction agent. Method for sensitization and elicitation of DNFB-induced CHS was modified from Xu et al. J. Exp. Med. 1996. 183:1001-1012. On Days 0 and 1 (sensitization), 20 μL of 0.5% v/v DNFB in acetone:olive oil (4:1) was applied to each hindpaw of 8 week old male mice (Balb/c, Charles River Laboratories; 10 mice per group unless otherwise noted). On Day 5 (elicitation) 10 μL of 0.2% v/v DNFB in acetone: olive oil (4:1) was applied to the dorsal surface of the right ear of each animal; 10 μL of acetone: olive oil (4:1) was applied to the dorsal surface of the left ear as control. Treatments were administered by group as indicated in the table below; on days when DNFB was used, the QD and the morning dose of BID as appropriate were administered 30 minutes-1 hr. prior to DNFB application. Positive treatment controls were dexamethasone (3 mg/kg PO, Sigma Aldrich #D1159, St. Louis, Mo.) and anti-IL17A monoclonal antibody (5 mg/kg IP, LEAF™ BioLegend #506923, San Diego, Calif.). Approximately 24 hours after elicitation (Day 6), a 7 mm tissue punch from the central portion of both ears was collected and wet weights measured. The primary functional assessment was the difference in wet weights of the right (DNFB) vs. left (vehicle) ears compared to the vehicle treatment (Vehicle1-S) as an indicator of inflammation (Table 17). Ear tissue punches were flash frozen, stored at −70° C. and analyzed for cytokines (see below). Body weights of the mice were measured at baseline and at end of study, and there were no significant changes in weight in any treatment group during the study (data not shown).

TABLE 17
Change in DNFB Induced Ear Weight with Treatment

| | | | | % Reduction | |
|---|---|---|---|---|---|
| | | | | Weight Increase | |
| | | Dose | Route of Admin, | Vs. Vehicle 1 | P |
| Treatment | N | (mg/kg) | Regimen | Weight Increase | value |
| Vehicle 1 | 10 | 5¹ | PO, BID Days 0-5, S² | | |
| (5% DMA + 95% PEG400) | | | | | |
| Vehicle 2 | 5 | 5¹ | PO, BID on Day 5, E³ | | |
| (5% DMA + 95% PEG400) | | | | | |
| Dexamethasone | 10 | 3 | PO, QD on Day 5, E | −60.0 | <0.01 |
| Anti-IL-17A mAB | 10 | 5 | IP, QD on Day 5, E | −47.1 | <0.01 |
| Compound 8 | 10 | 60 | PO, BID Days 0-5, S | −38.5 | <0.05 |
| | 10 | 60 | PO, BID on Day 5, E | −22.6 | <0.05 |
| | 10 | 25 | PO, BID on Day 5, E | −9.4 | >0.05 |
| Compound 46 | 9 | 60 | PO, BID Days 0-5, S | −39.5 | <0.05 |
| | 10 | 60 | PO, BID on Day 5, E | −40.5 | <0.05 |
| | 10 | 25 | PO, BID on Day 5, E | −39.5 | <0.05 |

¹mL/kg
²S = treatment administered from sensitization
³E = treatment administered at elicitation

Cytokine concentrations after DNFB induction and treatment were analyzed in the weighed and flash frozen tissues of the right (DNFB) and left (control) 7 mm ear punches. The ear tissues were stored at −70° C. until prepared for analysis as follows. Left or right ear tissues from the same treatment group were pooled and crushed with mortar and pestle under liquid nitrogen. Tissue representing 7-8 ear punches by weight was homogenized in Buffer A (50 mM HEPES pH 7.4, 100 mM NaCl) containing 1 tablet EDTA-free Protease Inhibitor (Thermo Fisher Scientific, Rockford, Ill.) per 10 mL, 1× Phosphatase Inhibitor Cocktail II (AG Scientific, San Diego, Calif.), and 1 mM PMSF (Sigma, St. Louis, Mo.). Homogenization was performed in Lysing Matrix D tubes (MP Biomedicals, Solon, Ohio), using the FastPrep-24 instrument (MP Biomedicals) at a speed setting of 6 for 4 cycles of 30 seconds, with cooling on ice in between cycles. The tubes were then centrifuged at 1,000×g for 10 min at 4° C. Supernatant was collected and briefly sonicated using the Microson probe sonicator at setting 3 (Misonix, Farmingdale, N.Y.) before centrifuging again at 10,000×g for 10 min at 4° C. The supernatant was ultracentrifuged at 100,000×g for 1 h at 4° C. in the Optima Max-E (Beckman Coulter, Indianapolis, Ind.) to separate soluble and membrane protein fractions. The soluble (supernatant) fraction was collected and protein concentrations determined using the Dc protein kit (Bio-Rad, Hercules, Calif.). Soluble ear proteins were analyzed in triplicate for cytokine response at a final protein concentration of 4 mg/ml using the Bio-Plex Pro Mouse Cytokine 23-Plex panel (Bio-Rad) as per manufacturer's protocols. Magnetic beads were read on the Bio-Plex MAGPIX multiplex reader instrument using the accompanying xPONENT 4.2 acquisition software (Bio-Rad Laboratories, Hercules, Calif.). Data were analyzed via the Bio-Plex Manager software v6.1 (Bio-Rad Laboratories). Response was calculated as a percentage of the cytokine concentrations measured in the right (DNFB) ears of vehicle 1 dosed (S regimen, i.e. dosed Day 0-5) mice (Table 18).

TABLE 18
Mean % Cytokine Concentrations in DNFB Treated Right Ears versus Vehicle 1-Dosed (S) Mice
(at least 50% change in any Treatment Group)

| | TREATMENT GROUP | | | | | | | | |
|---|---|---|---|---|---|---|---|---|---|
| | Mean % Change Normalized To Vehicle 1, S Regimen | | | | | | | | |
| | | | Anti IL-17 | Cmpnd 8 | Cmpnd 8 | Cmpnd 8 | Cmpnd 46 | Cmpnd 46 | Cmpnd 46 |
| | Vehicle 1 | Dex | mAb | 60 mg/kg | 60 mg/kg | 25 mg/kg | 60 mg/kg | 60 mg/kg | 25 mg/kg |
| Cytokine | S | E | E | S | E | E | S | E | E |
| IL-1α | 100 | 189 | 184 | 136 | 188 | 88 | 88 | 120 | 128 |
| IL-1β | 100 | 53 | 49 | 52 | 62 | 56 | 23 | 32 | 57 |
| IL-3 | 100 | 76 | 84 | 61 | 77 | 67 | 45 | 53 | 75 |
| IL-5 | 100 | 58 | 69 | 37 | 57 | 37 | 5 | 23 | 63 |
| IL-6 | 100 | 36 | 21 | 32 | 61 | 58 | 20 | 31 | 53 |
| IL-12 | 100 | 59 | 90 | 73 | 88 | 79 | 49 | 64 | 81 |
| (p40) | | | | | | | | | |
| IL-13 | 100 | 81 | 85 | 75 | 85 | 74 | 47 | 61 | 79 |
| IL-17A | 100 | 63 | 40 | 38 | 68 | 69 | 38 | 42 | 53 |
| G-CSF | 100 | 39 | 18 | 36 | 50 | 68 | 27 | 36 | 58 |
| GM-CSF | 100 | 65 | 66 | 52 | 48 | 40 | 0 | 0 | 29 |
| IFN-γ | 100 | 74 | 71 | 46 | 58 | 52 | 22 | 25 | 51 |
| KC | 100 | 29 | 17 | 35 | 29 | 39 | 19 | 21 | 30 |
| MCP-1 | 100 | 41 | 50 | 43 | 47 | 44 | 27 | 31 | 37 |
| MIP-1α | 100 | 53 | 63 | 57 | 62 | 62 | 29 | 42 | 62 |
| MIP-1β | 100 | 39 | 42 | 52 | 44 | 60 | 24 | 31 | 44 |
| RANTES | 100 | 42 | 47 | 51 | 53 | 66 | 27 | 37 | 55 |
| TNF-α | 100 | 74 | 69 | 65 | 81 | 53 | 39 | 48 | 67 |

Cytokines measured that did not have at least a 50% change in any group with treatment were IL-4, IL-10 and IL-12 (p70).

### Example 26

### Imiquimod (IMQ, Aldara™)-Induced Acute Model of Psoriasis in Mouse

Material and methods for the IMQ study were adapted from van der Fits et al. J Immunol., 2009, 182: 5836-5845. Eight week old male Balb/c mice (Harlan) received a daily topical dose of 62.5 mg 5% IMQ cream (Aldara™, Medicis) equivalent to 3.125 mg IMQ, on the shaved back (approximately 2×3.5 cm patch) and dorsal surface of the right ear for 6 consecutive days. Sham control mice were treated similarly with control cream (Hydrous Emulsified Base, HEB, Cream, Fagron). Treatments were administered by group as indicated in Table 1 below prior to daily application of IMQ (QD and morning dose of BID regimens), n=10 for all groups. Positive treatment controls were dexamethasone (3 mg/kg PO, Sigma Aldrich #D1159, St. Louis, Mo.) and anti-IL17A monoclonal antibody (5 mg/kg IP, LEAF™ BioLegend #506923, San Diego, Calif.). Body weights and scoring of induced clinical disease were performed after dosing (Days 1-7) but prior to IMQ application (Days 1-6) on Days 1 (baseline), 3, 4, 6, 8. Mice were sacrificed on Day 8 and treated back skin tissue collected and divided for fixation for histopathology (a 12 mm punch in mid-back) the remainder flash frozen; right (IMQ) and left (control) ears, spleen (after wet weight) and lung were also individually collected and flash frozen, and stored at −70° C. for subsequent biochemical analyses. Statistics were performed using one-way ANOVA with Dunnett's post-hoc test comparing treated groups to the vehicle group, using InStat software (GraphPad, La Jolla, Calif.).

The severity of clinical disease was assessed by

1) skin thickness dial gauge micrometer measurements (duplicate) of the ears and the dorsal skin at the midline of the back (equal to double true measure of dorsal skin thickness)

2) erythema and scaling, independently scored on a scale from 0-4: 0=none, 1=slight, 2=moderate, 3=marked, 4=very marked with intermediate increments of 0.5 allowed.

Clinical disease assessments included erythema, scaling, skin thickness and cumulative disease severity scores and the change in skin thickness of the ears and dorsal back skin over time compared to baseline measurements (Tables 19 and 20). Body weights of the mice were measured at baseline and at end of study, and there were no significant changes in weight in any treatment group during the study (data not shown).

TABLE 19
Treatment groups, doses, and regimens

| Group | | | Dose | | Dosing |
|---|---|---|---|---|---|
| (n = 10) | Topical TX | Test Article | mg/kg | Route | Schedule |
| 1 | HEB (sham) | Vehicle | N/A | PO | BID Days 1-7 |
| 2 | IMQ | Vehicle | N/A | PO | BID Days 1-7 |
| 3 | IMQ | Anti-IL-17A | 5 | IP | Day 1 |
| | | mAb | | | |
| 4 | IMQ | Dexamethasone | 3 | PO | QD Days 1-7 |
| 5 | IMQ | Compound 8 | 60 | PO | BID Days 1-7 |
| 6 | IMQ | Compound 46 | 60 | PO | QD Days 1-7 |
| 7 | IMQ | | 25 | PO | BID Days 1-7 |
| 8 | IMQ | | 60 | PO | BID Days 1-7 |

TABLE 20
% Mean ear and back skin thickness versus day 1 for A) groups
1-4 and B) treatment with compounds 8 and 46.

| | | Group 1: | | Group 2: | | Group 3: | | | |
|---|---|---|---|---|---|---|---|---|---|
| | | HEB | | IMQ | | IMQ | | Group 4: IMQ | |
| | | Vehicle | | Vehicle | | IL-17A mAb | | Dexamethasone | |
| A | DAY | AVG | SD | AVG | SD | AVG | SD | AVG | SD |
| LEFT EAR | 3 | −5.76 | 5.28 | −5.28 | 7.49 | −4.05 | 3.27 | −9.64 | 5.17 |
| THICKNESS | 4 | −0.73 | 3.91 | −3.42 | 6.57 | −4.45 | 3.5 | −10.38 | 5.44 |
| [Control] | 6 | 0.61 | 8.25 | 4.02 | 9.19 | −0.23 | 6.64 | −9.61 | 5.36 |
| | 8 | −3.67 | 4.16 | 1.6 | 6.69 | −0.27 | 4.84 | −14.05 | 6.99 |
| RIGHT EAR | 3 | −5.49 | 6.72 | 15.59 | 8.55 | −0.18 | 10.2 | −5.32 | 7.94 |
| THICKNESS | 4 | −2.24 | 8.39 | 28.88 | 12.6 | 4.91 | 13.6 | −2.08 | 6.67 |
| [IMQ] | 6 | −0.57 | 9.3 | 56.3 | 15.71 | 34.05** | 15.82 | 10.54** | 13.63 |
| | 8 | −2.47 | 9.21 | 77.95 | 37.77 | 47.12** | 17.47 | 10.40** | 13.31 |
| DORSAL | 3 | −1.6 | 3.82 | 44.37 | 8.82 | 41.87 | 8.93 | 3.08 | 3.62 |
| BACK | 4 | −3.27 | 5.31 | 59.54 | 8.13 | 61.78 | 18.47 | 13.71 | 6.82 |
| THICKNESS | 6 | −0.54 | 4.44 | 107.86 | 16.75 | 89.79^{NS} | 12.91 | 13.04** | 9.16 |
| | 8 | −2.73 | 6.62 | 45.72 | 17.32 | 75.89** | 13.13 | 14.32** | 12.88 |
| | | Group 5: IMQ | | Group 6: IMQ | | Group 7: IMQ | | Group 8: IMQ | |
| | | Compound 8 | | Compound 46 | | Compound 46 | | Compound 46 | |
| | | 60 mpk BID | | 60 mpk QD | | 25 mpk BID | | 60 mpk BID | |
| B | DAY | AVG | SD | AVG | SD | AVG | SD | AVG | SD |
| LEFT EAR | 3 | −5.16 | 3.63 | −8.74 | 5.22 | −7.42 | 4.19 | −8.07 | 5.29 |
| THICKNESS | 4 | −6.51 | 5.78 | −7.01 | 6.07 | −5.56 | 5.8 | −3.95 | 4.6 |
| [Control] | 6 | 1.15 | 8.69 | 8.65 | 25.82 | −1.35 | 6.41 | 1.37 | 7.38 |
| | 8 | −2.25 | 5.75 | 0.14 | 8.03 | −3.26 | 5.58 | −1.92 | 5.14 |
| RIGHT EAR | 3 | 0.09 | 4.62 | 3.71 | 11.68 | 6.71 | 10.99 | 3.35 | 7.68 |
| THICKNESS | 4 | 5.74 | 12.19 | 9.53 | 12.5 | 12.03 | 11.48 | 11.25 | 11.33 |
| [IMQ] | 6 | 36.67* | 18.04 | 38.11* | 8.84 | 37.60* | 11.54 | 33.34** | 10.77 |
| | 8 | 33.58** | 10.36 | 48.04** | 12.3 | 43.36** | 15.38 | 40.66** | 22.35 |
| DORSAL | 3 | 33.49 | 10.77 | 41.12 | 15.28 | 29.55 | 4.88 | 34.79 | 13.42 |
| BACK | 4 | 43.51 | 9.74 | 54.57 | 15.76 | 43.62 | 14.78 | 45.39 | 18.85 |
| THICKNESS | 6 | 74.47** | 8.54 | 90.77^{NS} | 25.28 | 84.46* | 17.54 | 72.41** | 22.11 |
| | 8 | 32.42^{NS} | 5.18 | 45.99^{NS} | 15.46 | 46.07^{NS} | 22.52 | 41.36^{NS} | 19.771 |

NS = not significant,
*P < 0.05;
**P < 0.01

TABLE 21
Clinical Scores: Erythema and Scaling Day 1 (baseline) - Day 8
[scale 0-4] A) groups 1-4 and B) treatment with compounds 8 and 46
A

| | Group 1: HEB | | Group 2: IMQ | | Group 3: IMQ | | Group 4: IMQ | |
|---|---|---|---|---|---|---|---|---|
| | Vehicle | | Vehicle | | IL-17A mAb | | Dexamethasone | |
| Day | AVG Score | SD | AVG Score | SD | AVG Score | SD | AVG Score | SD |

Erythema (0-4)

| | | | | | | | | |
|---|---|---|---|---|---|---|---|---|
| 1 | 0 | 0 | 0 | 0 | 0 | 0 | 0 | 0 |
| 3 | 0 | 0 | 1.55 | 0.16 | 0.6 | 0.32 | 0.05 | 0.16 |
| 4 | 0 | 0 | 2.85 | 0.34 | 1.45 | 0.44 | 0.65 | 0.47 |
| 6 | 0 | 0 | 3.2 | 0.26 | 1.3 | 0.35 | 0.45 | 0.37 |
| 8 | 0 | 0 | 2.65 | 0.41 | 1.6 | 0.52 | 0.45 | 0.50 |

Scaling (0-4)

| | | | | | | | | |
|---|---|---|---|---|---|---|---|---|
| 1 | 0 | 0 | 0 | 0 | 0 | 0 | 0 | 0 |
| 3 | 0 | 0 | 0.4 | 0.21 | 0.3 | 0.26 | 0 | 0 |
| 4 | 0 | 0 | 1.05 | 0.16 | 0.45 | 0.16 | 0.2 | 0.26 |
| 6 | 0 | 0 | 3.35 | 0.34 | 1.45 | 0.50 | 0.5 | 0.47 |
| 8 | 0 | 0 | 2.8 | 0.26 | 2.7 | 0.59 | 0.35 | 0.47 |

B

| | Group 5: IMQ | | Group 6: IMQ | | Group 7: | Group 8: | |
|---|---|---|---|---|---|---|---|
| | Compound 8 | | Compound 46 | | Compound 46 | Compound 46 | |
| | 60 mpk BID | | 60 mpk QD | | 25 mpk BID | 60 mpk BID | |
| Day | AVG Score | SD | AVG Score | SD | SD | AVG Score | SD |

Erythema (0-4)

| | | | | | | | |
|---|---|---|---|---|---|---|---|
| 1 | 0 | 0 | 0 | 0 | 0 | 0 | 0 |
| 3 | 1.25 | 0.26 | 1.05 | 0.37 | 0.16 | 1.15 | 0.24 |
| 4 | 1.4 | 0.32 | 1.3 | 0.35 | 0.41 | 1.3 | 0.35 |
| 6 | 2.15 | 0.34 | 2.25 | 0.68 | 0.34 | 1.65 | 0.41 |
| 8 | 1.95 | 0.50 | 1.1 | 0.39 | 0.44 | 1.3 | 0.54 |

Scaling (0-4)

| | | | | | | | |
|---|---|---|---|---|---|---|---|
| 1 | 0 | 0 | 0 | 0 | 0 | 0 | 0 |
| 3 | 0.5 | 0 | 0.5 | 0 | 0.24 | 0.5 | 0 |
| 4 | 0.6 | 0.21 | 0.6 | 0.21 | 0.21 | 0.55 | 0.16 |
| 6 | 2.8 | 0.35 | 3.05 | 0.64 | 0.41 | 2.55 | 0.28 |
| 8 | 1.7 | 0.26 | 1.7 | 0.35 | 0.75 | 1.45 | 0.37 |

### Example 27

### Inhibition of Pro-Inflammatory Cytokine Response to TLR2 or TLR4 Agonism in Primary Human Umbilical Vein Endothelial Cells

It has been reported in the literature that that ERK5 mediates TLR2-dependent inflammatory signaling in several cell types including human umbilical vein endothelial cells (HUVEC). See Wilhelmsen et al., J Biol Chem. 2012; 287:26478-94. Wilhelmsen et al. further reported that MEK1 negatively regulates TLR2 signaling in HUVECs, with pharmacological inhibition of MEK1 augmenting (IL-6, G-CSF, GM-CSF) or not changing (IL-8) pro-inflammatory cytokine release in response to TLR2 stimulation.

In this example, the following commercially available inhibitors were tested:

a. MEK1/2 (AS703026) (Selleck Chemicals, Houston, Tex.),

b. p38 (SB203580) (Selleck Chemicals) and

c. ERK5 (XMD8-92) (Tocris Bioscience, R&D Systems, Minneapolis, Minn.)

The results were compared with test compound 61.

Pooled primary human umbilical vein endothelial cells (HUVEC) were obtained from Lonza (Allendale, N.J.) and maintained in either complete EGM-2 defined media (Lonza) or Medium 200 supplemented with Low Serum Growth Supplement (Life Technologies, Grand Island, N.Y.). Cells at low passage (<5) were seeded at 4e5 cells/ml and allowed to adhere overnight at 37° C. in a humidified atmosphere at 5% CO₂. Cells were pre-treated with test compounds for 1 h prior to TLR2 or TLR4 stimulation. To test for the effects of TLR2 agonism, the TLR2 agonist peptide Pam3CysK4 (Santa Cruz Biotechnology, Dallas, Tex.) was used at 10 μg/ml. PHC-SKKK (Enzo Life Sciences, Farmingdale, N.Y.) served as the negative control peptide. To test for the effects of TLR4 agonism, LPS from E. coli 0111:B4 (EMD Millipore, Billerica, Mass.) was used at 100 ng/ml. Cells were incubated for 22 hr, after which the supernatant was collected and analyzed for cytokine concentrations by ELISA (Life Technologies). Absorbance at 450 nm was read using the Wallac 1420 Victor² multilabel microplate reader (Perkin Elmer, Waltham, Mass.).

TABLE 22
IL-8 response of primary human umbilical vein endothelial
cells to TLR2 (Pam3CysK4) or TLR4 (LPS) stimulation in the
presence of various MAPK inhibitors

| | IL-8 (pg/ml) | | |
|---|---|---|---|
| | +10 μg/ml | +10 μg/ml PHC- | |
| Treatment | Pam3CysK4 | SKKK | +100 ng/ml LPS |
| DMSO | 661 ± 22 | 165 ± 35 | 3314 ± 120 |
| 5 μM AS703026 | 732 ± 36 | 0 ± 23 | 1970 ± 126 |
| 10 μM SB203580 | 450 ± 23 | 54 ± 12 | 1526 ± 54 |
| 5 μM XMD8-92 | 199 ± 11 | 0 ± 25 | 1249 ± 42 |
| 5 μM test | 133 ± 9 | 0 ± 17 | 1105 ± 25 |
| compound 61 | | | |

In contrast to the moderate IL-8 augmentation seen with the MEK1/2 inhibitor (AS703026), the p38 inhibitor (SB203580) the ERK5 inhibitor XMD8-92 and compound 61 reduced the IL-8 response to TLR2 (Pam3CysK4) stimulation. All MAPK inhibitors blunted the IL-8 response to TLR4 (LPS) stimulation.

TABLE 23
G-CSF response of primary human umbilical vein endothelial
cells to TLR4 (LPS) stimulation in the presence of various
MAPK inhibitors.

G-CSF (pg/ml)

| Treatment | No Stimulation | +100 ng/ml LPS |
|---|---|---|
| DMSO | 0 ± 5 | 96 ± 54 |
| 10 μM AS703026 | 30 ± 1 | 90 ± 13 |
| 10 μM SB203580 | 7 ± 9 | 318 ± 33 |
| 5 μM XMD8-92 | 0 ± 1 | 111 ± 6 |
| 5 μM test compound 61 | 0 ± 25 | 14 ± 5 |

Only test compound 61 reduced the G-CSF response to TLR4 agonism.

TABLE 24
IL-6 response of primary human umbilical vein endothelial cells
to TLR4 (LPS) stimulation in the presence of various MAPK inhibitors

IL-6 (pg/ml)

| Treatment | No Stimulation | +100 ng/ml LPS |
|---|---|---|
| DMSO | 137 ± 4 | 336 ± 72 |
| 10 μM AS703026 | 529 ± 20 | 1368 ± 169 |
| 10 μM SB203580 | 18 ± 5 | 112 ± 10 |
| 5 μM XMD8-92 | 20 ± 1 | 170 ± 21 |
| 5 μM test compound 61 | 7 ± 1 | 113 ± 14 |

Cytokine augmentation was observed with MEK1/2 inhibitor (AS703026). IL-6 was reduced by the p38 inhibitor (SB203580) and the ERK5 inhibitors XMD8-92 and test compound 61.

### Example 28

### Inhibition of Pro-Inflammatory Cytokine Response and a Marker of Squamous Metaplasia in Primary Human Corneal Epithelial Cells Stimulated with IL-17A, IL-1β, or IFNγ

Primary corneal epithelial cells were obtained from Life Technologies (Carlsbad, Calif.) and maintained in Keratinocyte-SFM media supplemented with EGF and bovine pituitary extract (Life Technologies). Cells were seeded at 1e5 cells/ml and incubated at 37° C. in a humidified atmosphere at 5% CO₂ overnight to adhere. Cells were supplement-starved overnight in unsupplemented K-SFM media containing 0.5% BSA then treated with compound for 1 h at 37° C. in a humidified atmosphere at 5% CO₂. DMSO at a final concentration of 0.1% served as the non-inhibited control. Cells were stimulated with 20 ng/ml of human recombinant IFNγ (R & D Systems, Minneapolis, Minn.), IL-1β (R & D Systems), or IL-17A (eBioscience, San Diego, Calif.) for 24 h. Supernatant was then collected and analyzed for cytokine concentrations using the Bio-Plex Pro Human Cytokine 17-Plex Panel (Bio-Rad Laboratories, Hercules, Calif.) according to the manufacturer's instructions. Magnetic beads were measured on the Bio-Plex MAGPIX multiplex reader instrument using the accompanying xPONENT 4.2 acquisition software (Bio-Rad Laboratories). Data were analyzed via the Bio-Plex Manager software v6.1 (Bio-Rad Laboratories). Shown in the data table are the percent inhibition values for four cytokines that were in the range of detection from the multiplex which exhibited ≧50% inhibition by the highest concentration of compound tested (10 μM).

Percent inhibition of cytokines in culture supernatant during 5 μM compound treatment of primary corneal epithelial cells. Shown below are data from a representative experiment.

TABLE 25

| | Percent inhibition of cytokine relative to DMSO controls | | | | | |
|---|---|---|---|---|---|---|
| | +20 ng/ml IFNγ | | +20 ng/ml IL-1β | | +20 ng/ml IL-17A | |
| | com- | com- | com- | com- | com- | com- |
| | pound | pound | pound | pound | pound | pound |
| Cytokine | 46 | T62 | 46 | T62 | 46 | T62 |
| IL-6 | 84.4 | 67.9 | 92.2 | 88.7 | 94.7 | 89.6 |
| G-CSF | 42.1 | 28.0 | 86.3 | 89.3 | 90.3 | 86.4 |
| GM-CSF | 30.4 | 25.4 | 97.0 | 97.1 | 92.5 | 89.7 |
| MCP-1 | 93.4 | 89.5 | 52.1 | 58.0 | 42.7 | 36.0 |

### Example 29

### Bromodomain Binding Assay

T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05 Tween 20, 1 mM DTT) to remove unbound ligand and to reduce non-specific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds (T62 and 46) were prepared as 1000× stocks in 100% DMSO and subsequently diluted 1:10 in monoethylene glycol (MEG) to create stocks at 100× the screening concentration (resulting stock solution is 10% DMSO/90% MEG). The compounds were then diluted directly into the assays such that the final concentration of DMSO and MEG were 0.1% and 0.9%, respectively. All reactions were performed in polystyrene 96-well plates in a final volume of 0.135 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 2 μM non-biotinylated affinity ligand) and incubated at room temperature with shaking for 30 minutes. The bromodomain concentration in the eluates was measured by qPCR. Compounds were assayed for binding activity against a panel of 40 bromodomain-containing proteins.

Results of the binding assay for Kd <20,000 nM are provided in Table 26.

TABLE 26

| Entrez Gene | |
|---|---|
| Symbol | |
| | T62 |
| | Kd (nM) |
| TAF1L | 19000 |
| TAF1 | 12000 |
| CREBBP | 1200 |
| EP300 | 1100 |
| BRDT | 397 |
| BRD4 | 97 |
| BRD3 | 110 |
| BRD2 | 93 |
| | 46 |
| | Kd (nM) |
| EP300 | 7500 |
| CREBBP | 3300 |
| BRDT | 169 |
| BRD2 | 55 |
| BRD4 | 41 |
| BRD3 | 33 |

It was also determined that the Kd for binding of Compound T62 to the following bromodomain proteins were all >20,000 nM: ATAD2A, ATAD2B, BAZ2A, BAZ2B, BRD1, BRD7, BRD8, BRD9, BRPF1, BRPF3, CECR2, FALZ, GCN5L2, PBRM1, PCAF, SMARCA2, SMARCA4, TAF1, TAF1L, TRIM24, TRIM33, and WDR9.

In this Bromodomain binding assay, it was also determined that the Kd for binding of Compound 46 to the following bromodomain proteins were all >20,000 nM: ATAD2A, ATAD2B, BAZ2A, BAZ2B, BRD1, BRD7, BRD8, BRD9, BRPF1, BRPF3, CECR2, FALZ, GCN5L2, PBRM1, PCAF, SMARCA2, SMARCA4, TRIM24, TRIM33, and WDR9.

### Example 30

### BRD4 Bromodomain Domain 1 Inhibition Assay

The inhibitory activity of test compounds on the bromodomain, BRD4 bromodomain domain I [BRD4(1)], were determined via TR-FRET after a slight modification of the manufacturer's protocol (Cayman Chemicals, Ann Arbor, Mich.). Briefly, compounds were diluted to a 20× (2 mM) concentration in DMSO. 100 nL of 20× compounds were then dry stamped into triplicate wells of black, low volume 384 well plates (Aurora Biotechnologies, Carlsbad, Calif.) using the Mosquito liquid handler (TPP Labtech, Melbourn, UK). 20× compounds were then diluted to a 4× (40 μM) concentration in Assay Buffer to result in 2% DMSO content. The manufacturer's protocol was then followed to result in a final reaction volume of 20 μl per well, 1× (10 μM or 5 μM) compound final concentration, and 0.5% DMSO content. JQ1 at 10 μM or 0.5% DMSO final concentrations were used as the positive and negative controls, respectively, to set the assay window. The fluorescence was read in a time-resolved format on the Biotec Synergy 2 plate reader by exciting the signal at 340 nm and reading emissions at 620 and 670, using a 100 μs delay and 200 μs read window. Percent inhibition values at the indicated screening concentration were determined using the TR-FRET ratios (670 nm emission/620 nm emission). Briefly, TR-FRET ratio values were normalized to the average TR-FRET ratio value given by 10 μM JQ1. The normalized, average TR-FRET ratio value given by the 0.5% DMSO control was then set to 0% inhibition and all average % inhibition values for compounds tested indicated in Table 27 are relative this value.

In Table 27, % inhibition is expressed as follows: A is >85%; B is 85%-70%; and C is <70%.

TABLE 27

| | Screening | |
|---|---|---|
| | concentration | % inhibition |
| Compound | (μM) | [BRD4(1)] |
| T61 | 10 | A |
| 15 | 10 | A |
| T42 | 10 | A |
| 91 | 10 | A |
| 56 | 10 | A |
| 65 | 10 | A |
| 46 | 10 | A |
| 67 | 10 | A |
| T31 | 10 | A |
| T49 | 10 | A |
| 113 | 10 | A |
| T4 | 10 | A |
| 43 | 10 | A |
| 25 | 10 | A |
| T10 | 10 | A |
| 71 | 10 | A |
| 24 | 10 | A |
| T11 | 10 | A |
| 82 | 10 | A |
| 28 | 10 | A |
| T8 | 10 | A |
| 96 | 10 | A |
| 75 | 10 | A |
| T20 | 5 | A |
| T50 | 10 | A |
| T37 | 10 | A |
| 26 | 10 | A |
| 120 | 10 | A |
| 42 | 10 | A |
| 6 | 10 | A |
| T27 | 10 | A |
| 111 | 10 | A |
| T29 | 10 | A |
| 88 | 10 | A |
| T14 | 10 | A |
| T25 | 10 | A |
| 61 | 10 | A |
| T26 | 10 | A |
| 93 | 10 | A |
| 49 | 10 | A |
| 17 | 10 | A |
| 54 | 10 | A |
| 114 | 10 | A |
| 31 | 10 | A |
| T15 | 10 | A |
| 62 | 10 | B |
| T34 | 10 | B |
| 103 | 10 | B |
| 30 | 10 | B |
| 20 | 10 | B |
| 94 | 10 | B |
| 8 | 10 | B |
| 37 | 10 | B |
| 72 | 10 | B |
| 108 | 10 | B |
| 55 | 10 | B |
| 115 | 10 | B |
| 10 | 10 | B |
| 92 | 10 | B |
| T56 | 10 | B |
| 44 | 10 | B |
| T18 | 10 | B |
| T48 | 10 | B |
| T9 | 10 | B |
| 122 | 10 | B |
| 1 | 10 | B |
| T5 | 10 | B |
| T64 | 10 | B |
| 21 | 10 | B |
| 63 | 10 | B |
| 12 | 10 | B |
| 105 | 10 | B |
| 52 | 10 | B |
| 53 | 10 | B |
| T62 | 10 | B |
| T76 | 10 | B |
| 99 | 10 | B |
| 70 | 10 | B |
| 100 | 10 | B |
| 78 | 10 | B |
| 101 | 10 | B |
| 84 | 10 | C |
| 69 | 10 | C |
| T22 | 10 | C |
| 80 | 10 | C |
| 109 | 10 | C |
| 48 | 10 | C |
| 39 | 10 | C |
| 45 | 10 | C |
| 98 | 10 | C |
| 110 | 10 | C |
| 83 | 10 | C |
| 66 | 10 | C |
| 90 | 10 | C |
| 33 | 10 | C |
| 106 | 10 | C |
| 47 | 10 | C |
| 29 | 10 | C |
| T70 | 10 | C |
| 107 | 10 | C |
| 57 | 10 | C |
| 7 | 10 | C |
| T81 | 10 | C |
| 64 | 10 | C |
| 73 | 10 | C |
| 2 | 10 | C |
| 74 | 10 | C |
| 102 | 10 | C |
| 79 | 10 | C |
| 23 | 10 | C |
| 9 | 10 | C |
| 51 | 10 | C |
| 40 | 10 | C |

Additional compounds and their IC₅₀s (μM) for BRD4(1) are provided in Table 27A. The IC₅₀ values (μM) are represented as follows: A is <2; B is 2-5; and C is >5.

TABLE 27A

| Compound | BRD4(1) IC₅₀ (μM) |
|---|---|
| T117 | C |
| T114 | C |
| T96 | B |
| T116 | C |
| T92 | A |
| T93 | C |
| T115 | B |
| T137 | C |
| T95 | B |
| T118 | C |
| T119 | A |
| T94 | C |
| T97 | B |
| T110 | C |
| T113 | C |
| T122 | C |
| T120 | C |
| T127 | C |
| T128 | B |
| T129 | B |
| T136 | C |
| T89 | C |
| T98 | B |
| T106 | A |
| T99 | C |
| T102 | A |
| T103 | C |
| T105 | A |
| T124 | A |
| T125 | B |
| T104 | B |
| T107 | A |
| T126 | C |

### Example 30

### Effect on Collagen-Induced Arthritis in Mouse Model

To study the effect of test compounds on rheumatoid arthritis, a mouse model with collagen-induced arthritis (CIA model) was used. In this study, mice (8 week old male DBA/1J H2^{q}) were randomly assigned to groups on Day 0. Arthritis was induced by immunization on Day 0 by intradermal injection at the base of the tail of 0.1 ml emulsion containing 100 μg bovine type II collagen (CII) in Complete Freund's Adjuvant (CFA) (100 μg M. tuberculosis) (Chondrex). A booster on Day 21 of IP CII/ICFA induced disease onset within 1-3 days. Treatment began the morning of the booster, prior to IP injection, and continued for 21 days. Treatment groups, summarized in Table 28, were vehicle (2.5% DMA/47.5% PEG-400/50% water), positive controls (anti-IL17A monoclonal antibody (5 mg/kg IP, LEAF™ BioLegend, San Diego, Calif.), Tofacitinib 15 mg/kg BID PO (Selleckchem Houston Tex.) or test article.

TABLE 28
CIA: Group Treatments

| Group | | Immunize | | | |
|---|---|---|---|---|---|
| (n =) | Treatment | for CIA? | Dose | Route | Regimen |
| 1 | DMA/PEG | Yes | NA | PO | BID from |
| (11) | Vehicle | | | | D21-D42 |
| 2 | Tofacitinib | Yes | 15 mg/kg | PO | BID from |
| (10) | (CMC/Tween80) | | | | D21-D42 |
| 3 | 46 | Yes | 60 mg/kg | PO | BID from |
| (10) | (DMA/PEG) | | | | D21-D42 |
| 4 | Anti-IL-17A mAb | Yes | 5 mg/kg | IP | Once-weekly |
| (10) | | | | | for 3 weeks |
| | | | | | (Days 21, |
| | | | | | 28, 35) |
| 5 | Naïve (no CIA) | No | NA | PO | BID from |
| (5) | Vehicle | | | | D21-D42 |

All dose volumes were 5 mL/kg. On Day 42 only AM dose was administered.

Measurements of body weight (BW), total paw thickness by caliper (sum of 4 paws) and gross clinical disease occurred once weekly from Day 0-21 and then approximately every 3 days until terminal sacrifice on Day 42.

In addition to caliper measurement of the paws, the in-life disease response readout was visual assessment of joint (ankle through digits) inflammation and erythema. Maximum disease score/paw was 4 for a total possible maximum score of 16. To assure consistency, all in-life assessments were performed by the same person. Hind limbs were harvested for histopathological analysis of knee (tibiofemoral) and paw (tarsal/phalanges). The various organs were harvested, and only the spleen was wet-weighed prior to snap-freezing. Terminal blood samples and spleen were collected approximately two (2) hours post-dose on study day 42.

The extent of in-life disease induced was mild-moderate, typical for the induction protocol used sufficient to observe differential treatment effects. (see Yamanishi et al., Regulation of Joint Destruction and Inflammation by p53 in Collagen-Induced Arthritis, Am. J. Pathol. 2002, 160 (1): 123-130, and Lubberts et al, Treatment with a Neutralizing Anti-murine Interleukin-17 Antibody After Onset of Collagen-Induced Arthritis Reduces Joint Inflammation, Cartilage Destruction, and Bone Erosion. Arth. and Rheum. 2004, 50 (2): 650-659). For all gross clinical observations, anti-IL-17A mAb was highly effective resulting in near normalization; the responses to treatment with Tofacitinib and 46 were virtually identical with significant reductions of about 55% in both overall disease and total paw responses.

TABLE 29
change in group average disease score - mean
Day versus Day 21 (start of treatment)

| Day | Group 1 | Group 2 | 46 | Group 4 |
|---|---|---|---|---|
| 21 | 0.0 | 0.0 | 0.0 | 0.0 |
| 24 | 0.9 | 0.3 | 0.4 | 0.3 |
| 26 | 2.8 | 0.9 | 1.3 | 0.4 |
| 28 | 3.6 | 1.8 | 2.0 | 0.8 |
| 31 | 4.9 | 2.3 | 2.1 | 1.1 |
| 33 | 5.1 | 2.6 | 2.7 | 1.1 |
| 35 | 5.5 | 2.7 | 2.4 | 1.3 |
| 37 | 5.6 | 2.2 | 2.7 | 1.3 |
| 39 | 5.7 | 2.5 | 2.8 | 1.3 |

Decalcified, H&E stained tissues from both tibiofemoral and tarsal/phalangeal joints of every animal were evaluated. The histopathology of the hind-limbs revealed differences among all treatments. As with the in-life disease, the anti-IL17A mAb resulted in almost complete normalization of the effects of CIA at both the knee and paw joints in nearly all of the animals in that group. However, Tofacitinib was less effective at mitigating cartilage and periosteal erosion at the knee and ulceration and synovial hyperplasia than 46. Additionally, Compound 46 was nearly twice as effective as Tofacitinib at protecting the tarsal joints.

Treatment effects on histopathology
Number of animals with normal tarsal joints
(% of total n in group)

| Group 1 | Group 2 | Group 3 | Group 4 | Group 5 |
|---|---|---|---|---|
| 3 (27%) | 4 (40%) | 7 (70%) | 9 (90%) | 5 (100%) |

Tibiofemoral joint scores

| | | | | |
|---|---|---|---|---|
| Group 1 | Group 2 | Group 3 | Group 4 | Group 5 |

cartilage erosion/ulceration

| | | | | |
|---|---|---|---|---|
| 21.3 | 16.3 | 12.5 | 3.0 | 0 |

inflammation

| | | | | |
|---|---|---|---|---|
| 18.3 | 16.3 | 15.0 | 1.0 | 0 |

periosteal proliferation/erosion

| | | | | |
|---|---|---|---|---|
| 11.3 | 7.8 | 3.8 | 1.0 | 0 |

synovial hyperplasia

| | | | | |
|---|---|---|---|---|
| 6.3 | 5.0 | 3.0 | 3.0 | 0 |

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A compound of formula I: 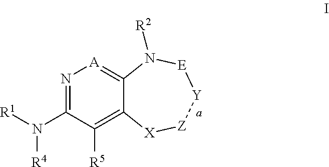
or a pharmaceutically acceptable salt thereof, wherein
bond a is a single bond or double bond;
R¹ and R⁴ are each independently selected from hydrogen, alkyl, alkenyl, alkynyl, aryl, heteroaryl, heterocyclyl and cycloalkyl;
R² is alkyl, deuteroalkyl, alkenyl, alkynyl, haloalkyl or cycloalkyl;
X is NR³, O, S(O)ₘ, or CR^{a}R^{b};
R^{a} and R^{b} are selected as follows:
(i) R^{a} and R^{b} are each independently selected from hydrogen, alkyl, alkenyl, alkynyl, haloalkyl, cycloalkyl, aryl, heterocyclyl and heteroaryl; or
(ii) R^{a} and R^{b} together form ═O;
R³ is alkyl, deuteroalkyl, alkenyl, alkynyl, haloalkyl, cycloalkyl, SO₂R¹⁹, COR², or —SO₂N(R¹⁴)(R¹⁵);
R⁵ is selected from hydrogen, alkyl, alkenyl, alkynyl and cycloalkyl;
A is CH, CR², or N;
E is CO, SO₂, CN(OR¹⁸), CN(CN), CS, CNR¹¹, or CR¹²CF₃;
Y is CR⁷ or CR⁷R⁸;
Z is CR⁹ or CR⁹R¹⁰;
R⁷ and R⁹ together with the atoms on which they are substituted form an optionally substituted 3 to 6-membered cycloalkyl, aryl, heterocyclyl or heteroaryl ring, where substituents, when present are selected from one or more Q¹ and Q³ groups;
R⁸ and R¹⁰, when present, are each independently selected from hydrogen, alkyl and cycloalkyl;
Q¹ is selected from alkyl, cycloalkyl, aryl and heteroaryl;
R¹¹ and R¹² are each independently selected from hydrogen, alkyl and cycloalkyl;
R¹⁸ is hydrogen, alkyl or cycloalkyl;
R¹⁹ is alkyl, alkenyl, alkynyl, haloalkyl, cycloalkyl, aryl or heteroaryl;
Q¹, R^{a}, R^{b}, R², R³, R⁴, R⁵, R⁸, R¹⁰ and R¹⁹ are optionally substituted with 1, 2, 3 or 4 substituents, each independently selected from Q², where Q² is selected from deutero, alkyl, alkenyl, alkynyl, haloalkyl, cycloalkyl, hydroxyl and halo;
R¹ is optionally substituted with 1, 2, 3 or 4 substituents Q³, each Q³ is independently selected from halo, cyano, oxo, thioxo, alkyl, haloalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}OR^{u}N(R^{y})(R^{z}), —R^{u}N(R^{y})(R^{z}), —R^{u}SR^{x}, —R^{u}C(J)R^{x}, —R^{u}C(J)OR^{x}, —R^{u}C(J)N(R^{y})(R^{z}), —R^{u}S(O)ₜN(R^{y})(R^{z}), —R^{u}S(O)ₜR^{w}, —R^{u}N(R^{x})C(J)R^{x}, —R^{u}N(R^{x})C(J)OR^{x}, —R^{u}N(R^{x})S(O)ₜR^{w}, and —C(═NR^{y})N(R^{y})OR^{x}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups;
each Q⁴ is independently selected from halo, hydroxyl, amino, alkyl, cycloalkyl, haloalkyl and hydroxyalkyl;
each R^{u} is independently alkylene, alkenylene or a direct bond;
R^{w} is alkyl, haloalkyl, hydroxyalkyl, alkoxyalkyl, amino, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;
each R^{x} is independently hydrogen, alkyl, haloalkyl, hydroxyalkyl, alkoxyalkyl, cyanoalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl;
R^{y} and R^{z} are each independently selected from (i) or (ii) below:
(i) R^{y} and R^{z} are each independently hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a heterocyclyl or heteroaryl, optionally substituted with one or more Q⁵ groups;
each Q⁵ is independently selected from halo, oxo, thioxo, hydroxy, cyano, amino, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}SR^{x}, —R^{u}C(J)R^{x}, —R^{u}N(R¹⁴)(R¹⁵), —R^{u}C(J)OR^{x}, —OC(J)R^{u}N(R¹⁴)(R¹⁵), —R^{u}C(J)R^{u}N(R¹⁴)(R¹⁵), —R^{u}S(O)ₜR^{w}, —R^{u}N(R^{x})C(J)R^{x}, —R^{u}N(R^{x})C(J)OR^{x}, —OP(O)(OH)₂, and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one or more, in one embodiment, one, two or three Q⁸ groups;
each Q⁸ is independently selected from halo, hydroxy, alkyl, alkoxy, and haloalkyl;
each of R¹⁴ and R¹⁵ is optionally substituted with one or two halo, hydroxy, alkyl, alkoxy or haloalkyl;
J is O, NR^{x} or S;
each t is independently an integer from 0-2; and
m is 0-2.

2. The compound of claim 1, wherein
Y is CR⁷ or CR⁷R⁸;
Z is CR⁹ or CR⁹R¹⁰; wherein R⁷ and R⁹ together with the atoms on which they are substituted form a 3 to 6-membered cycloalkyl, aryl, heterocyclyl or heteroaryl ring; and R⁸ and R¹⁰, when present, are each independently selected from hydrogen, alkyl and cycloalkyl.

3. The compound of claim 1, wherein R¹ is phenyl, pyridinyl, cyclohexyl, tetrahydropyranyl or pyrazolyl, where R¹ is optionally substituted with 1 or 2 substituents Q³.

4. The compound of claim 1, wherein R¹ is: 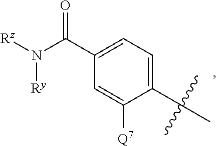
(i) R^{y} is selected from hydrogen and alkyl; and R^{z} is hydrogen, alkyl, aminoalkyl, hydroxyalkyl, alkoxyalkyl, cycloalkyl, cycloalkylalkyl, heterocyclyl, heterocyclylalkyl, aryl, heteroaryl or heteroarylalkyl, where R^{z} is optionally substituted with one or two alkyl, hydroxyl, alkoxy, —COOH or amino groups; or
(ii) R^{y} and R^{z}, together with the nitrogen atom to which they are attached, form a 5 to 7 membered heterocyclyl or heteroaryl, optionally substituted with one, two or three Q⁵ groups; each Q⁵ is independently selected from halo, hydroxy, amino, cyano, oxo, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —R^{u}N(R^{x})C(J)OR^{x}, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl, hydroxyalkyl, cyano and amino;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one, two or three Q⁸ groups;
each Q⁸ is independently selected from halo, hydroxy, alkyl, alkoxy, and haloalkyl;
each of R¹⁴ and R¹⁵ is optionally substituted with one or two halo, hydroxy, alkyl, alkoxy or haloalkyl;
J is O;
each R^{u} is independently alkylene or a direct bond;
R^{w} is alkyl;
each R^{x} is independently hydrogen, alkyl, hydroxyalkyl or alkoxyalkyl; and
t is an integer from 0-2.

5. The compound of claim 1, wherein R¹ is: 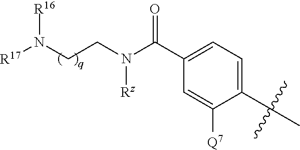
where Q⁷ is alkyl or alkoxy;
R^{z}, R¹⁶ and R¹⁷ are selected as follows:
(i) R^{z}, R¹⁶ and R¹⁷ are each independently hydrogen, alkyl, cycloalkyl or cycloalkylalkyl;
(ii) R^{z} is selected from hydrogen and alkyl; and R¹⁶ and R¹⁷ together with the nitrogen atom on which they are substituted form an optionally substituted 5-7 membered heterocyclyl or heteroaryl ring; where the substituents when present are selected from alkyl, cycloalkyl, cycloalkylalkyl, aminoalkyl, alkoxy, amino and hydroxyl;
(iii) R¹⁶ is selected from hydrogen and alkyl; and R^{z} and R¹⁷ together with the atoms on which they are substituted form an optionally substituted 5-7 membered heterocyclyl ring; where the substituents when present are selected from one, two or three Q⁵ groups;
each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, cycloalkenylalkyl, aryl, aralkyl, heteroaryl, heteroaralkyl, heterocyclyl, heterocyclylalkyl, —R^{u}OR^{x}, —R^{u}C(J)OR^{x}, —R^{u}N(R)C(J)OR^{x}, R^{u}C(J)N(R¹⁴)(R¹⁵), and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;
J is O;
each R^{u} is independently alkylene or a direct bond;
R^{w} is alkyl;
each R^{x} is independently hydrogen, alkyl, hydroxyalkyl or alkoxyalkyl;
t is an integer from 0-2; and
q is 1 or 2.

6. The compound of claim 4, wherein Q⁷ is alkoxy.

7. The compound of claim 4, wherein Q⁷ is ethoxy.

8. The compound of claim 1, wherein the compound is of Formula II-1: 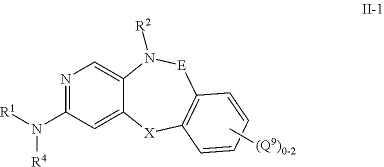
or a pharmaceutically acceptable salt thereof, wherein each Q⁹ is independently halo, alkyl, haloalkyl, hydroxyl or alkoxy.

9. The compound of claim 8, wherein
X is NR³, O or S(O)₀₋₂;
R² is alkyl or deuteroalkyl;
R³ is alkyl, deuteroalkyl, cycloalkyl or SO₂R¹⁹;
R⁴ hydrogen or alkyl;
R¹⁹ is alkyl;
E is CO or SO₂;
R¹ is aryl, heteroaryl, heterocyclyl or cycloalkyl;
R¹ is optionally substituted with 1 or 2 substituents Q³, each Q³ is independently selected from halo, cyano, alkyl, haloalkyl, alkenyl, alkynyl, cycloalkyl, cycloalkenylalkyl, heteroaryl, heterocyclyl, heterocyclylalkyl, —COOH, —R^{u}OR^{x}, —R^{u}N(R^{y})(R^{z}), —R^{u}C(J)N(R^{y})(R^{z}), —R^{u}S(O)ₜN(R^{y})(R^{z}), and —R^{u}N(R^{x})S(O)ₜR^{w}, where the alkyl, haloalkyl, aminoalkyl, alkenyl, alkynyl, cycloalkyl, cycloalkenyl, aryl, heteroaryl, and heterocyclyl groups are optionally substituted with one to six Q⁴ groups, each Q⁴ is independently selected from halo, hydroxyl, amino, alkyl, cycloalkyl, haloalkyl and hydroxyalkyl;
each R^{u} is independently alkylene or a direct bond;
R^{w} is alkyl or amino;
each R^{x} is independently hydrogen, alkyl or hydroxyalkyl;
R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups;
each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R^{x})C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one or more, in one embodiment, one, two or three Q⁸ groups;
each Q⁸ is independently selected from halo, hydroxy, alkyl, alkoxy, and haloalkyl;
each Q⁹ independently selected from halo, hydroxy, alkyl, alkoxy, and haloalkyl;
each of R¹⁴ and R¹⁵ is optionally substituted with one or two halo, hydroxy, alkyl, alkoxy or haloalkyl;
J is O; and
t is an integer from 0-2.

10. The compound of claim 1, wherein the compound is of Formula IIIA: 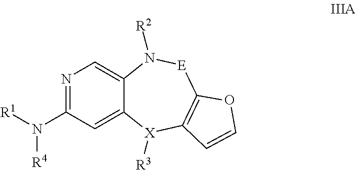
or a pharmaceutically acceptable salt thereof.

11. The compound of claim 10, wherein R² and R³ are alkyl; R⁴ hydrogen or alkyl;
E is CO;
R¹ is aryl or cycloalkyl;
R¹ is optionally substituted with 1 or 2 substituents Q³, each Q³ is independently selected from, —R^{u}OR^{x}, —R^{u}N(R^{y})(R^{z}), —R^{u}S(O)ₜN(R^{y})(R^{z}) and —R^{u}C(J)N(R^{y})(R^{z});
each R^{u} is independently alkylene or a direct bond;
each R^{x} is independently hydrogen, alkyl or hydroxyalkyl;
R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl, optionally substituted with one or more, in one embodiment, one, two or three Q⁵ groups; each Q⁵ is independently selected from amino and heterocyclyl, where each Q⁵ is optionally substituted with one or two alkyl groups; and J is O.

12. The compound of claim 1, wherein the compound is of Formula VI or VI-1: 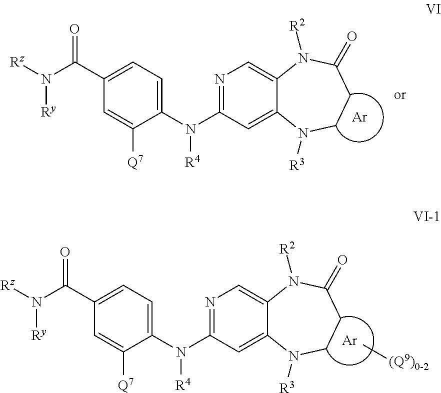
or a pharmaceutically acceptable salt thereof, where ring Ar is 5 or 6 membered aryl or heteroaryl ring; each Q⁹ is independently halo, alkyl, haloalkyl, hydroxyl or alkoxy; and Q⁷ is alkyl or alkoxy.

13. The compound of claim 12, wherein ring Ar is 5 or 6 membered aryl or heteroaryl ring;
R² is alkyl or deuteroalkyl;
R³ is alkyl, deuteroalkyl, cycloalkyl or SO₂R¹⁹;
R¹⁹ is alkyl;
Q⁷ is hydrogen, alkyl or alkoxy;
R^{y} and R^{z} are each independently selected from (i) or (ii) below:
(i) R^{y} is hydrogen or alkyl; and R^{z} is hydrogen, alkyl, haloalkyl, hydroxyalkyl, alkoxyalkyl, alkenyl, alkynyl, cycloalkyl, cycloalkenyl, cycloalkylalkyl, cycloalkenylalkyl, heterocyclyl, heterocyclylalkyl, aryl, aralkyl, heteroaryl, or heteroaralkyl; where R^{y} and R^{z} are each optionally substituted with one, two or three Q⁵ groups; or
(ii) R^{y} and R^{z}, together with the nitrogen atom to which they are attached, form a 5 to 7 membered heterocyclyl or heteroaryl, optionally substituted with one or more, in one embodiment, one, two or three Q⁵ groups; each Q⁵ is independently selected from halo, hydroxy, amino, cyano, alkoxy, alkyl, haloalkyl, hydroxyalkyl, aminoalkyl, alkenyl, alkynyl, cycloalkyl, cycloalkenyl, cycloalkylalkyl, aryl, aralkyl, heteroaryl, heteroaralkyl, heterocyclyl, heterocyclylalkyl, —R^{u}OR^{x}, —R^{u}C(J)R^{x}, —R^{u}C(J)OR^{x}, —R^{u}N(R^{x})C(J)OR^{x}, —R^{u}N(R¹⁴)(R¹⁵), —OC(J)R^{u}N(R¹⁴)(R¹⁵), —R^{u}C(J)R^{u}NR¹⁴R¹⁵, —OP(O)(OH)₂, —R^{u}S(O)ₜR^{w} and —R^{u}N(R^{x})S(O)ₜR^{w}, where when Q⁵ is amino, alkyl, cycloalkyl, aryl, heteroaryl or heterocyclyl, Q⁵ is optionally substituted with one, two or three Q⁶ groups selected from alkyl, alkenyl, alkynyl, cycloalkyl, halo, hydroxyl and amino;
R¹⁴ and R¹⁵ are each independently (i) or (ii) below:
(i) R¹⁴ and R¹⁵ are each independently hydrogen, alkyl, haloalkyl, hydroxyalkyl, alkoxyalkyl, alkenyl, alkynyl, cycloalkyl, heterocyclyl, aryl or heteroaryl; or
(ii) R¹⁴ and R¹⁵, together with the nitrogen atom to which they are attached, form a heterocyclyl or heteroaryl, optionally substituted with one or more, in one embodiment, one, two or three Q⁸ groups;
each Q⁸ is independently selected from halo, hydroxy, alkyl, alkoxy, and haloalkyl;
each of R¹⁴ and R¹⁵ is optionally substituted with one or two halo, hydroxy, alkyl, alkoxy or haloalkyl;
J is O;
each R^{u} is independently alkylene or a direct bond;
R^{w} is alkyl;
each R^{x} is independently hydrogen, alkyl or hydroxyalkyl; and
t is an integer from 0-2.

14. The compound of claim 1, wherein the compound is of formula XII 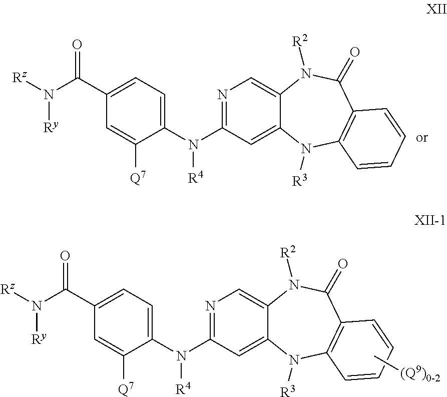
or a pharmaceutically acceptable salt thereof, where each Q⁹ is independently selected from halo, hydroxy, alkyl, alkoxy, and haloalkyl; and Q⁷ is alkyl or alkoxy.

15. The compound of claim 1, wherein the compound is of formula XIII 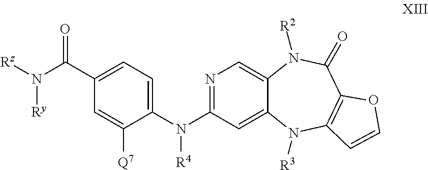
or a pharmaceutically acceptable salt thereof, where Q⁷ is alkyl or alkoxy.

16. The compound of claim 14, wherein Q⁷ is alkoxy.

17. The compound of claim 14, wherein Q⁷ is ethoxy.

18. The compound of claim 1, wherein the compound has formula IB: 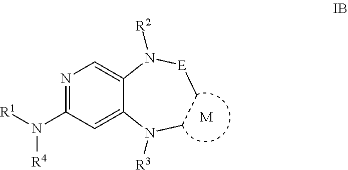
or a pharmaceutically acceptable salt thereof, wherein
E is CO, or SO₂;
M is 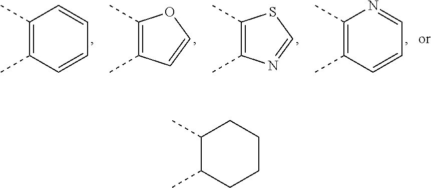
R¹ is phenyl, pyridyl, pyrazolyl, cyclohexyl, or tetrahydropyranyl ring, which is optionally substituted with 1 or 2 substituents Q³ or Q⁴, wherein Q³ and Q⁴ is independently selected from halo, cyano, hydroxy, C₁-C₄alkyl, amino(C₁-C₄)alkyl, C₁-C₄alkyloxy, halo(C₁-C₄)alkyloxyl, hydroxy(C₁-C₄)alkyl, C₁-C₄alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOR^{2a}, —COR^{3a}, or —CH₂R^{4a};
R^{2a} is C₁-C₄ alkyl, 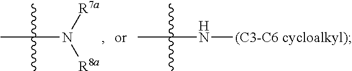
R^{3a} is selected from amino, hydroxy, 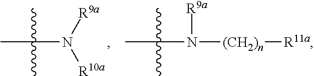
or 4 to 7 member heterocyclyl which may be substituted with halogen, hydroxy, cyano, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, or a 4 to 6 member heterocyclyl group;
R^{4a} is hydroxy, 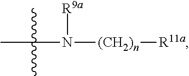
or 4 to 7 member heterocyclyl group,
which may be substituted with halogen, hydroxy, cyano, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, or a 4 to 6 member heterocyclyl group;
R², R³, R^{7a} and R^{8a} are independently C₁-C₄ alkyl or C₃-C₆ cycloalkyl;
R⁴ is hydrogen or C₁-C₄ alkyl;
R^{9a} and R^{10a} are independently selected from hydrogen, hydroxy, C₁-C₄ alkyl, hydroxy(C₁-C₄)alkyl, C₁-C₄ acyl, C₁-C₄ alkyloxy(C₁-C₄)alkyl, C₁-C₄ alkenyl, or, 4 to 7 member heterocyclyl group,
which may be substituted with halogen, hydroxy, cyano, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, or a 4 to 6 member heterocyclyl group;
R^{11a} is 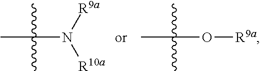
n is a natural number from 1 to 3.

19. The compound of claim 1, wherein the compound has formula ID: 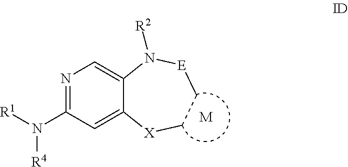
or a pharmaceutically acceptable salt thereof,
X is NR³, O, S(O)₀₋₂, or CR^{a}R^{b};
R^{a} and R^{b} are selected as follows:
(i) R^{a} and R^{b} are each independently selected from hydrogen, C₁₋₄alkyl, C₂₋₄alkenyl, C₂₋₄alkynyl, halo C₁₋₄alkyl, C₃₋₆cycloalkyl, aryl, heterocyclyl and heteroaryl; or
(ii) R^{a} and R^{b} together form ═O;
R³ is C₁₋₄alkyl, deutero C₁₋₄alkyl, C₂₋₄alkenyl, C₂₋₄alkynyl, halo C₁₋₄alkyl, C₃₋₆cycloalkyl, SO₂R¹⁹, COR², or —SO₂N(R¹⁴)(R¹⁵);
E is CO, SO₂ or CHCF₃;
ring M is or 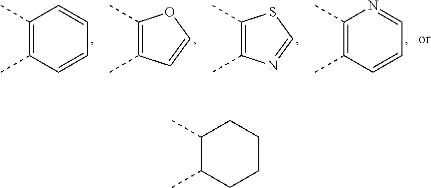
R² is C₁-C₄ alkyl or deuteroC₁₋₄alkyl;
R⁴ is hydrogen or C₁-C₄ alkyl;
R¹ is phenyl, pyridyl, pyrazolyl, cyclohexyl, cyclobutyl, or tetrahydropyranyl ring, which is optionally substituted with 1 or 2 substituents Q^{3a} or Q^{4a}, wherein each of Q^{3a} and Q^{4a} is independently selected from halo, cyano, hydroxy, C₁-C₄ alkyl, amino(C₁-C₄)alkyl, C₁-C₄ alkyloxy, halo(C₁-C₄)alkyloxyl, hydroxy(C₁-C₄)alkyl, C₁-C₄ alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOR^{2a}, —COR^{3a}, and —CH₂R^{4a};
R^{2a} is alkyl C₁-C₄ alkyl, 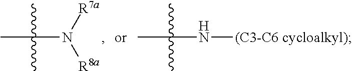
R^{3a} is selected from amino, hydroxy, 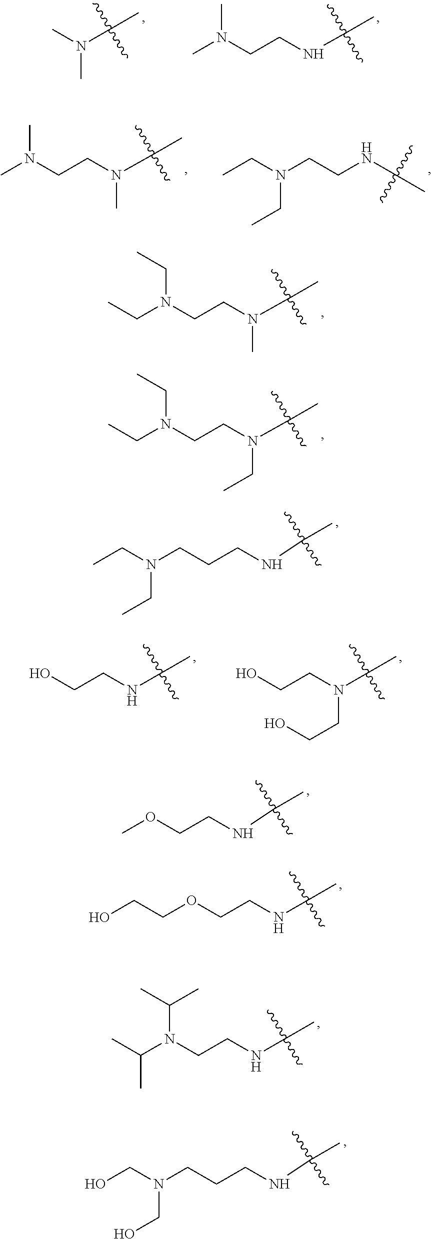 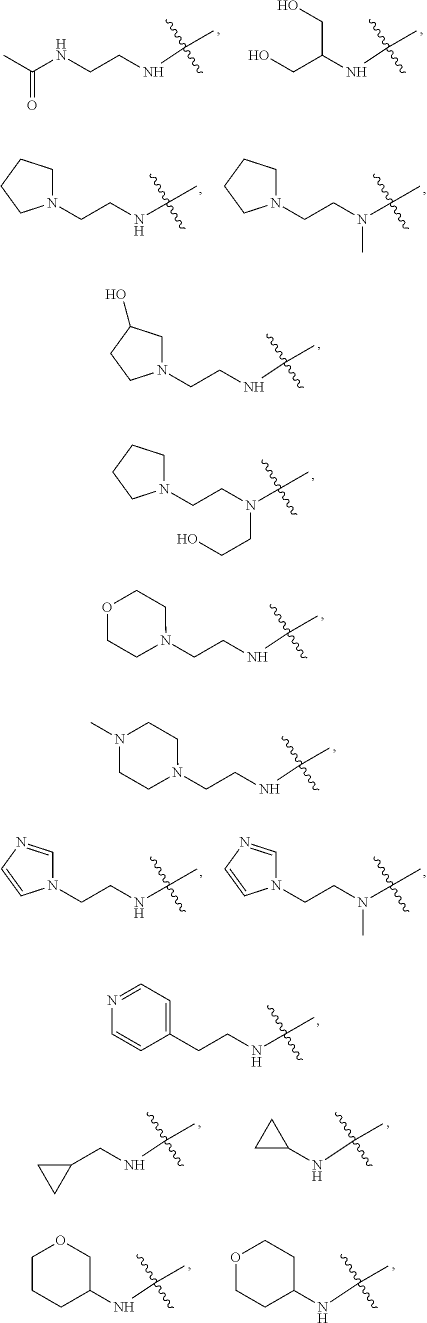 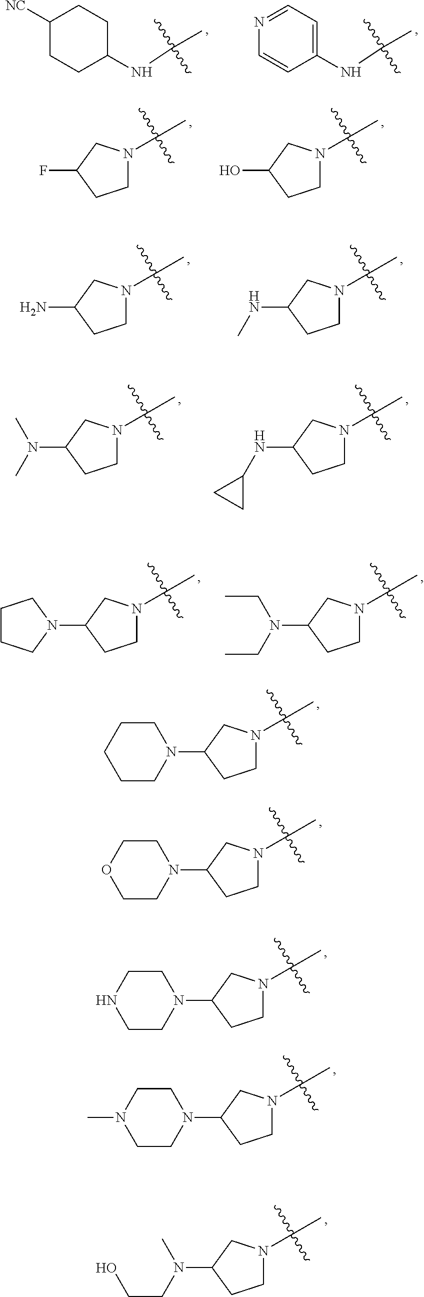 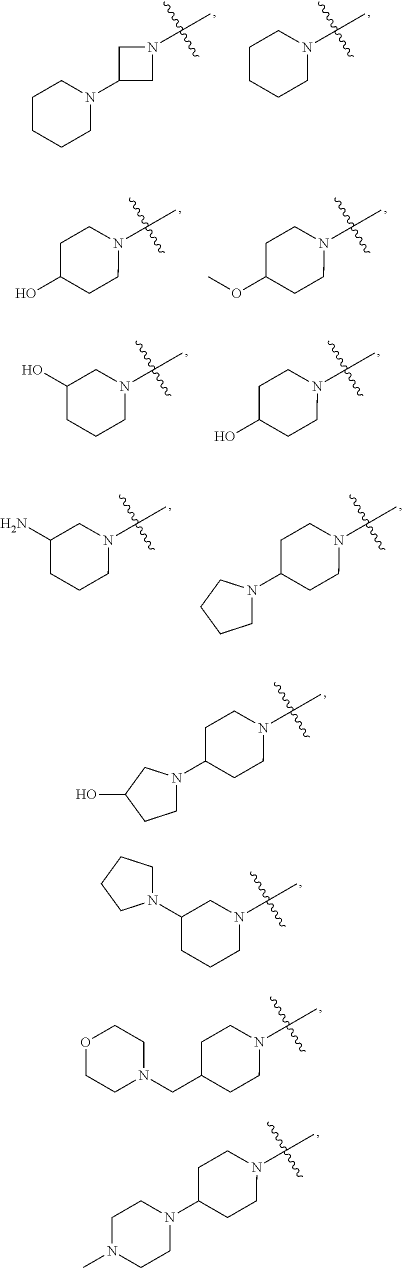 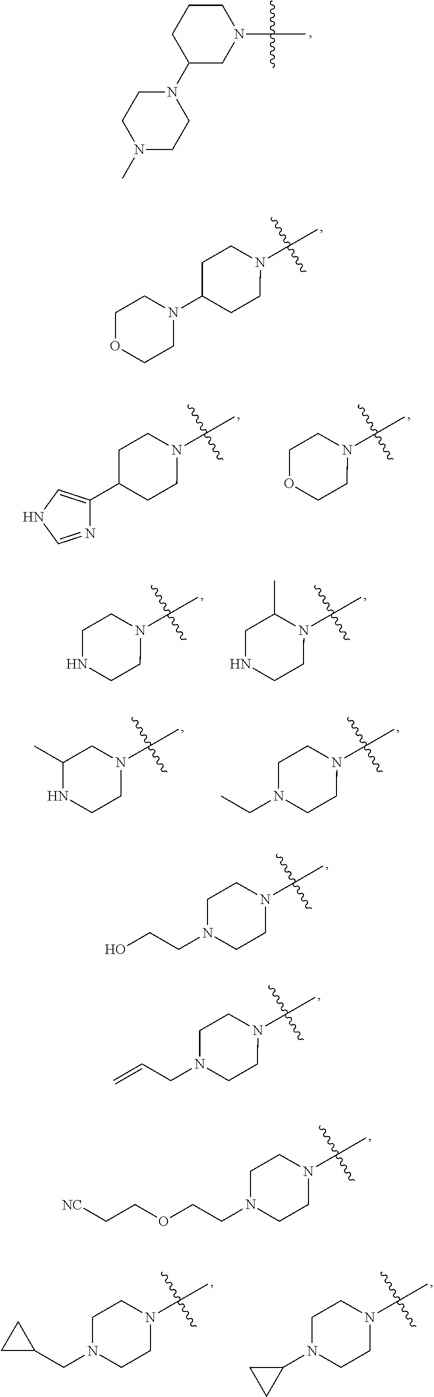 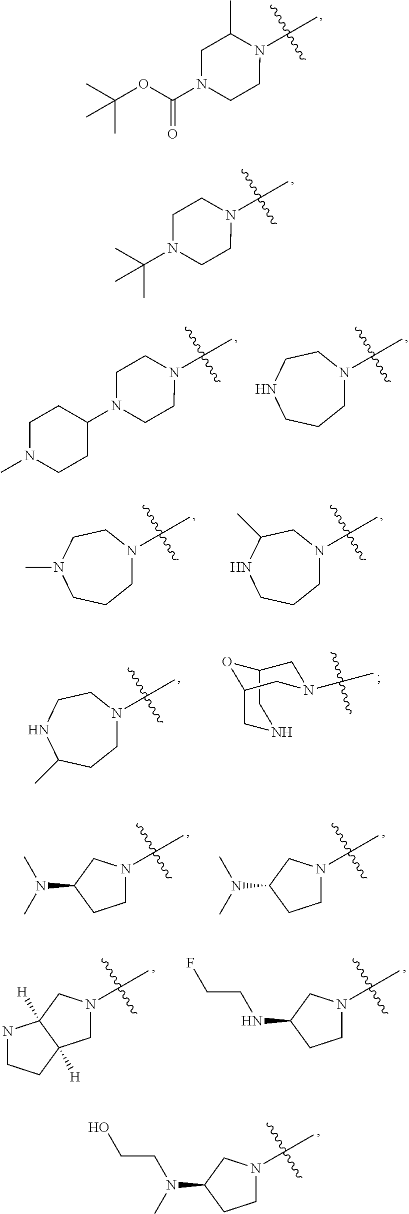 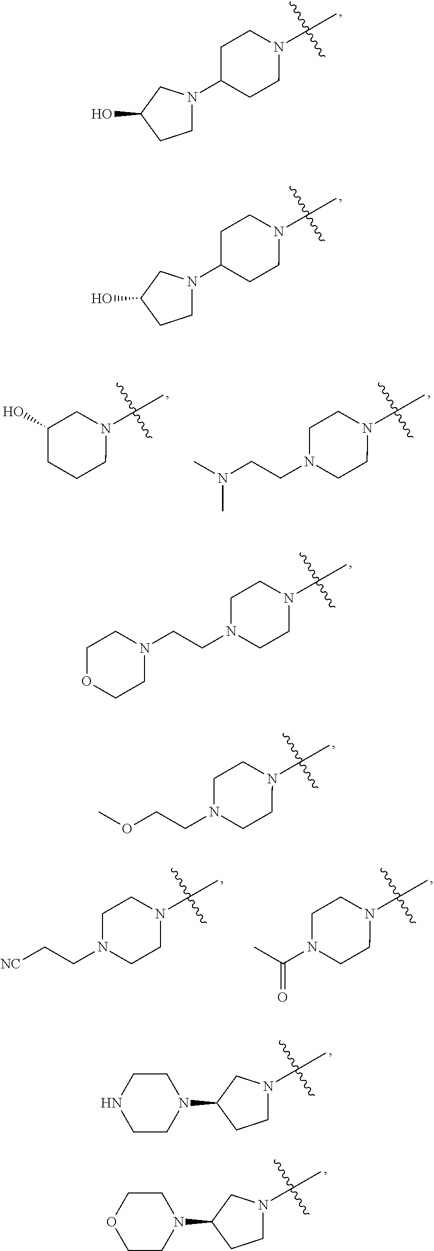 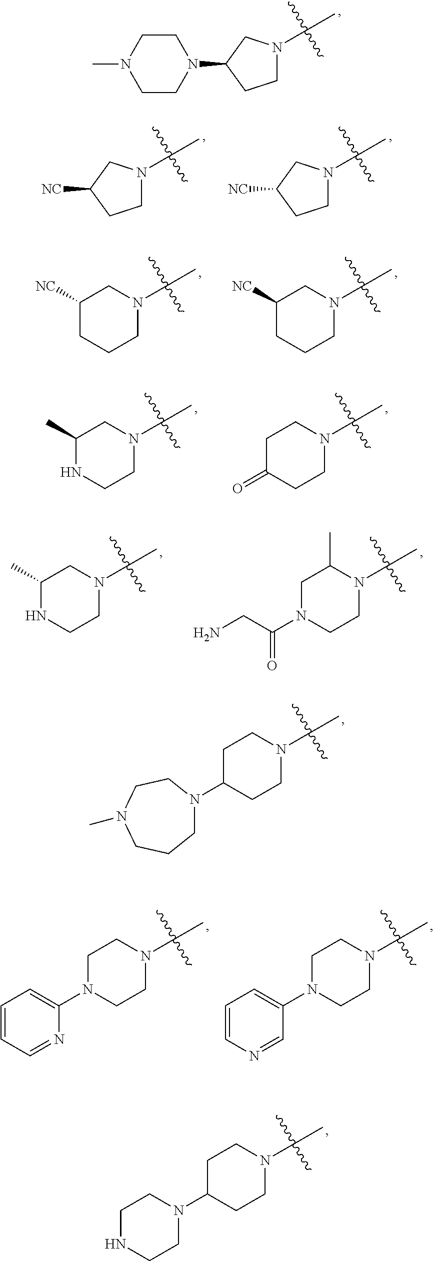 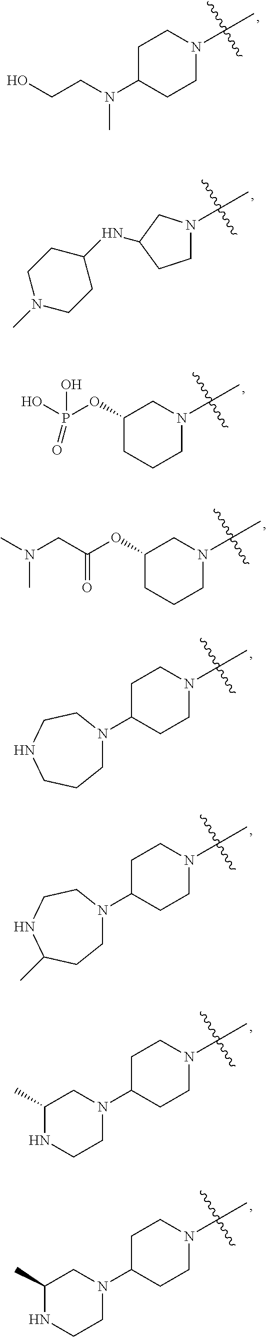 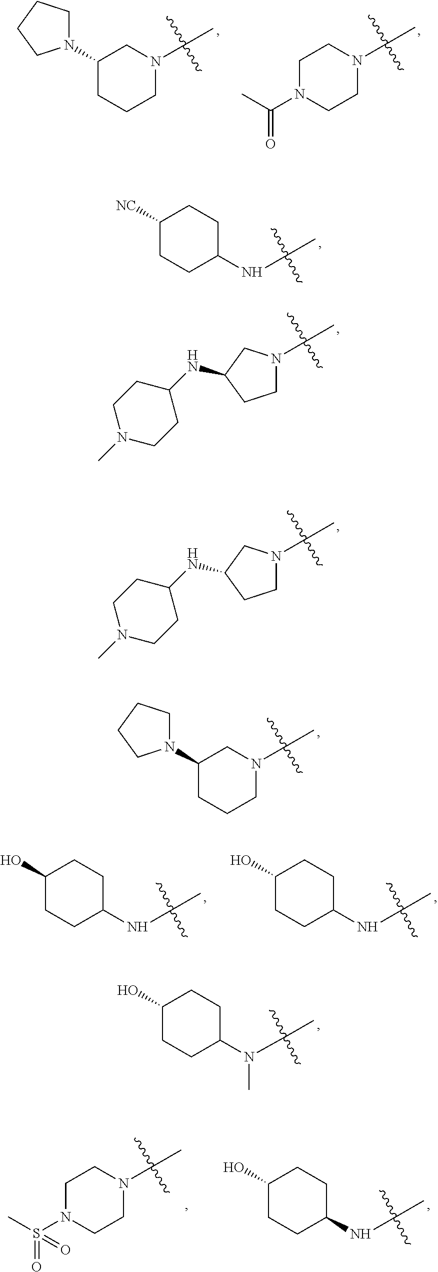 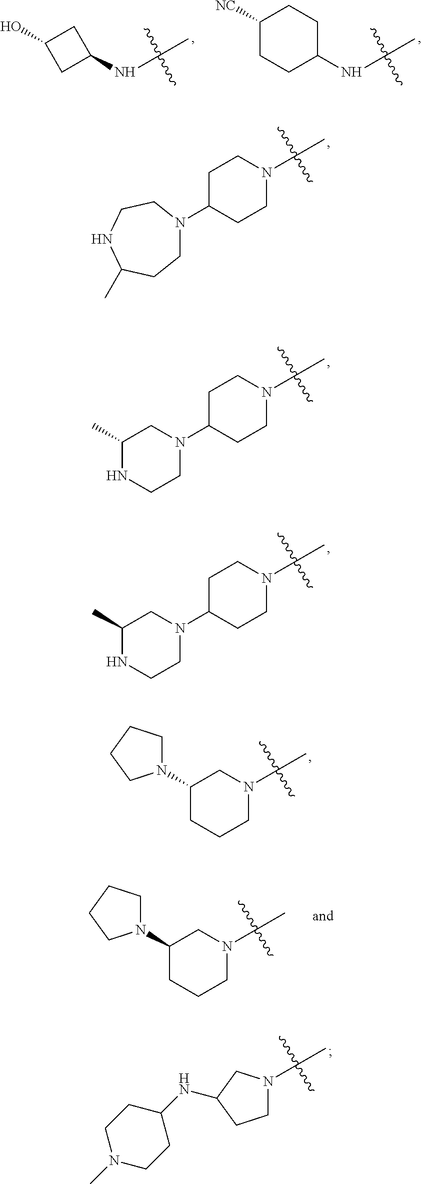
R^{4a} is hydroxyl, 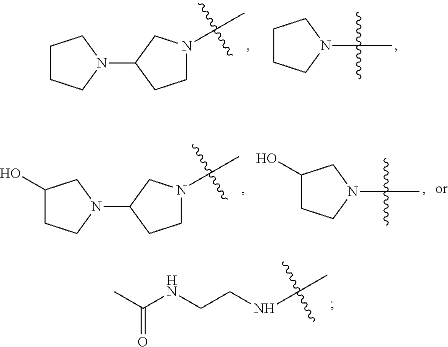
R^{7a} and R^{8a} are independently C₁-C₄ alkyl or C₃-C₆ cycloalkyl;
R^{9a} and R^{10a} are independently selected from hydrogen, hydroxy, C₁-C₄ alkyl, hydroxy(C₁-C₄)alkyl, C₁-C₄ acyl, C₁-C₄ alkyloxy(C₁-C₄)alkyl, C₁-C₄ alkenyl, or, 4 to 7 member heterocyclyl group,
which may be substituted with halogen, hydroxy, cyano, C₁-C₄ alkyl, C₁-C₄ alkoxy, C₁-C₄ acyl, C₃-C₆ cycloalkyl, C₄-C₇ cycloalkylmethyl, hydroxy(C₁-C₄)alkyl, C₁-C₄ alkenyl, amino(C₁-C₄)alkyl, amino(C₃-C₆)cycloalkyl, or a 4 to 6 member heterocyclyl group;
R^{11a} is 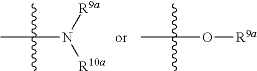
R¹⁴ and R¹⁵ are each independently hydrogen, C₁-C₄ alkyl, halo C₁-C₄ alkyl, hydroxy C₁-C₄ alkyl, C₁-C₄ alkoxy C₁-C₄ alkyl, C₂-C₄ alkenyl, C₂-C₄ alkynyl, C₃-C₆ cycloalkyl, heterocyclyl, aryl or heteroaryl; where R^{y} and R^{z} are each optionally substituted with one, two or three Q⁵ groups;
R¹⁹ is alkyl, alkenyl, alkynyl, haloalkyl, cycloalkyl, aryl or heteroaryl; and
n is a natural number from 1 to 3.

20. The compound of claim 18, wherein E is CO.

21. The compound of claim 18, wherein M is

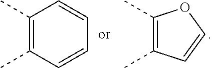

22. The compound of claim 1, wherein R¹ is phenyl.

23. The compound of claim 18, wherein Q^{3a} is alkyloxy; and Q^{4a} is selected from halo, cyano, hydroxy, alkyl, aminoalkyl, alkyloxy, haloalkyloxyl, hydroxyalkyl, alkylthio, 4,5-dihydrooxazol-2-yl amino, pyrimidin-2-amino, piperidin-1-yl, 1-methylpiperidin-4-yl, pyrrolidin-1-yl, —NH—SO₂R^{2a}, —NHCOCH₃, —COR^{3a}, and —CH₂R^{4a}.

24. The compound of claim 18, wherein R^{2a} is CH₃.

25. The compound of claim 1, wherein the compound is selected from

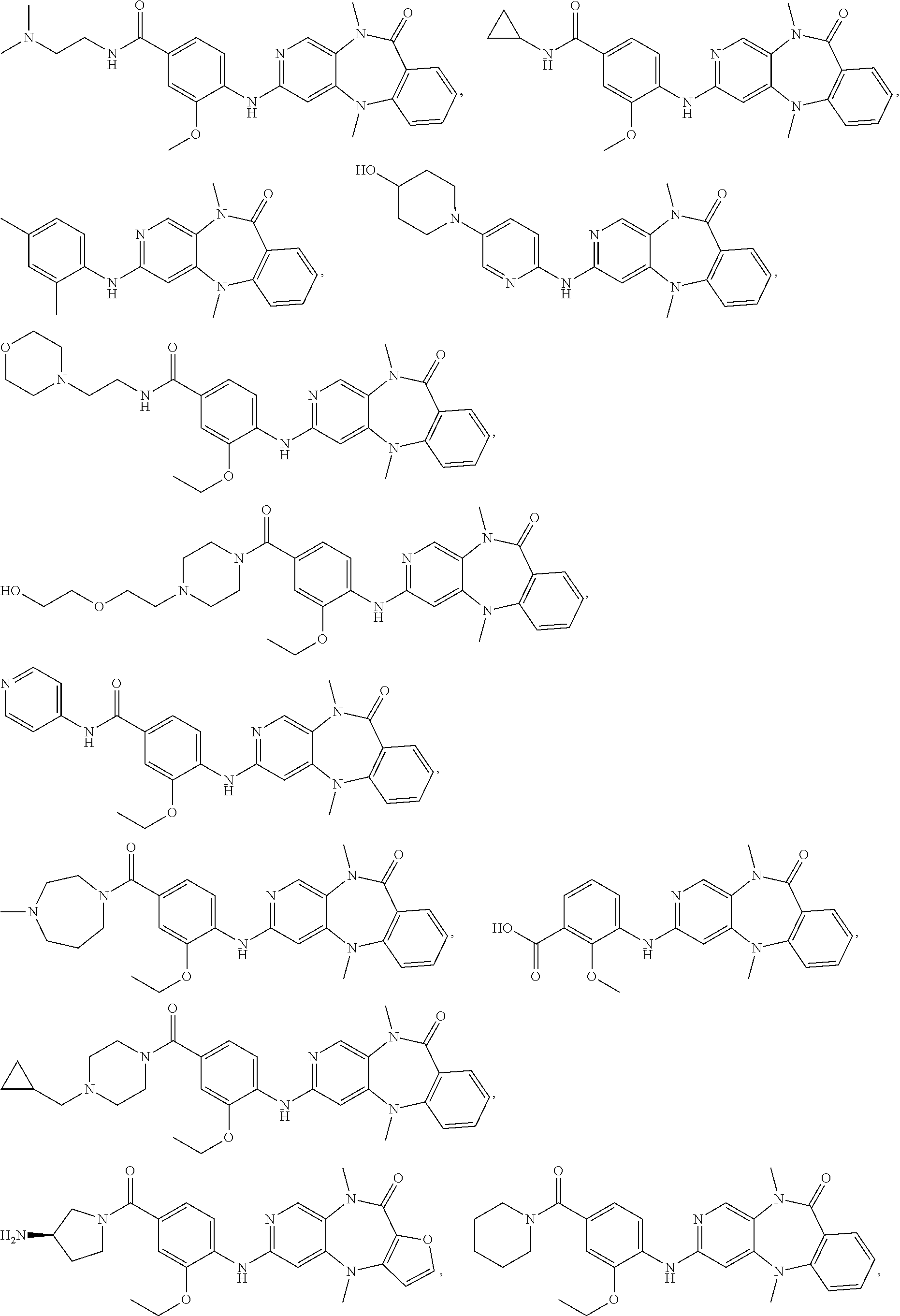

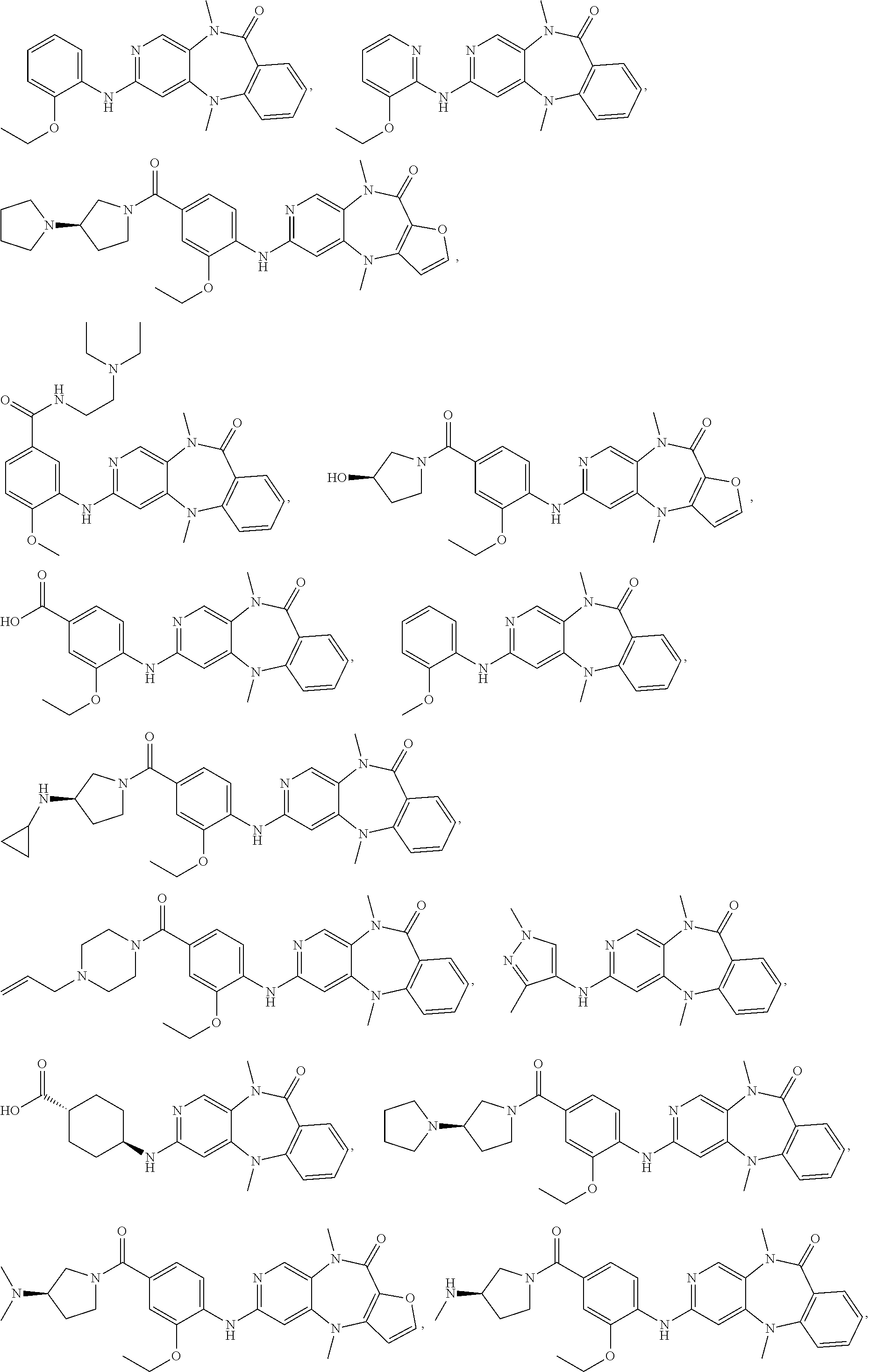

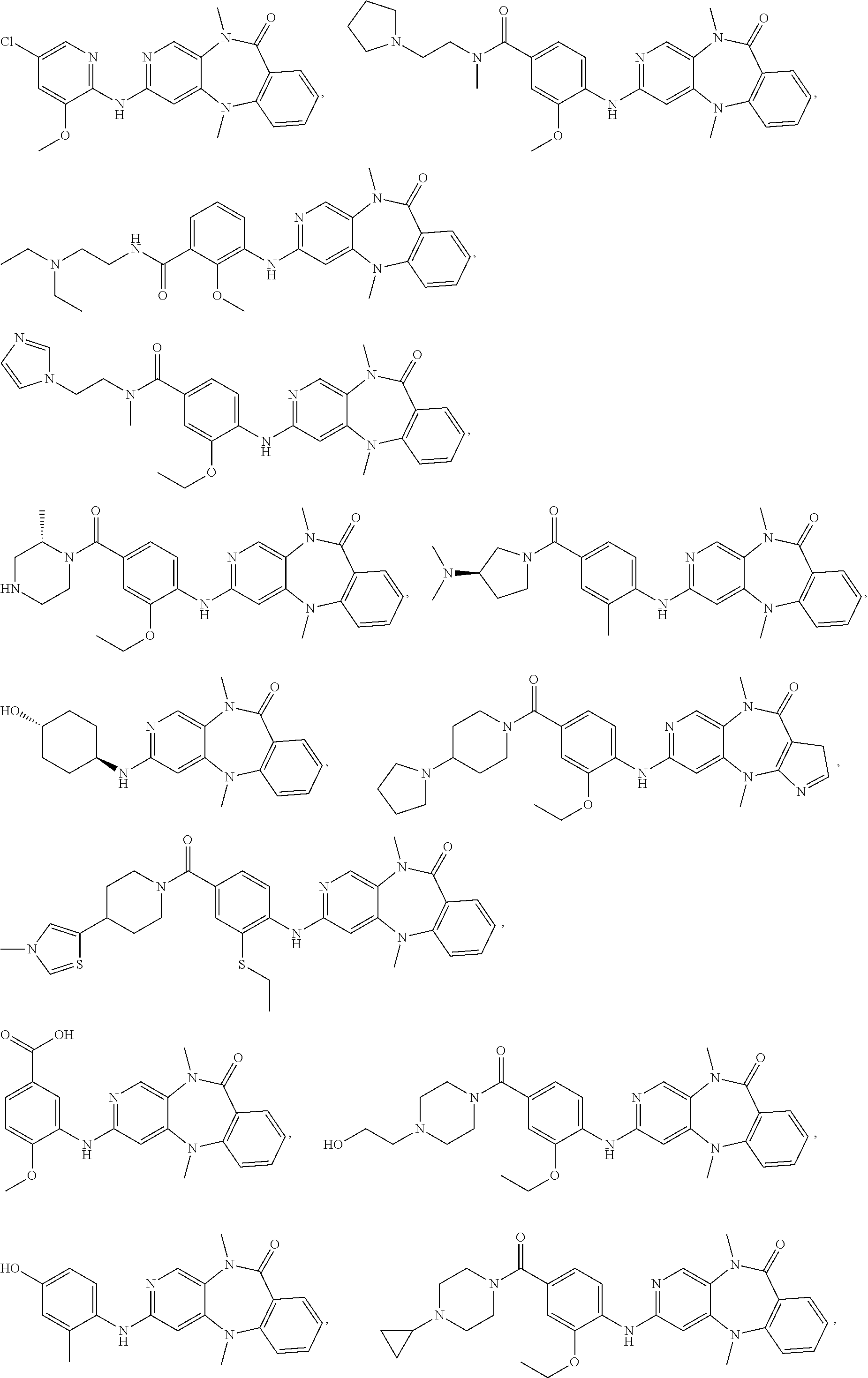

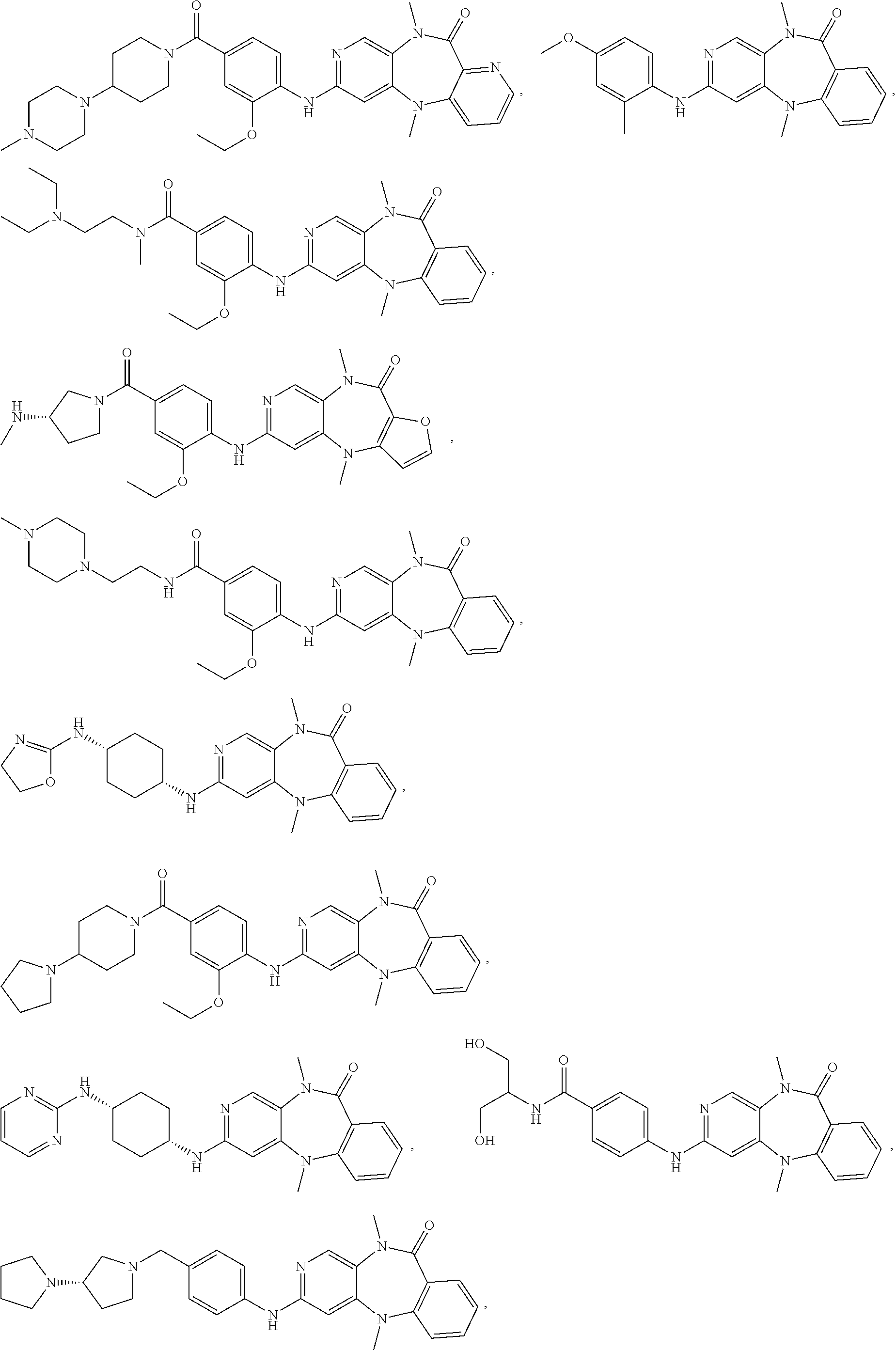

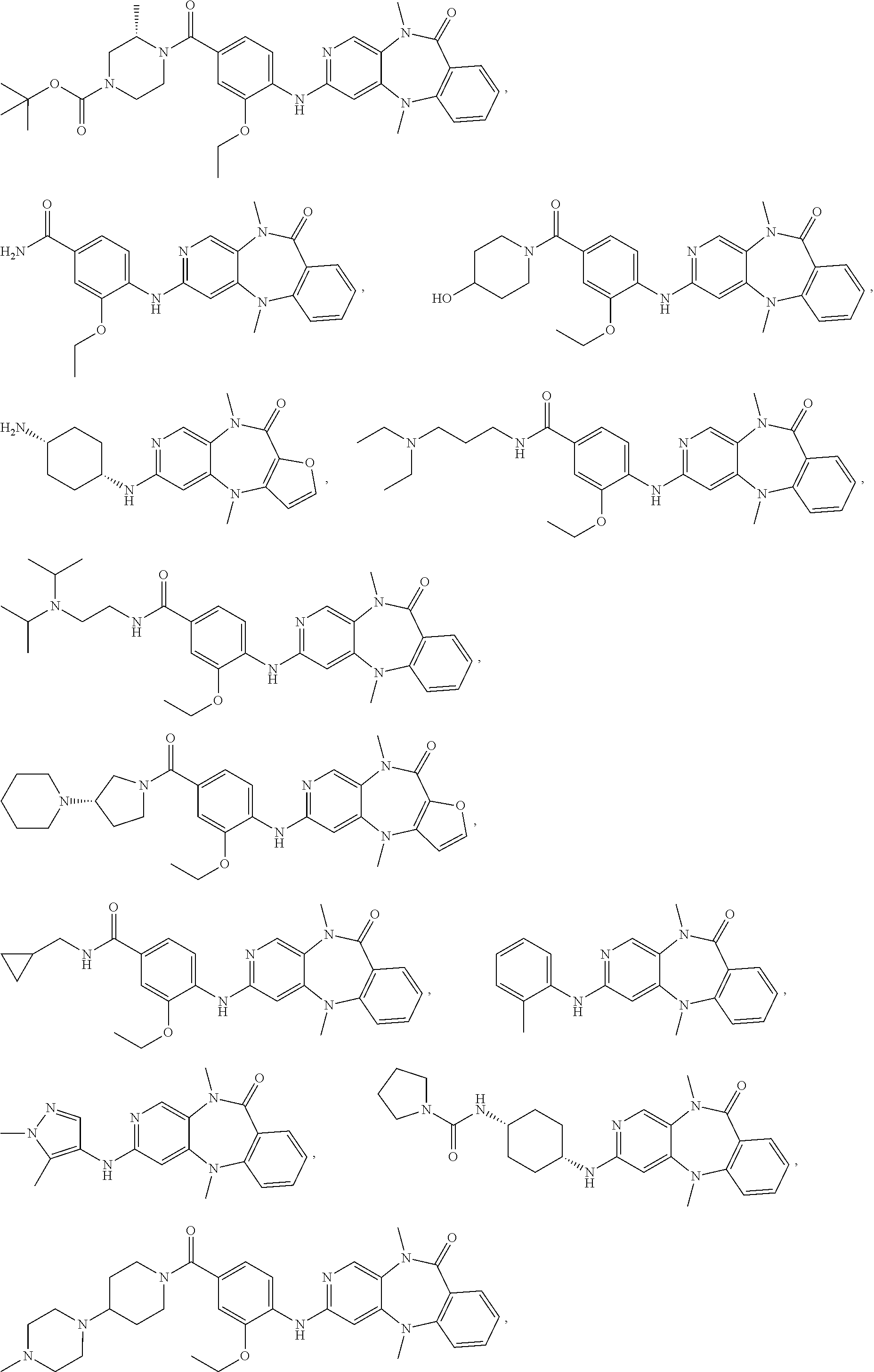

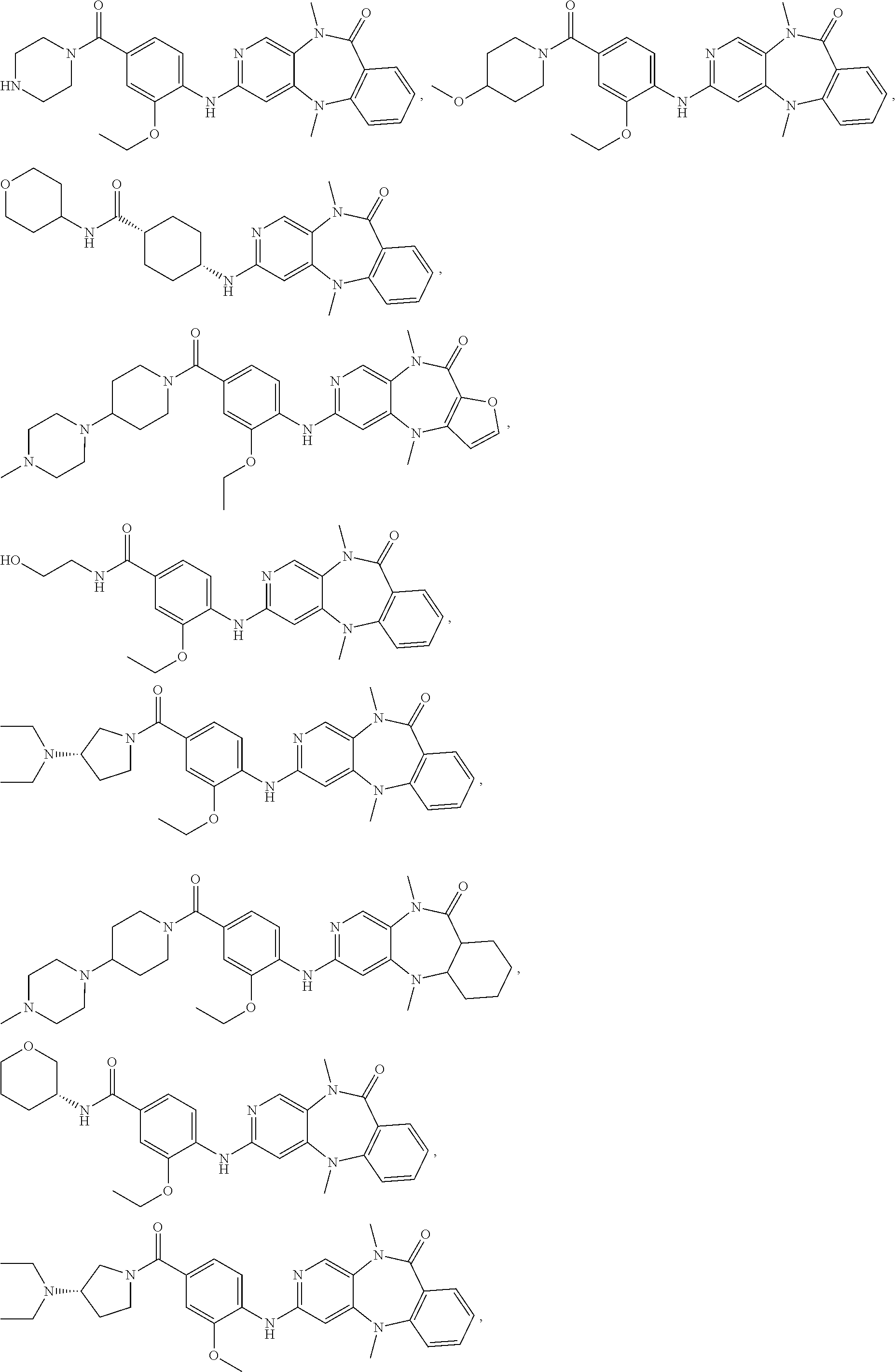

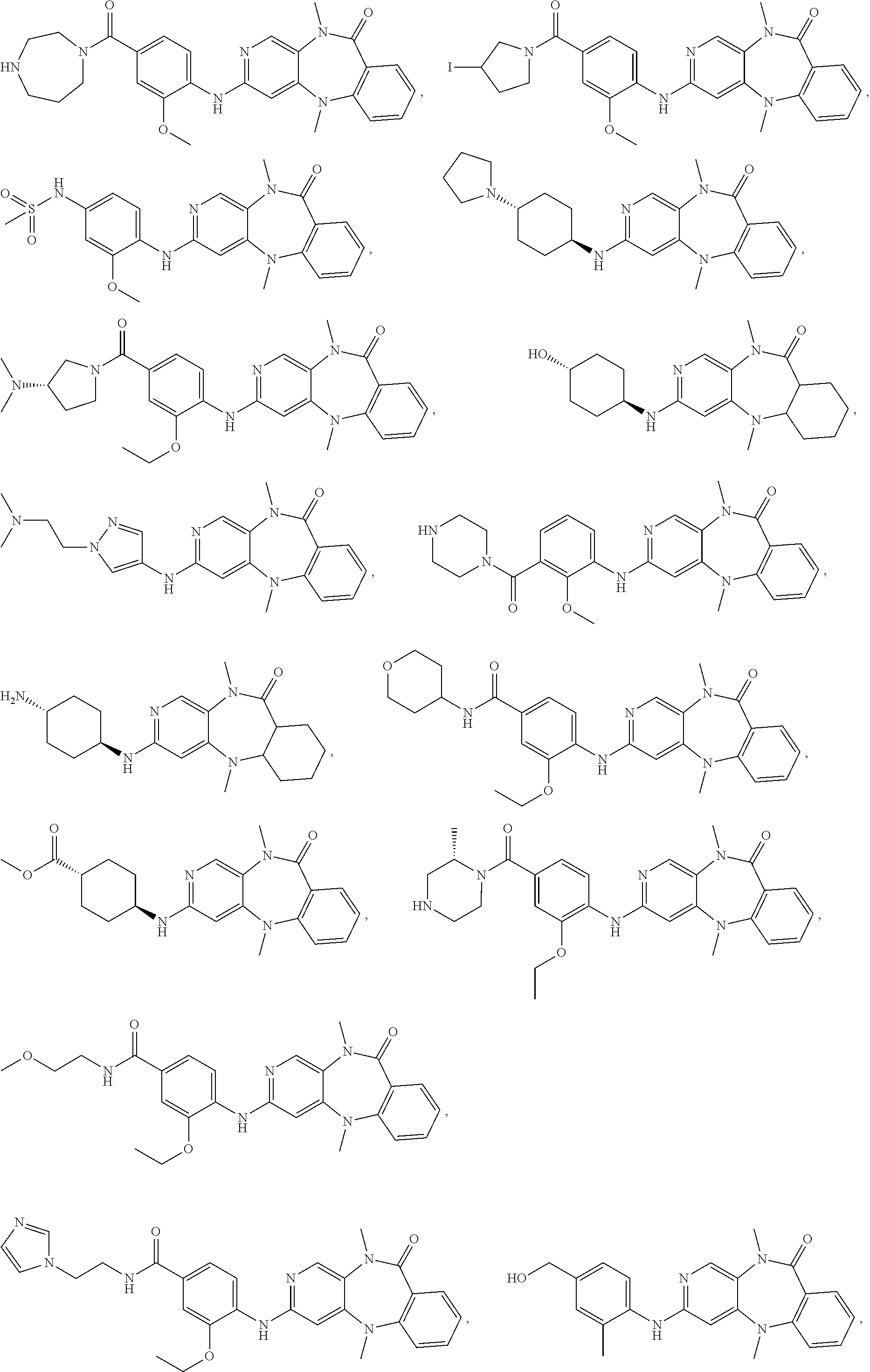

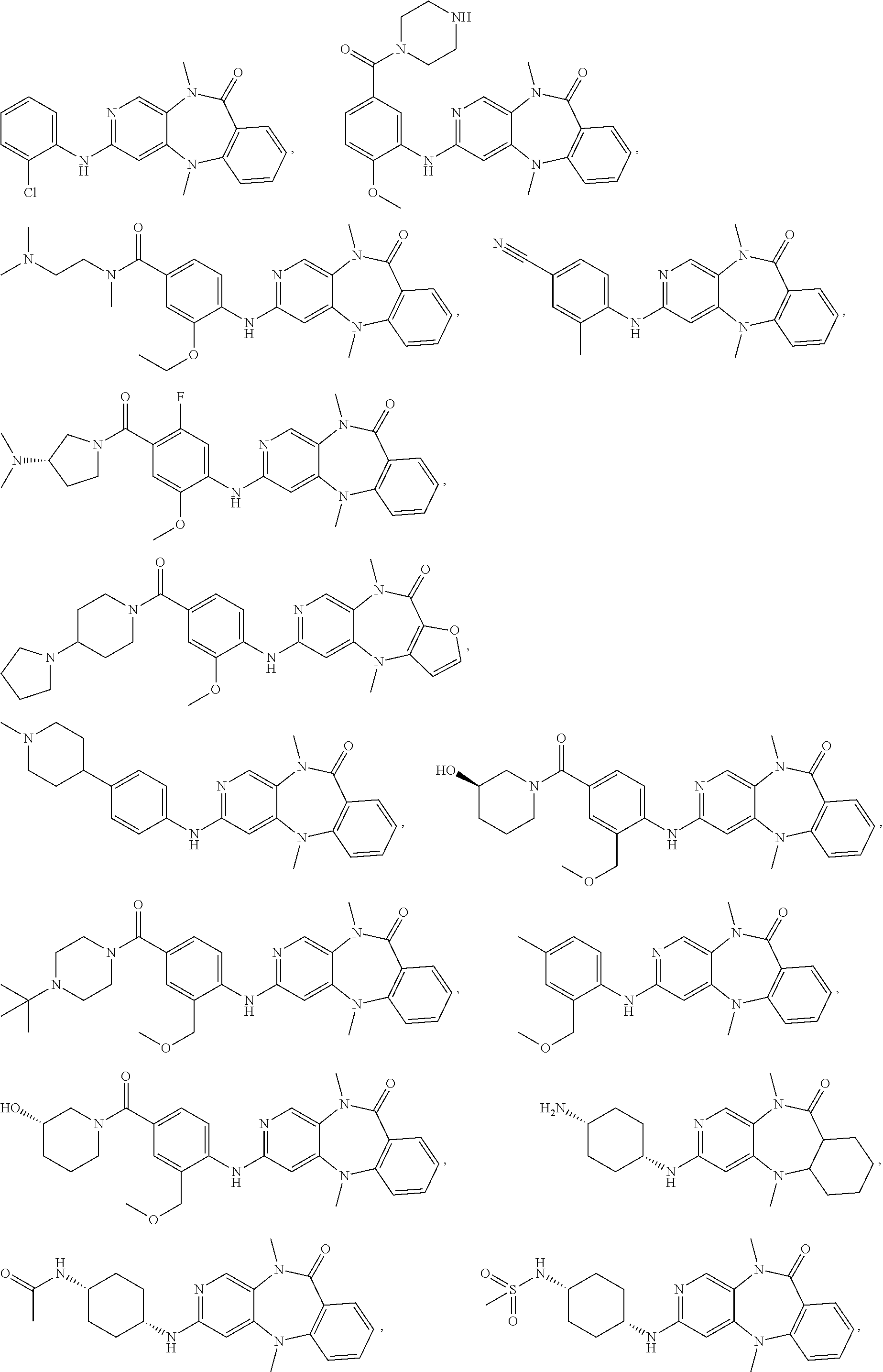

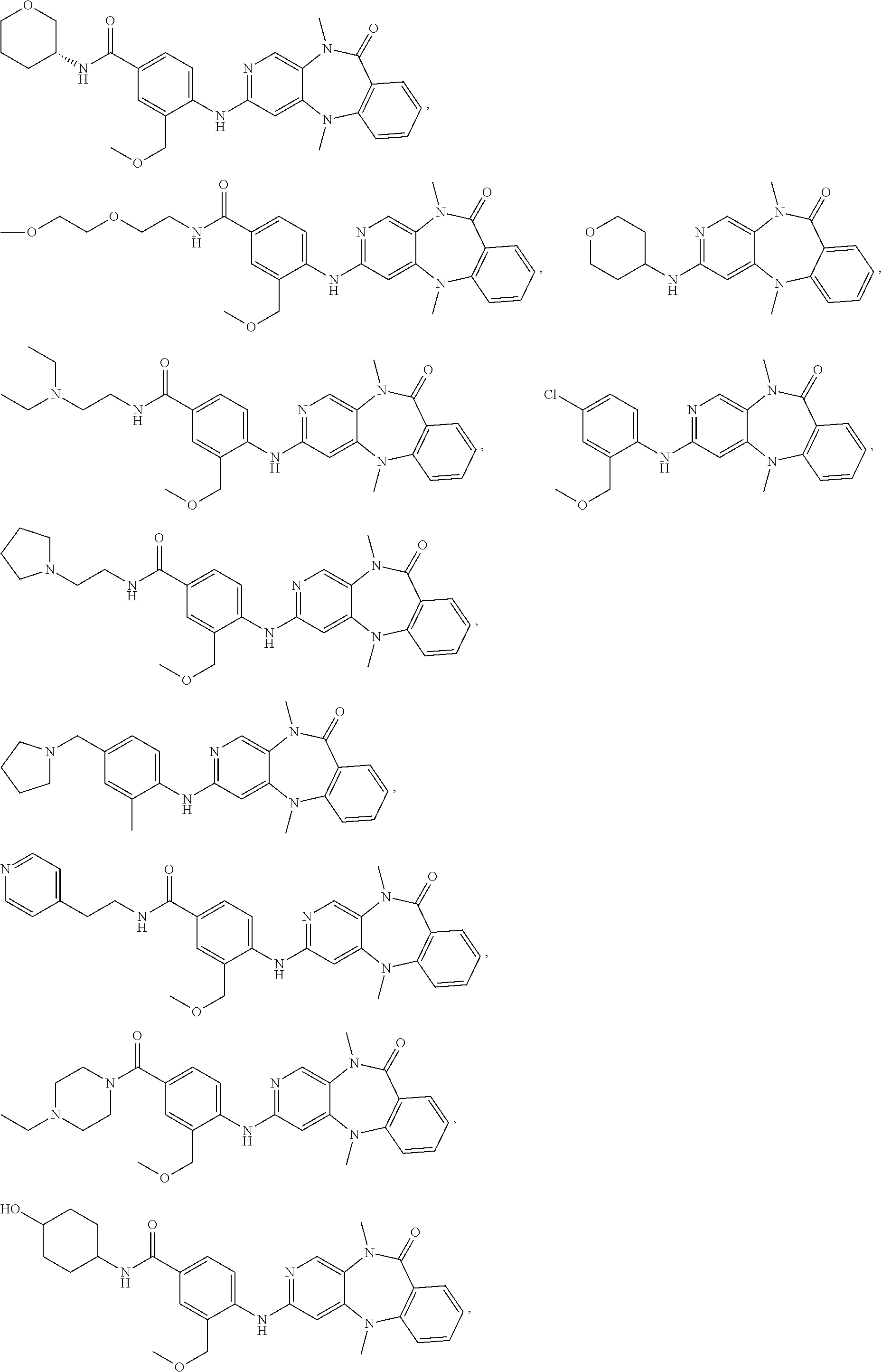

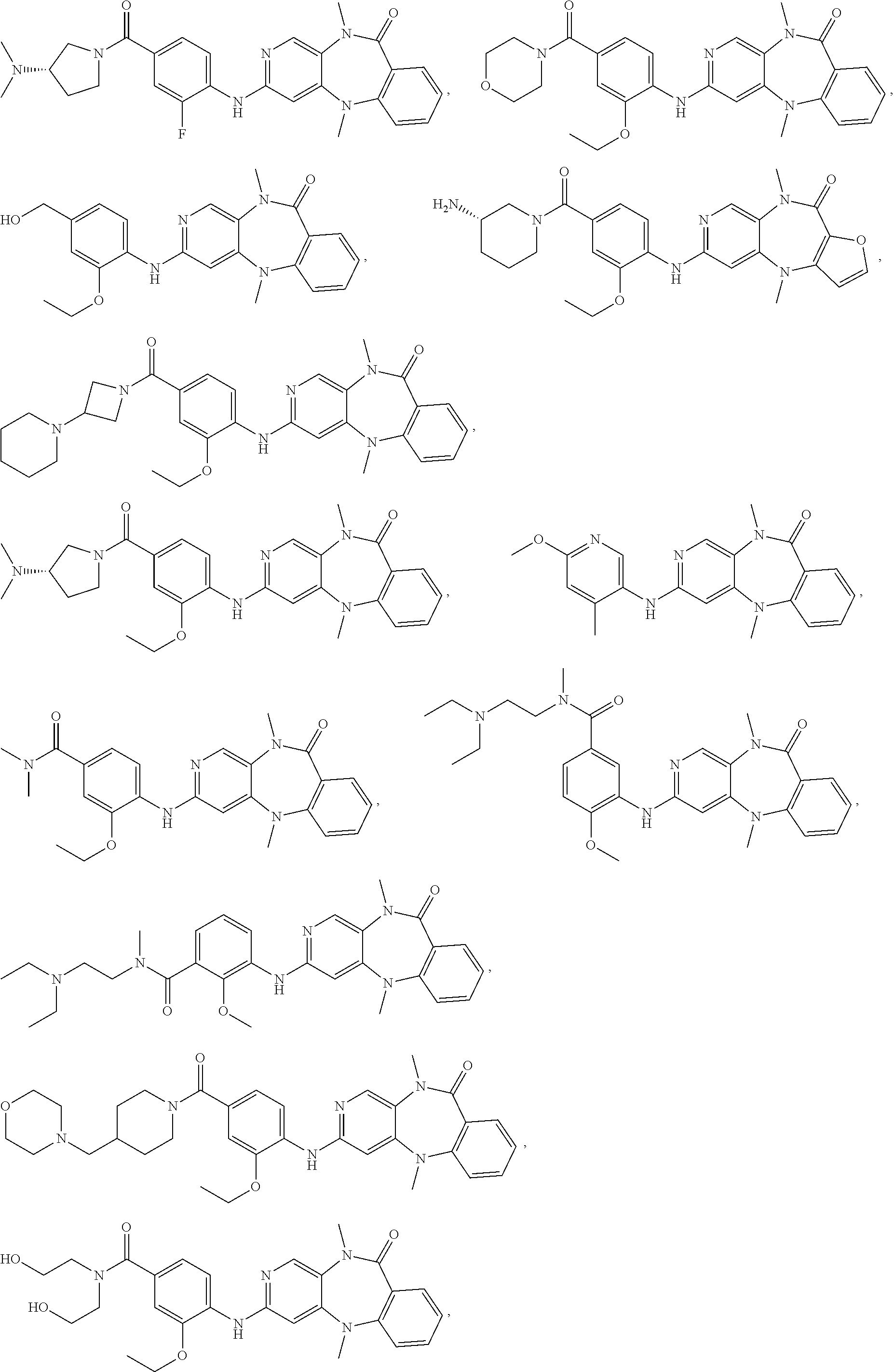

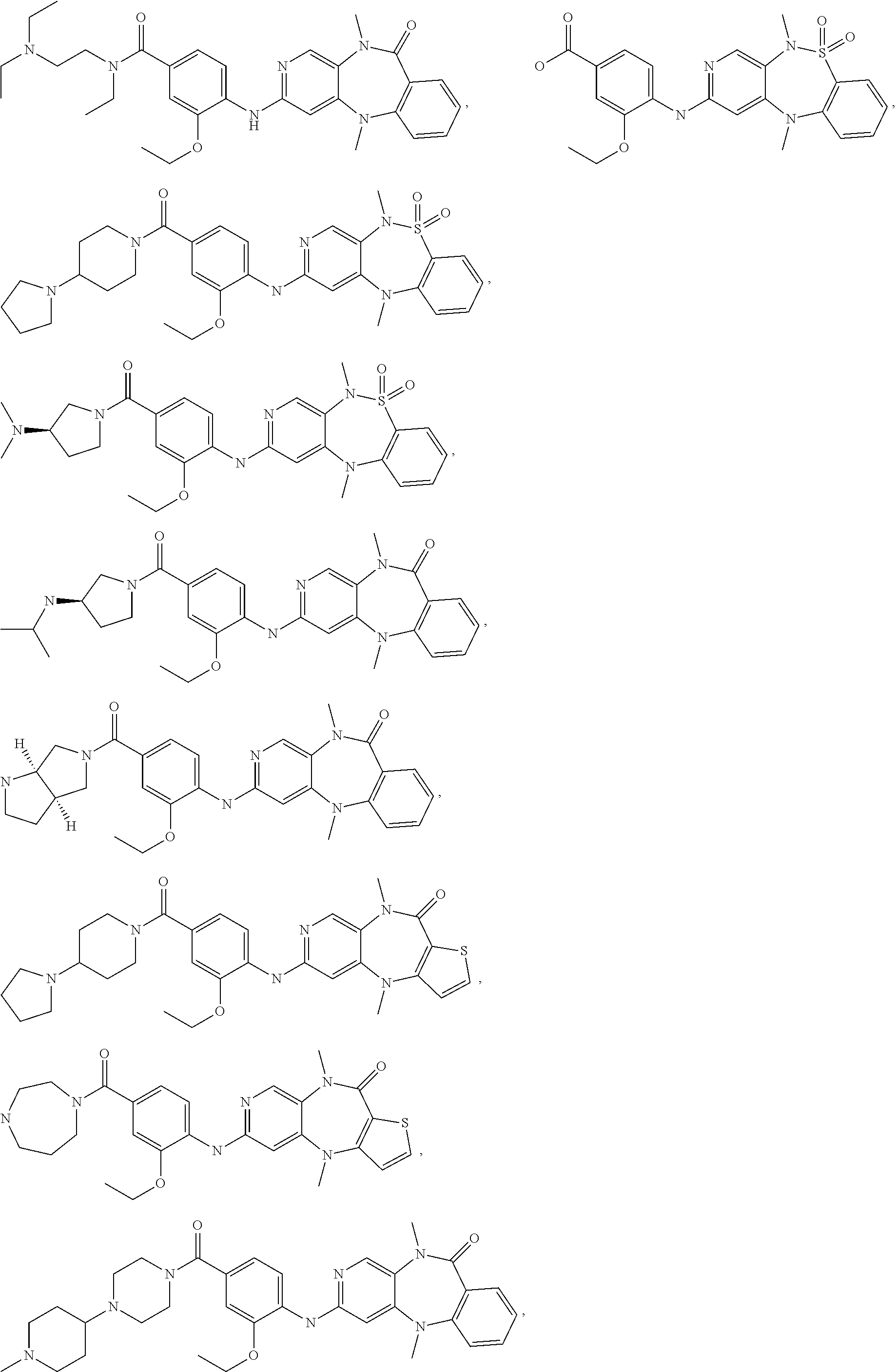

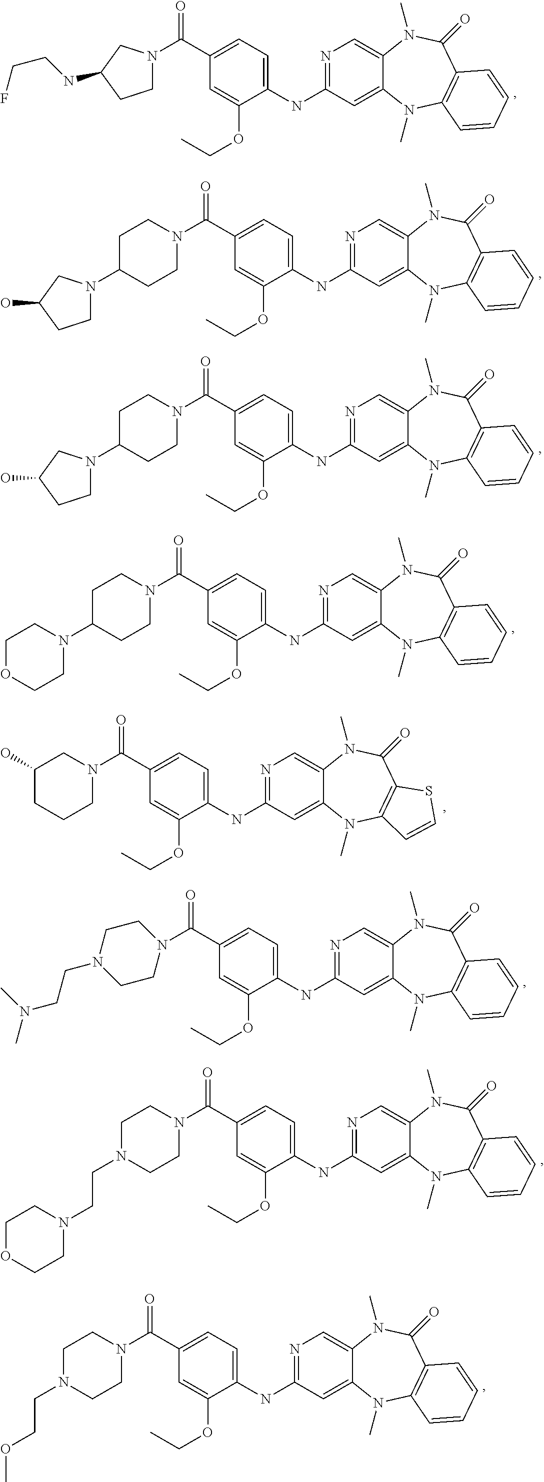

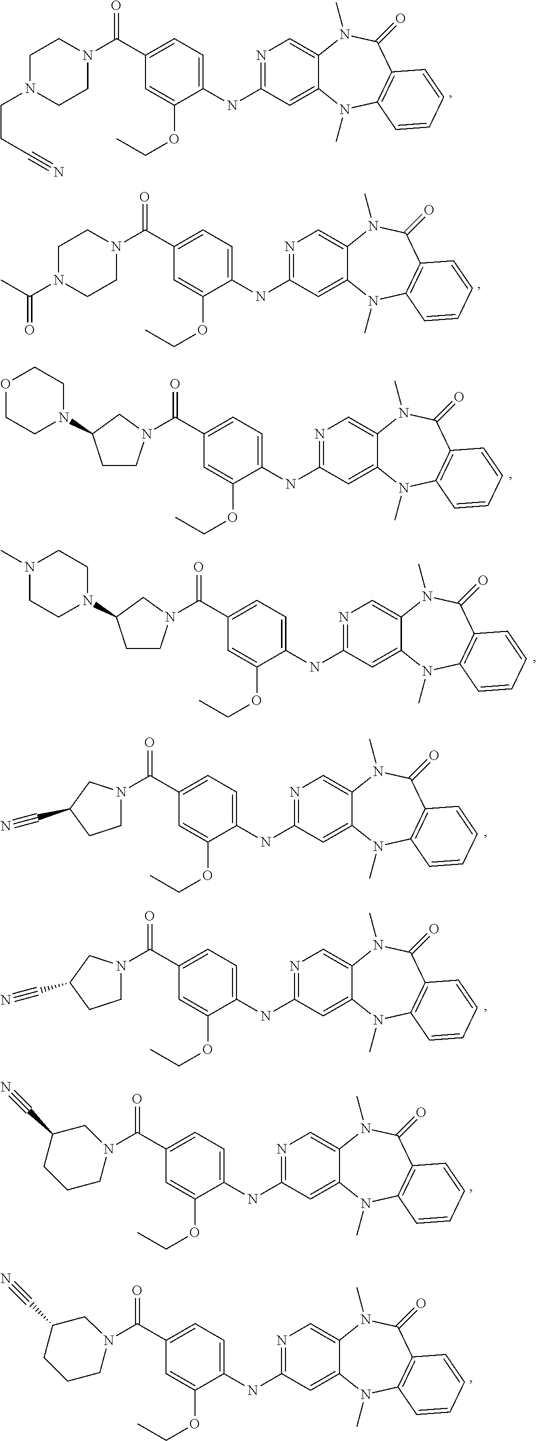

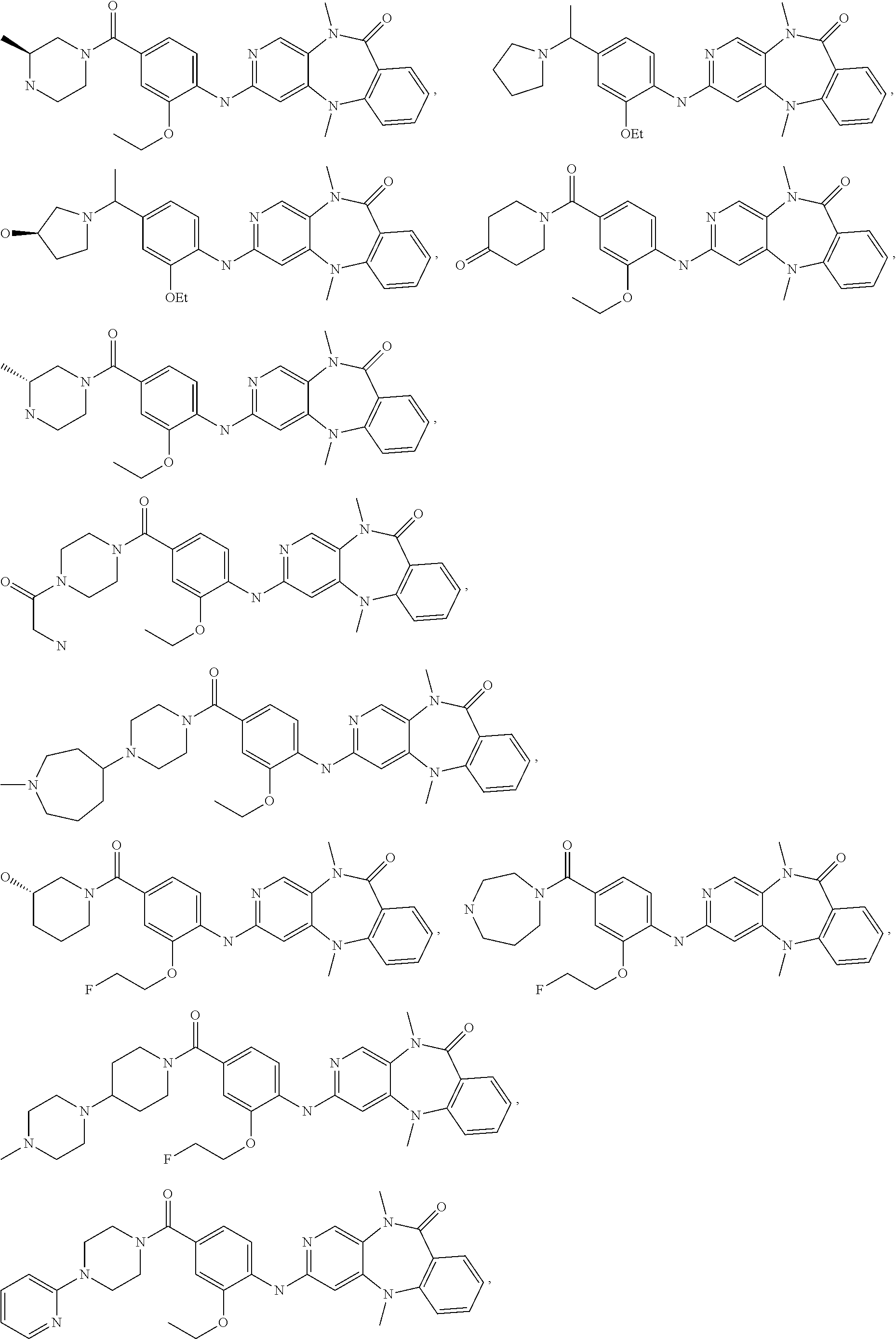

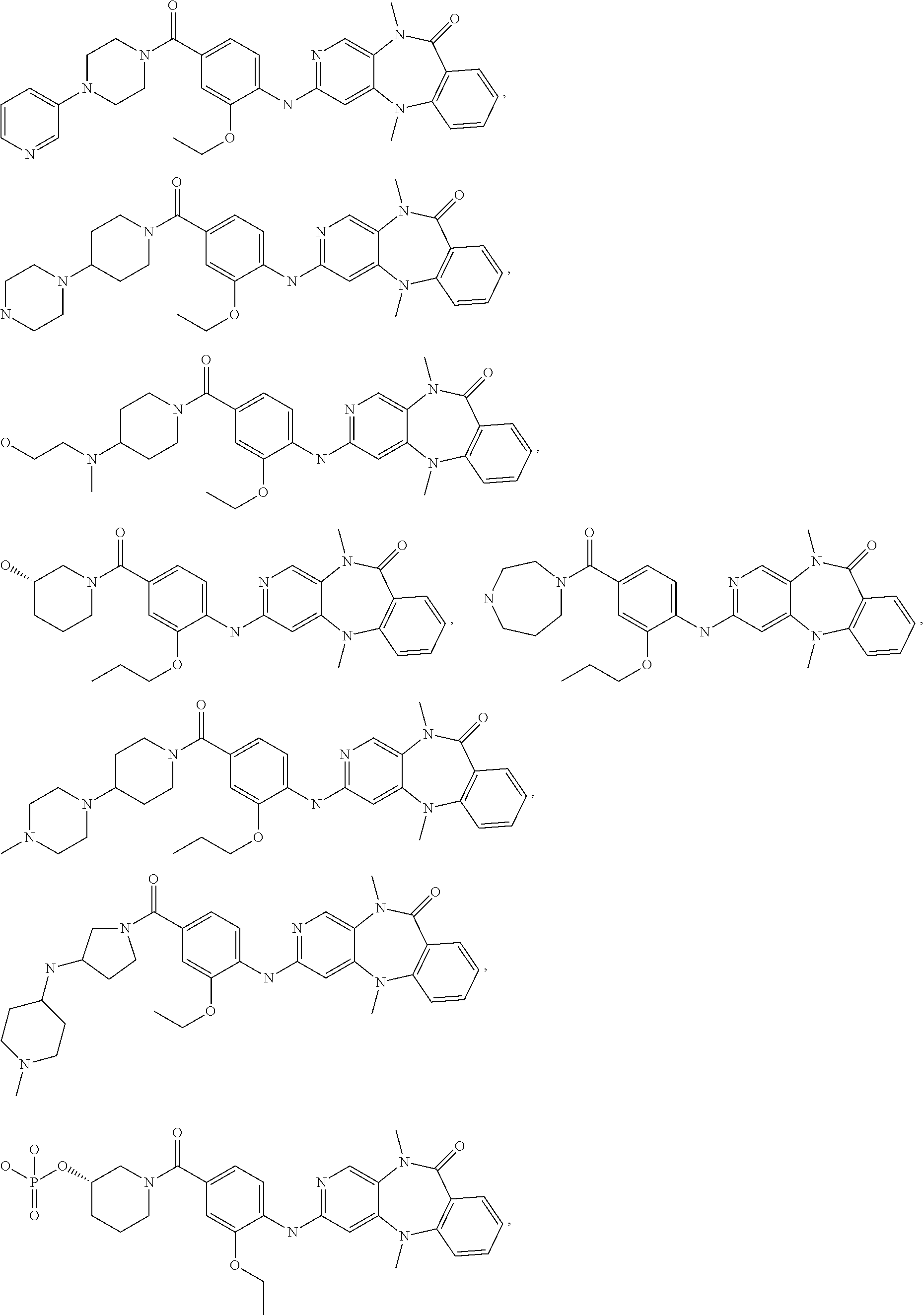

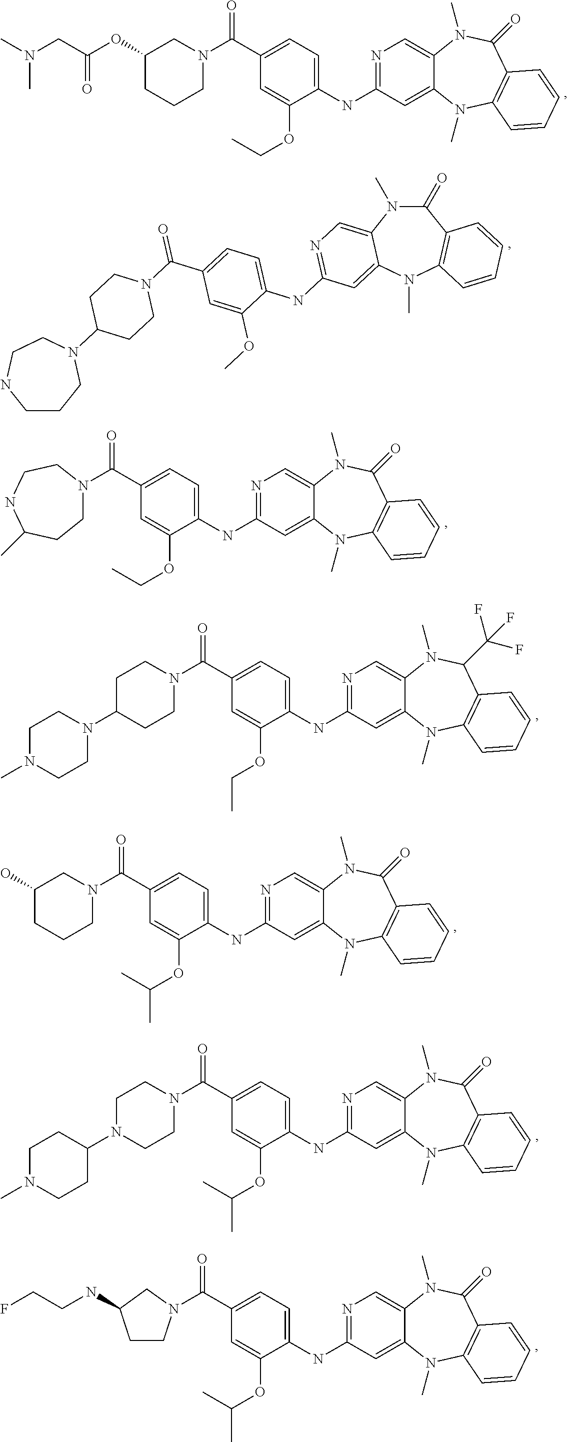

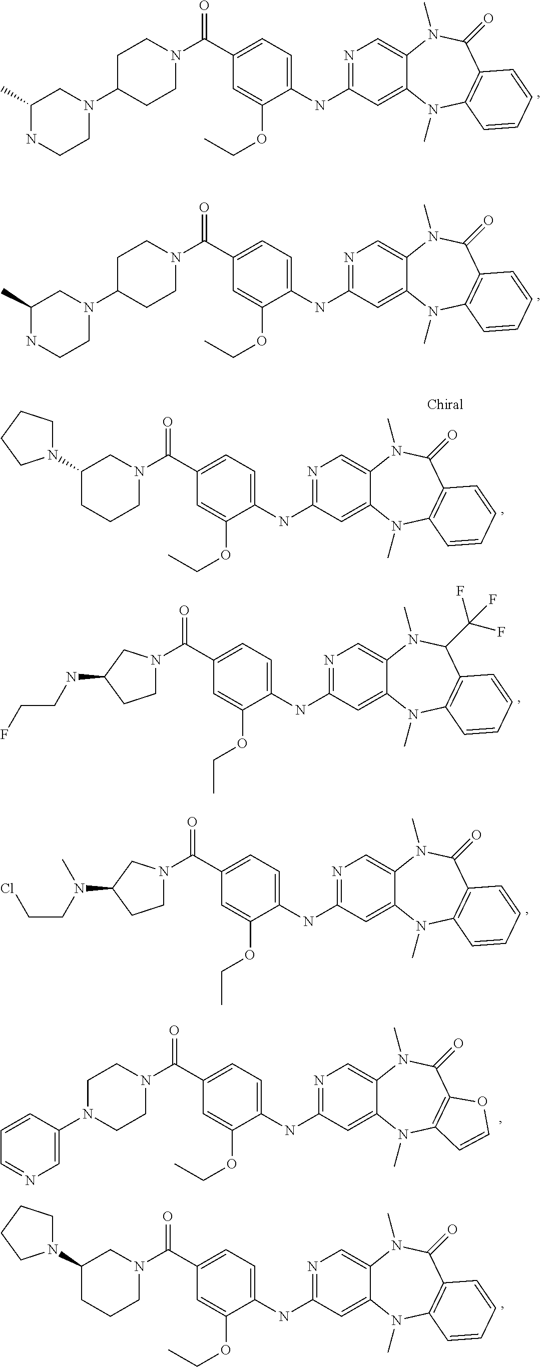

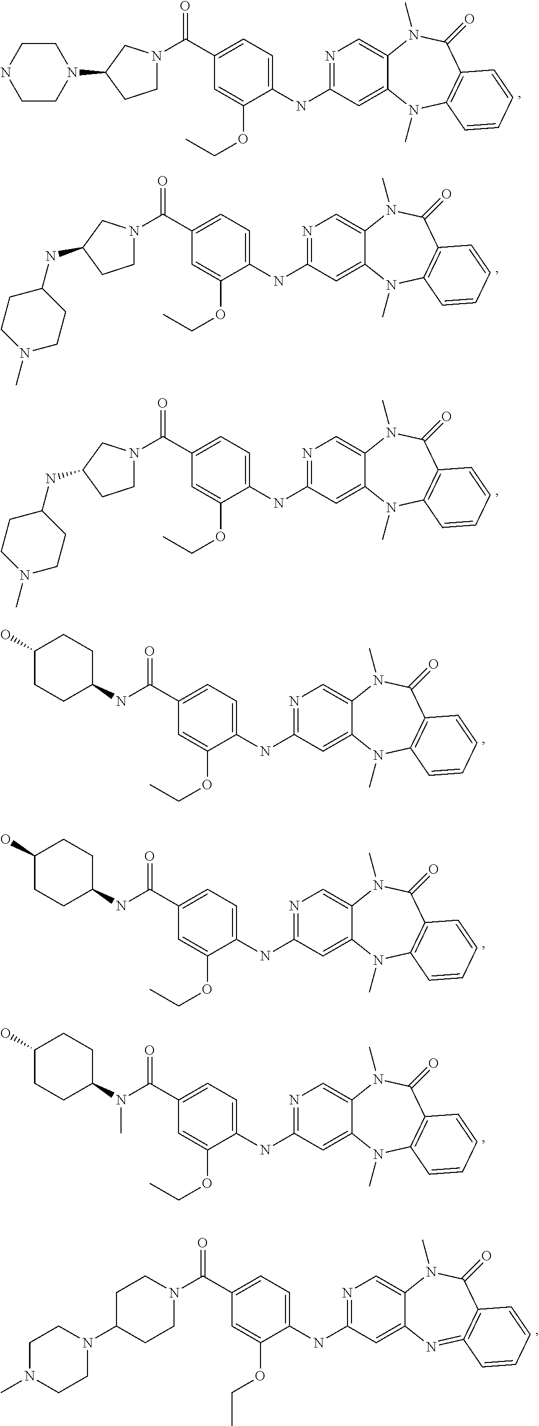

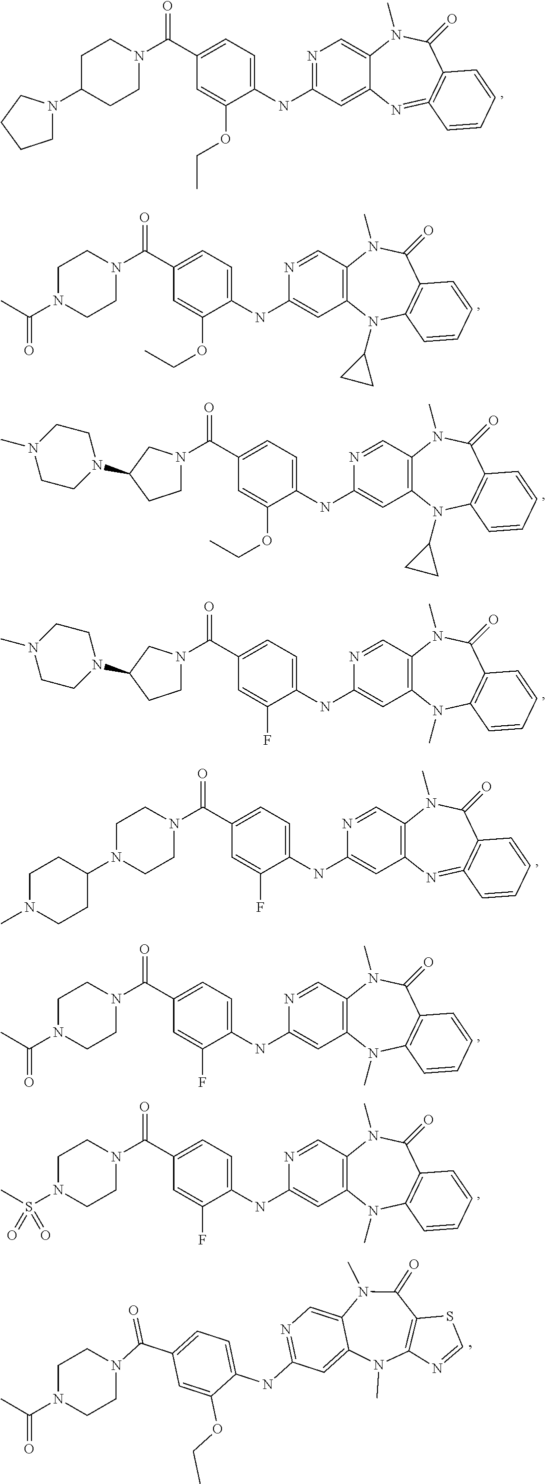

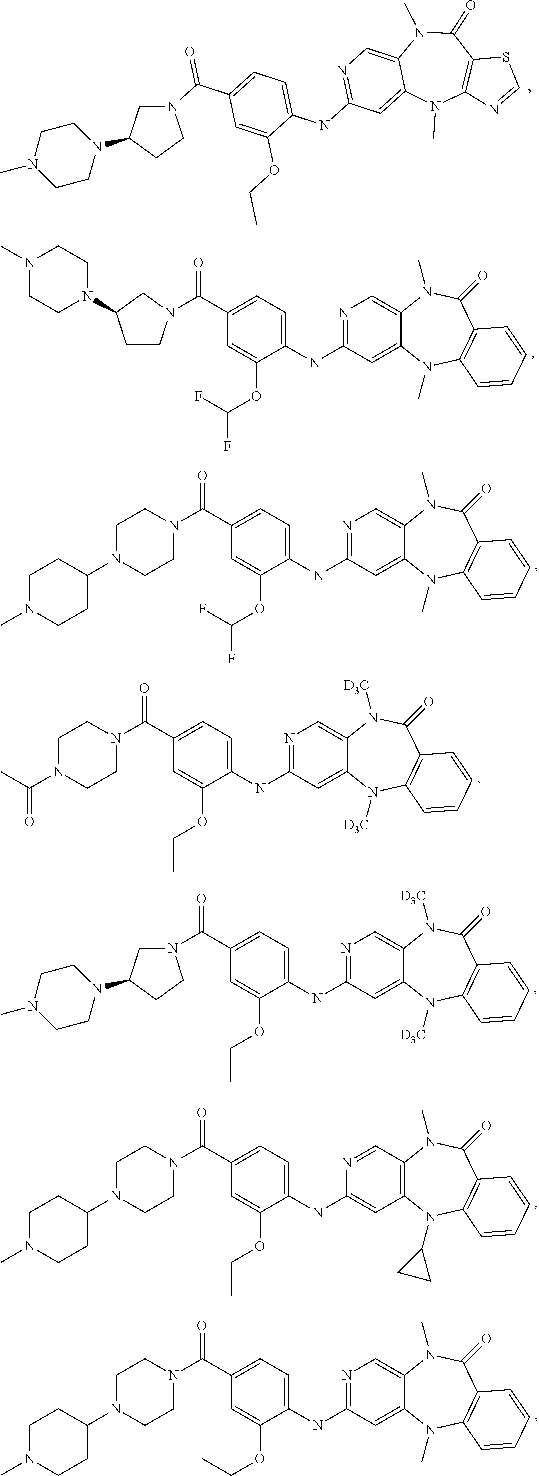

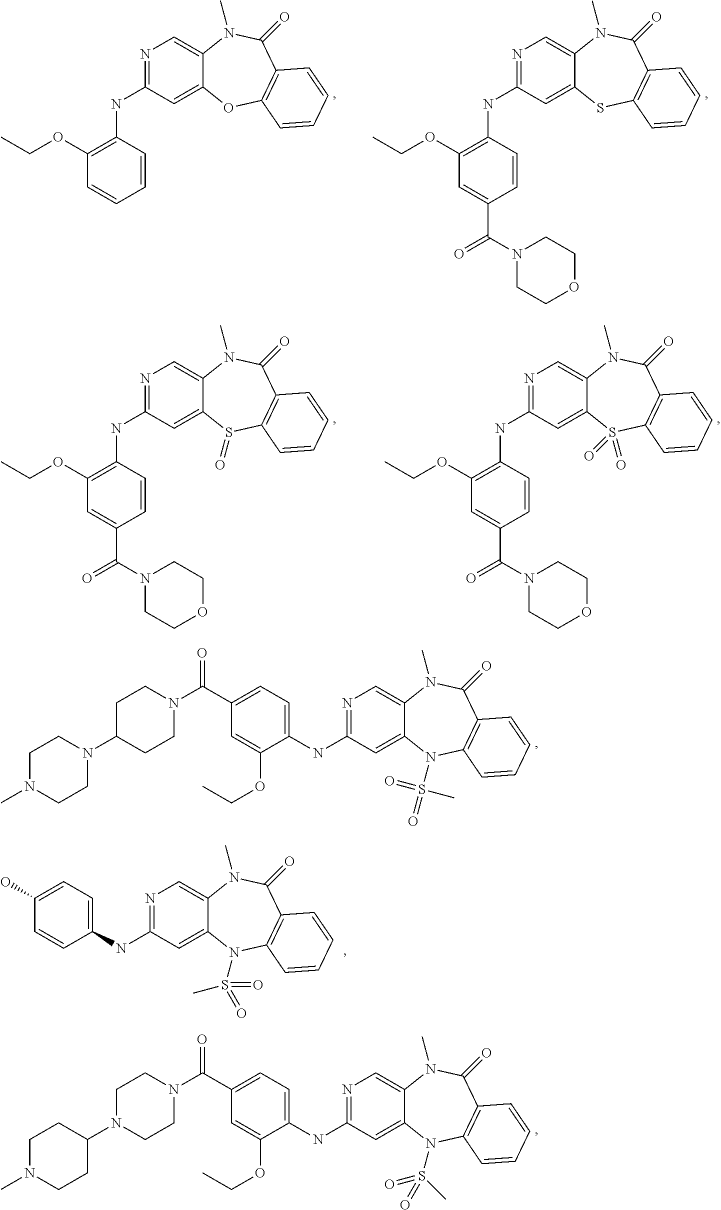

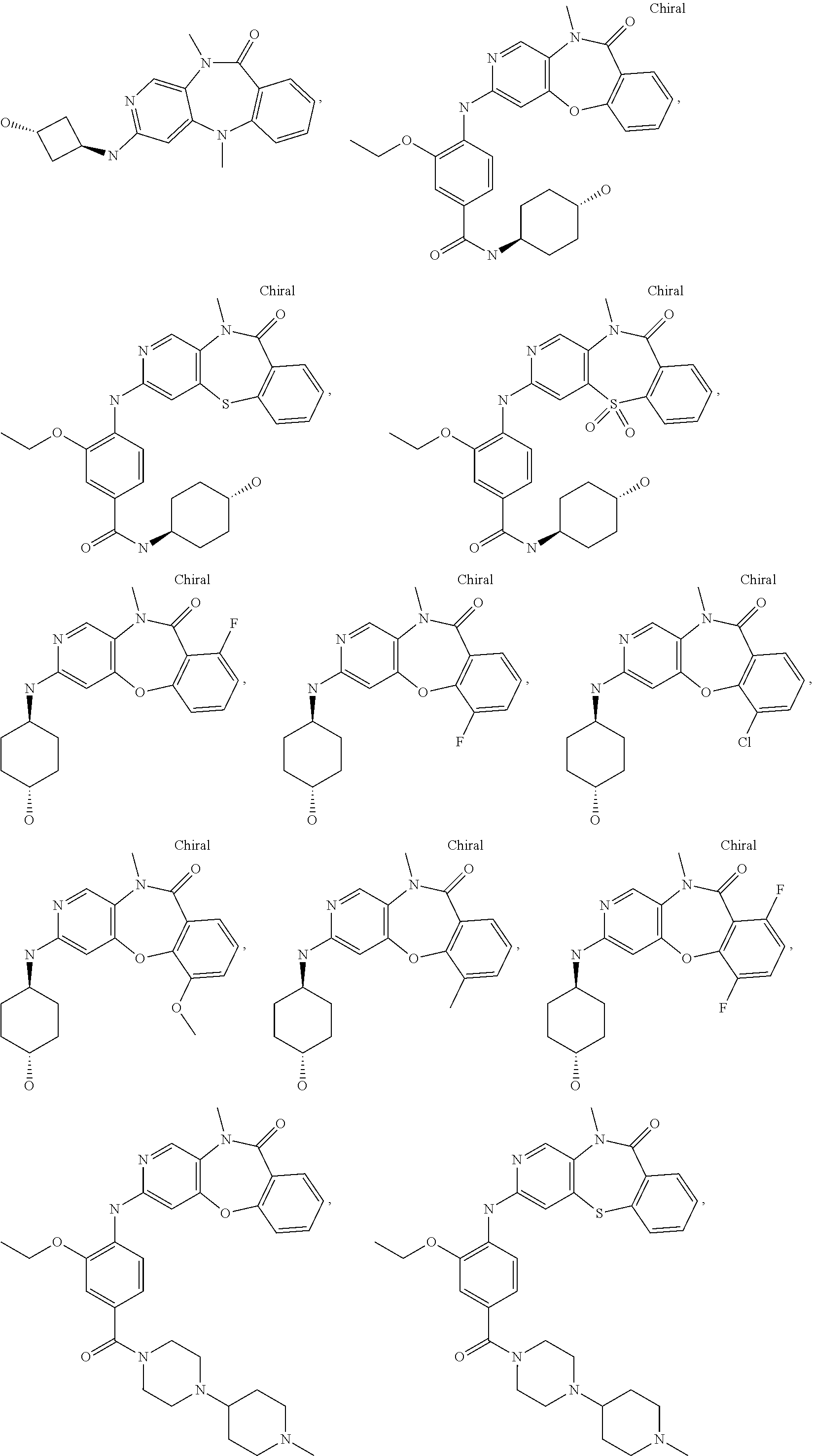

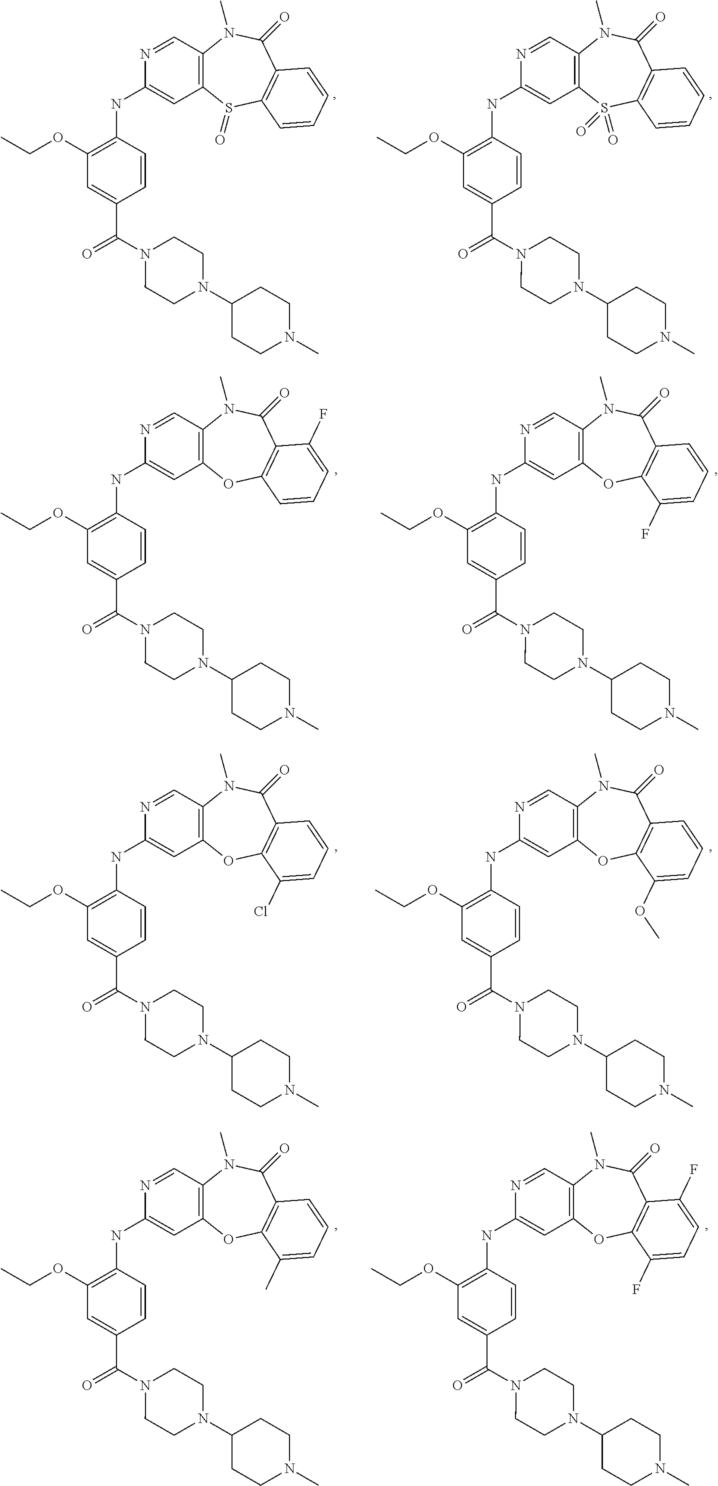

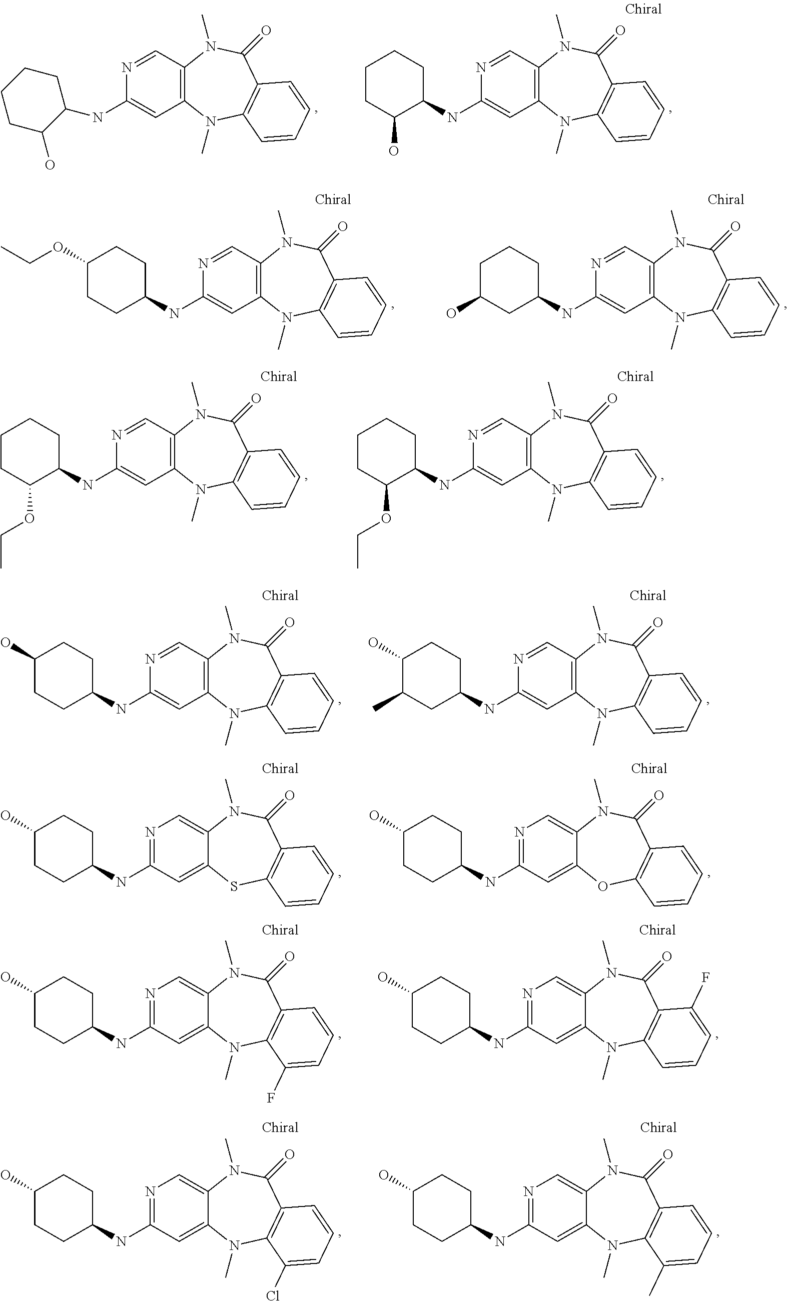

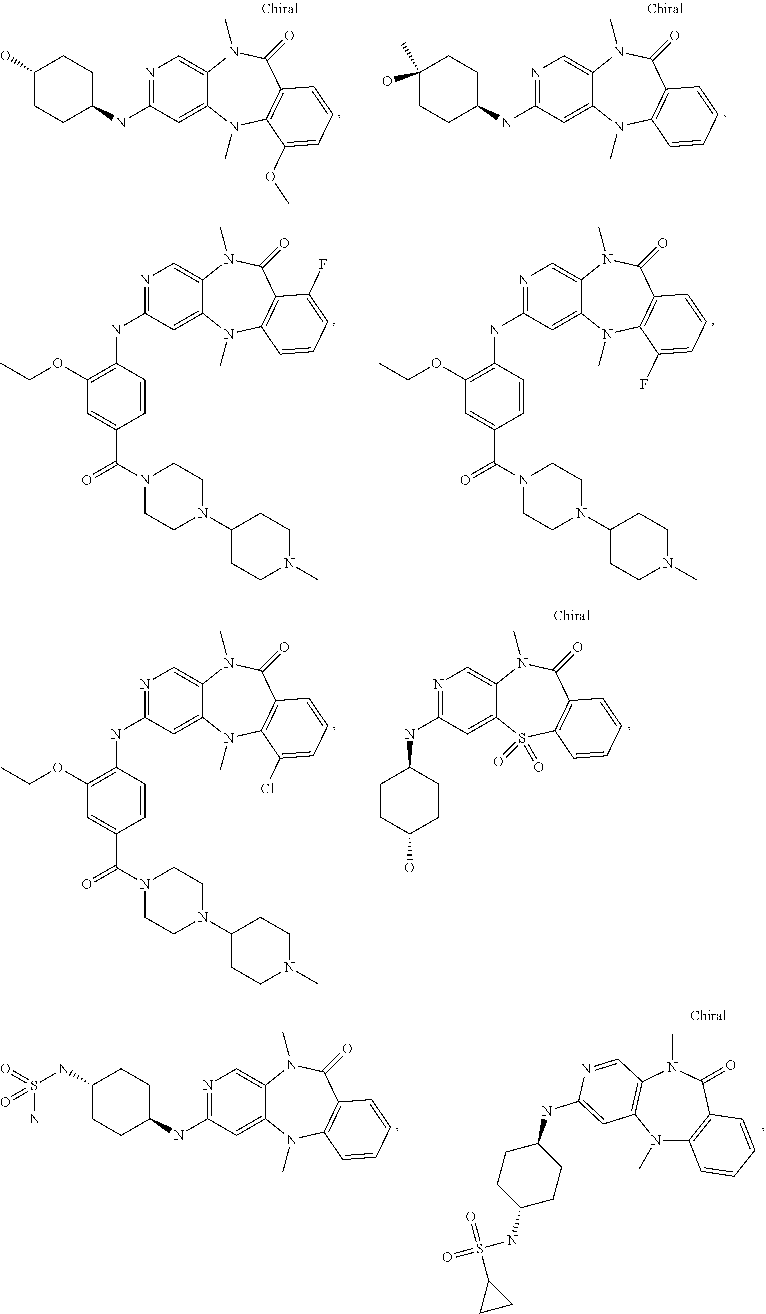

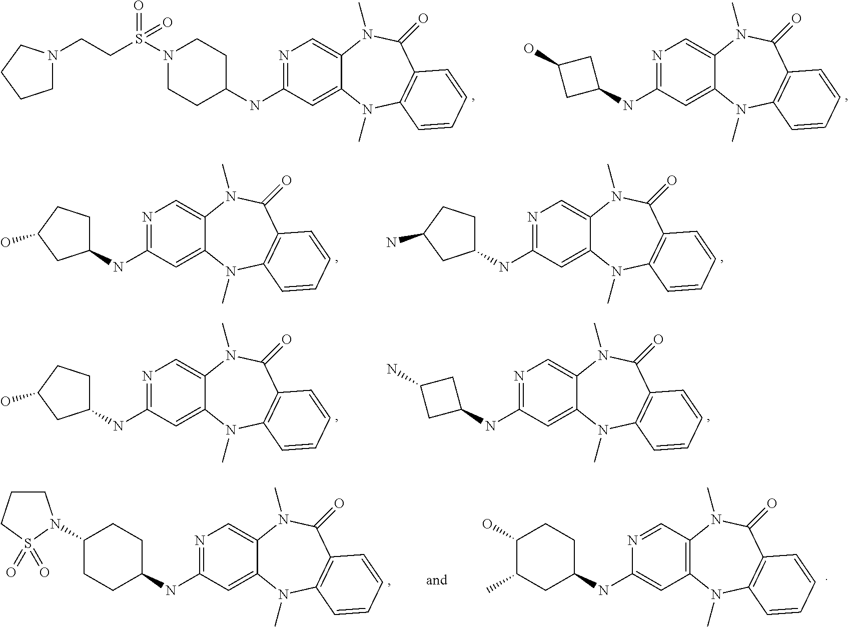

26. A pharmaceutical composition comprising a compound of claim 1 and a pharmaceutically acceptable carrier.

27. A method of treatment of a disease comprising administering a compound of claim 1, wherein the disease is a ERK5-mediated disease or a disease medicated by a BET family protein.

28. The method of claim 27, wherein the disease is modulated by a cytokine.

29. The method of claim 28, wherein the cytokine is IL-17, IL-6 or GCSF.

30. The method of claim 27, wherein the disease is an inflammatory disease in the airways.

31. The method of claim 30, wherein the disease is selected from nonspecific bronchial hyper-reactivity, chronic bronchitis, cystic fibrosis and acute respiratory distress syndrome.

32. The method of claim 27, wherein the disease is selected from asthma, chronic obstructive pulmonary disease, idiopathic pulmonary fibrosis, pulmonary fibrosis and interstitial lung disease.

33. The method of claim 27, wherein the disease is selected from psoriasis, chronic plaque psoriasis, psoriatic arthritis, acanthosis, atopic dermatitis, eczema, contact dermatitis, systemic sclerosis, wound healing, atopic dermatitis and drug eruption.

34. The method of claim 27, wherein the disease is selected from arthritis and osteoarthritis.

35. The method of claim 27, wherein the disease is dry eye.

36. The method of claim 27, wherein the disease is cancer.

37. The method of claim 27, wherein the cancer is selected from lung cancer, colon cancer, breast cancer, prostate cancer, liver cancer, pancreatic cancer, brain cancer, kidney cancer, ovarian cancer, stomach cancer, skin cancer, bone cancer, gastric cancer, breast cancer, glioma, hepatocellular carcinoma, papillary renal carcinoma, head and neck squamous cell carcinoma, leukemia, lymphoma and myeloma.

38. The method of claim 37, wherein the leukemia is selected from acute myeloid leukemia and chronic myeloid leukemia.

39. The method of claim 27, wherein the disease is allodynia, inflammatory pain, inflammatory hyperalgesia, post herpetic neuralgia, neuropathies, neuralgia, diabetic neuropathy, HIV-related neuropathy, nerve injury, rheumatoid arthritic pain, osteoarthritic pain, burns, back pain, ocular pain, visceral pain, cancer pain, dental pain, headache, migraine, carpal tunnel syndrome, fibromyalgia, neuritis, sciatica, pelvic hypersensitivity, pelvic pain, post operative pain, post stroke pain, or menstrual pain.

40. The method of claim 27, wherein the disease is Alzheimer's disease, mild cognitive impairment, age-associated memory impairment, multiple sclerosis, Parkinson's disease, vascular dementia, senile dementia, AIDS dementia, Pick's disease, dementia caused by cerebrovascular disorders, corticobasal degeneration, amyotrophic lateral sclerosis, Huntington's disease, or diminished CNS function associated with traumatic brain injury.

41. The method of claim 27 further comprising administering a second active agent.

42. The method of claim 41, wherein the second active agent is an anti-cancer agent or an anti-inflammatory agent or a disease-modifying antirheumatic drug.

43. The method of claim 42, wherein the anti-cancer agent is Ara-C.
